(19) United States
(12) Patent Application Publication
(10) Pub. No.: US 2025/0064981 A1
(43) Pub. Date: Feb. 27, 2025

(54) AAV VECTORS ENCODING BASE EDITORS AND USES THEREOF

(71) Applicants: The Broad Institute, Inc., Cambridge, MA (US); President and Fellows of Harvard College, Cambridge, MA (US)

(72) Inventors: David R. Liu, Cambridge, MA (US); Jonathan Ma Levy, Cambridge, MA (US); Jessie Rose Davis, Cambridge, MA (US); Tony P. Huang, Cambridge, MA (US); Isaac Witte, Cambridge, MA (US)

(73) Assignees: The Broad Institute, Inc., Cambridge, MA (US); President and Fellows of Harvard College, Cambridge, MA (US)

(21) Appl. No.: 18/927,415

(22) Filed: Oct. 25, 2024

Related U.S. Application Data
(63) Continuation of application No. PCT/US2023/066389, filed on Apr. 28, 2023.
(60) Provisional application No. 63/389,796, filed on Jul. 15, 2022.
(60) Provisional application No. 63/336,064, filed on Apr. 28, 2022.

(51) Int. Cl. A61K 48/00; C12N 9/22; C12N 9/80; C12N 15/11; C12N 15/86

(52) U.S. Cl. CPC A61K 48/0058; A61K 48/0075; A61K 48/0083; C12N 9/22; C12N 9/80; C12N 15/111; C12N 15/86; C12N 2310/20; C12N 2750/14143; C12N 2750/14151; C12N 2830/50; C12Y 305/04004

(57) ABSTRACT

Nucleic acid molecules, compositions, recombinant AAV (rAAV) particles, kits, and methods are described herein for delivering a base editor (or “nucleobase editor”) to cells, e.g., via AAV vectors. In particular, the disclosure provides compositions, methods, and uses for delivery of adenine base editors and cytosine base editors in a single AAV vector (or genome). Further described herein are improved AAV vectors containing size-minimized regulatory components that enable, e.g., the packaging of base editors. Provided herein are methods and compositions for delivering base editor proteins to a cell or tissue in a single recombinant AAV (rAAV) vector. Contemplated herein are improved methods and compositions for delivering these base editors in vivo, in a single rAAV particle. Further provided herein are base editors and compositions and cells comprising these base editors.

### RELATED APPLICATIONS

This U.S. national application is filed under 35 U.S.C. § 111 (a) as a continuation of and which claims priority under 35 U.S.C. §§ 120 and 365 (c) to International PCT Application, PCT/US2023/066389, filed Apr. 28, 2023, which claims priority under 35 U.S.C. § 119 (e) to U.S. Provisional Application, U.S. Ser. No. 63/336,064, filed Apr. 28, 2022 and to U.S. Provisional Application, U.S. Ser. No. 63/389,796, filed Jul. 15, 2022, each of which is incorporated herein by reference.

### GOVERNMENT SUPPORT CLAUSE

This invention was funded by Grant Nos. UG3AI150551, U01AI142756, R35GM118062, RM1HG009490, RO1EY009339, R01HL148769, and R35HL145203, awarded by the National Institutes of Health. The government has certain rights in the invention.

### REFERENCE TO AN ELECTRONIC SEQUENCE LISTING

The content of the electronic sequence listing (B119570158US02-SEQ-GJM.xml; Size: 448,229 bytes; and Date of Creation: Oct. 25, 2024) is herein incorporated by reference in its entirety.

### BACKGROUND OF INVENTION

Gene editing offers the clinically validated potential to treat a wide variety of genetic disorders for which few therapeutic options are available. Because the study and treatment of most genetic disorders through gene editing requires editing in vivo, clinically useful methods that mediate the efficient delivery of precision gene editing agents into cells of tissues in animals such as mammals^{1, 74} continue to play an importantrole in advancing the field.

Adeno-associated viruses (AAV) have been used to deliver genes encoding many therapeutic proteins in animal models of human disease^{3, 4}, in clinical trials⁵, and in FDA-approved drugs^{6, 7}. AAV has become a popular in vivo delivery method due to its clinical validation, its ability to target a variety of clinically relevant tissues, and its relatively well-understood and favorable safety profile.

### SUMMARY OF THE INVENTION

In some aspects, described herein are nucleic acid molecules, compositions, recombinant AAV (rAAV) particles, kits, and methods for delivering a complete base editor (or “nucleobase editor”) to cells, e.g., via a single AAV vector (or genome). In particular, the disclosure provides compositions, methods, and uses for delivery of size-minimized adenine base editors and cytosine base editors in a single AAV vector, wherein the adenine base editor and associated regulatory elements have a length shorter than the packaging capacity of AAV, of ˜4.9 kilobases (kb). Further described herein are improved AAV vectors containing size-minimized regulatory components that enable the packaging of base editors. The disclosure provides host cells and compositions comprising the disclosed rAAV particles. The disclosure further provides improved AAV vectors containing size-minimized regulatory components that enable the packaging of larger transgenes other than base editors.

Base editors^{8, 9} (BEs) can efficiently install targeted mutations in a variety of therapeutically relevant cell types in vitro and in animal models of human genetic diseases^{1, 10} BEs can also efficiently install targeted mutations in a variety of therapeutically relevant tissues in subjects, such as human subjects, including liver tissues. Unlike nuclease-mediated gene editing, base editing does not require double-strand DNA breaks and therefore generates minimal unwanted indel byproducts, chromosomal translocations¹¹, chromosomal ancuploidy¹², large deletions^{13, 14}, p53 activation^{15, 16}, or chromothripsis¹⁷. Base editors can correct point mutations that cause various genetic diseases, but their delivery to subjects in vivo is complicated by their large size (about 5.2 kb), which typically exceeds the maximum packaging capacity of an adeno-associated virus (AAV), which is ˜4.9 kb between the inverted terminal repeats (ITRs)^{18, 19}. As the optimal packaging capacity of an AAV is ˜4.7 kb, delivery of a base editor capable of acting on target DNA in a single AAV particle has presented major obstacles. For example, packaging BEs containing the commonly used Streptococcus pyogenes Cas9 protein (SpCas9, which is about 4.2 kb in length) and a single-guide RNA (sgRNA) in a single vector has not been workable. Although technically feasible, this approach leaves little room for customized expression and control elements, such as nuclear localization sequences (NLSs). In addition to the base editor itself, AAVs that deliver base editors must also include the guide RNA, promoters driving base editor and sgRNA expression, and cis regulatory elements.

In previous studies²⁰⁻²⁶, AAV was used to deliver base editors by dividing the base editor into two halves among two AAV nucleic acid vectors. See U.S. Patent Publication No. 2018/0127780, published May 10, 2018, PCT Publication No. WO 2020/236982, published Nov. 26, 2020, Levy, J. M., et al. Nat Biomed Eng 4, 97-110 (2020), Chen, Y., et al. Development of Highly Efficient Dual-AAV Split Adenosine Base Editor for In Vivo Gene Therapy. Small Methods 4, 2000309 (2020), and Villiger, L. et al. Nature Medicine 24, 1519-1525 (2018), each of which is incorporated herein by reference. In dual-AAV approaches, each “split” portion of the base editor transgene is fused to a small, trans-splicing intein²⁷, or each portion is expressed as mRNAs that undergoes trans-splicing²⁸. Typically, each of the two AAV vectors is packaged in a separate AAV viral particle (or virion). Dual AAV approaches rely on the incorporation of trans-splicing inteins that mediate reconstitution of the full-length BE from the split portions in the cell following delivery and transduction of the AAV particles. Because two AAV particles are required to deliver a single base editor, two successful and relatively simultaneous transductions of the target cell are necessary.

While dual-AAV delivery of base editors has supported therapeutic levels of editing including in mouse models of human disease, the development of a single AAV base editing system would further increase the potential impact by simplifying the application, characterization, and manufacturing of the base editor, and potentially increasing editing efficiency by obviating the need for simultaneous transduction of multiple AAVs. The single-AAV base editing system disclosed herein also lowers the required dose of AAV, an important advance since clinical applications of AAV are often constrained by dose-limiting toxicity²⁹. Thus, there is a need in the art for single-AAV in vivo base editor delivery, which only requires a single transduction of the target cell, that retains high editing efficiency. Single AAV delivery would offer advantages for research and clinical use, particularly in mammalian tissues that are more difficult to transduce effectively, such as heart, CNS, and muscle tissues.

Adenine base editors (ABEs) are a particularly useful class of editing agents because they install A•T-to-G•C conversions that correct approximately half of all known pathogenic SNPs⁹. Phage-assisted continuous evolution (PACE) of ABE7.10, an adenine base editor, recently yielded TadA-8e, a deoxyadenosine deaminase with increased activity and broadened compatibility with Cas domains other than SpCas930. ABE7.10, which contains the TadA7.10 deaminase, can perform clean and efficient A•T-to-G•C conversion in DNA with very low levels of undesired by-products, such as small insertions or deletions (indels), in cultured cells, adult mice, plants, and other organisms. Additional details about the TadA-8c and TadA7.10 deaminase can be found in PCT Publication No. WO 2021/158921, published Aug. 12, 2021; PCT Publication No. WO 2018/027078, published on Feb. 8, 2018, PCT Patent Publication No. WO 2019/079347, published on Apr. 25, 2019; Koblan et al., Nat Biotechnol 36, 843-846 (2018); and Gaudelli et al., Nature 551, 464-471 (2017), each of which is incorporated herein by reference. ABEs containing only a single TadA deaminase domain, rather than a single-chain dimer, allow for reduction in editor size^{30, 31}. Moreover, while SaCas9 is small enough (1053 amino acids in length, SEQ ID NO: 377) to provide a single AAV-compatible base editor, its utility is greatly limited by the rarity of its NNGRRT PAM. Since base editing requires the presence of a suitable PAM to place the target nucleotide within the editing window, ABEs that collectively offer broad PAM compatibility along with simple and efficient in vivo delivery would advance in vivo applications of base editing.

Likewise, cytosine base editors (CBEs) that offer broad PAM compatibility along with simple and efficient in vivo delivery would advance in vivo applications of base editing. Current CBEs contain a uracil glycosylase inhibitor domain, which is about 84 bp in length. Although not very large, these additional 84 base pairs render delivery of CBEs in a single AAV vector more difficult than ABEs.

Ran and colleagues engineered a size-minimized S. aureus Cas9 (SaCas9) for delivery in a single AAV vector in vivo to install double-strand breaks into target genomic DNA. See Ran, F. A., et al. (2015) Nature 520 (7546): 186-191, which is incorporated herein by reference. Ran's AAV cassette contained a U6 promoter-driven sgRNA and a cytomegalovirus (CMV) promoter- or thyroxine-binding globulin (TBG) promoter-driven SaCas9 transgene. Recently, Tran and colleagues engineered a single AAV vector containing a size-minimized SaCas9 ABE, microABE 1744, in which an ABE7.10 TadA deaminase monomer was inlaid (i.e., inserted) within an SaCas9 domain. See Tran et al., Nat. Commun. 11, 4871 (2020), which is incorporated herein by reference. However, this AAV-encoded ABE showed only <0.25% editing in vitro and was not assessed in vivo. More recently, Zhang, Sontheimer, and colleagues generated a single-AAV vector encoding an ABE containing an N. meningitidis 2 Cas9 (Nme2Cas9) protein. See Zhang et al., Adenine Base Editing in vivo with a Single Adeno-Associated Virus Vector. bioRxiv 2021.12.13.472434, which is incorporated herein by reference. The base editors of Zhang however exhibited significant edits at bystander adenines, and a maximum editing efficiency of 35%, following AAV delivery to liver tissue. However, single-AAV delivery of base editors containing other compact Cas9 protein domains that exhibit highly efficient base editing, as is the case for currently used ABEs and CBEs, has not been disclosed. In addition, single-AAV delivery of base editors of base editors to cardiac and muscular tissue has not been disclosed.

This disclosure provides size-minimized AAV vectors that have lengths of less than about 4.90 kb between the ITRs. This single-AAV base editing platform offers similar or improved editing efficiencies compared to dual-AAV ABE8e in a variety of tissues across multiple doses when delivered systemically into mice. Exemplary AAV vectors of the disclosure do not incorporate, or rely on the use of, trans-splicing inteins for successful delivery.

The AAV vectors of the disclosure are based, at least in part, on genetic engineering advances that generated vectors containing size-minimized components necessary for efficient expression in target cells and editing of target bases in vivo. Exemplary target cells include muscle cells, neurons, liver cells, neuromuscular cells, and cardiac cells. These vectors are smaller than the vectors disclosed in U.S. Patent Publication No. 2018/0127780, published May 10, 2018, and PCT Publication No. WO 2020/236982, published Nov. 26, 2020, and thus are adapted for incorporation into a single AAV particle. In particular, the disclosed AAV vectors are based in part on the discovery that a post-transcriptional response element, such as WPRE, in the transcriptional terminator (or polyadenylation signal) is not necessary for successful expression of the base editor in target tissues in vivo. The disclosed AAV vectors thus contain shorter (or size-minimized) terminators. The disclosed AAV vectors further contain other size-minimized regulatory elements, such as short promoters.

The disclosed AAV vectors are also based in part on the discovery that a guide RNA compatible with any of the disclosed Cas proteins can be encoded at the 3′ end of the vector and maintain a total ITR-to-ITR length of less than 4.9 kb, less than 4.8 kb, less than 4.7 kb, less than 4.6 kb, or less than 4.5 kb. As such, through the genetic engineering techniques described herein, a base editor and its guide can be efficiently packaged in a single rAAV particle for delivery in vivo. The guide RNA may be encoded in the vector in an orientation (3′ to 5′) reverse of that of the base editor (i.e., 5′ to 3′) and the promoter driving expression of the base editor transgene (and its origin of replication).

The disclosure also provides size-minimized base editors. These base editors were developed to enable efficient in vivo base editing mediated by single AAV particle. The disclosed AAV-encoded base editors that may comprise size-minimized Cas proteins. These Cas9 proteins are about 1000-1050 amino acids in length, which is about 350 amino acids shorter than a SpCas9 protein. These size-minimized Cas proteins include, but are not limited to S. aureus Cas9 (SaCas9), Nme2Cas9, C. jejuni Cas9 (CjCas9), S. auricularis Cas9 (SauriCas9), and variants of any of these Cas9 proteins. The disclosed base editors may contain an evolved or mutated variant of any of these Cas9 proteins, or any of the Cas9 proteins disclosed herein. Accordingly, in various embodiments, the disclosed AAV nucleic acid molecules do not comprise an intein, such as a trans-splicing intein (e.g., do not comprise a trans-splicing intein derived from Nostoc punctiforme, or Npu). In various embodiments, the disclosed AAV nucleic acid molecules comprise a transcriptional terminator that does not comprise a post-transcriptional response element. In some embodiments, the disclosed AAV nucleic acid molecules do not comprise an intein or a post-transcriptional response element. In some embodiments, the nucleic acid molecules comprise a first nucleic acid segment comprising: (i) a 5′ inverted terminal repeat (ITR); (ii) a first nucleic acid segment comprising sequence encoding a base editor operably linked to a first promoter, wherein the base editor comprises a nucleic acid programmable DNA binding protein (napDNAbp) domain and a deaminase domain; and a polyadenylation (polyA) signal; (iii) a second nucleic acid segment encoding a guide RNA (gRNA) operably linked to a second promoter; and (iv) a 3′ ITR.

In some aspects, provided herein are rAAV vectors having size-minimized regulatory elements that allow for packaging of a large transgenes. Accordingly, provided herein are rAAV nucleic acid molecules that comprise, in 5′ to 3′ order: (i) a 5′ inverted terminal repeat (ITR); (ii) a first nucleic acid segment comprising a transgene operably linked to a first promoter, wherein the first promoter has a length of less than 300 nucleotides; and a transcriptional terminator that does not contain a posttranscriptional response element; (iii) a second nucleic acid segment operably linked to a second promoter, wherein the direction of transcription of the second nucleic acid segment is reversed relative to the direction of transcription of the first nucleic acid segment; and (iv) a 3′ ITR. In various embodiments, the length the length between the 5′ ITR and the 3′ ITR is less than about 4.90 kb. In some embodiments, the length the length between the 5′ ITR and the 3′ ITR is less than about 4.85 kb, less than about 4.80 kb, less than about 4.75 kb, less than about 4.725 kb, less than about 4.70 kb, or less than 4.65 kb.

In some embodiments, the first nucleic acid segment encodes a base editor, and the second nucleic acid segment encodes a gRNA. In some embodiments, the first nucleic acid segment encodes a protein that is not a base editor.

Any of the disclosed base editors may comprise (i) a napDNAbp domain; and (ii) a deaminase domain. The disclosed base editors may comprise a wild-type napDNAbp domain (e.g., wild-type SaKKH-Cas9). The disclosed base editors may comprise a napDNAbp domain that has nickase activity (e.g., SaKKH-Cas9 nickase, or “SaKKH”). In some embodiments, the napDNAbp domain is a Cas9 nickase domain. In some embodiments, the napDNAbp domain is SaKKH-Cas9 nickase. The napDNAbp domain may be selected from an S. aureus Cas9 (SaCas9), an N. meningitidis 2 Cas9 (Nme2Cas9), a C. jejuni Cas9 (CjCas9), or an S. auricularis (SauriCas9) domain, and variants thereof. These Cas proteins have broader PAM compatibility than the standard SpCas9 protein. As such, the disclosed single-AAV encoded base editors can potentially target the vast majority of adenines across the genome, or the vast majority of cytosines across the genome. In exemplary embodiments, the napDNAbp domain is an SaCas9 domain, SaCas9 nickase domain, SaKKH domain, or SaKKH nickase domain.

The AAV-encoded ABEs of the disclosure contain an adenosine deaminase domain containing a single deaminase, i.e. a deaminase monomer (such as a TadA-8e monomer), rather than an adenosine deaminase dimer (i.e., two adenosine deaminases). Use of a deaminase monomer facilitates generation of size-minimized base editos. The TadA monomers are about 166 amino acids in length.

Any of the disclosed adenine base editors may comprise an adenosine deaminase domain that is a variant of E. coli TadA deaminase. In some embodiments, the adenosine deaminase is selected from a TadA-8c, a TadA-8c (V106W), a TadA9, a TadA20, and a TadA7.10 deaminase. In some embodiments, any of the base editors of the disclosure comprises an adenosine deaminase fused to the N-terminus of a napDNAbp domain, such as a Cas9 nickase. In some embodiments, the adenosine deaminase is TadA-8c.

In some aspects, the present disclosure provides size-minimized ABE8e variants. Each variant is compatible with single-AAV delivery, and three such variants collectively offer PAM compatibility sufficient to target 87% and edit 82% of adenines in the human genome. These three variants are Sauri-ABE8c, SaKKH-ABE8c, and SaABE8c. Each contain the TadA-8e adenosine deaminase and the nickase variant of SauriCas9, SaKKH-Cas9, and SaCas9, respectively. The present disclosure further provides ABE8e variants CjCas9-ABE8e and Nme2Cas9-ABE8e. The present disclosure further provides ABE variants SaKKH-ABE8c (V106W), SauriCas9-ABE8c (V106W), CjCas9-ABE8c (V106W), Nme2Cas9-ABE8c (V106W), and SaCas9-ABE8c (V106W); SaKKH-ABE9, SauriCas9-ABE9, CjCas9-ABE9, Nme2Cas9-ABE9, and SaCas9-ABE9; SaKKH-ABE20, SauriCas9-ABE20, CjCas9-ABE20, Nme2Cas9-ABE20, and SaCas9-ABE20; and SaKKH-ABE7.10, SauriCas9-ABE7.10, CjCas9-ABE7.10, Nme2Cas9-ABE7.10, and SaCas9-ABE7.10. In any of these disclosed base editors, the wild-type, or the nickase variant, of SauriCas9, SaKKH-Cas9, SaCas9, CjCas9, and Nme2Cas9, respectively, may be used.

The present disclosure further provides size-minimized CBE variants, and in particular size-minimized BE3.9 variants. Examples of these variants include CjCas9-BE3.9, CjCas9-FERNY-BE3.9, and CjCas9-evoFERNY-BE3.9. In some embodiments, the base editor further comprises a uracil glycosylase inhibitor (UGI) domain. The size of an exemplary cytidine deaminase (e.g., a rAPOBEC1 deaminase) of the disclosed CBEs is about 229 amino acids.

By integrating these developments, the single AAV-delivered base editors of the disclosure were used to treat a mouse model of high cholesterol (which is implicated in cardiovascular disease), resulting in correction of a casual mutation in cardiac tissue, and an increase in the animal's lifespan.

In the examples of this disclosure, single-AAV delivery of ABEs achieved editing of 66%, 33%, and 22% in liver, heart, and muscle tissues, respectively, at doses lower than or similar to those used in recent preclinical and clinical studies of AAV particles targeting these tissues (see, e.g., Clinical Trial Nos. NCT02122952 (SMA treatment) and NCT03375164 (DMD treatment)). In addition, it was demonstrated that single-AAV8 ABEs could efficiently edit therapeutically relevant targets in mice at efficiencies of 60-85%. These edits produced disruptions at native splice acceptors sites in the target human and mouse Pesk9 and mouse Angptl3 genes, which resulted in nearly complete (93% average) knockdown of these genes at doses lower than previously reported, resulting in substantial reduction in plasma cholesterol and triglycerides. Liver protein Proprotein Convertase Subtilisin/Kexin Type 9 (PCSK9) is a secreted, globular, auto-activating serine protease that acts as a protein-binding adaptor within endosomal vesicles to bridge a pH-dependent interaction with the low-density lipoprotein receptor (LDL-R) during endocytosis of LDL particles, preventing recycling of the LDL-R to the cell surface and leading to reduction of LDL-cholesterol clearance. Angiopoctin-like 3 protein (Angpt13) is an endogenous inhibitor of lipoprotein lipase (LPL), which is the main enzyme involved in hydrolysis of triglyceride-rich lipoproteins. Base editors targeting disease-causing mutations in the Pesk9 gene are disclosed in US Publication No. 2018/0237787, published on Aug. 23, 2018, which is incorporated herein by reference.

By minimizing the size of adenine base editors and AAV components, a suite of single-AAV adenine base editor systems were developed that had broad targeting capability due to their collective PAM compatibility and supported robust editing in vivo. The single-AAV BE vectors of the disclosure facilitate base editing for research and therapeutic applications by simplifying production and characterization, and by reducing the total dose of AAV required to achieve a desired level of editing. These single-AAV BEs offer several potential advantages over dual AAV approaches for clinical use: clinical-scale production of a single vector rather than two; increased potency, especially at lower doses; and reduced complexity from a simpler construct that obviates the need to use a trans-splicing intein. For these reasons, in vivo editing approaches compatible with single-AAV delivery may be more readily applied to large animal models and human therapeutics where systemic delivery is commonly used. Development of smaller promoters that provide sufficient expression of base editors allow further minimization of the elements of single-AAV ABEs, which should facilitate clinical translation by increasing the proportion of full-length packaged AAV genomes.

In other aspects, host cells comprising the compositions described herein are provided. The disclosed cells may comprise any of the disclosed nucleic acid molecules, rAAV vectors, or rAAV particles described herein. In still other aspects, kits comprising any of the disclosed rAAV particles and instructions for delivery to a cell (such as a host cell) are provided.

Still other aspects of the present disclosure provide methods comprising contacting a target nucleic acid molecule with any of the compositions described herein. In various embodiments, the target nucleic acid molecule is in a cell, such as a eukaryotic cell (e.g., a mammalian cell). In some embodiments, the target nucleic acid molecule is genomic DNA. In some embodiments, the genomic DNA is in a cell or tissue of a subject, such as a human subject. Accordingly, methods comprising contacting a cell with any of the rAAV particles or compositions disclosed herein are contemplated.

Still other aspects of the present disclosure provide methods comprising administering to a subject in need there of a therapeutically effective amount of any of the compositions (or rAAV particles) described herein. In some embodiments, the subject has a disease or disorder (e.g. a genetic disease). In some embodiments, the disease or condition is cardiovascular disease. In some embodiments, the compositions are administered to the liver tissue, cardiac tissue, or skeletal muscle tissue of the subject.

Still other aspects of the present disclosure provide methods of making any of the disclosed rAAV particles and compositions.

The details of certain embodiments of the invention are set forth in the Detailed Description of Certain Embodiments, as described below. Other features, objects, and advantages of the invention will be apparent from the Definitions, Examples, Figures, and Claims.

### BRIEF DESCRIPTION OF THE DRAWINGS

FIGS. 1A-1C. FIG. 1A shows an AAV construct evaluated in vivo. sgRNA targeting a Pesk9 gene having the W8 mutation was delivered with EGFP in one AAV that was co-injected with either one or two additional AAVs encoding either an intact or intein-split SaABE8e, respectively. A total of two AAVs were used to deliver the intact SaABE8c and sgRNA, and three AAVs were used to deliver the intein-split SaABE8c. Black boxes represent ITRs, EFS promoter is EF1α short, W3 is truncated WPRE, bGH is bovine growth hormone polyadenylation signal, the purple box is the U6 promoter-driven sgRNA cassette in the orientation indicated by the arrow, NpuN & NpuC split inteins from Nostoc punctiforme are shown in brown, and protein coding regions are indicated for EGFP and SaABE8c. FIG. 1B shows in vivo editing efficiency from injection of AAV encoding intein-split and intact SaABE8c. The total dose of base editor AAV administered to each mouse is shown. FIG. 1C shows a comparison of in vivo editing efficiency from injection of AAV9 encoding intact SaABE8e in five different AAV architectures when administered at the dose shown. In all cases, editor AAV dose was either 4×10¹¹ vector genomes (vg) or 4×10¹⁰ vg and sgRNA EGFP AAV dose was either 4×10¹¹ vg or 4×10¹⁰ vg for a 1:1 ratio of base editor AAV to sgRNA AAV. The sizes of the delivered editor AAV constructs (including ITRs) are shown in the legend. C57BL/6J mice aged 6-7 weeks old weighing 20-25 g were injected systemically by retroorbital injection. Dots represent individual mice. Values and error bars represent mean±SEM of n=3 different mice.

FIGS. 2A-2C. Development and characterization of a single AAV SaABE8c. FIG. 2A shows a schematic of the single AAV SaABE8e genome (5,064 bp including ITRs). Arrow indicates direction of the U6 sgRNA cassette. FIG. 2B shows a comparison of dual to single SaABE8c. Base editing activity of either a dual (SaABE8e with intact editor on one genome and sgRNA and EGFP on a second genome) or a single (SaABE8e and guide RNA in a single vector) AAV vector, both installing Pcsk9 W8R edit, packaged in AAV9 with matched promoter and terminator (polyA). Base editor AAVs were administered at the dose indicated in the legend (dual AAVs were delivered with the dose indicated of editor AAV and sgRNA AAV, while single AAVs were delivered at the dose indicated) to C57BL/6J mice aged 6- to 8-week-old weighing 20-25 grams via retroorbital injection, and tissues were harvested three weeks post injection and analyzed by high-throughput DNA sequencing (HTS). FIG. 2C shows the dose titration of single AAV SaABE8e. Dots represent individual mice and error bars represent mean±SEM of n=3 different mice.

FIGS. 3A-3D. Characterization of Nme2ABE8e (FIG. 3A), CjABE8c (FIG. 3B), and SauriABE8c (FIG. 3C) in HEK293T cells. Editing of each target adenine within the protospacer is shown. Target sites are indicated, with sequences of each target protospacer and PAM listed in Table 1. Target adenines are numbered with respect to a standard protospacer length for each editor (22 nucleotides for CjABE8e, 24 nucleotides for Nme2ABE8e, and 21 nucleotides for SauriABE8e). Dots represent values and error bars represent mean±SEM of n=3 replicates. FIG. 3D shows the percent of genomic adenines in the bg38 human reference genome targetable by size-minimized ABEs independently and collectively. A schematic showing a representative portion of the genome targetable by size-minimized ABEs is shown, with targetable PAMs denoted with colored lines and targetable adenines denoted with colored dots. The right-most pie chart of the schematic indicates that 87% of single nucleotide adenine polymorphisms are targetable (and 82% are editable) by all of the small AAV-encoded ABEs of this disclosure, collectively.

FIGS. 4A-4D. Characterization of Nme2ABE8e (FIG. 4A), CjABE8c (FIG. 4B), and SauriABE8c (FIG. 4C) in HEK293T cells. Base editing activity windows from 8-9 genomic target sites for each size-minimized ABE are shown. Positions that were not present in any tested site are shaded gray. Target adenines are numbered with respect to a standard protospacer length for each editor (24 nt for Nme2ABE8e, 22 nt for CjABE8e, and 21 nt for SauriABE8e). Dots represent values and error bars represent mean±SEM of n=3 replicates for each site, with each position representing 1-3 genomic sites. The dotted line corresponds to 25% of mean peak activity for each base editor and defines the activity threshold considered within the editing window. Individual sites that contained three or more adenines within a protospacer were included in this analysis. Sites with only one or two adenines within the protospacer were excluded from summary analysis, but data from all sites analyzed are shown in FIGS. 3A-3C. FIG. 4D shows the percent of genomic adenines in the hg38 human reference genome targetable by size-minimized ABEs, either independently or collectively. An example showing a representative portion of the human genome targetable by size-minimized ABEs is shown, with targetable PAMs denoted with colored lines and targetable adenines denoted with colored dots.

FIGS. 5A-5K. Assessment of genome editing and plasma lipids when targeting PCSK9, Pcsk9, and Angptl3 with single-AAV in vivo. FIG. 5A shows a strategy for assessing base editing and plasma analytes in AAV treated mice, and was created using BioRender. FIG. 5B shows bulk liver editing efficiencies at human PCSK9, and mouse Pesk9 and Angptl3 (n=3-5 mice, each dot represents one mouse, error bars represent SEM). Human PCSK9 editing was performed using humanized PCSK9 mice, while mouse Pesk9 and Angptl3 editing was performed at the endogenous mouse loci of wild-type C57BL/6J mice. AAV was administered by retroorbital injection at 6-8 weeks of age at a dose of 1×10¹¹ vg/mouse. FIG. 5C shows dose-dependent base editing for dual SpABE8e and single SaKKH-ABE8e at mouse Pesk9 exon 1 splice donor. The total AAV dose administered is indicated below each set of bars in vg/mouse. The total AAV dose administered is indicated below each set of bars in vg/mouse. The dual SpABE8e editing data was reported in another publication from our group². Each dot represents a different mouse (n=5). FIG. 5D shows direct comparison of editing efficiencies of dual-AAV8 intein-split SaKKH-ABE8e and single-AAV8 SaKKH-ABE8e targeting the Pesk9 exon 1 donor site in bulk liver at two doses. The total AAV dose administered is indicated below each set of bars in vg/mouse, ***P=0.0004. Each dot represents a different mouse (n=4). FIG. 5E shows plasma PCSK9 protein in humanized mice treated with 1×10¹¹ vg single-AAV8 SaKKH-ABE8c. FIG. 5F shows plasma Pesk9 protein in C57BL/6J mice treated with either 1×10¹¹ vg single-AAV8 SaKKH-ABE8c, dual-AAV8 SpABE8e or non-targeting control, ***P=0.0001. FIG. 5G shows plasma Angpt13 protein in C57BL/6J mice treated with 1×10¹¹ vg single-AAV8 SaKKH-ABE8e or non-targeting control, **P=0.0027. FIG. 5H shows plasma total cholesterol in humanized mice treated with 1×10¹¹ vg single-AAV8 SaKKH-ABE8c. FIG. 5I shows plasma total cholesterol in C57BL/6J mice treated with either 1×10¹¹ vg single-AAV8 SaKKH-ABE8c, dual-AAV8 SpABE8e or non-targeting control, ***P=0.0007. FIG. 5J shows plasma total cholesterol ***P-0.0007 and plasma triglycerides *P=0.0118. FIG. 5K shows C57BL/6J in mice treated with 1×10¹¹ vg single-AAV8 SaKKH-ABE8e or non-targeting control. For FIGS. 5E-5K, dots represent mean values and error bars represent SEM of n=5 different mice. Significance was calculated for FIGS. 5C-5D using two-way unpaired t-test. Significance for FIGS. 5F-5G and FIGS. 5I-5K was calculated using two-way repeated measures ANOVA with Tukey's or Šídák multiple comparisons, as applicable, and is shown for the week 4 time point for all graphs except for FIG. 5F, in which week 3 significance is shown as week 4 protein levels did not reach statistical significance. In all instances, non-targeting control is dual AAV8 SpABE7.10 with sgRNA targeting mouse Dnmt1, an unrelated site in the mouse genome, administered at the same timepoint, route, and dose.

FIG. 6 shows the validation of SaABE targets in mouse Neuro-2A and 3T3 cells. The base editor and PAM are noted below each set of bars. Dots represent independent biological replicates (n=3) and error bars show SEM.

FIG. 7 shows the titration of sgRNA cassette AAV in vivo. A constant dose of 4×10¹¹ vg of full-length SaABE8e editor AAV was delivered with varying proportion of sgRNA cassette containing AAV by retroorbital injection to C57BL/6J mice. Tissues were harvested three weeks post injection and analyzed by HTS. Dots represent individual mice (n=3) and error bars show SEM.

FIGS. 8A and 8B. FIG. 8A shows the SaABE8e activity window at Pesk9 W8R in liver. SaABE8e maintains a wide editing window in vivo, consistent with observations in cultured cells. FIG. 8B shows that indels remain low under all conditions, reaching 2.4%, 1.6%, and 1.1% indels in heart, muscle, and liver tissues, respectively, at a high dose of 8×10¹¹ vg single AAV SaABE8c. Dots represent individual mice (n=3) and error bars show SEM.

FIGS. 9A-9C. Validating guide targeting PCSK9 (FIG. 9A) in HEK293T cells and Pcsk9 (FIG. 9B) and Angptl3 (FIG. 9C) in mouse Neuro-2a cells. For each sgRNA, the exon, target type (start codon, splice donor, or splice acceptor), ABE8e variant (SaABE8e or SaKKH-ABE8c), and protospacer position that disrupts the indicated target with respect to a 22 nt protospacer length are indicated. Editing at the position that disrupts the indicated target is plotted. Dots represent independent replicates (n=2) and error bars show standard deviation (SD).

FIGS. 10A and 10B show editing in Neuro-2A cells with SauriABE8e at the Pcks9 exon 1 splice donor target. FIG. 10A shows targeting of mouse Pesk9 exon 1 splice donor with SauriABE8e and SaKKH-ABE8e in mouse Neuro-2A cells. The target adenine is A9 with respect to the Sauri protospacer, A₅ with respect to SaKKH protospacer. FIG. 10B shows a comparison of SaCas9 guide RNA scaffolds^{33, 74} on editing activity at the mouse Pcsk9 exon 1 splice donor. The SaCas9 sgRNA scaffold is used with the homologous SauriCas9 protein, as the native sgRNA for SauriCas9 is not known.

FIGS. 11A and 11B. In vivo editing of control AAVs for lipid modification experiments. 6- to 8-week-old C57BL/6J mice were injected by retroorbital injection and whole liver was analyzed by HTS after four weeks. FIG. 11A shows editing at Dnmt1 A41A (silent edit) with dual SpABE7.10 at a dose of 1×10¹¹ vg dual AAV8. FIG. 11B shows installation of the Pesk9 W8R substitution using single SaKKH-ABE8e at a dose of 1×10¹¹ vg single AAV8.

FIGS. 12A-12D. Dose response of single-AAV8 SaKKH-ABE8e and dual-AAV8 SpABE8e (with Pesk9 exon 1 splice donor site-targeting sgRNA) on plasma Pcsk9 and total cholesterol. FIG. 12A shows circulating Pesk9 protein and FIG. 12B shows total cholesterol from plasma taken weekly, normalized to baseline. FIG. 12C shows circulating Pcsk9 protein and FIG. 12D shows total cholesterol from plasma taken weekly, raw (unnormalized). Dots represent mean values and error bars represent SEM of n=5 different mice. All mice were administered the total dose of AAV8 indicated in the legend systemically by retro-orbital injection at 6-8 weeks of age and blood samples were removed serially over four weeks.

FIGS. 13A-13E. Raw (unnormalized) levels of plasma analytes of either single-AAV ABE or nontargeting control dual-AAV ABE mice, for human PCSK9 and mouse Angpt13 targets. FIG. 13A shows ELISA of human PCSK9 in plasma from humanized mice. FIG. 13B shows total plasma cholesterol in humanized PCSK9 mice. FIG. 13C shows ELISA of mouse Angpt13 in plasma from C57BL/6J mice. FIG. 13D shows total plasma cholesterol in C57BL/6J mice. FIG. 13E shows plasma triglycerides from C57BL/6J mice. Dots represent mean values and error bars represent SEM of n=5 different mice. The nontargeting control is dual-AAV ABE7.10 targeting Dnmt1. All mice were administered a dose of 1×10¹¹ vg AAV8 systemically by retro-orbital injection at 6-8 weeks of age, and blood samples were removed serially over four weeks.

FIG. 14 is a schematic showing a construct for a single AAV vector expressing a cytosine base editor, which includes a uracil glycosylase domain. The Cas9 domain of the cytosine base editor is a CjCas9. A U6-controlled sgRNA is positioned at the 3′ end, in the reverse orientation. This construct has a total length of 5.012 kb, including the ITRs.

FIG. 15 shows a base editor-matched comparison of guide-dependent on-target editing between single-AAV SaKKH-ABE8e and dual-AAV SaKKH-ABE8e at the Pesk9 exon 1 splice donor site.

FIGS. 16A-16C shows guide-dependent off-target DNA editing analyses in vivo and in culture between single-AAV8 SaKKH and each half of an AAV8 intein-split SaKKH-ABE8c. FIG. 16A shows in vivo editing in liver tissue from single-AAV ABE treated mice. The top three predicted off target (“OT”) sites for SaKKH-ABE8e targeting Pesk9 exon 1 splice donor were sequenced from liver tissue. FIG. 16B shows the editing observed at OT2 is dose-dependent. OT, off-target; NT, non-targeting dose-matched dual AAV8 ABE7.10 targeting Dnmt1, an unrelated gene. FIG. 16C shows editing in cell culture. On- and off-target sites were sequenced after plasmid transfection of N2A cells with sgRNA targeting Pcsk9 exon 1 donor site and full-length or intein-split SpABE8c. Full-length and intein-split SpABE8c did not significantly differ in efficiency at on- or off-target edits by multiple unpaired t tests with Holm-Šidák method for correction for multiple comparisons.

FIG. 17 shows that in vivo mRNA off-target editing is undetectable in single-AAV ABE8e treated mice. Dots represent single adenines across each amplicon of n=4 mice.

FIG. 18 shows a comparison of the editing windows of exemplary single AAV-encoded ABEs in the liver.

FIGS. 19A-19B. Histopathological assessment by hematoxylin and eosin staining of livers from untreated mice, FIG. 19A, and mice four weeks after treatment with 1×10¹¹ vg of single AAV8 SaKKH-ABE8e targeting human PCSK9, FIG. 19B. Representative images are shown. Scale bar, 50 μm.

FIG. 20 shows the quantification of AAV genomes from tissue encoding SaABE8c dual AAV (SaABE8c with intact editor on one genome and sgRNA and EGFP on a second genome) or single AAV (SaABE8e and guide RNA all-in-one), both installing Pcsk9 W8R packaged in AAV9. Editors were packaged in AAV9 and administered by retro-orbital injection at 6-8 weeks of age at the dose indicated in the legend (dual AAVs were delivered at the dose indicated in the legend of editor AAV and sgRNA AAV, while single AAVs were delivered at the total dose indicated). Tissues were harvested 3 weeks post injection then editor AAV genomes were quantified by ddPCR using SaCas9 primers and probe, normalized to Gapdh. Dots represent individual mice (n=1-3) and error bars show SEM.

FIG. 21 shows the alkaline gel electrophoresis of packaged AAV genomes.

FIG. 22A-22D show the off-target mRNA editing. RNA was extracted from mouse livers treated with single-AAV8 SaKKH-ABE8e and untreated mouse livers, reverse transcribed, and cDNA amplicons from Aars, Canx, Ctnnb, and Usp38 mRNA were analyzed by HTS. Dots represent individual adenines across the sequenced amplicon (n=3 mice).

### DETAILED DESCRIPTION OF THE INVENTION

Provided herein are methods and compositions for delivering base editor proteins to a cell or tissue in a single recombinant AAV (rAAV) vector. Contemplated herein are improved methods and compositions for delivering these base editors in vivo (such as to a subject in need thereof) in a single rAAV particle. Delivery in a single rAAV particle has advantages of requiring fewer injections and lower doses for delivery to target tissues, as well as reducing in half the number of successful transductions of target tissue necessary for expression of the base editor in target cells. These rAAV vectors comprise size-minimized base editors and regulatory components that enable the vector to have a length within the 4.7 kb-4.9 kb packaging capacity of rAAV particles. rAAV particles that contain any of the disclosed rAAV vectors and a capsid protein are also provided, as well as compositions and cells comprising same. Methods of administering such compositions, and cells, to a subject are further provided. Further provided are base editors and compositions and cells comprising these base editors. The disclosed single-AAV adenine base editors provide comparable or enhanced editing efficiencies compared to dual-AAV editors in a variety of tissues in vivo.

Recombinant AAV vectors (or AAV genomes) are widely used for transgene delivery. Transgenes are inserted into the AAV genome between the inverted terminal repeat (ITR) sequences and packaged into AAV viral particles, which are used to transduce a host cell (e.g., mammalian cell, human cell). AAV has been used to deliver genes encoding many therapeutic proteins in animal models of human disease, in clinical trials and in FDA-approved drugs. A suite of available AAV serotypes provide access to a variety of clinically relevant cell types in mice, nonhuman primates, and humans.

The disclosure provides rAAV vectors having size-minimized regulatory elements that allow for packaging of a larger transgene than the vectors of the prior art. In some embodiments, the transgene encodes a base editor, such as an adenine base editor. In some embodiments, the transgene is not a base editor. In some embodiments, the base editor contains a napDNAbp domain that is a compact protein, such as an S. aureus Cas9 (SaCas9), an N. meningitidis 2 Cas9 (Nme2Cas9), a C. jejuni Cas9 (CjCas9), or an S. auricularis (SauriCas9) domain, or a variant thereof.

In some aspects, provided herein are rAAV vectors that contain a first nucleic acid segment comprising: (i) a 5′ITR; (ii) a first nucleic acid segment comprising sequence encoding a base editor operably linked to a first promoter, wherein the base editor comprises a nucleic acid programmable DNA binding protein (napDNAbp) domain and a deaminase domain; and a polyadenylation (polyA) signal; (iii) a second nucleic acid segment encoding a guide RNA (gRNA) operably linked to a second promoter; and (iv) a 3′ ITR, wherein the length between the 5′ ITR and the 3′ ITR is less than about 4.90 kb. In some embodiments, the rAAV vectors consist essentially of components (i)-(iv).

In some embodiments, the nucleic acid vector is the genome of an adeno-associated virus packaged in a rAAV particle. In some embodiments, the first and/or the second nucleic acid segment is operably linked to a first promoter. In some embodiments, the first promoter is a constitutive promoter. In some embodiments, the first promoter is an inducible promoter.

In various embodiments, the first promoter is a short promoter. In various embodiments, the first promoter has a length of less than 325 nucleotides, less than 300 nucleotides, less than 285 nucleotides, less than 270 nucleotides, less than 265 nucleotides, or less than 250 nucleotides. This short length ensures optimal packaging capacity for the base editor and additional regulatory elements. In some embodiments, the first promoter has a length of between 200 and 250 nucleotides, 225 and 255 nucleotides, 250 and 275 nucleotides, 275 and 300 nucleotides, or 300 and 325 nucleotides. In certain embodiments, the first promoter has a length of 280 nucleotides. In some embodiments the first promoter has a length of 229 nucleotides, 253 nucleotides, or 324 nucleotides.

In some embodiments, the first promoter is a tissue-specific promoter. The first promoter may be a cardiac tissue-specific promoter, a muscle tissue-specific promoter, or a neuronal tissue-specific promoter. The first promoter may be active in a tissue other than liver, muscle, and neurons, such as ocular tissue. In some embodiments, the first promoter is active in neuromuscular tissue.

In some embodiments, the first promoter is an EF-1α (short) (“EFS”) promoter, which is the intron-less form of EF-1α. In some embodiments, the first promoter is an MeCP2 promoter, which is active in neuronal tissues. In some embodiments, the first promoter is a P3 promoter, which is active in liver tissues. In some embodiments, the first promoter is a U1a promoter, which is active in liver tissues. Additional details about the MeCP2 promoter, P3 promoter, and U1a promoter is found in Gray et al., Human Gene Therapy.September 2011.1143-1153; Viccelli et al., Hepatology, 60:1035-1043 (2014); and Ibrahcim et al., Genome Biol. 19, 137 (2018), respectively, each of which is incorporated herein by reference.

In some embodiments, the first nucleic acid segment comprises a transcriptional terminator. In various embodiments, the first nucleic acid segment does not contain a posttranscriptional response element (e.g., W3). In some embodiments, the transcriptional terminator is a polyA signal selected from a bovine growth hormone (bGH) signal, human growth hormone (hGH) signal, or SV40 signal. In some embodiments, the terminator is a bGH polyA signal. In some embodiments, the terminator is a SV40 late polyA signal.

In some embodiments, the first nucleic acid segment comprises a minimal minute virus of mice (MVM) intron. In some embodiments, the MVM is positioned 5′ of the promoter and 3′ of the transgene.

In some embodiments, the second nucleic acid segment comprises a nucleotide sequence encoding a gRNA operably linked to a second promoter. In some embodiments, the second promoter is a constitutive promoter. In some embodiments, the second promoter is an inducible promoter. In some embodiments, the second promoter is a U6 promoter, such as a human U6 promoter. In some embodiments, the direction of transcription of the second nucleic acid segment is reversed relative to the direction of transcription of the first nucleic acid segment.

The disclosed rAAV vectors-which contain a first nucleic acid segment that contains a promoter and terminator and a second nucleic acid segment that may encode a guide RNA—have packaging capacities between the 5′ ITR and the 3′ ITR of lengths that fit a transgene encoding one or more of the disclosed base editors. For example, the disclosed AAV vectors may contain a length between the 5′ ITR and the 3′ ITR of between 4.7 kb and 4.9 kb. The disclosed AAV vectors may contain a length between the 5′ ITR and the 3′ ITR of between 4.7 kb and 5.1 kb. The disclosed AAV vectors may contain a length between the 5′ ITR and the 3′ ITR of between 4.6 kb and 4.9 kb, or between 4.6 kb and 4.8 kb. In certain embodiments, the length between the 5′ ITR and the 3′ ITR is about 4.60 kb, about 4.65 kb, about 4.70 kb, about 4.725 kb, about 4.75 kb, about 4.80 kb, about 4.825 kb, about 4.85 kb, about 4.90 kb, or about 4.95 kb.

In various embodiments, the length the length between the 5′ ITR and the 3′ ITR is less than about 4.90 kb. In some embodiments, the length between the 5′ ITR and the 3′ ITR is about 4.80 kb. In some embodiments, the length between the ITRs is 4.804 kb (4804 bp). In some embodiments, the length between the ITRs is 4.828 kb. In some embodiments, the length between the ITRs is 4.722 kb.

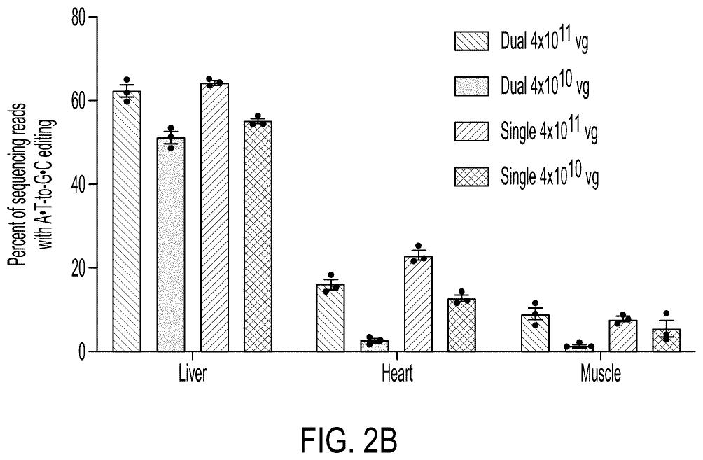

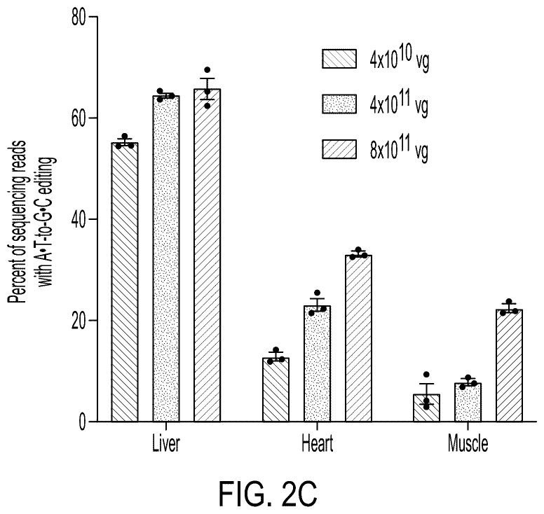

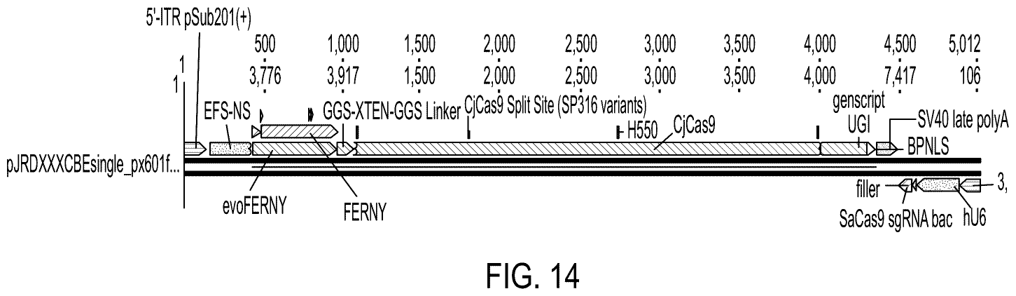

The disclosure provides rAAV vectors containing size-minimized adenine base editors, and rAAV vectors containing size-minimized cytosine base editors. Exemplary AAV vectors of the disclosure are shown in FIGS. 2A and 14.

In some aspects, provided herein are rAAV vectors that comprise: (i) a 5′ ITR; (ii) a first nucleic acid segment comprising sequence encoding a SaKKH-ABE8e, a SauriCas9-ABE8c, a CjCas9-ABE8e base editor, or a Nme2Cas9 base editor operably linked to a first promoter, wherein the first promoter is selected from the EFS, MeCP2, P3, and U1A promoters; and a bGH polyadenylation (polyA) signal; (iii) a second nucleic acid segment encoding a guide RNA (gRNA) operably linked to a U6 promoter, wherein the direction of transcription of the second nucleic acid segment is reversed relative to the direction of transcription of the nucleic acid molecule; and (iv) a 3′ ITR. In some embodiments, the rAAV vectors contain a first nucleic acid segment comprising: (i) a 5′ITR; (ii) a first nucleic acid segment comprising sequence encoding a Sauri-ABE8e base editor operably linked to an EFS promoter; and a polyadenylation (polyA) signal; (iii) a second nucleic acid segment encoding a guide RNA (gRNA) operably linked to a U6 promoter, wherein the direction of transcription of the second nucleic acid segment is reversed relative to the direction of transcription of the nucleic acid molecule; and (iv) a 3′ ITR. In various embodiments, the length between the 5′ ITR and the 3′ ITR is less than about 4.90 kb. In some embodiments, the poly (A) signal is a bGH poly (A) signal.

In certain embodiments, the rAAV vectors comprise, from 5′ to 3′: (i) a 5′ ITR; (ii) a first nucleic acid segment comprising sequence encoding a SaKKH-ABE8e base editor operably linked to an EFS promoter; and a bGH polyadenylation (polyA) signal; (iii) a second nucleic acid segment encoding a guide RNA (gRNA) operably linked to a U6 promoter, wherein the direction of transcription of the second nucleic acid segment is reversed relative to the direction of transcription of the nucleic acid molecule; and (iv) a 3′ ITR. In some embodiments, the rAAV vectors contain a first nucleic acid segment comprising: (i) a 5′ITR; (ii) a first nucleic acid segment comprising sequence encoding a Sauri-ABE8c base editor operably linked to an EFS promoter; and a bGH polyadenylation (polyA) signal; (iii) a second nucleic acid segment encoding a guide RNA (gRNA) operably linked to a U6 promoter, wherein the direction of transcription of the second nucleic acid segment is reversed relative to the direction of transcription of the nucleic acid molecule; and (iv) a 3′ ITR. In some embodiments, the base editor is SauriCas9-ABE8e. In some embodiments, the base editor is CjCas9-ABE8c.

In some embodiments, the rAAV vectors encode a CBE and comprise, from 5′ to 3′: (i) a 5′ ITR; (ii) a first nucleic acid segment comprising sequence encoding a CjCas9-FERNY-BE3.9 or CjCas9-evoFERNY-BE3 base editor operably linked to a first promoter that is an EFS promoter; and a polyadenylation (polyA) signal; (iii) a second nucleic acid segment encoding a guide RNA (gRNA) operably linked to a U6 promoter, wherein the direction of transcription of the second nucleic acid segment is reversed relative to the direction of transcription of the nucleic acid molecule; and (iv) a 3′ ITR. In some embodiments, the rAAV vectors encode a CBE and comprise, from 5′ to 3′: (i) a 5′ ITR; (ii) a first nucleic acid segment comprising sequence encoding a CjCas9-FERNY-BE3.9 or CjCas9-evoFERNY-BE3 base editor operably linked to a first promoter, wherein the first promoter is selected from the EFS, MeCP2, P3, and U1A promoters; and a polyadenylation (polyA) signal; (iii) a second nucleic acid segment encoding a guide RNA (gRNA) operably linked to a U6 promoter, wherein the direction of transcription of the second nucleic acid segment is reversed relative to the direction of transcription of the nucleic acid molecule; and (iv) a 3′ ITR. In some embodiments, the poly (A) signal is a bGH poly (A) signal. In some embodiments, the poly (A) signal is a SV40 poly (A) signal.

In various embodiments, any of the disclosed rAAV vectors are encapsidated in an AAV8 capsid. In some embodiments, the disclosed rAAV vectors are encapsidated in an AAV9 capsid.

Further provided herein are empirical testing of regulatory elements in the disclosed AAV vectors for high expression levels of the encoded base editor.

### Definitions

As used herein and in the claims, the singular forms “a,” “an,” and “the” include the singular and the plural unless the context clearly indicates otherwise. Thus, for example, a reference to “an agent” includes a single agent and a plurality of such agents.

An “adeno-associated virus” or “AAV” is a virus which infects humans and some other primate species. The wild-type AAV genome is a single-stranded deoxyribonucleic acid (ssDNA), either positive- or negative-sensed. The genome comprises two inverted terminal repeats (ITRs), one at each end of the DNA strand, and two open reading frames (ORFs): rep and cap between the ITRs. The rep ORF comprises four overlapping genes encoding Rep proteins required for the AAV life cycle. The cap ORF comprises overlapping genes encoding capsid proteins: VP1, VP2 and VP3, which interact together to form the viral capsid. VP1, VP2 and VP3 are translated from one mRNA transcript, which can be spliced in two different manners: either a longer or shorter intron can be excised resulting in the formation of two isoforms of mRNAs: a ˜2.3 kb- and a ˜2.6 kb-long mRNA isoform. The capsid forms a supramolecular assembly of approximately 60 individual capsid protein subunits into a non-enveloped, T-1 icosahedral lattice capable of protecting the AAV genome. The mature capsid is composed of VP1, VP2, and VP3 (molecular masses of approximately 87, 73, and 62 kDa respectively) in a ratio of about 1:1:10.

rAAV particles may comprise a nucleic acid vector (e.g., a recombinant genome), which may comprise at a minimum: (a) one or more heterologous nucleic acid regions (or transgenes) comprising a sequence encoding a protein or polypeptide of interest (e.g., a base editor) or an RNA of interest (e.g., a gRNA); and (b) one or more regions comprising inverted terminal repeat (ITR) sequences (e.g., wild-type ITR sequences or engineered ITR sequences) flanking the one or more nucleic acid regions (e.g., heterologous nucleic acid regions). In some embodiments, the nucleic acid vector is between 4 kb and 5 kb in size (e.g., 4.2 to 4.7 kb in size). In some embodiments, the nucleic acid vector is circular. In some embodiments, the nucleic acid vector is single-stranded. In some embodiments, the nucleic acid vector is double-stranded. In some embodiments, a double-stranded nucleic acid vector may be, for example, a self-complimentary vector that contains a region of the nucleic acid vector that is complementary to another region of the nucleic acid vector, initiating the formation of the double-strandedness of the nucleic acid vector.

As used herein, the term “adenosine deaminase” or “adenosine deaminase domain” refers to a protein or enzyme that catalyzes a deamination reaction of an adenosine (or adenine). The terms are used interchangeably. In certain embodiments, the disclosure provides base editors comprising one or more adenosine deaminase domains. For instance, an adenosine deaminase domain may comprise a heterodimer of a first adenosine deaminase and a second deaminase domain, connected by a linker. Adenosine deaminases (e.g., engineered adenosine deaminases or evolved adenosine deaminases) provided herein may be may be enzymes that convert adenine (A) to inosine (I) in DNA or RNA. Such adenosine deaminase can lead to an A:T to G:C base pair conversion. In some embodiments, the deaminase is a variant of a naturally-occurring deaminase from an organism. In some embodiments, the deaminase does not occur in nature. For example, in some embodiments, the deaminase is at least 50%, at least 55%, at least 60%, at least 65%, at least 70%, at least 75% at least 80%, at least 85%, at least 90%, at least 95%, at least 96%, at least 97%, at least 98%, at least 99%, or at least 99.5% identical to a naturally-occurring deaminase.

In some embodiments, the adenosine deaminase is derived from a bacterium, such as, E. coli, S. aureus, S. typhi, S. putrefaciens, H. influenzae, or C. crescentus. In some embodiments, the adenosine deaminase is a TadA deaminase. In some embodiments, the TadA deaminase is an E. coli TadA deaminase (ecTadA). In some embodiments, the TadA deaminase is a truncated E. coli TadA deaminase. For example, the truncated ecTadA may be missing one or more N-terminal amino acids relative to a full-length ecTadA. In some embodiments, the truncated ecTadA may be missing 1, 2, 3, 4, 5, 6, 7, 8, 9, 10, 11, 12, 13, 14, 15, 6, 17, 18, 19, or 20 N-terminal amino acid residues relative to the full length ecTadA. In some embodiments, the truncated ecTadA may be missing 1, 2, 3, 4, 5, 6, 7, 8, 9, 10, 11, 12, 13, 14, 15, 6, 17, 18, 19, or 20 C-terminal amino acid residues relative to the full length ecTadA. In some embodiments, the ecTadA deaminase does not comprise an N-terminal methionine. Reference is made to U.S. Patent Publication No. 2018/0073012, published Mar. 15, 2018, which is incorporated herein by reference.

In genetics, the “antisense” strand of a segment within double-stranded DNA is the template strand, and which is considered to run in the 3′ to 5′ orientation. By contrast, the “sense” strand is the segment within double-stranded DNA that runs from 5′ to 3′, and which is complementary to the antisense strand of DNA, or template strand, which runs from 3′ to 5′. In the case of a DNA segment that encodes a protein, the sense strand is the strand of DNA that has the same sequence as the mRNA, which takes the antisense strand as its template during transcription, and eventually undergoes (typically, not always) translation into a protein. The antisense strand is thus responsible for the RNA that is later translated to protein, while the sense strand possesses a nearly identical makeup to that of the mRNA. Note that for each segment of dsDNA, there will possibly be two sets of sense and antisense, depending on which direction one reads (since sense and antisense is relative to perspective). It is ultimately the gene product, or mRNA, that dictates which strand of one segment of dsDNA is referred to as sense or antisense.

“Base editing” refers to genome editing technology that involves the conversion of a specific nucleic acid base into another at a targeted genomic locus. In certain embodiments, this can be achieved without requiring double-stranded DNA breaks (DSB), or single stranded breaks (i.e., nicking). To date, other genome editing techniques, including CRISPR-based systems, begin with the introduction of a DSB at a locus of interest. Subsequently, cellular DNA repair enzymes mend the break, commonly resulting in random insertions or deletions (indels) of bases at the site of the DSB. However, when the introduction or correction of a point mutation at a target locus is desired rather than stochastic disruption of the entire gene, these genome editing techniques are unsuitable, as correction rates are low (e.g. typically 0.1% to 5%), with the major genome editing products being indels. In order to increase the efficiency of gene correction without simultaneously introducing random indels, the present inventors previously modified the CRISPR/Cas9 system to directly convert one DNA base into another without DSB formation. See, Komor, A. C., et al., Programmable editing of a target base in genomic DNA without double-stranded DNA cleavage. Nature 533, 420-424 (2016), the entire contents of which is incorporated herein by reference.

The term “base editor (BE),” as used herein, refers to an agent comprising a polypeptide that is capable of making a modification to a base (e.g., A, T, C, G, or U) within a nucleic acid sequence (e.g., DNA or RNA) that converts one base to another (e.g., A to G, A to C, A to T, C to T, C to G, C to A, G to A, G to C, G to T, T to A, T to C, T to G). In some embodiments, the base editor is capable of deaminating a base within a nucleic acid such as a base within a DNA molecule. In the case of an adenine base editor, the base editor is capable of deaminating an adenine (A) in DNA. Such base editors may include a nucleic acid programmable DNA binding protein (napDNAbp) fused to an adenosine deaminase. Some base editors include CRISPR-mediated fusion proteins that are utilized in the base editing methods described herein. In some embodiments, the base editor comprises a nuclease-inactive Cas9 (dCas9) fused to a deaminase which binds a nucleic acid in a guide RNA-programmed manner via the formation of an R-loop, but does not cleave the nucleic acid. For example, the dCas9 domain of the fusion protein may include a D10A and a H840A mutation (which renders Cas9 capable of cleaving only one strand of a nucleic acid duplex), as described in PCT/US2016/058344, which published as WO 2017/070632 on Apr. 27, 2017 and is incorporated herein by reference in its entirety. The DNA cleavage domain of S. pyogenes Cas9 includes two subdomains, the HNH nuclease subdomain and the RuvC1 subdomain. The HNH subdomain cleaves the strand complementary to the gRNA (the “targeted strand”, or the strand in which editing or deamination occurs), whereas the RuvC1 subdomain cleaves the non-complementary strand containing the PAM sequence (the “non-edited strand”). The RuvC1 mutant D10A generates a nick in the targeted strand, while the HNH mutant H840A generates a nick on the non-edited strand (see Jinek et al., Science, 337:816-821 (2012); Qi et al., Cell. 28; 152 (5):1173-83 (2013), each of which are incorporated herein by reference).

In some embodiments, a base editor is a macromolecule or macromolecular complex that results primarily (e.g., more than 80%, more than 85%, more than 90%, more than 95%, more than 99%, more than 99.9%, or 100%) in the conversion of a nucleobase in a polynucleic acid sequence into another nucleobase (i.e., a transition or transversion) using a combination of 1) a nucleotide-, nucleoside-, or nucleobase-modifying enzyme and 2) a nucleic acid binding protein that can be programmed to bind to a specific nucleic acid sequence.

In some embodiments, the base editor comprises a DNA binding domain (e.g., a programmable DNA binding domain such as a dCas9 or nCas9) that directs it to a target sequence. In some embodiments, the base editor comprises a nucleobase modifying enzyme fused to a programmable DNA binding domain (e.g., a dCas9 or nCas9). A “nucleobase modifying enzyme” is an enzyme that can modify a nucleobase and convert one nucleobase to another (e.g., a deaminase such as a adenosine deaminase). Base editors that carry out certain types of base conversions (e.g., adenosine (A) to guanine (G), C to G) are contemplated.

In some embodiments, a base editor converts an A to G. In some embodiments, the base editor comprises an adenosine deaminase. An “adenosine deaminase” is an enzyme involved in purine metabolism. It is needed for the breakdown of adenosine from food and for the turnover of nucleic acids in tissues. Its primary function in humans is the development and maintenance of the immune system. An adenosine deaminase catalyzes hydrolytic deamination of adenosine (forming inosine, which base pairs as G) in the context of DNA. There are no known natural adenosine deaminases that act on DNA. Instead, known adenosine deaminase enzymes only act on RNA (tRNA or mRNA). Evolved deoxyadenosine deaminase enzymes that accept DNA substrates and deaminate dA to deoxyinosine have been described, e.g., in PCT Application PCT/US2017/045381, filed Aug. 3, 2017, which published as WO 2018/027078, and PCT Application No. PCT/US2019/033848, filed May 23, 2019, which published on Nov. 28, 2019 as WO 2019/226953, U.S. Patent Publication No. 2018/0073012, published Mar. 15, 2018, which issued as U.S. Pat. No. 10,113,163; on Oct. 30, 2018; U.S. Patent Publication No. 2017/0121693, published May 4, 2017, which issued as U.S. Pat. No. 10,167,457 on Jan. 1, 2019; PCT Publication No. WO 2017/070633, published Apr. 27, 2017; U.S. Patent Publication No. 2015/0166980, published Jun. 18, 2015; U.S. Pat. No. 9,840,699, issued Dec. 12, 2017; U.S. Pat. No. 10,077,453, issued Sep. 18, 2018; PCT Publication No. WO 2019/023680, published Jan. 31, 2019; International Application No. PCT/US2019/033848, filed May 23, 2019, which published as Publication No. WO 2019/226593 on Nov. 28, 2019; PCT Publication No. WO 2018/0176009, published Sep. 27, 2018, PCT Publication No. WO 2020/041751, published Feb. 27, 2020; PCT Publication No. WO 2020/051360, published Mar. 12, 2020; PCT Publication No. WO 2020/102659, published May 22, 2020; PCT Publication No. WO 2020/086908, published Apr. 30, 2020; PCT Publication No. WO 2020/181180, published Sep. 10, 2020; PCT Publication No. WO 2020/214842, published Oct. 22, 2020; PCT Publication No. WO 2020/092453, published May 7, 2020; PCT Publication No. WO 2020/236982, published Nov. 26, 2020; PCT Publication No. WO 2021/108717, published Jun. 3, 2021, and PCT Publication No. WO 2021/158921, published Aug. 12, 2021, the contents of each of which are incorporated herein by reference in their entireties.

In some embodiments, a base editor converts a C to a T. In some embodiments, the base editor comprises a cytidine deaminase. A “cytosine deaminase”, or “cytidine deaminase,” refers to an enzyme that catalyzes the chemical reaction “cytosine+H₂O→uracil+NH₃” or “5-methyl-cytosine+H₂O→thymine+NH₃.” As it may be apparent from the reaction formula, such chemical reactions result in a C to U/T nucleobase change. In the context of a gene, such a nucleotide change, or mutation, may in turn lead to an amino acid change in the protein, which may affect the protein's function, e.g., loss-of-function or gain-of-function. In some embodiments, the cytosine base editor comprises a dCas9 or nCas9 fused to a cytidine deaminase. In some embodiments, the cytidine deaminase domain is fused to the N-terminus of the dCas9 or nCas9. In some embodiments, the base editor further comprises a domain that inhibits uracil glycosylase, and/or a nuclear localization signal. Such base editors have been described in the art, e.g., in Rees & Liu, Nat Rev Genet. 2018; 19 (12): 770-788, Rees, et al. Sci. Advances 5, caax5717 (2019), and Koblan et al., Nat Biotechnol. 2018; 36 (9): 843-846; as well as U.S. Patent Publication No. 2018/0073012, published Mar. 15, 2018, which issued as U.S. Pat. No. 10,113,163 on Oct. 30, 2018; U.S. Patent Publication No. 2017/0121693, published May 4, 2017, which issued as U.S. Pat. No. 10,167,457 on Jan. 1, 2019; PCT Publication No. WO 2017/070633, published Apr. 27, 2017; U.S. Patent Publication No. 2015/0166980, published Jun. 18, 2015; U.S. Pat. No. 9,840,699, issued Dec. 12, 2017; U.S. Pat. No. 10,077,453, issued Sep. 18, 2018; PCT Publication No. WO 2019/023680, published Jan. 31, 2019; PCT Publication No. WO 2018/0176009, published Sep. 27, 2018, PCT Application No PCT/US2019/033848, filed May 23, 2019, PCT Application No. PCT/US2019/47996, filed Aug. 23, 2019; PCT Application No. PCT/US2020/028568, filed Apr. 17, 2020; PCT Application No. PCT/US2019/61685, filed Nov. 15, 2019; PCT Application No. PCT/US2019/57956, filed Oct. 24, 2019; PCT Publication No. PCT/US2019/58678, filed Oct. 29, 2019; and PCT Publication No. WO 2021/108717, published Jun. 3, 2021, the contents of each of which are incorporated herein by reference in their entireties.

Exemplary adenine and cytosine base editors are also described in Rees & Liu, Base editing: precision chemistry on the genome and transcriptome of living cells, Nat. Rev. Genet. 2018; 19 (12): 770-788; as well as U.S. Patent Publication No. 2018/0073012, published Mar. 15, 2018, which issued as U.S. Pat. No. 10,113,163, on Oct. 30, 2018; U.S. Patent Publication No. 2017/0121693, published May 4, 2017, which issued as U.S. Pat. No. 10,167,457 on Jan. 1, 2019; PCT Publication No. WO 2017/070633, published Apr. 27, 2017; U.S. Patent Publication No. 2015/0166980, published Jun. 18, 2015; U.S. Pat. No. 9,840,699, issued Dec. 12, 2017; and U.S. Pat. No. 10,077,453, issued Sep. 18, 2018, the contents of each of which are incorporated herein by reference in their entireties.

The term “Cas9” or “Cas9 nuclease” refers to an RNA-guided nuclease comprising a Cas9 domain, or a fragment thereof (e.g., a protein comprising an active or inactive DNA cleavage domain of Cas9, and/or the gRNA binding domain of Cas9). A “Cas9 domain” as used herein, is a protein fragment comprising an active or inactive cleavage domain of Cas9 and/or the gRNA binding domain of Cas9. A “Cas9 protein” is a full length Cas9 protein. A Cas9 nuclease is also referred to sometimes as a casn1 nuclease or a CRISPR (Clustered Regularly Interspaced Short Palindromic Repeat)-associated nuclease. CRISPR is an adaptive immune system that provides protection against mobile genetic elements (viruses, transposable elements, and conjugative plasmids). CRISPR clusters contain spacers, sequences complementary to antecedent mobile elements, and target invading nucleic acids. CRISPR clusters are transcribed and processed into CRISPR RNA (crRNA). In type II CRISPR systems correct processing of pre-crRNA requires a trans-encoded small RNA (tracrRNA), endogenous ribonuclease 3 (rnc) and a Cas9 domain. The tracrRNA serves as a guide for ribonuclease 3-aided processing of pre-crRNA. Subsequently, Cas9/crRNA/tracrRNA endonucleolytically cleaves linear or circular dsDNA target complementary to the spacer. The target strand not complementary to crRNA is first cut endonucleolytically, then trimmed 3′-5′ exonucleolytically. In nature, DNA-binding and cleavage typically requires protein and both RNAs. However, single guide RNAs (“sgRNA”, or simply “gRNA”) can be engineered so as to incorporate aspects of both the crRNA and tracrRNA into a single RNA species. See, e.g., Jinck M., et al. Science 337:816-821 (2012), the entire contents of which are incorporated herein by reference. Cas9 recognizes a short motif in the CRISPR repeat sequences (the PAM or protospacer adjacent motif) to help distinguish self versus non-self. Cas9 nuclease sequences and structures are well known to those of skill in the art (see, e.g., “Complete genome sequence of an MI strain of Streptococcus pyogenes.” Ferretti et al., J. J., McShan W. M., Ajdic D. J., Savic D. J., Savic G., Lyon K., Primeaux C., Sezate S., Suvorov A. N., Kenton S., Lai H. S., Lin S. P., Qian Y., Jia H. G., Najar F. Z., Ren Q., Zhu H., Song L., White J., Yuan X., Clifton S. W., Roc B. A., McLaughlin R. E., Proc. Natl. Acad. Sci. U.S.A. 98:4658-4663 (2001); “CRISPR RNA maturation by trans-encoded small RNA and host factor RNase III.” Deltcheva E., Chylinski K., Sharma C. M., Gonzales K., Chao Y., Pirzada Z. A., Eckert M. R., Vogel J., Charpentier E., Nature 471:602-607 (2011); and “A programmable dual-RNA-guided DNA endonuclease in adaptive bacterial immunity.” Jinek M., Chylinski K., Fonfara I., Hauer M., Doudna J. A., Charpentier E. Science 337:816-821 (2012), the entire contents of each of which are incorporated herein by reference). Cas9 orthologs have been described in various species, including, but not limited to, S. pyogenes and S. thermophilus (e.g., StCas9 or St1Cas9). Additional suitable Cas9 nucleases and sequences will be apparent to those of skill in the art based on this disclosure, and such Cas9 nucleases and sequences include Cas9 sequences from the organisms and loci disclosed in Chylinski, Rhun, and Charpentier, “The tracrRNA and Cas9 families of type II CRISPR-Cas immunity systems” (2013) RNA Biology 10:5, 726-737; the entire contents of which are incorporated herein by reference. In some embodiments, a Cas9 nuclease comprises one or more mutations that partially impair or inactivate the DNA cleavage domain.

A nuclease-inactivated Cas9 domain may interchangeably be referred to as a “dCas9” protein (for nuclease-“dead” Cas9). Methods for generating a Cas9 domain (or a fragment thereof) having an inactive DNA cleavage domain are known (see, e.g., Jinek et al., Science. 337:816-821 (2012); Qi et al., “Repurposing CRISPR as an RNA-Guided Platform for Sequence-Specific Control of Gene Expression” (2013) Cell. 28; 152 (5):1173-83, the entire contents of each of which are incorporated herein by reference). For example, the DNA cleavage domain of Cas9 is known to include two subdomains, the HNH nuclease subdomain and the RuvC1 subdomain. The HNH subdomain cleaves the strand complementary to the gRNA, whereas the RuvC1 subdomain cleaves the non-complementary strand. Mutations within these subdomains can silence the nuclease activity of Cas9. For example, the mutations D10A and H840A completely inactivate the nuclease activity of S. pyogenes Cas9 (Jinek et al., Science. 337:816-821 (2012); Qi et al., Cell. 28; 152 (5):1173-83 (2013)). In some embodiments, proteins comprising fragments of Cas9 are provided. For example, in some embodiments, a protein comprises one of two Cas9 domains: (1) the gRNA binding domain of Cas9; or (2) the DNA cleavage domain of Cas9. In some embodiments, proteins comprising Cas9 or fragments thereof are referred to as “Cas9 variants.” A Cas9 variant shares homology to Cas9, or a fragment thereof. For example, a Cas9 variant is at least about 70% identical, at least about 80% identical, at least about 90% identical, at least about 95% identical, at least about 96% identical, at least about 97% identical, at least about 98% identical, at least about 99% identical, at least about 99.5% identical, at least about 99.8% identical, or at least about 99.9% identical to wild type Cas9 (e.g., SpCas9 of SEQ ID NO: 74). In some embodiments, the Cas9 variant may have 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or more amino acid changes compared to wild type Cas9 (e.g., SpCas9 of SEQ ID NO: 74). In some embodiments, the Cas9 variant comprises a fragment of Cas9 (e.g., a gRNA binding domain or a DNA-cleavage domain), such that the fragment is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to the corresponding fragment of wild type Cas9 (e.g., SpCas9 of SEQ ID NO: 74). In some embodiments, the fragment is at least 30%, at least 35%, at least 40%, at least 45%, at least 50%, at least 55%, at least 60%, at least 65%, at least 70%, at least 75%, at least 80%, at least 85%, at least 90%, at least 95% identical, at least 96%, at least 97%, at least 98%, at least 99%, or at least 99.5% of the amino acid length of a corresponding wild type Cas9 (e.g., SpCas9 of SEQ ID NO: 74).

As used herein, the term “nCas9” or “Cas9 nickase” refers to a Cas9 or a variant thereof, which cleaves or nicks only one of the strands of a target cut site thereby introducing a nick in a double strand DNA molecule rather than creating a double strand break. This can be achieved by introducing appropriate mutations in a wild-type Cas9 which inactivates one of the two endonuclease activities of the Cas9. Any suitable mutation which inactivates one Cas9 endonuclease activity but leaves the other intact is contemplated, such as one of D10A or H840A mutations in the wild-type S. pyogenes Cas9 amino acid sequence, or a D10A mutation in the wild-type S. aureus Cas9 amino acid sequence, may be used to form the nCas9.

The term “cDNA” refers to a strand of DNA copied from an RNA template. cDNA is complementary to the RNA template.

CRISPR is a family of DNA sequences (i.e., CRISPR clusters) in bacteria and archaea that represent snippets of prior infections by a virus that have invaded the prokaryote. The snippets of DNA are used by the prokaryotic cell to detect and destroy DNA from subsequent attacks by similar viruses and effectively compose, along with an array of CRISPR-associated proteins (including Cas9 and homologs thereof) and CRISPR-associated RNA, a prokaryotic immune defense system. In nature, CRISPR clusters are transcribed and processed into CRISPR RNA (crRNA). In certain types of CRISPR systems (e.g., type II CRISPR systems), correct processing of pre-crRNA requires a trans-encoded small RNA (tracrRNA), endogenous ribonuclease 3 (rnc) and a Cas9 protein. The tracrRNA serves as a guide for ribonuclease 3-aided processing of pre-crRNA. Subsequently, Cas9/crRNA/tracrRNA endonucleolytically cleaves linear or circular dsDNA target complementary to the RNA. Specifically, the target strand not complementary to crRNA is first cut endonucleolytically, then trimmed 3′-5′ exonucleolytically. In nature, DNA-binding and cleavage typically requires protein and both RNAs. However, single guide RNAs (“sgRNA”, or simply “gRNA”) can be engineered so as to incorporate aspects of both the crRNA and tracrRNA into a single RNA species—the guide RNA. Sec, e.g., Jinck M., Chylinski K., Fonfara I., Hauer M., Doudna J. A., Charpentier E. Science 337:816-821 (2012), the entire contents of which is hereby incorporated by reference. Cas9 recognizes a short motif in the CRISPR repeat sequences (the PAM or protospacer adjacent motif) to help distinguish self versus non-self. CRISPR biology, as well as Cas9 nuclease sequences and structures are well known to those of skill in the art (see, e.g., “Complete genome sequence of an MI strain of Streptococcus pyogenes.” Ferretti et al., J. J., McShan W. M., Ajdic D. J., Savic D. J., Savic G., Lyon K., Primeaux C., Sezate S., Suvorov A. N., Kenton S., Lai H. S., Lin S. P., Qian Y., Jia H. G., Najar F. Z., Ren Q., Zhu H., Song L., White J., Yuan X., Clifton S. W., Roc B. A., Mclaughlin R. E., Proc. Natl. Acad. Sci. U.S.A. 98:4658-4663 (2001); “CRISPR RNA maturation by trans-encoded small RNA and host factor RNase III.” Deltcheva E., Chylinski K., Sharma C. M., Gonzales K., Chao Y., Pirzada Z. A., Eckert M. R., Vogel J., Charpentier E., Nature 471:602-607 (2011); and “A programmable dual-RNA-guided DNA endonuclease in adaptive bacterial immunity.” Jinek M., Chylinski K., Fonfara I., Hauer M., Doudna J. A., Charpentier E. Science 337:816-821 (2012), the entire contents of each of which are incorporated herein by reference). Cas9 orthologs have been described in various species, including, but not limited to, S. pyogenes and S. thermophilus. Additional suitable Cas9 nucleases and sequences will be apparent to those of skill in the art based on this disclosure, and such Cas9 nucleases and sequences include Cas9 sequences from the organisms and loci disclosed in Chylinski, Rhun, and Charpentier, “The tracrRNA and Cas9 families of type II CRISPR-Cas immunity systems” (2013) RNA Biology 10:5, 726-737; the entire contents of which are incorporated herein by reference.

The term “deaminase” or “deaminase domain” refers to a protein or enzyme that catalyzes a deamination reaction. In some embodiments, the deaminase is an adenosine (or adenine) deaminase, which catalyzes the hydrolytic deamination of adenine or adenosine. In some embodiments, the adenosine deaminase catalyzes the hydrolytic deamination of adenine or adenosine in deoxyribonucleic acid (DNA) to inosine. Ins some embodiments, the deaminase is a cytidine (or cytosine) deaminase, which catalyzes the hydrolytic deamination of cytidine to uracil.

The deaminases described herein may be from any organism, such as a bacterium. In some embodiments, the deaminase or deaminase domain is a variant of a naturally-occurring deaminase from an organism. In some embodiments, the deaminase or deaminase domain does not occur in nature. For example, in some embodiments, the deaminase or deaminase domain is at least 50%, at least 55%, at least 60%, at least 65%, at least 70%, at least 75% at least 80%, at least 85%, at least 90%, at least 95%, at least 96%, at least 97%, at least 98%, at least 99%, or at least 99.5% identical to a naturally-occurring deaminase.

As used herein, the term “DNA binding protein” or “DNA binding protein domain” refers to any protein that localizes to and binds a specific target DNA nucleotide sequence (e.g. a gene locus of a genome). This term embraces RNA-programmable proteins, which associate (e.g. form a complex) with one or more nucleic acid molecules (i.e., which includes, for example, guide RNA in the case of Cas systems) that direct or otherwise program the protein to localize to a specific target nucleotide sequence (e.g., DNA sequence) that is complementary to the one or more nucleic acid molecules (or a portion or region thereof) associated with the protein. Exemplary RNA-programmable proteins are CRISPR-Cas9 proteins, as well as Cas9 equivalents, homologs, orthologs, or paralogs, whether naturally occurring or non-naturally occurring (e.g. engineered or modified), and may include a Cas9 equivalent from any type of CRISPR system (e.g. type II, V, VI), including Cpf1 (a type-V CRISPR-Cas systems) (now known as Cas12a), C2c1 (a type V CRISPR-Cas system), C2c2 (a type VI CRISPR-Cas system), C2c3 (a type V CRISPR-Cas system), dCas9, GeoCas9, CjCas9, Nme2Cas9, SaCas9, SaKKH-Cas9, SauriCas9, Cas12b, Cas12c, Cas12d, Cas12g, Cas12h, Cas12i, Cas13d, Cas14, Argonaute, and nCas9. Further Cas-equivalents are described in Makarova et al., “C2c2 is a single-component programmable RNA-guided RNA-targeting CRISPR effector,” Science 2016; 353 (6299), the contents of which are incorporated herein by reference.

The term “DNA editing efficiency,” as used herein, refers to the number or proportion of intended base pairs that are edited. For example, if a base editor edits 10% of the base pairs that it is intended to target (e.g., within a cell or within a population of cells), then the base editor can be described as being 10% efficient. Some aspects of editing efficiency embrace the modification (e.g. deamination) of a specific nucleotide within DNA, without generating a large number or percentage of insertions or deletions (i.e., indels). It is generally accepted that editing while generating less than 5% indels (as measured over total target nucleotide substrates) is high editing efficiency. The generation of more than 20% indels is generally accepted as poor or low editing efficiency. Indel formation may be measured by techniques known in the art, including high-throughput screening of sequencing reads.

The term “off-target editing frequency,” as used herein, refers to the number or proportion of unintended base pairs, e.g., DNA base pairs, that are edited. On-target and off-target editing frequencies may be measured by the methods and assays described herein, further in view of techniques known in the art, including high-throughput sequencing reads. As used herein, high-throughput sequencing involves the hybridization of nucleic acid primers (e.g., DNA primers) with complementarity to nucleic acid (e.g., DNA) regions just upstream or downstream of the target sequence or off-target sequence of interest. Because the DNA target sequence and the Cas9-independent off-target sequences are known a priori in the methods disclosed herein, nucleic acid primers with sufficient complementarity to regions upstream or downstream of the target sequence and Cas9-independent off-target sequences of interest may be designed using techniques known in the art, such as the PhusionU PCR kit (Life Technologies), Phusion HS II kit (Life Technologies), and Illumina MiSeq kit. The number of off-target DNA edits may be measured by techniques known in the art, including high-throughput screening of sequencing reads, EndoV-Seq, GUIDE-Seq, CIRCLE-Seq, and Cas-OFFinder. Since many of the Cas9-dependent off-target sites have high sequence identity to the target site of interest, nucleic acid primers with sufficient complementarity to regions upstream or downstream of the Cas9-dependent off-target site may likewise be designed using techniques and kits known in the art. These kits make use of polymerase chain reaction (PCR) amplification, which produces amplicons as intermediate products. The target and off-target sequences may comprise genomic loci that further comprise protospacers and PAMs. Accordingly, the term “amplicons,” as used herein, may refer to nucleic acid molecules that constitute the aggregates of genomic loci, protospacers and PAMs. High-throughput sequencing techniques used herein may further include Sanger sequencing and Illumina-based next-generation genome sequencing (NGS).

The term “on-target editing,” as used herein, refers to the introduction of intended modifications (e.g., deaminations) to nucleotides (e.g., adenine) in a target sequence, such as using the base editors described herein. The term “off-target DNA editing,” as used herein, refers to the introduction of unintended modifications (e.g. deaminations) to nucleotides (e.g. adenine) in a sequence outside the canonical base editor binding window (i.e., from one protospacer position to another, typically 2 to 8 nucleotides long). Off-target DNA editing can result from weak or non-specific binding of the gRNA sequence to the target sequence. As used herein, the term “bystander editing” refers to synonymous off-target point mutations at nucleobases that are near (proximate to) the target base and do not change the outcome of the intended mutation (e.g., the intended disruption of a splice acceptor site, incorporation of a premature stop codon, or reversal of a mutant codon). Bystander edits may encompass non-silent mutations in the relevant codon of the transcript that do not result in a different translated protein.

As used herein, the terms “purity” and “product purity” of a base editor refer to the mean the percentage of edited sequencing reads (reads in which the target nucleobase has been converted to a different base) in which the intended target conversion occurs (e.g., in which the target A, and only the target A, is converted to a G). See Komor et al., Sci Adv 3 (2017).

As used herein, the terms “upstream” and “downstream” are terms of relativity that define the linear position of at least two elements located in a nucleic acid molecule (whether single or double-stranded) that is orientated in a 5′-to-3′ direction. In particular, a first element is upstream of a second element in a nucleic acid molecule where the first element is positioned somewhere that is 5′ to the second element. For example, a SNP is upstream of a Cas9-induced nick site if the SNP is on the 5′ side of the nick site. Conversely, a first element is downstream of a second element in a nucleic acid molecule where the first element is positioned somewhere that is 3′ to the second element. For example, a SNP is downstream of a Cas9-induced nick site if the SNP is on the 3′ side of the nick site. The nucleic acid molecule can be a DNA (double or single stranded). RNA (double or single stranded), or a hybrid of DNA and RNA. The analysis is the same for single strand nucleic acid molecule and a double strand molecule since the terms upstream and downstream are in reference to only a single strand of a nucleic acid molecule, except that one needs to select which strand of the double stranded molecule is being considered. Often, the strand of a double stranded DNA which can be used to determine the positional relativity of at least two elements is the “sense” or “coding” strand. In genetics, a “sense” strand is the segment within double-stranded DNA that runs from 5′ to 3′, and which is complementary to the antisense strand of DNA, or template strand, which runs from 3′ to 5′. Thus, as an example, a SNP nucleobase is “downstream” of a promoter sequence in a genomic DNA (which is double-stranded) if the SNP nucleobase is on the 3′ side of the promoter on the sense or coding strand.

The term “effective amount,” as used herein, refers to an amount of a biologically active agent that is sufficient to elicit a desired biological response. For example, in some embodiments, an effective amount of a base editor may refer to the amount of the editor that is sufficient to edit a target site nucleotide sequence, e.g., a genome. In some embodiments, an effective amount of a base editor described herein, e.g., of a base editor comprising a nickase Cas9 domain and a guide RNA may refer to the amount of the base editor that is sufficient to induce editing of a target site specifically bound and edited by the base editor. As will be appreciated by the skilled artisan, the effective amount of an agent, e.g., a base editor, a nuclease, a hybrid protein, a protein dimer, a complex of a protein (or protein dimer) and a polynucleotide, or a polynucleotide, may vary depending on various factors as, for example, on the desired biological response, e.g., on the specific allele, genome, or target site to be edited, on the cell or tissue being targeted, and on the agent being used.

The term “functional equivalent” refers to a second biomolecule that is equivalent in function, but not necessarily equivalent in structure to a first biomolecule. For example, a “Cas9 equivalent” refers to a protein that has the same or substantially the same functions as Cas9, but not necessarily the same amino acid sequence. In the context of the disclosure, the specification refers throughout to “a protein X, or a functional equivalent thereof.” In this context, a “functional equivalent” of protein X embraces any homolog, paralog, fragment, naturally occurring, engineered, circular permutant, mutated, or synthetic version of protein X which bears an equivalent function.

The term “fusion protein” as used herein refers to a hybrid polypeptide which comprises protein domains from at least two different proteins. One protein may be located at the amino-terminal (N-terminal) portion of the fusion protein or at the carboxy-terminal (C-terminal) protein thus forming an “amino-terminal fusion protein” or a “carboxy-terminal fusion protein,” respectively. A protein may comprise different domains, for example, a nucleic acid binding domain (e.g., the gRNA binding domain of Cas9 that directs the binding of the protein to a target site) and a nucleic acid cleavage domain or a catalytic domain of a nucleic-acid editing protein. Another example includes a Cas9 or equivalent thereof fused to an adenosine deaminae. Any of the proteins described herein may be produced by any method known in the art. For example, the proteins described herein may be produced via recombinant protein expression and purification, which is especially suited for fusion proteins comprising a peptide linker. Methods for recombinant protein expression and purification are well known, and include those described by Green and Sambrook, Molecular Cloning: A Laboratory Manual (4th ed., Cold Spring Harbor Laboratory Press, Cold Spring Harbor, N.Y. (2012)), the entire contents of which are incorporated herein by reference.

The term “guide nucleic acid” or “napDNAbp-programming nucleic acid molecule” or equivalently “guide sequence” refers to one or more nucleic acid molecules which associate with and direct or otherwise program a napDNAbp protein to localize to a specific target nucleotide sequence (e.g., a gene locus of a genome) that is complementary to the one or more nucleic acid molecules (or a portion or region thereof) associated with the protein, thereby causing the napDNAbp protein to bind to the nucleotide sequence at the specific target site. A non-limiting example is a guide RNA of a Cas protein of a CRISPR-Cas genome editing system. Chemically, guide nucleic acids can be all RNA, all DNA, or a chimeric of RNA and DNA. The guide nucleic acids may also include nucleotide analogs. Guide nucleic acids can be expressed as transcription products or can be synthesized.

As used herein, a “guide RNA”, or “gRNA,” refers to a synthetic fusion of the endogenous bacterial crRNA and tracrRNA that provides both targeting specificity and a scaffold and/or binding ability for Cas9 nuclease to a target DNA. This synthetic fusion does not exist in nature and is also commonly referred to as an sgRNA. However, the term, guide RNA, also embraces equivalent guide nucleic acid molecules that associate with Cas9 equivalents, homologs, orthologs, or paralogs, whether naturally occurring or non-naturally occurring (e.g., engineered or recombinant), and which otherwise program the Cas9 equivalent to localize to a specific target nucleotide sequence. The Cas9 equivalents may include other napDNAbp from any type of CRISPR system (e.g., type II, V, VI), including Cpf1 (a type-V CRISPR-Cas systems), C2c1 (a type V CRISPR-Cas system), C2c2 (a type VI CRISPR-Cas system) and C2c3 (a type V CRISPR-Cas system). Further Cas-equivalents are described in Makarova et al., “C2c2 is a single-component programmable RNA-guided RNA-targeting CRISPR effector,” Science 2016; 353 (6299), the contents of which are incorporated herein by reference. Exemplary sequences are and structures of guide RNAs are provided herein. In addition, methods for designing appropriate guide RNA sequences are provided herein.

A guide RNA is a particular type of guide nucleic acid which is mostly commonly associated with a Cas protein of a CRISPR-Cas9 and which associates with Cas9, directing the Cas9 protein to a specific sequence in a DNA molecule that includes complementarity to the protospacer sequence for the guide RNA. Functionally, guide RNAs associate with Cas9, directing (or programming) the Cas9 protein to a specific sequence in a DNA molecule that includes a sequence complementary to the protospacer sequence for the guide RNA.

As used herein, a “spacer sequence” is the sequence of the guide RNA (˜20 nts in length) which has the same sequence (with the exception of uridine bases in place of thymine bases) as the protospacer of the PAM strand of the target (DNA) sequence, and which is complementary to the target strand (or non-PAM strand) of the target sequence.

As used herein, the “target sequence” refers to the ˜20 nucleotides in the target DNA sequence that have complementarity to the protospacer sequence in the PAM strand. The target sequence is the sequence that anneals to or is targeted by the spacer sequence of the guide RNA. The spacer sequence of the guide RNA and the protospacer have the same sequence (except the spacer sequence is RNA, and the protospacer is DNA).

As used herein, the terms “guide RNA core,” “guide RNA scaffold sequence” and “backbone sequence” refer to the sequence within the gRNA that is responsible for Cas9 binding, it does not include the 20 bp spacer sequence that is used to guide Cas9 to target DNA.

The term “host cell,” as used herein, refers to a cell that can host and replicate a vector encoding a base editor, guide RNA, and/or combination thereof, as described herein. In some embodiments, host cells are mammalian cells, such as human cells. Provided herein are methods of transducing and transfecting a host cell, such as a human cell, e.g., a human cell in a subject, with one or more vectors provided herein, such as one or more viral (e.g., rAAV) vectors provided herein.

It should be appreciated that any of the base editors, guide RNAs, and or combinations thereof, described herein may be introduced into a host cell in any suitable way, either stably or transiently. In some embodiments, a base editor may be transfected into the host cell. In some embodiments, the host cell may be transduced or transfected with a nucleic acid construct that encodes a base editor. For example, a host cell may be transduced (e.g., with a viral particle encoding a base editor) with a nucleic acid that encodes a base editor, or the translated base editor. As an additional example, a host cell may be transfected with a nucleic acid (e.g., a plasmid) that encodes a base editor or the translated base editor. Such transductions or transfections may be stable or transient. In some embodiments, host cells expressing a base editor or containing a base editor may be transduced or transfected with one or more gRNA molecules, for example when the base editor comprises a Cas9 (e.g., nCas9) domain. In some embodiments, a plasmid expressing a base editor may be introduced into host cells through electroporation, transient transfection (e.g., lipofection, such as with Lipofectamine 3000®), stable genome integration (e.g., piggyback), viral transduction, or other methods known to those of skill in the art.

Also provided herein are host cells for packaging of viral particles. In embodiments where the vector is a viral vector, a suitable host cell is a cell that may be infected by the viral vector, can replicate it, and can package it into viral particles that can infect fresh host cells. A cell can host a viral vector if it supports expression of genes of viral vector, replication of the viral genome, and/or the generation of viral particles. In some embodiments, the host cell is a eukaryotic cell, for example, a yeast cell, an insect cell, or a mammalian cell. The type of host cell, will, of course, depend on the vector employed, and suitable host cell/vector combinations will be readily apparent to those of skill in the art.

An “intein” is a segment of a protein that is able to excise itself and join the remaining portions (the exteins) with a peptide bond in a process known as protein splicing. Inteins are also referred to as “protein introns.” The process of an intein excising itself and joining the remaining portions of the protein is herein termed “protein splicing” or “intein-mediated protein splicing.” In some embodiments, an intein of a precursor protein (an intein containing protein prior to intein-mediated protein splicing) comes from two genes. Such intein is referred to herein as a split intein. For example, in cyanobacteria, DnaE, the catalytic subunit a of DNA polymerase III, is encoded by two separate genes, dnaE-n and dnaE-c. The intein encoded by the dnaE-n gene is herein referred as “intein-N.” The intein encoded by the dnaE-c gene is herein referred as “intein-C.” In various embodiments of the disclosed nucleic acid molecules, the nucleic acid molecules do not contain an intein. In various embodiments of the disclosed nucleic acid molecules, the nucleic acid molecules do not contain a trans-splicing intein.

Other intein systems may also be used. For example, a synthetic intein based on the dnaE intein, the Cfa-N and Cfa-C intein pair, has been described (e.g., in Stevens et al., J Am Chem Soc. 2016 Feb. 24; 138 (7): 2162-5, incorporated herein by reference). As another example, a synthetic intein based on the dnaE intein, the Nostoc punctiforme (Npu) intein pair, has been described (see Zettler, J., Schutz, V. & Mootz, H. D., The naturally split Npu DnaE intein exhibits an extraordinarily high rate in the protein trans-splicing reaction. FEBS letters 583, 909-914 (2009), incorporated herein by reference). In some embodiments, the intein is a fast-splicing gp41 intein, such as a gp41-8 intein. Reference is made to Carvajal-Vallejos et al, Journal of Biological Chemistry 287 (34): 28686-28696 (2012), and Pinto, Thornton & Wang, Nat. Comm. (2020) 11:1529, each of which are incorporated herein by reference. Non-limiting examples of intein pairs that may be used in accordance with the present disclosure include: Cfa DnaE intein, Npu DnaE intein, gp41-8 intein, Ssp GyrB intein, Ssp DnaX intein, Ter DnaE3 intein, Ter ThyX intein, Rma DnaB intein and Cne Prp8 intein (e.g., as described in U.S. Pat. No. 8,394,604, incorporated herein by reference).

The term “linker,” as used herein, refers to a chemical group or a molecule linking two molecules or domains, e.g. dCas9 and a deaminase. Typically, the linker is positioned between, or flanked by, two groups, molecules, or other domains and connected to each one via a covalent bond, thus connecting the two. In some embodiments, the linker is an amino acid or a plurality of amino acids (e.g. a peptide or protein). In some embodiments, the linker is an organic molecule, group, polymer, or chemical domain. Chemical groups include, but are not limited to, disulfide, hydrazone, and azide domains. In some embodiments, the linker is 5-100 amino acids in length, for example, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. Longer or shorter linkers are also contemplated. In some embodiments, the linker is an XTEN linker, which is 32 amino acids in length. In some embodiments, the linker is a 32-amino acid linker. In other embodiments, the linker is a 30-, 31-, 33- or 34-amino acid linker.

The term “mutation,” as used herein, refers to a substitution of a residue within a sequence, e.g. a nucleic acid or amino acid sequence, with another residue; a deletion or insertion of one or more residues within a sequence; or a substitution of a residue within a sequence of a genome in a subject to be corrected. Mutations are typically described herein by identifying the original residue followed by the position of the residue within the sequence and by the identity of the newly substituted residue. Various methods for making the amino acid substitutions (mutations) provided herein are well known in the art, and are provided by, for example, Green and Sambrook, Molecular Cloning: A Laboratory Manual (4^{th} ed., Cold Spring Harbor Laboratory Press, Cold Spring Harbor, N.Y. (2012)). Mutations can include a variety of categories, such as single base polymorphisms, microduplication regions, indel, and inversions, and is not meant to be limiting in any way. Mutations can include “loss-of-function” mutations which are mutations that reduce or abolish a protein activity. Most loss-of-function mutations are recessive, because in a heterozygote the second chromosome copy carries an unmutated version of the gene coding for a fully functional protein whose presence compensates for the effect of the mutation. There are some exceptions where a loss-of-function mutation is dominant, one example being haploinsufficiency, where the organism is unable to tolerate the approximately 50% reduction in protein activity suffered by the heterozygote. This is the explanation for a few genetic diseases in humans, including Marfan syndrome, which results from a mutation in the gene for the connective tissue protein called fibrillin. Mutations also embrace “gain-of-function” mutations, which is one which confers an abnormal activity on a protein or cell that is otherwise not present in a normal condition. Many gain-of-function mutations are in regulatory sequences rather than in coding regions, and can therefore have a number of consequences. Because of their nature, gain-of-function mutations are usually dominant. Many loss-of-function mutations are recessive, such as autosomal recessive. Many of the USH2A mutations for which the presently disclosed base editing methods aim to correct are autosomal recessive.

The term “napDNAbp” which stand for “nucleic acid programmable DNA binding protein” refers to any protein that may associate (e.g., form a complex) with one or more nucleic acid molecules (i.e., which may broadly be referred to as a “napDNAbp-programming nucleic acid molecule” and includes, for example, guide RNA in the case of Cas systems) which direct or otherwise program the protein to localize to a specific target nucleotide sequence (e.g., a gene locus of a genome) that is complementary to the one or more nucleic acid molecules (or a portion or region thereof) associated with the protein, thereby causing the protein to bind to the nucleotide sequence at the specific target site. This term napDNAbp embraces CRISPR-Cas9 proteins, as well as Cas9 equivalents, homologs, orthologs, or paralogs, whether naturally occurring or non-naturally occurring (e.g., engineered or modified), and may include a Cas9 equivalent from any type of CRISPR system (e.g., type II, V, VI), including Cpf1 (a type-V CRISPR-Cas systems), C2c1 (a type V CRISPR-Cas system), C2c2 (a type VI CRISPR-Cas system), C2c3 (a type V CRISPR-Cas system), dCas9, GeoCas9, CjCas9, Cas12a, Cas12b, Cas12c, Cas12d, Cas12g, Cas12h, Cas12i, Cas13d, Cas14, Argonaute, and nCas9. Further Cas-equivalents are described in Makarova et al., “C2c2 is a single-component programmable RNA-guided RNA-targeting CRISPR effector,” Science 2016; 353 (6299), the contents of which are incorporated herein by reference. However, the nucleic acid programmable DNA binding protein (napDNAbp) that may be used in connection with this invention are not limited to CRISPR-Cas systems. The invention embraces any such programmable protein, such as the Argonaute protein from Natronobacterium gregoryi (NgAgo) which may also be used for DNA-guided genome editing. NgAgo-guide DNA system does not require a PAM sequence or guide RNA molecules, which means genome editing can be performed simply by the expression of generic NgAgo protein and introduction of synthetic oligonucleotides on any genomic sequence. See Gao et al., DNA-guided genome editing using the Natronobacterium gregoryi Argonaute. Nature Biotechnology 2016; 34 (7): 768-73, which is incorporated herein by reference.

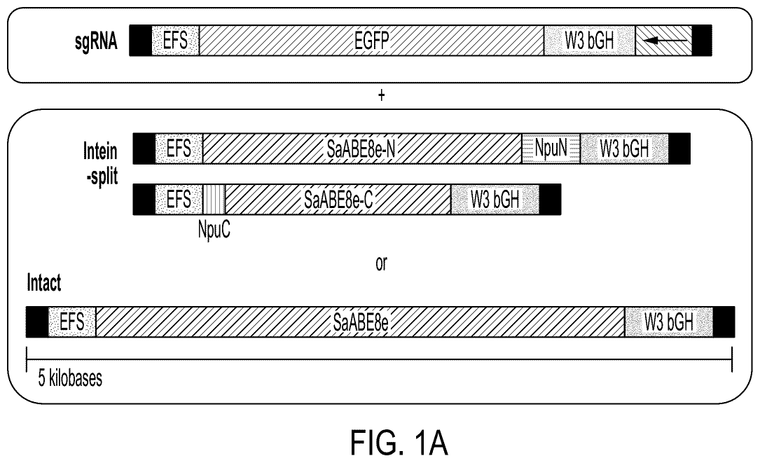

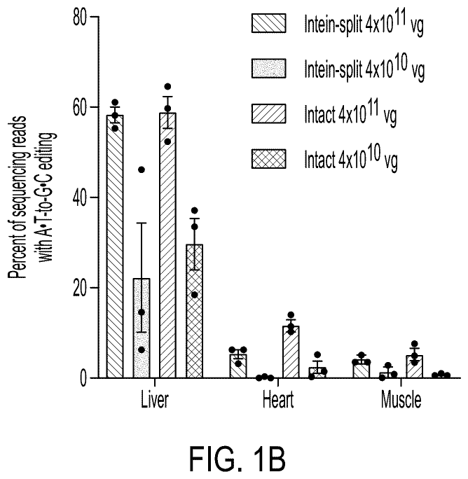

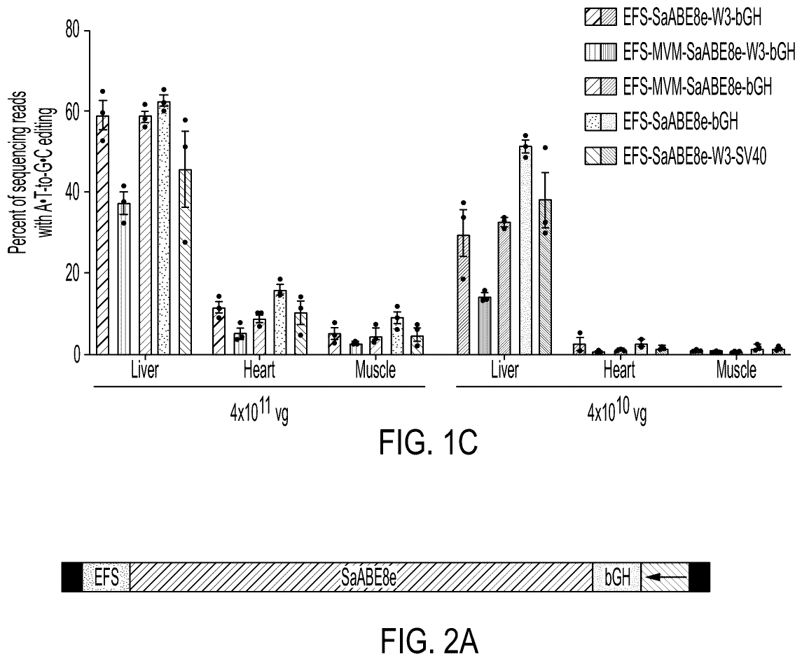

In some embodiments, the napDNAbp is a RNA-programmable nuclease, when in a complex with an RNA, may be referred to as a nuclease: RNA complex. Typically, the bound RNA(s) is referred to as a guide RNA (gRNA). gRNAs can exist as a complex of two or more RNAs, or as a single RNA molecule. gRNAs that exist as a single RNA molecule may be referred to as single-guide RNAs (sgRNAs), though “gRNA” is used interchangeably to refer to guide RNAs that exist as either single molecules or as a complex of two or more molecules. Typically, gRNAs that exist as single RNA species comprise two domains: (1) a domain that shares homology to a target nucleic acid (e.g., and directs binding of a Cas9 (or equivalent) complex to the target); and (2) a domain that binds a Cas9 protein. In some embodiments, domain (2) corresponds to a sequence known as a tracrRNA, and comprises a stem-loop structure. For example, in some embodiments, domain (2) is homologous to a tracrRNA as depicted in FIG. 1E of Jinek et al., Science 337:816-821 (2012), the entire contents of which is incorporated herein by reference. Other examples of gRNAs (e.g., those including domain 2) can be found in U.S. Pat. No. 9,340,799, entitled “mRNA-Sensing Switchable gRNAs,” and PCT Application No. PCT/US2014/054247, filed Sep. 6, 2013, published as WO 2015/035136 and entitled “Delivery System For Functional Nucleases,” the entire contents of each are incorporated herein by reference. In some embodiments, a gRNA comprises two or more of domains (1) and (2), and may be referred to as an “extended gRNA.” For example, an extended gRNA will, e.g., bind two or more Cas9 proteins and bind a target nucleic acid at two or more distinct regions, as described herein. The gRNA comprises a nucleotide sequence that complements a target site, which mediates binding of the nuclease/RNA complex to said target site, providing the sequence specificity of the nuclease: RNA complex. In some embodiments, the RNA-programmable nuclease is the (CRISPR-associated system) Cas9 endonuclease, for example Cas9 (Csn1) from Streptococcus pyogenes (see, e.g., “Complete genome sequence of an MI strain of Streptococcus pyogenes.” Ferretti J. J. et al., Proc. Natl. Acad. Sci. U.S.A. 98:4658-4663 (2001); “CRISPR RNA maturation by trans-encoded small RNA and host factor RNase III.” Deltcheva E. et al., Nature 471:602-607 (2011); and “A programmable dual-RNA-guided DNA endonuclease in adaptive bacterial immunity.” Jinek M. et al., Science 337:816-821 (2012), the entire contents of each of which are incorporated herein by reference.

The napDNAbp nucleases (e.g., Cas9) use RNA: DNA hybridization to target DNA cleavage sites, these proteins are able to be targeted, in principle, to any sequence specified by the guide RNA. Methods of using napDNAbp nucleases, such as Cas9, for site-specific cleavage (e.g., to modify a genome) are known in the art (see e.g., Cong, L. et al. Multiplex genome engineering using CRISPR/Cas systems. Science 339, 819-823 (2013); Mali, P. et al. RNA-guided human genome engineering via Cas9. Science 339, 823-826 (2013); Hwang, W. Y. et al. Efficient genome editing in zebrafish using a CRISPR-Cas system. Nature Biotechnology 31, 227-229 (2013); Jinek, M. et al. RNA-programmed genome editing in human cells. eLife 2, c00471 (2013); Dicarlo, J. E. et al., Genome engineering in Saccharomyces cerevisiae using CRISPR-Cas systems. Nucleic Acid Res. (2013); Jiang, W. et al. RNA-guided editing of bacterial genomes using CRISPR-Cas systems. Nature Biotechnology 31, 233-239 (2013); the entire contents of each of which are incorporated herein by reference).

The term “nickase” refers to a napDNAbp (e.g., a Cas9) having only a single nuclease activity that cuts only one strand of a target DNA, rather than both strands. Thus, a nickase type napDNAbp does not leave a double-strand break. Exemplary nickases include SpCas9 and SaCas9 nickases. An exemplary nickase comprises a sequence having at least 99%, or 100%, identity to the amino acid sequence of SEQ ID NO: 107.

A nuclear localization signal or sequence (NLS) is an amino acid sequence that tags, designates, or otherwise marks a protein for import into the cell nucleus by nuclear transport. Typically, this signal consists of one or more short sequences of positively charged lysines or arginines exposed on the protein surface. Different nuclear localized proteins may share the same NLS. An NLS has the opposite function of a nuclear export signal (NES), which targets proteins out of the nucleus. Thus, a single nuclear localization signal can direct the entity with which it is associated to the nucleus of a cell. Such sequences may be of any size and composition, for example, more than 25, 25, 15, 12, 10, 8, 7, 6, 5, or 4 amino acids, but will preferably comprise at least a four to eight amino acid sequence known to function as a nuclear localization signal (NLS).

The term “nucleic acid molecule” as used herein, refers to RNA as well as single and/or double-stranded DNA. Nucleic acid molecules may be naturally occurring, for example, in the context of a genome, a transcript, an mRNA, tRNA, rRNA, siRNA, snRNA, a plasmid, cosmid, chromosome, chromatid, or other naturally occurring nucleic acid molecule. On the other hand, a nucleic acid molecule may be a non-naturally occurring molecule, e.g. a recombinant DNA or RNA, an artificial chromosome, an engineered genome, or fragment thereof, or a synthetic DNA, RNA, DNA/RNA hybrid, or including non-naturally occurring nucleotides or nucleosides.

Furthermore, the terms “nucleic acid,” “DNA,” “RNA,” and/or similar terms include nucleic acid analogs, e.g. analogs having other than a phosphodiester backbone. Nucleic acids may be purified from natural sources, produced using recombinant expression systems and optionally purified, chemically synthesized, etc. Where appropriate, e.g. in the case of chemically synthesized molecules, nucleic acids may comprise nucleoside analogs such as analogs having chemically modified bases or sugars, and backbone modifications. A nucleic acid sequence is presented in the 5′ to 3′ direction unless otherwise indicated. In some embodiments, a nucleic acid is or comprises natural nucleosides (e.g. adenosine, thymidine, guanosine, cytidine, uridine, deoxyadenosine, deoxythymidine, deoxyguanosine, and deoxycytidine); nucleoside analogs (e.g. 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, inosinedenosine, 8-oxoguanosine, O(6)-methylguanine, and 2-thiocytidine); chemically modified bases; biologically modified bases (e.g., methylated bases, such as 2′-O-methylated bases); intercalated bases; modified sugars (e.g. 2′-fluororibose, ribose, 2′-deoxyribose, arabinose, and hexose); and/or modified phosphate groups (e.g. phosphorothioates and 5′-N-phosphoramidite linkages).

The term “phage-assisted continuous evolution (PACE),” as used herein, refers to continuous evolution that employs phage as viral vectors. The general concept of PACE technology has been described, for example, in PCT Application No. PCT/US2009/056194, filed Sep. 8, 2009, published as WO 2010/028347 on Mar. 11, 2010; PCT Application No. PCT/US2011/066747, filed Dec. 22, 2011, published as WO 2012/088381 on Jun. 28, 2012; U.S. Application, U.S. Pat. No. 9,023,594, issued May 5, 2015, PCT Application No. PCT/US2015/012022, filed Jan. 20, 2015, published as WO 2015/134121 on Sep. 11, 2015, and PCT Application No. PCT/US2016/027795, filed Apr. 15, 2016, published as WO 2016/168631 on Oct. 20, 2016, the entire contents of each of which are incorporated herein by reference.

The term “promoter” is art-recognized and refers to a nucleic acid molecule with a sequence recognized by the cellular transcription machinery and able to initiate transcription of a downstream gene. A promoter may be constitutively active, meaning that the promoter is always active in a given cellular context, or conditionally active, meaning that the promoter is only active in the presence of a specific condition. For example, a conditional promoter may only be active in the presence of a specific protein that connects a protein associated with a regulatory element in the promoter to the basic transcriptional machinery, or only in the absence of an inhibitory molecule. A subclass of conditionally active promoters is inducible promoters that require the presence of a small molecule “inducer” for activity. Examples of inducible promoters include, but are not limited to, arabinose-inducible promoters, Tet-on promoters, and tamoxifen-inducible promoters. A variety of constitutive, conditional, and inducible promoters are well known to the skilled artisan, and the skilled artisan will be able to ascertain a variety of such promoters useful in carrying out the instant invention, which is not limited in this respect. In various embodiments, the disclosure provides vectors with appropriate promoters for driving expression of the nucleic acid sequences encoding the base editors (or one or more individual components thereof).

As used herein, the term “protospacer” refers to the sequence (e.g., a ˜20 bp sequence) in DNA adjacent to the PAM (protospacer adjacent motif) sequence which shares the same sequence as the spacer sequence of the guide RNA, and which is complementary to the target sequence of the non-PAM strand. The spacer sequence of the guide RNA anneals to the target sequence located on the non-PAM strand. In order for Cas9 to function it also requires a specific protospacer adjacent motif (PAM) that varies depending on the bacterial species of the Cas9 gene. The most commonly used Cas9 nuclease, derived from S. pyogenes, recognizes a PAM sequence of NGG that is found directly downstream of the protospacer sequence in the genomic DNA, on the non-target strand. The skilled person will appreciate that the literature in the state of the art sometimes refers to the “protospacer” as the ˜20-nt target-specific guide sequence on the guide RNA itself, rather than referring to it as a “spacer” (and that the protospacer (DNA) and the spacer (RNA) have the same sequence). Thus, the term “protospacer” as used herein may be used interchangeably with the term “spacer.” The context of the description surrounding the appearance of either “protospacer” or “spacer” will help inform the reader as to whether the term is reference to the gRNA or the DNA sequence. Both usages of these terms are acceptable since the state of the art uses both terms in each of these ways.

As used herein, the term “protospacer adjacent sequence” or “PAM” refers to an approximately 2-6 base pair DNA sequence that is an important targeting component of a Cas9 nuclease. Typically, the PAM sequence is on either strand, and is downstream in the 5′ to 3′ direction of Cas9 cut site. The canonical PAM sequence (i.e., the PAM sequence that is associated with the Cas9 nuclease of Streptococcus pyogenes or SpCas9) is 5′-NGG-3′ wherein “N” is any nucleobase followed by two guanine (“G”) nucleobases. Different PAM sequences can be associated with different Cas9 nucleases or equivalent proteins from different organisms. In addition, any given Cas9 nuclease, e.g., SpCas9, may be modified to alter the PAM specificity of the nuclease such that the nuclease recognizes alternative PAM sequence.

For example, with reference to the canonical SpCas9 amino acid sequence is SEQ ID NO: 74, the PAM sequence can be modified by introducing one or more mutations, including (a) D1135V, R1335Q, and T1337R “the VRQR variant”, which alters the PAM specificity to NGAN or NGNG, (b) D1135E, R1335Q, and T1337R “the EQR variant”, which alters the PAM specificity to NGAG, and (c) D1135V, G1218R, R1335E, and T1337R “the VRER variant”, which alters the PAM specificity to NGCG. In addition, the D1135E variant of canonical SpCas9 still recognizes NGG, but it is more selective compared to the wild type SpCas9 protein.

It will also be appreciated that Cas9 enzymes from different bacterial species (i.e., Cas9 orthologs) can have varying PAM specificities. For example, Cas9 from Staphylococcus aureus (SaCas9) recognizes NGRRT or NGRRN. In addition, Cas9 from Neisseria meningitis (NmeCas) recognizes NNNNGATT. A Cas9 from Staphylococcus auricularis (SauriCas9) recognizes NNGG and NNNGG. A Cas9 from Streptococcus thermophilis (StCas9) recognizes NNAGAAW. A Cas9 from Treponema denticola (TdCas) recognizes NAAAAC. These are example are not meant to be limiting. It will be further appreciated that non-SpCas9s bind a variety of PAM sequences, which makes them useful when no suitable SpCas9 PAM sequence is present at the desired target cut site. Furthermore, non-SpCas9s may have other characteristics that make them more useful than SpCas9. For example, Cas9 from Staphylococcus aureus (SaCas9) is about 1 kilobase smaller than SpCas9, so it can be packaged into adeno-associated virus (AAV). Further reference may be made to Shah et al., “Protospacer recognition motifs: mixed identities and functional diversity,” RNA Biology, 10 (5): 891-899 (which is incorporated herein by reference).

The terms “protein,” “peptide,” and “polypeptide” are used interchangeably herein, and refer to a polymer of amino acid residues linked together by peptide (amide) bonds. The terms refer to a protein, peptide, or polypeptide of any size, structure, or function. Typically, a protein, peptide, or polypeptide will be at least three amino acids long. A protein, peptide, or polypeptide may refer to an individual protein or a collection of proteins. One or more of the amino acids in a protein, peptide, or polypeptide may be modified, for example, by the addition of a chemical entity such as a carbohydrate group, a hydroxyl group, a phosphate group, a farnesyl group, an isofarnesyl group, a fatty acid group, a linker for conjugation, functionalization, or other modification, etc. A protein, peptide, or polypeptide may also be a single molecule or may be a multi-molecular complex. A protein, peptide, or polypeptide may be just a fragment of a naturally occurring protein or peptide. A protein, peptide, or polypeptide may be naturally occurring, recombinant, or synthetic, or any combination thereof. It should be appreciated that the disclosure provides any of the polypeptide sequences provided herein without an N-terminal methionine (M) residue.

In genetics, a “sense” strand is the segment within double-stranded DNA that runs from 5′ to 3′, and which is complementary to the antisense strand of DNA, or template strand, which runs from 3′ to 5′. In the case of a DNA segment that encodes a protein, the sense strand is the strand of DNA that has the same sequence as the mRNA, which takes the antisense strand as its template during transcription, and eventually undergoes (typically, not always) translation into a protein. The antisense strand is thus responsible for the RNA that is later translated to protein, while the sense strand possesses a nearly identical makeup to that of the mRNA. Note that for each segment of dsDNA, there will possibly be two sets of sense and antisense, depending on which direction one reads (since sense and antisense is relative to perspective). It is ultimately the gene product, or mRNA, that dictates which strand of one segment of dsDNA is referred to as sense or antisense.

A “split Cas9 protein” or “split Cas9” refers to a Cas9 protein that is provided as an N-terminal portion (which is referred to herein interchangeably as an N-terminal half) and a C-terminal portion (which is referred to herein interchangeably as a C-terminal half) encoded by two separate nucleotide sequences. The polypeptides corresponding to the N-terminal portion and the C-terminal portion of the Cas9 protein may be combined (joined) to form a complete Cas9 protein. A Cas9 protein is known to consist of a bi-lobed structure linked by a disordered linker (e.g., as described in Nishimasu et al., Cell, Volume 156, Issue 5, pp. 935-949, 2014, incorporated herein by reference). In some embodiments, the “split” occurs between the two lobes, generating two portions of a Cas9 protein, each containing one lobe.

The term “subject,” as used herein, refers to an individual organism, for example, an individual mammal. In some embodiments, the subject is a human. In some embodiments, the subject is a non-human mammal. In some embodiments, the subject is a non-human primate. In some embodiments, the subject is a rodent. In some embodiments, the subject is a sheep, a goat, cattle, a cat, or a dog. In some embodiments, the subject is a vertebrate, an amphibian, a reptile, a fish, an insect, a fly, or a nematode. In some embodiments, the subject is a research animal. In some embodiments, the subject is genetically engineered, e.g., a genetically engineered non-human subject. The subject may be of either sex and at any stage of development. In some embodiments, the subject is a domesticated animal. In some embodiments, the subject is a plant.

The term “target site” refers to a sequence within a nucleic acid molecule that is edited by a base editor (BE) disclosed herein. The term “target site,” in the context of a single strand, also can refer to the “target strand” which anneals or binds to the spacer sequence of the guide RNA. The target site can refer, in certain embodiments, to a segment of double-stranded DNA that includes the protospacer (i.e., the strand of the target site that has the same nucleotide sequence as the spacer sequence of the guide RNA) on the PAM-strand (or non-target strand) and target strand, which is complementary to the protospacer and the spacer alike, and which anneals to the spacer of the guide RNA, thereby targeting or programming a Cas9 base editor to target the target site.

A “transcriptional terminator” is a nucleic acid sequence that causes transcription to stop. A transcriptional terminator may be unidirectional or bidirectional. It is comprised of a DNA sequence involved in specific termination of an RNA transcript by an RNA polymerase. A transcriptional terminator sequence prevents transcriptional activation of downstream nucleic acid sequences by upstream promoters. A transcriptional terminator may be necessary in vivo to achieve desirable expression levels or to avoid transcription of certain sequences. A transcriptional terminator is considered to be “operably linked to” a nucleotide sequence when it is able to terminate the transcription of the sequence it is linked to.

In eukaryotic systems, the terminator region may comprise specific DNA sequences that permit site-specific cleavage of the new transcript so as to expose a polyadenylation site. This signals a specialized endogenous polymerase to add a stretch of about 200 A residues (polyA) to the 3′ end of the transcript. RNA molecules modified with this polyA tail (signal) appear to more stable and are translated more efficiently. Thus, in some embodiments involving eukaryotes, a terminator may comprise a signal for the cleavage of the RNA. In some embodiments, the terminator signal promotes polyadenylation of the message. The terminator and/or polyadenylation site elements may serve to enhance output nucleic acid levels and/or to minimize read through between nucleic acids.

In some embodiments, the transcriptional terminator contains a posttranscriptional response element, a sequence that, when transcribed, creates a tertiary structure enhancing expression. In some embodiments, the posttranscriptional response element is derived from woodchuck hepatitis virus (WHV), i.e., is a WPRE. In some embodiments, the terminator contains the gamma subunit of a WPRE, or a W3, as first reported in Choi, J. H., et al. (2014), Mol. Brain 7:17, incorporated herein by reference. The WPRE also has alpha and beta subunits. Typically, the posttranscriptional response element is inserted 5′ of the transcriptional terminator. In certain embodiments, the WPRE is a truncated WPRE sequence. In certain embodiments, the WPRE is a full-length WPRE.

Terminators for use in accordance with the present disclosure include any terminator of transcription described herein or known to one of ordinary skill in the art. Examples of terminators include, without limitation, the termination sequences of genes such as, for example, the bovine growth hormone terminator, and viral termination sequences such as, for example, the SV40 terminator and bGH terminator. In some embodiments, the termination signal may be a sequence that cannot be transcribed or translated, such as those resulting from a sequence truncation.

As used herein, “transitions” refer to the interchange of purine nucleobases (A↔G) or the interchange of pyrimidine nucleobases (C↔T). This class of interchanges involves nucleobases of similar shape. The compositions and methods disclosed herein are capable of inducing one or more transitions in a target DNA molecule. The compositions and methods disclosed herein are also capable of inducing both transitions and transversion in the same target DNA molecule. These changes involve A↔G, G↔A, C↔T, or T↔C. In the context of a double-strand DNA with Watson-Crick paired nucleobases, transitions refer to the following base pair exchanges: A:T↔G:C, G:G↔A:T, C:G↔T:A, or T:A↔C:G. The compositions and methods disclosed herein are capable of inducing one or more transitions in a target DNA molecule. The compositions and methods disclosed herein are also capable of inducing both transitions and transversion in the same target DNA molecule, as well as other nucleotide changes, including deletions and insertions.

As used herein, “transversions” refer to the interchange of purine nucleobases for pyrimidine nucleobases, or in the reverse and thus, involve the interchange of nucleobases with dissimilar shape. These changes involve T↔A, T↔G, C↔G, C↔A, A↔T, A↔C, G↔C, and G↔T. In the context of a double-strand DNA with Watson-Crick paired nucleobases, transversions refer to the following base pair exchanges: T:A↔A:T, T:A↔G:C, C:G↔+G:C, C:G↔A:T, A:T↔T:A, A:T↔C:G, G:C↔C:G, and G:C↔T:A.

The terms “treatment,” “treat,” and “treating,” refer to a clinical intervention aimed to reverse, alleviate, delay the onset of, or inhibit the progress of a disease or disorder, or one or more symptoms thereof, as described herein. As used herein, the terms “treatment,” “treat,” and “treating” refer to a clinical intervention aimed to reverse, alleviate, delay the onset of, or inhibit the progress of a disease or disorder, or one or more symptoms thereof, as described herein. In some embodiments, treatment may be administered after one or more symptoms have developed and/or after a disease has been diagnosed. In other embodiments, treatment may be administered in the absence of symptoms, e.g., to prevent or delay onset of a symptom or inhibit onset or progression of a disease. For example, treatment may be administered to a susceptible individual prior to the onset of symptoms (e.g., in light of a history of symptoms and/or in light of genetic or other susceptibility factors). Treatment may also be continued after symptoms have resolved, for example, to prevent or delay their recurrence.

As used herein, the terms “upstream” and “downstream” are terms of relativity that define the linear position of at least two elements located in a nucleic acid molecule (whether single or double-stranded) that is orientated in a 5′-to-3′ direction. In particular, a first element is upstream of a second element in a nucleic acid molecule where the first element is positioned somewhere that is 5′ to the second element. For example, a SNP is upstream of a Cas9-induced nick site if the SNP is on the 5′ side of the nick site. Conversely, a first element is downstream of a second element in a nucleic acid molecule where the first element is positioned somewhere that is 3′ to the second element. For example, a SNP is downstream of a Cas9-induced nick site if the SNP is on the 3′ side of the nick site. The nucleic acid molecule can be a DNA (double or single stranded). RNA (double or single stranded), or a hybrid of DNA and RNA. The analysis is the same for single strand nucleic acid molecule and a double strand molecule since the terms upstream and downstream are in reference to only a single strand of a nucleic acid molecule, except that one needs to select which strand of the double stranded molecule is being considered. Often, the strand of a double stranded DNA which can be used to determine the positional relativity of at least two elements is the “sense” or “coding” strand. In genetics, a “sense” strand is the segment within double-stranded DNA that runs from 5′ to 3′, and which is complementary to the antisense strand of DNA, or template strand, which runs from 3′ to 5′. Thus, as an example, a SNP nucleobase is “downstream” of a promoter sequence in a genomic DNA (which is double-stranded) if the SNP nucleobase is on the 3′ side of the promoter on the sense or coding strand.

As used herein, the term “variant” refers to a protein having characteristics that deviate from what occurs in nature that retains at least one functional i.e. binding, interaction, or enzymatic ability and/or therapeutic property thereof. A “variant” is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to the wild type protein. For instance, a variant of Cas9 may comprise a Cas9 that has one or more changes in amino acid residues as compared to a wild type Cas9 amino acid sequence. As another example, a variant of a deaminase may comprise a deaminase that has one or more changes in amino acid residues as compared to a wild type deaminase amino acid sequence, e.g. following ancestral sequence reconstruction of the deaminase. These changes include chemical modifications, including substitutions of different amino acid residues truncations, covalent additions (e.g. of a tag), and any other mutations. The term also encompasses circular permutants, mutants, truncations, or domains of a reference sequence, and which display the same or substantially the same functional activity or activities as the reference sequence. This term also embraces fragments of a wild type protein.

The level or degree of which the property is retained may be reduced relative to the wild type protein but is typically the same or similar in kind. Generally, variants are overall very similar, and in many regions, identical to the amino acid sequence of the protein described herein. A skilled artisan will appreciate how to make and use variants that maintain all, or at least some, of a functional ability or property. The variant proteins may comprise, or alternatively consist of, an amino acid sequence which is at least 80%, 85%, 90%, 95%, 96%, 97%, 98%, 99%, or 100%, identical to, for example, the amino acid sequence of a wild-type protein, or any protein provided herein.

By a polypeptide having an amino acid sequence at least, for example, 95% “identical” to a query amino acid sequence, it is intended that the amino acid sequence of the subject polypeptide is identical to the query sequence except that the subject polypeptide sequence may include up to five amino acid alterations per each 100 amino acids of the query amino acid sequence. In other words, to obtain a polypeptide having an amino acid sequence at least 95% identical to a query amino acid sequence, up to 5% of the amino acid residues in the subject sequence may be inserted, deleted, or substituted with another amino acid. These alterations of the reference sequence may occur at the amino- or carboxy-terminal positions of the reference amino acid sequence or anywhere between those terminal positions, interspersed either individually among residues in the reference sequence or in one or more contiguous groups within the reference sequence.

As a practical matter, whether any particular polypeptide is at least 80%, 85%, 90%, 95%, 96%, 97%, 98%, or 99% identical to, for instance, the amino acid sequence of a fusion protein, can be determined conventionally using known computer programs. A preferred method for determining the best overall match between a query sequence (a sequence of the present invention) and a subject sequence, also referred to as a global sequence alignment, can be determined using the FASTDB computer program based on the algorithm of Brutlag et al. (Comp. App. Biosci. 6:237-245 (1990)). In a sequence alignment the query and subject sequences are either both nucleotide sequences or both amino acid sequences. The result of said global sequence alignment is expressed as percent identity. Preferred parameters used in a FASTDB amino acid alignment are: Matrix=PAM 0, k-tuple=2, Mismatch Penalty=1, Joining Penalty=20, Randomization Group Length=0, Cutoff Score=1, Window Size=sequence length, Gap Penalty=5, Gap Size Penalty=0.05, Window Size=500 or the length of the subject amino acid sequence, whichever is shorter.

If the subject sequence is shorter than the query sequence due to N- or C-terminal deletions, not because of internal deletions, a manual correction must be made to the results. This is because the FASTDB program does not account for N- and C-terminal truncations of the subject sequence when calculating global percent identity. For subject sequences truncated at the N- and C-termini, relative to the query sequence, the percent identity is corrected by calculating the number of residues of the query sequence that are N- and C-terminal of the subject sequence, which are not matched/aligned with a corresponding subject residue, as a percent of the total bases of the query sequence. Whether a residue is matched/aligned is determined by results of the FASTDB sequence alignment. This percentage is then subtracted from the percent identity, calculated by the above FASTDB program using the specified parameters, to arrive at a final percent identity score. This final percent identity score is what is used for the purposes of the present invention. Only residues to the N- and C-termini of the subject sequence, which are not matched/aligned with the query sequence, are considered for the purposes of manually adjusting the percent identity score. That is, only query residue positions outside the farthest N- and C-terminal residues of the subject sequence.

The term “vector,” as used herein, refers to a nucleic acid that can be modified to encode a gene of interest and that is able to enter into a host cell and replicate within the host cell, and then transfer a replicated form of the vector into another host cell. Exemplary suitable vectors include viral vectors, such as AAV vectors or bacteriophages and filamentous phage, and conjugative plasmids. Additional suitable vectors will be apparent to those of skill in the art based on the present disclosure.

As used herein the term “wild type” is a term of the art understood by skilled persons and means the typical form of an organism, strain, gene or characteristic as it occurs in nature as distinguished from mutant or variant forms.

napDNAbp Domains

The base editors described herein comprise a nucleic acid programmable DNA binding (napDNAbp) domain. The napDNAbp is associated with at least one guide nucleic acid (e.g., guide RNA), which localizes the napDNAbp to a DNA sequence that comprises a DNA strand (i.e., a target strand) that is complementary to the guide nucleic acid, or a portion thereof (e.g., the protospacer of a guide RNA). In other words, the guide nucleic-acid “programs” the napDNAbp domain to localize and bind to a complementary sequence of the target strand. Binding of the napDNAbp domain to a complementary sequence enables the nucleobase modification domain (i.e., the adenosine deaminase domain) of the base editor to access and enzymatically deaminate a target base in the target strand.

The napDNAbp can be a CRISPR (clustered regularly interspaced short palindromic repeat)-associated nuclease. As outlined above, CRISPR is an adaptive immune system that provides protection against mobile genetic elements (viruses, transposable elements and conjugative plasmids). CRISPR clusters contain spacers, sequences complementary to antecedent mobile elements, and target invading nucleic acids. CRISPR clusters are transcribed and processed into CRISPR RNA (crRNA). In type II CRISPR systems correct processing of pre-crRNA requires a trans-encoded small RNA (tracrRNA), endogenous ribonuclease 3 (rnc) and a Cas9 protein. The tracrRNA serves as a guide for ribonuclease 3-aided processing of pre-crRNA. Subsequently, Cas9/crRNA/tracrRNA endonucleolytically cleaves linear or circular dsDNA target complementary to the spacer. The target strand not complementary to crRNA is first cut endonucleolytically, then trimmed 3′-5′ exonucleolytically. In nature, DNA-binding and cleavage typically requires protein and both RNAs. However, single guide RNAs (“sgRNA”, or simply “gNRA”) can be engineered so as to incorporate aspects of both the crRNA and tracrRNA into a single RNA species. See, e.g., Jinek et al., Science 337:816-821 (2012), the entire contents of which is hereby incorporated by reference.

The below description of various napDNAbps which can be used in connection with the disclosed adenosine deaminases is not meant to be limiting in any way. The base editors may comprise the canonical SpCas9, or any ortholog Cas9 protein, or any variant Cas9 protein-including any naturally occurring variant, mutant, or otherwise engineered version of Cas9—that is known or which can be made or evolved through a directed evolutionary or otherwise mutagenic process. In various embodiments, the napDNAbp has a nickase activity, i.e., only cleave one strand of the target DNA sequence. In other embodiments, the napDNAbp has an inactive nuclease, e.g., are “dead” proteins. Other variant Cas9 proteins that may be used are those having a smaller molecular weight than the canonical SpCas9 (e.g., for easier delivery) or having modified or rearranged primary amino acid sequence (e.g., the circular permutant forms). The base editors described herein may also comprise Cas9 equivalents, including Cas12a/Cpf1 proteins. The napDNAbps used herein (e.g., SpCas9, SaCas9, or SaCas9 variant or SpCas9 variant) may also may also contain various modifications that alter/enhance their PAM specifities. The disclosure contemplates any Cas9, Cas9 variant, or Cas9 equivalent which has at least 70%, at least 75%, at least 80%, at least 85%, at least 90%, at least 91%, at least 92%, at least 93%, at least 94%, at least 95%, at least 96%, at least 97%, at least 98%, at least 99%, or at least 99.9% sequence identity to any of the Cas9 proteins disclosed herein. In some embodiments, the napDNAbp domain comprises a nickase variant of a wild-type Cas9. In some embodiments, the napDNAbp domain comprises any of the Cas9 nickases disclosed herein.

In some embodiments, the napDNAbp directs cleavage of one or both strands at the location of a target sequence, such as within the target sequence and/or within the complement of the target sequence. In some embodiments, the napDNAbp directs cleavage of one or both strands within about 1, 2, 3, 4, 5, 6, 7, 8, 9, 10, 15, 20, 25, 50, 100, 200, 500, or more base pairs from the first or last nucleotide of a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). Other examples of mutations that render Cas9 a nickase include, without limitation, H840A, N854A, and N863A in reference to the canonical SpCas9 sequence, or to equivalent amino acid positions in other Cas9 variants or Cas9 equivalents.

As used herein, the term “Cas protein” refers to a full-length Cas protein obtained from nature, a recombinant Cas protein having a sequences that differs from a naturally occurring Cas protein, or any fragment of a Cas protein that nevertheless retains all or a significant amount of the requisite basic functions needed for the disclosed methods, i.e., (i) possession of nucleic-acid programmable binding of the Cas protein to a target DNA, and (ii) ability to nick the target DNA sequence on one strand. The Cas proteins contemplated herein embrace CRISPR Cas9 proteins, as well as Cas9 equivalents, variants (e.g., Cas9 nickase (nCas9) or nuclease inactive Cas9 (dCas9)) homologs, orthologs, or paralogs, whether naturally occurring or non-naturally occurring (e.g., engineered or recombinant), and may include a Cas9 equivalent from any type of CRISPR system (e.g., type II, V, VI), including Cpf1 (a type-V CRISPR-Cas systems), C2c1 (a type V CRISPR-Cas system), C2c2 (a type VI CRISPR-Cas system) and C2c3 (a type V CRISPR-Cas system). Further Cas-equivalents are described in Makarova et al., “C2c2 is a single-component programmable RNA-guided RNA-targeting CRISPR effector,” Science 2016; 353 (6299), the contents of which are incorporated herein by reference.

The term “Cas9” or “Cas9 domain” embraces any naturally occurring Cas9 from any organism, any naturally-occurring Cas9 equivalent or functional fragment thereof, any Cas9 homolog, ortholog, or paralog from any organism, and any mutant or variant of a Cas9, naturally-occurring or engineered. The term Cas9 is not meant to be particularly limiting and may be referred to as a “Cas9 or equivalent.” Exemplary Cas9 proteins are further described herein and/or are described in the art and are incorporated herein by reference. The present disclosure is unlimited with regard to the particular napDNAbp that is employed in the base editors of the disclosure.

As used herein, the terms “compact Cas9 protein”, “compact napDNAbp” and “compact variant [of a Cas protein]” refers to a Cas9 protein or variant that has an amino acid length of less than about 1250 amino acids. In some embodiments, a compact Cas9 protein or compact napDNAbp contains less than 1250 amino acids, less than 1240 amino acids, less than 1230 amino acids, less than 1220 amino acids, less than 1210 amino acids, less than 1200 amino acids, less than 1190 amino acids, less than 1180 amino acids, less than 1170 amino acids, less than 1160 amino acids, less than 1150 amino acids, less than 1140 amino acids, less than 1130 amino acids, less than 1120 amino acids, less than 1110 amino acids, less than 1100 amino acids, less than 1050 amino acids, less than 1000 amino acids, less than 950 amino acids, less than 900 amino acids, less than 850 amino acids, less than 800 amino acids, less than 750 amino acids, less than 700 amino acids, less than 650 amino acids, less than 600 amino acids, less than 550 amino acids, or less than 500 amino acids in length. These terms also embrace any Cas9 protein or variant encoded by a nucleic acid sequence having a length of less than about 3750 nucleotides. The base editors of the disclosure may comprise compact napDNAbps and/or compact Cas9 proteins. In some embodiments, the compact Cas9 protein is about 350 amino acids shorter than a SpCas9. In some embodiments, the compact Cas9 protein is about 1000 amino acids in length. In some embodiments, the compact protein is a compact variant of S. pyogenes Cas9 (SpCas9), Cpf1, CasX, CasY, C2c1, C2c2, C2c3, Cas12a, Cas12b, Cas12g, Cas12h, Cas12i, Cas13b, Cas13c, Cas13d, Cas14, Csn2, Cas3, or CasΦ. A “compact variant” may refer to a Cas9 protein hat has one or more truncations, or one or more deletions, relative to a wild-type Cas9 protein, such as a wild-type SpCas9 or Cpf1.

Additional Cas9 sequences and structures are well known to those of skill in the art (see, e.g., “Complete genome sequence of an MI strain of Streptococcus pyogenes.” Ferretti et al., J. J., McShan W. M., Ajdic D. J., Savic D. J., Savic G., Lyon K., Primeaux C., Sezate S., Suvorov A. N., Kenton S., Lai H. S., Lin S. P., Qian Y., Jia H. G., Najar F. Z., Ren Q., Zhu H., Song L., White J., Yuan X., Clifton S. W., Roc B. A., Mclaughlin R. E., Proc. Natl. Acad. Sci. U.S.A. 98:4658-4663 (2001); “CRISPR RNA maturation by trans-encoded small RNA and host factor RNase III.” Deltcheva E., Chylinski K., Sharma C. M., Gonzales K., Chao Y., Pirzada Z. A., Eckert M. R., Vogel J., Charpentier E., Nature 471:602-607 (2011); and “A programmable dual-RNA-guided DNA endonuclease in adaptive bacterial immunity.” Jinek M., Chylinski K., Fonfara I., Hauer M., Doudna J. A., Charpentier E. Science 337:816-821 (2012), the entire contents of each of which are incorporated herein by reference), and also provided below.

Examples of Cas9 and Cas9 equivalents are provided as follows; however, these specific examples are not meant to be limiting. The base editors of the present disclosure may use any suitable napDNAbp, including any suitable Cas9 or Cas9 equivalent.

napDNAbp Nickases

In some embodiments, the disclosed base editors may comprise a napDNAbp domain that comprises a nickase. In some embodiments, the base editors described herein comprise a Cas9 nickase. The term “Cas9 nickase” of “nCas9” refers to a variant of Cas9 which is capable of introducing a single-strand break in a double strand DNA molecule target. In some embodiments, the Cas9 nickase comprises only a single functioning nuclease domain. The wild type Cas9 (e.g., the canonical SpCas9) comprises two separate nuclease domains, namely, the RuvC domain (which cleaves the non-protospacer DNA strand) and HNH domain (which cleaves the protospacer DNA strand). In one embodiment, the Cas9 nickase comprises a mutation in the RuvC domain which inactivates the RuvC nuclease activity. For example, mutations in aspartate (D) 10, histidine (H) 983, aspartate (D) 986, or glutamate (E) 762, have been reported as loss-of-function mutations of the RuvC nuclease domain and the creation of a functional Cas9 nickase (e.g., Nishimasu et al., “Crystal structure of Cas9 in complex with guide RNA and target DNA,” Cell 156 (5), 935-949, which is incorporated herein by reference). Thus, nickase mutations in the RuvC domain could include D10X, H983X, D986X, or E762X, wherein X is any amino acid other than the wild type amino acid. In certain embodiments, the nickase could be D10A, of H983A, or D986A, or E762A, or a combination thereof.

In some embodiments, the napDNAbp domain of any of the disclosed base editors comprises an S. pyogenes Cas9 nickase (SpCas9n). In some embodiments, the napDNAbp domain of any of the disclosed based editors is comprises at least 80%, at least 85%, at least 90%, at least 95%, or at least 99% sequence identity to SEQ ID NO: 365 or 370. In some embodiments, the napDNAbp domain of any of the disclosed base editors comprises the amino acid sequence of SEQ ID NO: 365. In some embodiments, the napDNAbp domain of any of the disclosed base editors comprises the amino acid sequence of SEQ ID NO: 370.

In some embodiments, the napDNAbp domain of any of the disclosed base editors comprises an S. aureus Cas9 nickase (SaCas9n). In some embodiments, the napDNAbp domain of any of the disclosed based editors is comprises at least 80%, at least 85%, at least 90%, at least 95%, or at least 99% sequence identity to SEQ ID NO: 438. In some embodiments, the napDNAbp domain of any of the disclosed base editors comprises the amino acid sequence of SEQ ID NO: 438.

In various embodiments, the Cas9 nickase can having a mutation in the RuvC nuclease domain and have one of the following amino acid sequences, or a variant thereof having an amino acid sequence that has at least 80%, at least 85%, at least 90%, at least 95%, or at least 99% sequence identity thereto.

| Description | Sequence | SEQ ID NO: |
|---|---|---|
| Cas9 nickase | MDKKYSIGLXIGTNSVGWAVITDEYKVPSKKFKVLGNTDRHSIKKNLIGALLF | SEQ ID NO: 361 |
| Streptococcus | DSGETAEATRLKRTARRRYTRRKNRICYLQEIFSNEMAKVDDSFFHRLEESFLV | |
| pyogenes | EEDKKHERHPIFGNIVDEVAYHEKYPTIYHLRKKLVDSTDKADLRLIYLALAH | |
| Q99ZW2 Cas9 | MIKFRGHFLIEGDLNPDNSDVDKLFIQLVQTYNQLFEENPINASGVDAKAILSA | |
| with D10X, | RLSKSRRLENLIAQLPGEKKNGLFGNLIALSLGLTPNFKSNFDLAEDAKLQLSK | |
| wherein X is any | DTYDDDLDNLLAQIGDQYADLFLAAKNLSDAILLSDILRVNTEITKAPLSASMI | |
| alternate amino | KRYDEHHQDLTLLKALVRQQLPEKYKEIFFDQSKNGYAGYIDGGASQEEFYK | |
| acid | FIKPILEKMDGTEELLVKLNREDLLRKQRTFDNGSIPHQIHLGELHAILRRQED | |
| | FYPFLKDNREKIEKILTFRIPYYVGPLARGNSRFAWMTRKSEETITPWNFEEVV | |
| | DKGASAQSFIERMTNFDKNLPNEKVLPKHSLLYEYFTVYNELTKVKYVTEGM | |
| | RKPAFLSGEQKKAIVDLLFKTNRKVTVKQLKEDYFKKIECFDSVEISGVEDRF | |
| | NASLGTYHDLLKIIKDKDFLDNEENEDILEDIVLTLTLFEDREMIEERLKTYAH | |
| | LFDDKVMKQLKRRRYTGWGRLSRKLINGIRDKQSGKTILDFLKSDGFANRNF | |
| | MQLIHDDSLTFKEDIQKAQVSGQGDSLHEHIANLAGSPAIKKGILQTVKVVDE | |
| | LVKVMGRHKPENIVIEMARENQTTQKGQKNSRERMKRIEEGIKELGSQILKEH | |
| | PVENTQLQNEKLYLYYLQNGRDMYVDQELDINRLSDYDVDHIVPQSFLKDDS | |
| | IDNKVLTRSDKNRGKSDNVPSEEVVKKMKNYWRQLLNAKLITQRKFDNLTK | |
| | AERGGLSELDKAGFIKRQLVETRQITKHVAQILDSRMNTKYDENDKLIREVKV | |
| | ITLKSKLVSDFRKDFQFYKVREINNYHHAHDAYLNAVVGTALIKKYPKLESEF | |
| | VYGDYKVYDVRKMIAKSEQEIGKATAKYFFYSNIMNFFKTEITLANGEIRKRP | |
| | LIETNGETGEIVWDKGRDFATVRKVLSMPQVNIVKKTEVQTGGFSKESILPKR | |
| | NSDKLIARKKDWDPKKYGGFDSPTVAYSVLVVAKVEKGKSKKLKSVKELLGI | |
| | TIMERSSFEKNPIDFLEAKGYKEVKKDLIIKLPKYSLFELENGRKRMLASAGEL | |
| | QKGNELALPSKYVNFLYLASHYEKLKGSPEDNEQKQLFVEQHKHYLDEIIEQI | |
| | SEFSKRVILADANLDKVLSAYNKHRDKPIREQAENIIHLFTLTNLGAPAAFKYF | |
| | DTTIDRKRYTSTKEVLDATLIHQSITGLYETRIDLSQLGGD | |
| Cas9 nickase | MDKKYSIGLDIGTNSVGWAVITDEYKVPSKKFKVLGNTDRHSIKKNLIGALLF | SEQ ID NO: 362 |
| Streptococcus | DSGETAEATRLKRTARRRYTRRKNRICYLQEIFSNEMAKVDDSFFHRLEESFLV | |
| pyogenes | EEDKKHERHPIFGNIVDEVAYHEKYPTIYHLRKKLVDSTDKADLRLIYLALAH | |
| Q99ZW2 Cas9 | MIKFRGHFLIEGDLNPDNSDVDKLFIQLVQTYNQLFEENPINASGVDAKAILSA | |
| with E762X, | RLSKSRRLENLIAQLPGEKKNGLFGNLIALSLGLTPNFKSNFDLAEDAKLQLSK | |
| wherein X is any | DTYDDDLDNLLAQIGDQYADLFLAAKNLSDAILLSDILRVNTEITKAPLSASMI | |
| alternate amino | KRYDEHHQDLTLLKALVRQQLPEKYKEIFFDQSKNGYAGYIDGGASQEEFYK | |
| acid | FIKPILEKMDGTEELLVKLNREDLLRKQRTFDNGSIPHQIHLGELHAILRRQED | |
| | FYPFLKDNREKIEKILTFRIPYYVGPLARGNSRFAWMTRKSEETITPWNFEEVV | |
| | DKGASAQSFIERMTNFDKNLPNEKVLPKHSLLYEYFTVYNELTKVKYVTEGM | |
| | RKPAFLSGEQKKAIVDLLFKTNRKVTVKQLKEDYFKKIECFDSVEISGVEDRF | |
| | NASLGTYHDLLKIIKDKDFLDNEENEDILEDIVLTLTLFEDREMIEERLKTYAH | |
| | LFDDKVMKQLKRRRYTGWGRLSRKLINGIRDKQSGKTILDFLKSDGFANRNF | |
| | MQLIHDDSLTFKEDIQKAQVSGQGDSLHEHIANLAGSPAIKKGILQTVKVVDE | |
| | LVKVMGRHKPENIVIXMARENQTTQKGQKNSRERMKRIEEGIKELGSQILKE | |
| | HPVENTQLQNEKLYLYYLQNGRDMYVDQELDINRLSDYDVDHIVPQSFLKD | |
| | DSIDNKVLTRSDKNRGKSDNVPSEEVVKKMKNYWRQLLNAKLITQRKFDNL | |
| | TKAERGGLSELDKAGFIKRQLVETRQITKHVAQILDSRMNTKYDENDKLIREV | |
| | KVITLKSKLVSDFRKDFQFYKVREINNYHHAHDAYLNAVVGTALIKKYPKLES | |
| | EFVYGDYKVYDVRKMIAKSEQEIGKATAKYFFYSNIMNFFKTEITLANGEIRK | |
| | RPLIETNGETGEIVWDKGRDFATVRKVLSMPQVNIVKKTEVQTGGFSKESILP | |
| | KRNSDKLIARKKDWDPKKYGGFDSPTVAYSVLVVAKVEKGKSKKLKSVKEL | |
| | LGITIMERSSFEKNPIDFLEAKGYKEVKKDLIIKLPKYSLFELENGRKRMLASA | |
| | GELQKGNELALPSKYVNFLYLASHYEKLKGSPEDNEQKQLFVEQHKHYLDEI | |
| | IEQISEFSKRVILADANLDKVLSAYNKHRDKPIREQAENIIHLFTLTNLGAPAAF | |
| | KYFDTTIDRKRYTSTKEVLDATLIHQSITGLYETRIDLSQLGGD | |
| Cas9 nickase | MDKKYSIGLDIGTNSVGWAVITDEYKVPSKKFKVLGNTDRHSIKKNLIGALLF | SEQ ID NO: 363 |
| Streptococcus | DSGETAEATRLKRTARRRYTRRKNRICYLQEIFSNEMAKVDDSFFHRLEESFLV | |
| pyogenes | EEDKKHERHPIFGNIVDEVAYHEKYPTIYHLRKKLVDSTDKADLRLIYLALAH | |
| Q99ZW2 Cas9 | MIKFRGHFLIEGDLNPDNSDVDKLFIQLVQTYNQLFEENPINASGVDAKAILSA | |
| with H983X, | RLSKSRRLENLIAQLPGEKKNGLFGNLIALSLGLTPNFKSNFDLAEDAKLQLSK | |
| wherein X is any | DTYDDDLDNLLAQIGDQYADLFLAAKNLSDAILLSDILRVNTEITKAPLSASMI | |
| alternate amino | KRYDEHHQDLTLLKALVRQQLPEKYKEIFFDQSKNGYAGYIDGGASQEEFYK | |
| acid | FIKPILEKMDGTEELLVKLNREDLLRKQRTFDNGSIPHQIHLGELHAILRRQED | |
| | FYPFLKDNREKIEKILTFRIPYYVGPLARGNSRFAWMTRKSEETITPWNFEEVV | |
| | DKGASAQSFIERMTNFDKNLPNEKVLPKHSLLYEYFTVYNELTKVKYVTEGM | |
| | RKPAFLSGEQKKAIVDLLFKTNRKVTVKQLKEDYFKKIECFDSVEISGVEDRF | |
| | NASLGTYHDLLKIIKDKDFLDNEENEDILEDIVLTLTLFEDREMIEERLKTYAH | |
| | LFDDKVMKQLKRRRYTGWGRLSRKLINGIRDKQSGKTILDFLKSDGFANRNF | |
| | MQLIHDDSLTFKEDIQKAQVSGQGDSLHEHIANLAGSPAIKKGILQTVKVVDE | |
| | LVKVMGRHKPENIVIEMARENQTTQKGQKNSRERMKRIEEGIKELGSQILKEH | |
| | PVENTQLQNEKLYLYYLQNGRDMYVDQELDINRLSDYDVDHIVPQSFLKDDS | |
| | IDNKVLTRSDKNRGKSDNVPSEEVVKKMKNYWRQLLNAKLITQRKFDNLTK | |
| | AERGGLSELDKAGFIKRQLVETRQITKHVAQILDSRMNTKYDENDKLIREVKV | |
| | ITLKSKLVSDFRKDFQFYKVREINNYHXAHDAYLNAVVGTALIKKYPKLESEF | |
| | VYGDYKVYDVRKMIAKSEQEIGKATAKYFFYSNIMNFFKTEITLANGEIRKRP | |
| | LIETNGETGEIVWDKGRDFATVRKVLSMPQVNIVKKTEVQTGGFSKESILPKR | |
| | NSDKLIARKKDWDPKKYGGFDSPTVAYSVLVVAKVEKGKSKKLKSVKELLGI | |
| | TIMERSSFEKNPIDFLEAKGYKEVKKDLIIKLPKYSLFELENGRKRMLASAGEL | |
| | QKGNELALPSKYVNFLYLASHYEKLKGSPEDNEQKQLFVEQHKHYLDEIIEQI | |
| | SEFSKRVILADANLDKVLSAYNKHRDKPIREQAENIIHLFTLTNLGAPAAFKYF | |
| | DTTIDRKRYTSTKEVLDATLIHQSITGLYETRIDLSQLGGD | |
| Cas9 nickase | MDKKYSIGLDIGTNSVGWAVITDEYKVPSKKFKVLGNTDRHSIKKNLIGALLF | SEQ ID NO: 364 |
| Streptococcus | DSGETAEATRLKRTARRRYTRRKNRICYLQEIFSNEMAKVDDSFFHRLEESFLV | |
| pyogenes | EEDKKHERHPIFGNIVDEVAYHEKYPTIYHLRKKLVDSTDKADLRLIYLALAH | |
| Q99ZW2 Cas9 | MIKFRGHFLIEGDLNPDNSDVDKLFIQLVQTYNQLFEENPINASGVDAKAILSA | |
| with D986X, | RLSKSRRLENLIAQLPGEKKNGLFGNLIALSLGLTPNFKSNFDLAEDAKLQLSK | |
| wherein X is any | DTYDDDLDNLLAQIGDQYADLFLAAKNLSDAILLSDILRVNTEITKAPLSASMI | |
| alternate amino | KRYDEHHQDLTLLKALVRQQLPEKYKEIFFDQSKNGYAGYIDGGASQEEFYK | |
| acid | FIKPILEKMDGTEELLVKLNREDLLRKQRTFDNGSIPHQIHLGELHAILRRQED | |
| | FYPFLKDNREKIEKILTFRIPYYVGPLARGNSRFAWMTRKSEETITPWNFEEVV | |
| | DKGASAQSFIERMTNFDKNLPNEKVLPKHSLLYEYFTVYNELTKVKYVTEGM | |
| | RKPAFLSGEQKKAIVDLLFKTNRKVTVKQLKEDYFKKIECFDSVEISGVEDRF | |
| | NASLGTYHDLLKIIKDKDFLDNEENEDILEDIVLTLTLFEDREMIEERLKTYAH | |
| | LFDDKVMKQLKRRRYTGWGRLSRKLINGIRDKQSGKTILDFLKSDGFANRNF | |
| | MQLIHDDSLTFKEDIQKAQVSGQGDSLHEHIANLAGSPAIKKGILQTVKVVDE | |
| | LVKVMGRHKPENIVIEMARENQTTQKGQKNSRERMKRIEEGIKELGSQILKEH | |
| | PVENTQLQNEKLYLYYLQNGRDMYVDQELDINRLSDYDVDHIVPQSFLKDDS | |
| | IDNKVLTRSDKNRGKSDNVPSEEVVKKMKNYWRQLLNAKLITQRKFDNLTK | |
| | AERGGLSELDKAGFIKRQLVETRQITKHVAQILDSRMNTKYDENDKLIREVKV | |
| | ITLKSKLVSDFRKDFQFYKVREINNYHHAHXAYLNAVVGTALIKKYPKLESEF | |
| | VYGDYKVYDVRKMIAKSEQEIGKATAKYFFYSNIMNFFKTEITLANGEIRKRP | |
| | LIETNGETGEIVWDKGRDFATVRKVLSMPQVNIVKKTEVQTGGFSKESILPKR | |
| | NSDKLIARKKDWDPKKYGGFDSPTVAYSVLVVAKVEKGKSKKLKSVKELLGI | |
| | TIMERSSFEKNPIDFLEAKGYKEVKKDLIIKLPKYSLFELENGRKRMLASAGEL | |
| | QKGNELALPSKYVNFLYLASHYEKLKGSPEDNEQKQLFVEQHKHYLDEIIEQI | |
| | SEFSKRVILADANLDKVLSAYNKHRDKPIREQAENIIHLFTLTNLGAPAAFKYF | |
| | DTTIDRKRYTSTKEVLDATLIHQSITGLYETRIDLSQLGGD | |
| Cas9 nickase | MDKKYSIGLAIGTNSVGWAVITDEYKVPSKKFKVLGNTDRHSIKKNLIGALLF | SEQ ID NO: 365 |
| Streptococcus | DSGETAEATRLKRTARRRYTRRKNRICYLQEIFSNEMAKVDDSFFHRLEESFLV | |
| pyogenes | EEDKKHERHPIFGNIVDEVAYHEKYPTIYHLRKKLVDSTDKADLRLIYLALAH | |
| Q99ZW2 Cas9 | MIKFRGHFLIEGDLNPDNSDVDKLFIQLVQTYNQLFEENPINASGVDAKAILSA | |
| with D10A | RLSKSRRLENLIAQLPGEKKNGLFGNLIALSLGLTPNFKSNFDLAEDAKLQLSK | |
| | DTYDDDLDNLLAQIGDQYADLFLAAKNLSDAILLSDILRVNTEITKAPLSASMI | |
| | KRYDEHHQDLTLLKALVRQQLPEKYKEIFFDQSKNGYAGYIDGGASQEEFYK | |
| | FIKPILEKMDGTEELLVKLNREDLLRKQRTFDNGSIPHQIHLGELHAILRRQED | |
| | FYPFLKDNREKIEKILTFRIPYYVGPLARGNSRFAWMTRKSEETITPWNFEEVV | |
| | DKGASAQSFIERMTNFDKNLPNEKVLPKHSLLYEYFTVYNELTKVKYVTEGM | |
| | RKPAFLSGEQKKAIVDLLFKTNRKVTVKQLKEDYFKKIECFDSVEISGVEDRF | |
| | NASLGTYHDLLKIIKDKDFLDNEENEDILEDIVLTLTLFEDREMIEERLKTYAH | |
| | LFDDKVMKQLKRRRYTGWGRLSRKLINGIRDKQSGKTILDFLKSDGFANRNF | |
| | MQLIHDDSLTFKEDIQKAQVSGQGDSLHEHIANLAGSPAIKKGILQTVKVVDE | |
| | LVKVMGRHKPENIVIEMARENQTTQKGQKNSRERMKRIEEGIKELGSQILKEH | |
| | PVENTQLQNEKLYLYYLQNGRDMYVDQELDINRLSDYDVDHIVPQSFLKDDS | |
| | IDNKVLTRSDKNRGKSDNVPSEEVVKKMKNYWRQLLNAKLITQRKFDNLTK | |
| | AERGGLSELDKAGFIKRQLVETRQITKHVAQILDSRMNTKYDENDKLIREVKV | |
| | ITLKSKLVSDFRKDFQFYKVREINNYHHAHDAYLNAVVGTALIKKYPKLESEF | |
| | VYGDYKVYDVRKMIAKSEQEIGKATAKYFFYSNIMNFFKTEITLANGEIRKRP | |
| | LIETNGETGEIVWDKGRDFATVRKVLSMPQVNIVKKTEVQTGGFSKESILPKR | |
| | NSDKLIARKKDWDPKKYGGFDSPTVAYSVLVVAKVEKGKSKKLKSVKELLGI | |
| | TIMERSSFEKNPIDFLEAKGYKEVKKDLIIKLPKYSLFELENGRKRMLASAGEL | |
| | QKGNELALPSKYVNFLYLASHYEKLKGSPEDNEQKQLFVEQHKHYLDEIIEQI | |
| | SEFSKRVILADANLDKVLSAYNKHRDKPIREQAENIIHLFTLTNLGAPAAFKYF | |
| | DTTIDRKRYTSTKEVLDATLIHQSITGLYETRIDLSQLGGD | |
| Cas9 nickase | MDKKYSIGLDIGTNSVGWAVITDEYKVPSKKFKVLGNTDRHSIKKNLIGALLF | SEQ ID NO: 366 |
| Streptococcus | DSGETAEATRLKRTARRRYTRRKNRICYLQEIFSNEMAKVDDSFFHRLEESFLV | |
| pyogenes | EEDKKHERHPIFGNIVDEVAYHEKYPTIYHLRKKLVDSTDKADLRLIYLALAH | |
| Q99ZW2 Cas9 | MIKFRGHFLIEGDLNPDNSDVDKLFIQLVQTYNQLFEENPINASGVDAKAILSA | |
| with E762A | RLSKSRRLENLIAQLPGEKKNGLFGNLIALSLGLTPNFKSNFDLAEDAKLQLSK | |
| | DTYDDDLDNLLAQIGDQYADLFLAAKNLSDAILLSDILRVNTEITKAPLSASMI | |
| | KRYDEHHQDLTLLKALVRQQLPEKYKEIFFDQSKNGYAGYIDGGASQEEFYK | |
| | FIKPILEKMDGTEELLVKLNREDLLRKQRTFDNGSIPHQIHLGELHAILRRQED | |
| | FYPFLKDNREKIEKILTFRIPYYVGPLARGNSRFAWMTRKSEETITPWNFEEVV | |
| | DKGASAQSFIERMTNFDKNLPNEKVLPKHSLLYEYFTVYNELTKVKYVTEGM | |
| | RKPAFLSGEQKKAIVDLLFKTNRKVTVKQLKEDYFKKIECFDSVEISGVEDRF | |
| | NASLGTYHDLLKIIKDKDFLDNEENEDILEDIVLTLTLFEDREMIEERLKTYAH | |
| | LFDDKVMKQLKRRRYTGWGRLSRKLINGIRDKQSGKTILDFLKSDGFANRNF | |
| | MQLIHDDSLTFKEDIQKAQVSGQGDSLHEHIANLAGSPAIKKGILQTVKVVDE | |
| | LVKVMGRHKPENIVIAMARENQTTQKGQKNSRERMKRIEEGIKELGSQILKE | |
| | HPVENTQLQNEKLYLYYLQNGRDMYVDQELDINRLSDYDVDHIVPQSFLKD | |
| | DSIDNKVLTRSDKNRGKSDNVPSEEVVKKMKNYWRQLLNAKLITQRKFDNL | |
| | TKAERGGLSELDKAGFIKRQLVETRQITKHVAQILDSRMNTKYDENDKLIREV | |
| | KVITLKSKLVSDFRKDFQFYKVREINNYHHAHDAYLNAVVGTALIKKYPKLES | |
| | EFVYGDYKVYDVRKMIAKSEQEIGKATAKYFFYSNIMNFFKTEITLANGEIRK | |
| | RPLIETNGETGEIVWDKGRDFATVRKVLSMPQVNIVKKTEVQTGGFSKESILP | |
| | KRNSDKLIARKKDWDPKKYGGFDSPTVAYSVLVVAKVEKGKSKKLKSVKEL | |
| | LGITIMERSSFEKNPIDFLEAKGYKEVKKDLIIKLPKYSLFELENGRKRMLASA | |
| | GELQKGNELALPSKYVNFLYLASHYEKLKGSPEDNEQKQLFVEQHKHYLDEI | |
| | IEQISEFSKRVILADANLDKVLSAYNKHRDKPIREQAENIIHLFTLTNLGAPAAF | |
| | KYFDTTIDRKRYTSTKEVLDATLIHQSITGLYETRIDLSQLGGD | |
| Cas9 | MDKKYSIGLDIGTNSVGWAVITDEYKVPSKKFKVLGNTDRHSIKKNLIGALLF | SEQ ID NO: 367 |
| nickase | DSGETAEATRLKRTARRRYTRRKNRICYLQEIFSNEMAKVDDSFFHRLEESFLV | |
| Streptococcus | EEDKKHERHPIFGNIVDEVAYHEKYPTIYHLRKKLVDSTDKADLRLIYLALAH | |
| pyogenes | MIKFRGHFLIEGDLNPDNSDVDKLFIQLVQTYNQLFEENPINASGVDAKAILSA | |
| Q99ZW2 Cas9 | RLSKSRRLENLIAQLPGEKKNGLFGNLIALSLGLTPNFKSNFDLAEDAKLQLSK | |
| with H983A | DTYDDDLDNLLAQIGDQYADLFLAAKNLSDAILLSDILRVNTEITKAPLSASMI | |
| | KRYDEHHQDLTLLKALVRQQLPEKYKEIFFDQSKNGYAGYIDGGASQEEFYK | |
| | FIKPILEKMDGTEELLVKLNREDLLRKQRTFDNGSIPHQIHLGELHAILRRQED | |
| | FYPFLKDNREKIEKILTFRIPYYVGPLARGNSRFAWMTRKSEETITPWNFEEVV | |
| | DKGASAQSFIERMTNFDKNLPNEKVLPKHSLLYEYFTVYNELTKVKYVTEGM | |
| | RKPAFLSGEQKKAIVDLLFKTNRKVTVKQLKEDYFKKIECFDSVEISGVEDRF | |
| | NASLGTYHDLLKIIKDKDFLDNEENEDILEDIVLTLTLFEDREMIEERLKTYAH | |
| | LFDDKVMKQLKRRRYTGWGRLSRKLINGIRDKQSGKTILDFLKSDGFANRNF | |
| | MQLIHDDSLTFKEDIQKAQVSGQGDSLHEHIANLAGSPAIKKGILQTVKVVDE | |
| | LVKVMGRHKPENIVIEMARENQTTQKGQKNSRERMKRIEEGIKELGSQILKEH | |
| | PVENTQLQNEKLYLYYLQNGRDMYVDQELDINRLSDYDVDHIVPQSFLKDDS | |
| | IDNKVLTRSDKNRGKSDNVPSEEVVKKMKNYWRQLLNAKLITQRKFDNLTK | |
| | AERGGLSELDKAGFIKRQLVETRQITKHVAQILDSRMNTKYDENDKLIREVKV | |
| | ITLKSKLVSDFRKDFQFYKVREINNYHAAHDAYLNAVVGTALIKKYPKLESEF | |
| | VYGDYKVYDVRKMIAKSEQEIGKATAKYFFYSNIMNFFKTEITLANGEIRKRP | |
| | LIETNGETGEIVWDKGRDFATVRKVLSMPQVNIVKKTEVQTGGFSKESILPKR | |
| | NSDKLIARKKDWDPKKYGGFDSPTVAYSVLVVAKVEKGKSKKLKSVKELLGI | |
| | TIMERSSFEKNPIDFLEAKGYKEVKKDLIIKLPKYSLFELENGRKRMLASAGEL | |
| | QKGNELALPSKYVNFLYLASHYEKLKGSPEDNEQKQLFVEQHKHYLDEIIEQI | |
| | SEFSKRVILADANLDKVLSAYNKHRDKPIREQAENIIHLFTLTNLGAPAAFKYF | |
| | DTTIDRKRYTSTKEVLDATLIHQSITGLYETRIDLSQLGGD | |
| Cas9 nickase | MDKKYSIGLDIGTNSVGWAVITDEYKVPSKKFKVLGNTDRHSIKKNLIGALLF | SEQ ID NO: 368 |
| Streptococcus | DSGETAEATRLKRTARRRYTRRKNRICYLQEIFSNEMAKVDDSFFHRLEESFLV | |
| pyogenes | EEDKKHERHPIFGNIVDEVAYHEKYPTIYHLRKKLVDSTDKADLRLIYLALAH | |
| Q99ZW2 Cas9 | MIKFRGHFLIEGDLNPDNSDVDKLFIQLVQTYNQLFEENPINASGVDAKAILSA | |
| with D986A | RLSKSRRLENLIAQLPGEKKNGLFGNLIALSLGLTPNFKSNFDLAEDAKLQLSK | |
| | DTYDDDLDNLLAQIGDQYADLFLAAKNLSDAILLSDILRVNTEITKAPLSASMI | |
| | KRYDEHHQDLTLLKALVRQQLPEKYKEIFFDQSKNGYAGYIDGGASQEEFYK | |
| | FIKPILEKMDGTEELLVKLNREDLLRKQRTFDNGSIPHQIHLGELHAILRRQED | |
| | FYPFLKDNREKIEKILTFRIPYYVGPLARGNSRFAWMTRKSEETITPWNFEEVV | |
| | DKGASAQSFIERMTNFDKNLPNEKVLPKHSLLYEYFTVYNELTKVKYVTEGM | |
| | RKPAFLSGEQKKAIVDLLFKTNRKVTVKQLKEDYFKKIECFDSVEISGVEDRF | |
| | NASLGTYHDLLKIIKDKDFLDNEENEDILEDIVLTLTLFEDREMIEERLKTYAH | |
| | LFDDKVMKQLKRRRYTGWGRLSRKLINGIRDKQSGKTILDFLKSDGFANRNF | |
| | MQLIHDDSLTFKEDIQKAQVSGQGDSLHEHIANLAGSPAIKKGILQTVKVVDE | |
| | LVKVMGRHKPENIVIEMARENQTTQKGQKNSRERMKRIEEGIKELGSQILKEH | |
| | PVENTQLQNEKLYLYYLQNGRDMYVDQELDINRLSDYDVDHIVPQSFLKDDS | |
| | IDNKVLTRSDKNRGKSDNVPSEEVVKKMKNYWRQLLNAKLITQRKFDNLTK | |
| | AERGGLSELDKAGFIKRQLVETRQITKHVAQILDSRMNTKYDENDKLIREVKV | |
| | ITLKSKLVSDFRKDFQFYKVREINNYHHAHAAYLNAVVGTALIKKYPKLESEF | |
| | VYGDYKVYDVRKMIAKSEQEIGKATAKYFFYSNIMNFFKTEITLANGEIRKRP | |
| | LIETNGETGEIVWDKGRDFATVRKVLSMPQVNIVKKTEVQTGGFSKESILPKR | |
| | NSDKLIARKKDWDPKKYGGFDSPTVAYSVLVVAKVEKGKSKKLKSVKELLGI | |
| | TIMERSSFEKNPIDFLEAKGYKEVKKDLIIKLPKYSLFELENGRKRMLASAGEL | |
| | QKGNELALPSKYVNFLYLASHYEKLKGSPEDNEQKQLFVEQHKHYLDEIIEQI | |
| | SEFSKRVILADANLDKVLSAYNKHRDKPIREQAENIIHLFTLTNLGAPAAFKYF | |
| | DTTIDRKRYTSTKEVLDATLIHQSITGLYETRIDLSQLGGD | |
| Cas9 nickase | MGKRNYILGLAIGITSVGYGIIDYETRDVIDAGVRLFKEANVENNEGRRSKRGARRLKR | SEQ ID NO: 438 |
| Staphylococcus | RRRHRIQRVKKLLFDYNLLTDHSELSGINPYEARVKGLSQKLSEEEFSAALLHLAKRRGV | |
| aureus (SaCas9) | HNVNEVEEDTGNELSTKEQISRNSKALEEKYVAELQLERLKKDGEVRGSINRFKTSDYV | |
| with D10A | KEAKQLLKVQKAYHQLDQSFIDTYIDLLETRRTYYEGPGEGSPFGWKDIKEWYEMLMG | |
| | HCTYFPEELRSVKYAYNADLYNALNDLNNLVITRDENEKLEYYEKFQIIENVFKQKKKPT | |
| | LKQIAKEILVNEEDIKGYRVTSTGKPEFTNLKVYHDIKDITARKEIIENAELLDQIAKIL | |
| | TIYQSSEDIQEELTNLNSELTQEEIEQISNLKGYTGTHNLSLKAINLILDELWHTNDNQL | |
| | AIFNRLKLVPKKVDLSQQKEIPTTLVDDFILSPVVKRSFIQSIKVINAIIKKYGLPNDII | |
| | IELAREKNSKDAQKMINEMQKRNRQTNERIEEIIRTTGKENAKYLIEKIKLHDMQEGKCL | |
| | YSLEAIPLEDLLNNPFNYEVDHIIPRSVSFDNSFNNKVLVKQEENSKKGNRTPFQYLSSS | |
| | DSKISYETFKKHILNLAKGKGRISKTKKEYLLEERDINRFSVQKDFINRNLVDTRYATRG | |
| | LMNLLRSYFRVNNLDVKVKSINGGFTSFLRRKWKFKKERNKGYKHHAEDALIIANADFIF | |
| | KEWKKLDKAKKVMENQMFEEKQAESMPEIETEQEYKEIFITPHQIKHIKDFKDYKYSHRV | |
| | DKKPNRKLINDTLYSTRKDDKGNTLIVNNLNGLYDKDNDKLKKLINKSPEKLLMYHHDPQ | |
| | TYQKLKLIMEQYGDEKNPLYKYYEETGNYLTKYSKKDNGPVIKKIKYYGNKLNAHLDITD | |
| | DYPNSRNKVVKLSLKPYRFDVYLDNGVYKFVTVKNLDVIKKENYYEVNSKCYEEAKKLKK | |
| | ISNQAEFIASFYKNDLIKINGELYRVIGVNNDLLNRIEVNMIDITYREYLENMNDKRPPH | |
| | IIKTIASKTQSIKKYSTDILGNLYEVKSKKHPQIIKK | |

Compact Cas9 Variants with Modified PAM Specificities

In some embodiments, the napDNAbp comprises a compact Cas protein, such as a Cas9 derived from C. jejuni, S. auricularis, N. meningitidis, or S. aureus. In exemplary embodiments, the napDNAbp comprises a CjCas9 nickase, a SauriCas9 nickase, an Nme2Cas9 nickase, an SaCas9 nickase, or an SaKKH-Cas9 nickase. In some embodiments, the napDNAbp is not an Nme2Cas9 protein or nickase. In some embodiments, the napDNAbp is not a SaCas9 protein or nickase.

The base editors of the present disclosure may also comprise Cas9 variants with modified PAM specificities. Some aspects of this disclosure provide Cas9 proteins that exhibit activity on a target sequence that does not comprise the canonical PAM (5′-NGG-3′, where N is A, C, G, or T) at its 3′-end. In some embodiments, the Cas9 protein exhibits activity on a target sequence comprising a 5′-NGG-3′ PAM sequence at its 3′-end. In some embodiments, the Cas9 protein exhibits activity on a target sequence comprising a 5′-NNG-3′ PAM sequence at its 3′-end. In some embodiments, the Cas9 protein exhibits activity on a target sequence comprising a 5′-NNA-3′ PAM sequence at its 3′-end. In some embodiments, the Cas9 protein exhibits activity on a target sequence comprising a 5′-NNC-3′ PAM sequence at its 3′-end. In some embodiments, the Cas9 protein exhibits activity on a target sequence comprising a 5′-NNT-3′ PAM sequence at its 3′-end. In some embodiments, the Cas9 protein exhibits activity on a target sequence comprising a 5′-NGT-3′ PAM sequence at its 3′-end. In some embodiments, the Cas9 protein exhibits activity on a target sequence comprising a 5′-NGA-3′ PAM sequence at its 3′-end. In some embodiments, the Cas9 protein exhibits activity on a target sequence comprising a 5′-NGC-3′ PAM sequence at its 3′-end. In some embodiments, the Cas9 protein exhibits activity on a target sequence comprising a 5′-NAA-3′ PAM sequence at its 3′-end. In some embodiments, the Cas9 protein exhibits activity on a target sequence comprising a 5′-NAC-3′ PAM sequence at its 3′-end. In some embodiments, the Cas9 protein exhibits activity on a target sequence comprising a 5′-NAT-3′ PAM sequence at its 3′-end. In still other embodiments, the Cas9 protein exhibits activity on a target sequence comprising a 5′-NAG-3′ PAM sequence at its 3′-end.

In some embodiments, the disclosed base editors comprise a napDNAbp domain comprising a SpCas9-NG, which has a PAM that corresponds to NGN. In some embodiments, the disclosed base editors comprise a napDNAbp domain that has a sequence that is at least 90%, at least 95%, at least 98%, or at least 99% identical to SpCas9-NG. The sequence of SpCas9-NG is illustrated below:

(SEQ ID NO: 477)
MDKKYSIGLAIGTNSVGWAVITDEYKVPSKKFKVLGNTDRHSIKKNLIGALLFDSGE
TAEATRLKRTARRRYTRRKNRICYLQEIFSNEMAKVDDSFFHRLEESFLVEEDKKHE
RHPIFGNIVDEVAYHEKYPTIYHLRKKLVDSTDKADLRLIYLALAHMIKFRGHFLIEG
DLNPDNSDVDKLFIQLVQTYNQLFEENPINASGVDAKAILSARLSKSRRLENLIAQLP
GEKKNGLFGNLIALSLGLTPNFKSNFDLAEDAKLQLSKDTYDDDLDNLLAQIGDQYA
DLFLAAKNLSDAILLSDILRVNTEITKAPLSASMIKRYDEHHQDLTLLKALVRQQLPE
KYKEIFFDQSKNGYAGYIDGGASQEEFYKFIKPILEKMDGTEELLVKLNREDLLRKQR
TFDNGSIPHQIHLGELHAILRRQEDFYPFLKDNREKIEKILTFRIPYYVGPLARGNSRFA
WMTRKSEETITPWNFEEVVDKGASAQSFIERMTNFDKNLPNEKVLPKHSLLYEYFTV
YNELTKVKYVTEGMRKPAFLSGEQKKAIVDLLFKTNRKVTVKQLKEDYFKKIECFD
SVEISGVEDRFNASLGTYHDLLKIIKDKDFLDNEENEDILEDIVLTLTLFEDREMIEERL
KTYAHLFDDKVMKQLKRRRYTGWGRLSRKLINGIRDKQSGKTILDFLKSDGFANRN
FMQLIHDDSLTFKEDIQKAQVSGQGDSLHEHIANLAGSPAIKKGILQTVKVVDELVK
VMGRHKPENIVIEMARENQTTQKGQKNSRERMKRIEEGIKELGSQILKEHPVENTQL
QNEKLYLYYLQNGRDMYVDQELDINRLSDYDVDHIVPQSFLKDDSIDNKVLTRSDK
NRGKSDNVPSEEVVKKMKNYWRQLLNAKLITQRKFDNLTKAERGGLSELDKAGFIK
RQLVETRQITKHVAQILDSRMNTKYDENDKLIREVKVITLKSKLVSDFRKDFQFYKV
REINNYHHAHDAYLNAVVGTALIKKYPKLESEFVYGDYKVYDVRKMIAKSEQEIGK
ATAKYFFYSNIMNFFKTEITLANGEIRKRPLIETNGETGEIVWDKGRDFATVRKVLSM
PQVNIVKKTEVQTGGFSKESIRPKRNSDKLIARKKDWDPKKYGGFVSPTVAYSVLVV
AKVEKGKSKKLKSVKELLGITIMERSSFEKNPIDFLEAKGYKEVKKDLIIKLPKYSLFE
LENGRKRMLASARFLQKGNELALPSKYVNFLYLASHYEKLKGSPEDNEQKQLFVEQ
HKHYLDEIIEQISEFSKRVILADANLDKVLSAYNKHRDKPIREQAENIIHLFTLTNLGA
PRAFKYFDTTIDRKVYRSTKEVLDATLIHQSITGLYETRIDLSQLGGD

In some embodiments, the disclosed base editors comprise a napDNAbp domain comprising a S. aureus Cas9 nickase KKH, or SaCas9-KKH, which has a PAM that corresponds to NNNRRT, or NNGRRT. This Cas9 variant contains the amino acid substitutions D10A, E782K, N968K, and R1015H (“KKH”) relative to wild-type SaCas9, set forth as SEQ ID NO: 377. In some embodiments, the disclosed base editors comprise a napDNAbp domain that has a sequence that is at least 90%, at least 95%, at least 98%, or at least 99% identical to SaCas9-KKH. The length of SaCas9 (and SaKKH-Cas9) is 1053 amino acids. The sequence of SaCas9-KKH (nickase) is illustrated below:

S. aureus Cas9 Nickase KKH (SaCas9-KKH)

(SEQ ID NO: 478)
MGKRNYILGLAIGITSVGYGIIDYETRDVIDAGVRLFKEANVENNEGRRSKRGARRL
KRRRRHRIQRVKKLLFDYNLLTDHSELSGINPYEARVKGLSQKLSEEEFSAALLHLAK
RRGVHNVNEVEEDTGNELSTKEQISRNSKALEEKYVAELQLERLKKDGEVRGSINRF
KTSDYVKEAKQLLKVQKAYHQLDQSFIDTYIDLLETRRTYYEGPGEGSPFGWKDIKE
WYEMLMGHCTYFPEELRSVKYAYNADLYNALNDLNNLVITRDENEKLEYYEKFQII
ENVFKQKKKPTLKQIAKEILVNEEDIKGYRVTSTGKPEFTNLKVYHDIKDITARKEIIE
NAELLDQIAKILTIYQSSEDIQEELTNLNSELTQEEIEQISNLKGYTGTHNLSLKAINLIL
DELWHTNDNQIAIFNRLKLVPKKVDLSQQKEIPTTLVDDFILSPVVKRSFIQSIKVINAI
IKKYGLPNDIIIELAREKNSKDAQKMINEMQKRNRQTNERIEEIIRTTGKENAKYLIEKI
KLHDMQEGKCLYSLEAIPLEDLLNNPFNYEVDHIIPRSVSFDNSFNNKVLVKQEENSK
KGNRTPFQYLSSSDSKISYETFKKHILNLAKGKGRISKTKKEYLLEERDINRFSVQKDF
INRNLVDTRYATRGLMNLLRSYFRVNNLDVKVKSINGGFTSFLRRKWKFKKERNKG
YKHHAEDALIIANADFIFKEWKKLDKAKKVMENQMFEEKQAESMPEIETEQEYKEIF
ITPHQIKHIKDFKDYKYSHRVDKKPNRKLINDTLYSTRKDDKGNTLIVNNLNGLYDK
DNDKLKKLINKSPEKLLMYHHDPQTYQKLKLIMEQYGDEKNPLYKYYEETGNYLTK
YSKKDNGPVIKKIKYYGNKLNAHLDITDDYPNSRNKVVKLSLKPYRFDVYLDNGVY
KFVTVKNLDVIKKENYYEVNSKCYEEAKKLKKISNQAEFIASFYKNDLIKINGELYRV
IGVNNDLLNRIEVNMIDITYREYLENMNDKRPPHIIKTIASKTQSIKKYSTDILGNLYE
VKSKKHPQIIKKG

In some embodiments, the disclosed base editors comprise a napDNAbp comprising a Cas9 protein derived from Staphylococcus Auricularis (S. auri Cas9, or SauriCas9). In some embodiments, the disclosed base editors comprise a SauriCas9 nickase. SauriCas9 recognizes NNGG and NNNGG PAMs. The sequence of SauriCas9 (nickase) is set forth as SEQ ID NO: 479. In some embodiments, the disclosed base editors comprise a napDNAbp domain that has a sequence that is at least 90%, at least 95%, at least 98%, or at least 99% identical to SEQ ID NO: 479. In some embodiments, the disclosed base editors comprise a napDNAbp comprising SEQ ID NO: 479. The length of this protein is 1061 amino acids.

(SEQ ID NO: 479)
MQENQQKQNYILGLAIGITSVGYGLIDSKTREVIDAGVRLFPEADSENNSNRRSKRGA
RRLKRRRIHRLNRVKDLLADYQMIDLNNVPKSTDPYTIRVKGLREPLTKEEFAIALLH
IAKRRGLHNISVSMGDEEQDNELSTKQQLQKNAQQLQDKYVCELQLERLTNINKVR
GEKNRFKTEDFVKEVKQLCETQRQYHNIDDQFIQQYIDLVSTRREYFEGPGNGSPYG
WDGDLLKWYEKLMGRCTYFPEELRSVKYAYSADLFNALNDLNNLVVTRDDNPKLE
YYEKYHIIENVFKQKKNPTLKQIAKEIGVQDYDIRGYRITKSGKPQFTSFKLYHDLKNI
FEQAKYLEDVEMLDEIAKILTIYQDEISIKKALDQLPELLTESEKSQIAQLTGYTGTHR
LSLKCIHIVIDELWESPENQMEIFTRLNLKPKKVEMSEIDSIPTTLVDEFILSPVVKRAFI
QSIKVINAVINRFGLPEDIIIELAREKNSKDRRKFINKLQKQNEATRKKIEQLLAKYGN
TNAKYMIEKIKLHDMQEGKCLYSLEAIPLEDLLSNPTHYEVDHIIPRSVSFDNSLNNK
VLVKQSENSKKGNRTPYQYLSSNESKISYNQFKQHILNLSKAKDRISKKKRDMLLEE
RDINKFEVQKEFINRNLVDTRYATRELSNLLKTYFSTHDYAVKVKTINGGFTNHLRK
VWDFKKHRNHGYKHHAEDALVIANADFLFKTHKALRRTDKILEQPGLEVNDTTVK
VDTEEKYQELFETPKQVKNIKQFRDFKYSHRVDKKPNRQLINDTLYSTREIDGETYV
VQTLKDLYAKDNEKVKKLFTERPQKILMYQHDPKTFEKLMTILNQYAEAKNPLAAY
YEDKGEYVTKYAKKGNGPAIHKIKYIDKKLGSYLDVSNKYPETQNKLVKLSLKSFRF
DIYKCEQGYKMVSIGYLDVLKKDNYYYIPKDKYEAEKQKKKIKESDLFVGSFYYND
LIMYEDELFRVIGVNSDINNLVELNMVDITYKDFCEVNNVTGEKRIKKTIGKRVVLIE
KYTTDILGNLYKTPLPKKPQLIFKRGEL

In some embodiments, the napDNAbp comprises a SauriCas9-KKH variant, or a SauriCas9-KKH nickase variant. SauriCas9-KKH contains corresponding triple KKH mutations: Q788K, Y973K, and R1020H. See Hu et al. (2020) PLoS Biol. 18 (3): e3000686, which is incorporated herein by reference.

In some embodiments, the disclosed base editors comprise a napDNAbp domain comprising an S. pyogenes Cas9 nickase KKH, or SpCas9-KKH, which has a PAM that corresponds to NNNRRT.

In some embodiments, the disclosed base editors comprise a napDNAbp comprising a compact Cas9 ortholog from derived from Neisseria meningitidis (Nme, or Nme2). In some embodiments, the napDNAbp comprises Nme2Cas9. In some embodiments, the disclosed base editors comprise an Nme2Cas9 nickase. Nme2Cas9 recognizes a simple dinucleotide PAM, NNNNCC, or N4CC (where N is any nucleotide), as described in Edraki et al., Molecular Cell 73, 714-726, incorporated herein by reference. The sequence of Nme2Cas9 is set forth as SEQ ID NO: 5. In some embodiments, the disclosed base editors comprise a napDNAbp domain that has a sequence that is at least 90%, at least 95%, at least 98%, or at least 99% identical to SEQ ID NO: 5. In some embodiments, the disclosed base editors comprise a napDNAbp comprising SEQ ID NO: 5. The length of this protein is 1082 amino acids.

(SEQ ID NO: 5)
MAAFKPNPINYILGLAIGIASVGWAMVEIDEEENPIRLIDLGVRVFERAEVPKTGDSLA
MARRLARSVRRLTRRRAHRLLRARRLLKREGVLQAADFDENGLIKSLPNTPWQLRA
AALDRKLTPLEWSAVLLHLIKHRGYLSQRKNEGETADKELGALLKGVANNAHALQT
GDFRTPAELALNKFEKESGHIRNQRGDYSHTFSRKDLQAELILLFEKQKEFGNPHVSG
GLKEGIETLLMTQRPALSGDAVQKMLGHCTFEPAEPKAAKNTYTAERFIWLTKLNN
LRILEQGSERPLTDTERATLMDEPYRKSKLTYAQARKLLGLEDTAFFKGLRYGKDNA
EASTLMEMKAYHAISRALEKEGLKDKKSPLNLSSELQDEIGTAFSLFKTDEDITGRLK
DRVQPEILEALLKHISFDKFVQISLKALRRIVPLMEQGKRYDEACAEIYGDHYGKKNT
EEKIYLPPIPADEIRNPVVLRALSQARKVINGVVRRYGSPARIHIETAREVGKSFKDRK
EIEKRQEENRKDREKAAAKFREYFPNFVGEPKSKDILKLRLYEQQHGKCLYSGKEIN
LVRLNEKGYVEIDHALPFSRTWDDSFNNKVLVLGSENQNKGNQTPYEYFNGKDNSR
EWQEFKARVETSRFPRSKKQRILLQKFDEDGFKECNLNDTRYVNRFLCQFVADHILL
TGKGKRRVFASNGQITNLLRGFWGLRKVRAENDRHHALDAVVVACSTVAMQQKIT
RFVRYKEMNAFDGKTIDKETGKVLHQKTHFPQPWEFFAQEVMIRVFGKPDGKPEFE
EADTPEKLRTLLAEKLSSRPEAVHEYVTPLFVSRAPNRKMSGAHKDTLRSAKRFVKH
NEKISVKRVWLTEIKLADLENMVNYKNGREIELYEALKARLEAYGGNAKQAFDPKD
NPFYKKGGQLVKAVRVEKTQESGVLLNKKNAYTIADNGDMVRVDVFCKVDKKGK
NQYFIVPIYAWQVAENILPDIDCKGYRIDDSYTFCFSLHKYDLIAFQKDEKSKVEFAY
YINCDSSNGRFYLAWHDKGSKEQQFRISTQNLVLIQKYQVNELGKEIRPCRLKKRPP
VR.

The amino acid sequence of NmeCas9 is provided below, as SEQ ID NO: 6. In some embodiments, the disclosed base editors comprise a napDNAbp domain that has a sequence that is at least 90%, at least 95%, at least 98%, or at least 99% identical to SEQ ID NO: 6. In some embodiments, the disclosed base editors comprise a napDNAbp comprising SEQ ID NO: 6. The length of this protein is 1083 amino acids.

(SEQ ID NO: 6)
MAAFKPNSINYILGLAIGIASVGWAMVEIDEEENPIRLIDLGVRVFERAEVPKTGDSLA
MARRLARSVRRLTRRRAHRLLRTRRLLKREGVLQAANFDENGLIKSLPNTPWQLRA
AALDRKLTPLEWSAVLLHLIKHRGYLSQRKNEGETADKELGALLKGVAGNAHALQT
GDFRTPAELALNKFEKESGHIRNQRSDYSHTFSRKDLQAELILLFEKQKEFGNPHVSG
GLKEGIETLLMTQRPALSGDAVQKMLGHCTFEPAEPKAAKNTYTAERFIWLTKLNN
LRILEQGSERPLTDTERATLMDEPYRKSKLTYAQARKLLGLEDTAFFKGLRYGKDNA
EASTLMEMKAYHAISRALEKEGLKDKKSPLNLSPELQDEIGTAFSLFKTDEDITGRLK
DRIQPEILEALLKHISFDKFVQISLKALRRIVPLMEQGKRYDEACAEIYGDHYGKKNT
EEKIYLPPIPADEIRNPVVLRALSQARKVINGVVRRYGSPARIHIETAREVGKSFKDRK
EIEKRQEENRKDREKAAAKFREYFPNFVGEPKSKDILKLRLYEQQHGKCLYSGKEIN
LGRLNEKGYVEIDAALPFSRTWDDSFNNKVLVLGSENQNKGNQTPYEYFNGKDNSR
EWQEFKARVETSRFPRSKKQRILLQKFDEDGFKERNLNDTRYVNRFLCQFVADRMR
LTGKGKKRVFASNGQITNLLRGFWGLRKVRAENDRHHALDAVVVACSTVAMQQKI
TRFVRYKEMNAFDGKTIDKETGEVLHQKTHFPQPWEFFAQEVMIRVFGKPDGKPEF
EEADTLEKLRTLLAEKLSSRPEAVHEYVTPLFVSRAPNRKMSGQGHMETVKSAKRL
DEGVSVLRVPLTQLKLKDLEKMVNREREPKLYEALKARLEAHKDDPAKAFAEPFYK
YDKAGNRTQQVKAVRVEQVQKTGVWVRNHNGIADNATMVRVDVFEKGDKYYLV
PIYSWQVAKGILPDRAVVQGKDEEDWQLIDDSFNFKFSLHPNDLVEVITKKARMFGY
FASCHRGTGNINIRIHDLDHKIGKNGILEGIGVKTALSFQKYQIDELGKEIRPCRLKKR
PPVR

In some embodiments, the disclosed base editors comprise a napDNAbp comprising a compact Cas9 ortholog from derived from Campylobacter jejuni (CjCas9). In some embodiments, the napDNAbp comprises CjCas9. In some embodiments, the disclosed base editors comprise a CjCas9 nickase. CjCas9 recognizes NNNNACA and NNNNACAC PAMs. See Kim et al., Nature Communications 8 (14500): 1-12 (2017), which is incorporated herein by reference. The sequence of CjCas9 (nickase) is set forth as SEQ ID NO: 379. In some embodiments, the disclosed base editors comprise a napDNAbp domain that has a sequence that is at least 90%, at least 95%, at least 98%, or at least 99% identical to SEQ ID NO: 379. In some embodiments, the disclosed base editors comprise a napDNAbp comprising SEQ ID NO: 379. The length of this protein is 984 amino acids.

(SEQ ID NO: 379)
MARILAFAIGISSIGWAFSENDELKDCGVRIFTKVENPKTGESLALPRRLARSARKRL
ARRKARLNHLKHLIANEFKLNYEDYQSFDESLAKAYKGSLISPYELRFRALNELLSK
QDFARVILHIAKRRGYDDIKNSDDKEKGAILKAIKQNEEKLANYQSVGEYLYKEYFQ
KFKENSKEFTNVRNKKESYERCIAQSFLKDELKLIFKKQREFGFSFSKKFEEEVLSVAF
YKRALKDFSHLVGNCSFFTDEKRAPKNSPLAFMFVALTRIINLLNNLKNTEGILYTKD
DLNALLNEVLKNGTLTYKQTKKLLGLSDDYEFKGEKGTYFIEFKKYKEFIKALGEHN
LSQDDLNEIAKDITLIKDEIKLKKALAKYDLNQNQIDSLSKLEFKDHLNISFKALKLVT
PLMLEGKKYDEACNELNLKVAINEDKKDFLPAFNETYYKDEVTNPVVLRAIKEYRK
VLNALLKKYGKVHKINIELAREVGKNHSQRAKIEKEQNENYKAKKDAELECEKLGL
KINSKNILKLRLFKEQKEFCAYSGEKIKISDLQDEKMLEIDHIYPYSRSFDDSYMNKVL
VFTKQNQEKLNQTPFEAFGNDSAKWQKIEVLAKNLPTKKQKRILDKNYKDKEQKNF
KDRNLNDTRYIARLVLNYTKDYLDFLPLSDDENTKLNDTQKGSKVHVEAKSGMLTS
ALRHTWGFSAKDRNNHLHHAIDAVIIAYANNSIVKAFSDFKKEQESNSAELYAKKIS
ELDYKNKRKFFEPFSGFRQKVLDKIDEIFVSKPERKKPSGALHEETFRKEEEFYQSYG
GKEGVLKALELGKIRKVNGKIVKNGDMFRVDIFKHKKTNKFYAVPIYTMDFALKVL
PNKAVARSKKGEIKDWILMDENYEFCFSLYKDSLILIQTKDMQEPEFVYYNAFTSSTV
SLIVSKHDNKFETLSKNQKILFKNANEKEVIAKSIGIQNLKVFEKYIVSALGEVTKAEF
RQREDFKK

### Additional Compact Cas Proteins

In various embodiments, the nucleic acid programmable DNA binding proteins include, without limitation, compact variants of a Cas9 (e.g., dCas9 and nCas9), a CasX, a CasY, a Cpf1, a C2c1, a C2c2, a C2c3, a GeoCas9, a CjCas9, a Cas12a, a Cas12b, a Cas12g, a Cas12h, a Cas12i, a Cas13b, a Cas13c, a Cas13d, a Cas14, a Csn2, an xCas9, an SpCas9-NG, a circularly permuted Cas9 domain such as CP1012, CP1028, CP1041, CP1249, and CP1300, an Argonaute (Ago) domain, a Cas9-KKH, a SmacCas9, a SpRY, a SpRY-HF1, a Spy-macCas9, an SpCas9-VRQR, an SpCas9-VRER, an SpCas9-VQR, an SpCas9-EQR, an SpCas9-NRRH, an SpaCas9-NRTH, an SpCas9-NRCH, an LbCas12a, an AsCas12a, a CeCas12a, an MbCas12a, a CasΦ, an SpCas9-NG-CP1041, an SpCas9-NG-VRQR.

In still other embodiments, the napDNAbp may comprise a compact Cas9 ortholog from Staphylococcus lugdunensis Cas9 (SlugCas9), Staphylococcus lutrae Cas9 (SlutrCas9), or Staphylococcus haemolyticus Cas9 (ShaCas9). See Hu et al., Nucleic Acids Research, 49 (7), April 2021, 4008-4019, which is incorporated herein by reference. The SlugCas9, SlutrCas9, and ShaCas9 proteins recognize NNGG, NNGG/NNGA, and NNGG PAMs, respectively.

In still other embodiments, the Cas protein may include any CRISPR associated protein, including but not limited to, Cas12a, Cas12b, Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof, and preferably comprising a nickase mutation (e.g., a mutation corresponding to the D10A mutation of the wild type SpCas9 polypeptide of SEQ ID NO: 326).

In certain embodiments, the base editors contemplated herein can include a Cas9 protein that is of smaller molecular weight than the canonical SpCas9 sequence. In some embodiments, the smaller-sized Cas9 variants may facilitate delivery to cells, e.g., by an AAV vector, expression vector, or other means of delivery. The canonical SpCas9 protein is 1368 amino acids in length and has a predicted molecular weight of 158 kilodaltons. The term “small-sized Cas9 variant”, as used herein, refers to any Cas9 variant—naturally occurring, engineered, or otherwise—that is less than about 1300 amino acids, or at least less than 1290 amino acids, or than less than 1280 amino acids, or less than 1270 amino acid, or less than 1260 amino acid, or less than 1250 amino acids, or less than 1240 amino acids, or less than 1230 amino acids, or less than 1220 amino acids, or less than 1210 amino acids, or less than 1200 amino acids, or less than 1190 amino acids, or less than 1180 amino acids, or less than 1170 amino acids, or less than 1160 amino acids, or less than 1150 amino acids, or less than 1140 amino acids, or less than 1130 amino acids, or less than 1120 amino acids, or less than 1110 amino acids, or less than 1100 amino acids, or less than 1050 amino acids, or less than 1000 amino acids, or less than 950 amino acids, or less than 900 amino acids, or less than 850 amino acids, or less than 800 amino acids, or less than 750 amino acids, or less than 700 amino acids, or less than 650 amino acids, or less than 600 amino acids, or less than 550 amino acids, or less than 500 amino acids, but at least larger than about 400 amino acids and retaining the required functions of the Cas9 protein.

In various embodiments, the base editors disclosed herein may comprise one of the small-sized Cas9 variants described as follows, or a Cas9 variant thereof having at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to any reference small-sized Cas9 protein. Exemplary small-sized Cas9 variants include, but are not limited to, SauriCas9, SaCas9, CjCas9, Nme2Cas9, AsCas 12a, and LbCas12a.

In some embodiments, the napDNAbp domain of any of the disclosed base editors comprises an LbCas12a, such as a wild-type LbCas12a. In some embodiments, the napDNAbp domain of any of the disclosed based editors is comprises at least 80%, at least 85%, at least 90%, at least 95%, or at least 99% sequence identity to SEQ ID NO: 381. In some embodiments, the napDNAbp domain of any of the disclosed base editors comprises the amino acid sequence of SEQ ID NO: 381.

In some embodiments, the napDNAbp domain of any of the disclosed base editors comprises an AsCas 12a, such as a wild-type AsCas12a. In some embodiments, the napDNAbp domain of any of the disclosed base editors comprises a mutant AsCas12a, such as an engineered AsCas12a, or enAsCas12a. In some embodiments, the napDNAbp domain of any of the disclosed based editors is comprises at least 80%, at least 85%, at least 90%, at least 95%, or at least 99% sequence identity to SEQ ID NO: 383. In some embodiments, the napDNAbp domain of any of the disclosed base editors comprises the amino acid sequence of SEQ ID NO: 383.

| Description | Sequence | SEQ ID NO: |
|---|---|---|
| SaCas9 | MGKRNYILGLDIGITSVGYGIIDYETRDVIDAGVRLFKEANVENNEGRRSKR | SEQ ID NO: 377 |
| Staphylococcus | ARRLKRRRRHRIQRVKKLLFDYNLLTDHSELSGINPYEARVKGLSQKLSEEEF | |
| aureus | SAALLHLAKRRGVHNVNEVEEDTGNELSTKEQISRNSKALEEKYVAELQLER | |
| 1053 AA | LKKDGEVRGSINRFKTSDYVKEAKQLLKVQKAYHQLDQSFIDTYIDLLETRRT | |
| 123 kDa | YYEGPGEGSPFGWKDIKEWYEMLMGHCTYFPEELRSVKYAYNADLYNALND | |
| | LNNLVITRDENEKLEYYEKFQIIENVFKQKKKPTLKQIAKEILVNEEDIKGYRV | |
| | TSTGKPEFTNLKVYHDIKDITARKEIIENAELLDQIAKILTIYQSSEDIQEELTNL | |
| | NSELTQEEIEQISNLKGYTGTHNLSLKAINLILDELWHTNDNQIAIFNRLKLVPK | |
| | KVDLSQQKEIPTTLVDDFILSPVVKRSFIQSIKVINAIIKKYGLPNDIIIELAREKN | |
| | SKDAQKMINEMQKRNRQTNERIEEIIRTTGKENAKYLIEKIKLHDMQEGKCLY | |
| | SLEAIPLEDLLNNPFNYEVDHIIPRSVSFDNSFNNKVLVKQEENSKKGNRTPFQ | |
| | YLSSSDSKISYETFKKHILNLAKGKGRISKTKKEYLLEERDINRFSVQKDFINR | |
| | NLVDTRYATRGLMNLLRSYFRVNNLDVKVKSINGGFTSFLRRKWKFKKERNK | |
| | GYKHHAEDALIIANADFIFKEWKKLDKAKKVMENQMFEEKQAESMPEIETEQ | |
| | EYKEIFITPHQIKHIKDFKDYKYSHRVDKKPNRKLINDTLYSTRKDDKGNTLIV | |
| | NNLNGLYDKDNDKLKKLINKSPEKLLMYHHDPQTYQKLKLIMEQYGDEKNP | |
| | LYKYYEETGNYLTKYSKKDNGPVIKKIKYYGNKLNAHLDITDDYPNSRNKVV | |
| | KLSLKPYRFDVYLDNGVYKFVTVKNLDVIKKENYYEVNSKCYEEAKKLKKIS | |
| | NQAEFIASFYKNDLIKINGELYRVIGVNNDLLNRIEVNMIDITYREYLENMNDK | |
| | RPPHIIKTIASKTQSIKKYSTDILGNLYEVKSKKHPQIIKK | |
| GeoCas9 | MRYKIGLDIGITSVGWAVMNLDIPRIEDLGVRIFDRAENPQTGESLALPRRLAR | SEQ ID NO: 380 |
| G. | SARRRLRRRKHRLERIRRLVIREGILTKEELDKLFEEKHEIDVWQLRVEALDRK | |
| stearothermo- | LNNDELARVLLHLAKRRGFKSNRKSERSNKENSTMLKHIEENRAILSSYRTVG | |
| philus | EMIVKDPKFALHKRNKGENYTNTIARDDLEREIRLIFSKQREFGNMSCTEEFE | |
| 1087 AA | NEYITIWASQRPVASKDDIEKKVGFCTFEPKEKRAPKATYTFQSFIAWEHINKL | |
| 127 kDa | RLISPSGARGLTDEERRLLYEQAFQKNKITYHDIRTLLHLPDDTYFKGIVYDRG | |
| | ESRKQNENIRFLELDAYHQIRKAVDKVYGKGKSSSFLPIDFDTFGYALTLFKDD | |
| | ADIHSYLRNEYEQNGKRMPNLANKVYDNELIEELLNLSFTKFGHLSLKALRSI | |
| | LPYMEQGEVYSSACERAGYTFTGPKKKQKTMLLPNIPPIANPVVMRALTQAR | |
| | KVVNAIIKKYGSPVSIHIELARDLSQTFDERRKTKKEQDENRKKNETAIRQLM | |
| | EYGLTLNPTGHDIVKFKLWSEQNGRCAYSLQPIEIERLLEPGYVEVDHVIPYSR | |
| | SLDDSYTNKVLVLTRENREKGNRIPAEYLGVGTERWQQFETFVLTNKQFSKK | |
| | KRDRLLRLHYDENEETEFKNRNLNDTRYISRFFANFIREHLKFAESDDKQKVY | |
| | TVNGRVTAHLRSRWEFNKNREESDLHHAVDAVIVACTTPSDIAKVTAFYQRRE | |
| | QNKELAKKTEPHFPQPWPHFADELRARLSKHPKESIKALNLGNYDDQKLESL | |
| | QPVFVSRMPKRSVTGAAHQETLRRYVGIDERSGKIQTVVKTKLSEIKLDASGH | |
| | FPMYGKESDPRTYEAIRQRLLEHNNDPKKAFQEPLYKPKKNGEPGPVIRTVKII | |
| | DTKNQVIPLNDGKTVAYNSNIVRVDVFEKDGKYYCVPVYTMDIMKGILPNKA | |
| | IEPNKPYSEWKEMTEDYTFRFSLYPNDLIRIELPREKTVKTAAGEEINVKDVFV | |
| | YYKTIDSANGGLELISHDHRFSLRGVGSRTLKRFEKYQVDVLGNIYKVRGEK | |
| | RVGLASSAHSKPGKTIRPLQSTRD | |
| LbCas12a | MSKLEKFTNCYSLSKTLRFKAIPVGKTQENIDNKRLLVEDEKRAEDYKGVKK | SEQ ID NO: 381 |
| L. bacterium | LLDRYYLSFINDVLHSIKLKNLNNYISLFRKKTRTEKENKELENLEINLRKEIA | |
| 1228 AA | KAFKGNEGYKSLFKKDIIETILPEFLDDKDEIALVNSFNGFTTAFTGFFDNREN | |
| 143.9 kDa | MFSEEAKSTSIAFRCINENLTRYISNMDIFEKVDAIFDKHEVQEIKEKILNSDYD | |
| | VEDFFEGEFFNFVLTQEGIDVYNAIIGGFVTESGEKIKGLNEYINLYNQKTKQK | |
| | LPKFKPLYKQVLSDRESLSFYGEGYTSDEEVLEVFRNTLNKNSEIFSSIKKLEK | |
| | LFKNFDEYSSAGIFVKNGPAISTISKDIFGEWNVIRDKWNAEYDDIHLKKKAV | |
| | VTEKYEDDRRKSFKKIGSFSLEQLQEYADADLSVVEKLKEIIIQKVDEIYKVYG | |
| | SSEKLFDADFVLEKSLKKNDAVVAIMKDLLDSVKSFENYIKAFFGEGKETNRD | |
| | ESFYGDFVLAYDILLKVDHIYDAIRNYVTQKPYSKDKFKLYFQNPQFMGGWD | |
| | KDKETDYRATILRYGSKYYLAIMDKKYAKCLQKIDKDDVNGNYEKINYKLLP | |
| | GPNKMLPKVFFSKKWMAYYNPSEDIQKIYKNGTFKKGDMFNLNDCHKLIDF | |
| | FKDSISRYPKWSNAYDFNFSETEKYKDIAGFYREVEEQGYKVSFESASKKEVD | |
| | KLVEEGKLYMFQIYNKDFSDKSHGTPNLHTMYFKLLFDENNHGQIRLSGGAE | |
| | LFMRRASLKKEELVVHPANSPIANKNPDNPKKTTTLSYDVYKDKRFSEDQYE | |
| | LHIPIAINKCPKNIFKINTEVRVLLKHDDNPYVIGIDRGERNLLYIVVVDGKGNI | |
| | VEQYSLNEIINNFNGIRIKTDYHSLLDKKEKERFEARQNWTSIENIKELKAGYI | |
| | SQVVHKICELVEKYDAVIALEDLNSGFKNSRVKVEKQVYQKFEKMLIDKLNY | |
| | MVDKKSNPCATGGALKGYQITNKFESFKSMSTQNGFIFYIPAWLTSKIDPSTGF | |
| | VNLLKTKYTSIADSKKFISSFDRIMYVPEEDLFEFALDYKNFSRTDADYIKKW | |
| | KLYSYGNRIRIFRNPKKNNVFDWEEVCLTSAYKELFNKYGINYQQGDIRALLC | |
| | EQSDKAFYSSFMALMSLMLQMRNSITGRTDVDFLISPVKNSDGIFYDSRNYEA | |
| | QENAILPKNADANGAYNIARKVLWAIGQFKKAEDEKLDKVKIAISNKEWLEY | |
| | AQTSVKH | |
| BhCas 12b | MATRSFILKIEPNEEVKKGLWKTHEVLNHGIAYYMNILKLIRQEAIYEHHEQD | SEQ ID NO: 382 |
| B. hisashii | PKNPKKVSKAEIQAELWDFVLKMQKCNSFTHEVDKDEVFNILRELYEELVPSS | |
| 1108 AA | VEKKGEANQLSNKFLYPLVDPNSQSGKGTASSGRKPRWYNLKIAGDPSWEEE | |
| 130.4kDa | KKKWEEDKKKDPLAKILGKLAEYGLIPLFIPYTDSNEPIVKEIKWMEKSRNQS | |
| | VRRLDKDMFIQALERFLSWESWNLKVKEEYEKVEKEYKTLEERIKEDIQALK | |
| | ALEQYEKERQEQLLRDTLNTNEYRLSKRGLRGWREIIQKWLKMDENEPSEKY | |
| | LEVFKDYQRKHPREAGDYSVYEFLSKKENHFIWRNHPEYPYLYATFCEIDKK | |
| | KKDAKQQATFTLADPINHPLWVRFEERSGSNLNKYRILTEQLHTEKLKKKLTV | |
| | QLDRLIYPTESGGWEEKGKVDIVLLPSRQFYNQIFLDIEEKGKHAFTYKDESIK | |
| | FPLKGTLGGARVQFDRDHLRRYPHKVESGNVGRIYFNMTVNIEPTESPVSKSL | |
| | KIHRDDFPKVVNFKPKELTEWIKDSKGKKLKSGIESLEIGLRVMSIDLGQRQA | |
| | AAASIFEVVDQKPDIEGKLFFPIKGTELYAVHRASFNIKLPGETLVKSREVLRK | |
| | AREDNLKLMNQKLNFLRNVLHFQQFEDITEREKRVTKWISRQENSDVPLVYQ | |
| | DELIQIRELMYKPYKDWVAFLKQLHKRLEVEIGKEVKHWRKSLSDGRKGLYG | |
| | ISLKNIDEIDRTRKFLLRWSLRPTEPGEVRRLEPGQRFAIDQLNHLNALKEDRL | |
| | KKMANTIIMHALGYCYDVRKKKWQAKNPACQIILFEDLSNYNPYEERSRFEN | |
| | SKLMKWSRREIPRQVALQGEIYGLQVGEVGAQFSSRFHAKTGSPGIRCSVVTK | |
| | EKLQDNRFFKNLQREGRLTLDKIAVLKEGDLYPDKGGEKFISLSKDRKCVTTH | |
| | ADINAAQNLQKRFWTRTHGFYKVYCKAYQVDGQTVYIPESKDQKQKIIEEFG | |
| | EGYFILKDGVYEWVNAGKLKIKKGSSKQSSSELVDSDILKDSFDLASELKGEK | |
| | LMLYRDPSGNVFPSDKWMAAGVFFGKLERILISKLTNQYSISTIEDDSSKQSM | |

Additional exemplary Cas9 equivalent protein sequences can include the following:

| Description | Sequence | |
|---|---|---|
| AsCas12a | MTQFEGFTNLYQVSKTLRFELIPQGKTLKHIQEQGFIEEDKARNDHYKELKPIIDRIYKT | |
| (previously known | YADQCLQLVQLDWENLSAAIDSYRKEKTEETRNALIEEQATYRNAIHDYFIGRTDNLTDA | |
| as Cpf1) | INKRHAEIYKGLFKAELFNGKVLKQLGTVTTTEHENALLRSFDKFTTYFSGFYENRKNVF | |
| Acidaminococcus | SAEDISTAIPHRIVQDNFPKFKENCHIFTRLITAVPSLREHFENVKKAIGIFVSTSIEEV | |
| sp. (strain BV3L6) | FSFPFYNQLLTQTQIDLYNQLLGGISREAGTEKIKGLNEVLNLAIQKNDETAHIIASLPH | |
| UniProtKB | RFIPLFKQILSDRNTLSFILEEFKSDEEVIQSFCKYKTLLRNENVLETAEALFNELNSID | |
| U2UMQ6 | LTHIFISHKKLETISSALCDHWDTLRNALYERRISELTGKITKSAKEKVQRSLKHEDINL | |
| | QEIISAAGKELSEAFKQKTSEILSHAHAALDQPLPTTLKKQEEKEILKSQLDSLLGLYHL | |
| | LDWFAVDESNEVDPEFSARLTGIKLEMEPSLSFYNKARNYATKKPYSVEKFKLNFQMPTL | |
| | ASGWDVNKEKNNGAILFVKNGLYYLGIMPKQKGRYKALSFEPTEKTSEGFDKMYYDYFPD | |
| | AAKMIPKCSTQLKAVTAHFQTHTTPILLSNNFIEPLEITKEIYDLNNPEKEPKKFQTAYA | |
| | KKTGDQKGYREALCKWIDFTRDFLSKYTKTTSIDLSSLRPSSQYKDLGEYYAELNPLLYH | |
| | ISFQRIAEKEIMDAVETGKLYLFQIYNKDFAKGHHGKPNLHTLYWTGLFSPENLAKTSIK | |
| | LNGQAELFYRPKSRMKRMAHRLGEKMLNKKLKDQKTPIPDTLYQELYDYVNHRLSHDLSD | |
| | EARALLPNVITKEVSHEIIKDRRFTSDKFFFHVPITLNYQAANSPSKFNQRVNAYLKEHP | |
| | ETPIIGIDRGERNLIYITVIDSTGKILEQRSLNTIQQFDYQKKLDNREKERVAARQAWSV | |
| | VGTIKDLKQGYLSQVIHEIVDLMIHYQAVVVLENLNFGFKSKRTGIAEKAVYQQFEKMLI | |
| | DKLNCLVLKDYPAEKVGGVLNPYQLTDQFTSFAKMGTQSGFLFYVPAPYTSKIDPLTGFV | |
| | DPFVWKTIKNHESRKHFLEGFDFLHYDVKTGDFILHFKMNRNLSFQRGLPGFMPAWDIVF | |
| | EKNETQFDAKGTPFIAGKRIVPVIENHRFTGRYRDLYPANELIALLEEKGIVFRDGSNIL | |
| | PKLLENDDSHAIDTMVALIRSVLQMRNSNAATGEDYINSPVRDLNGVCFDSRFQNPEWPM | |
| | DADANGAYHIALKGQLLLNHLKESKDLKLQNGISNQDWLAYIQELRN (SEQ ID NO: 383) | |
| AsCas12a nickase | MTQFEGFTNLYQVSKTLRFELIPQGKTLKHIQEQGFIEEDKARNDHYKELKPIIDRIYKT | |
| (e.g., R1226A) | YADQCLQLVQLDWENLSAAIDSYRKEKTEETRNALIEEQATYRNAIHDYFIGRTDNLTDA | |
| | INKRHAEIYKGLFKAELFNGKVLKQLGTVTTTEHENALLRSFDKFTTYFSGFYENRKNVF | |
| | SAEDISTAIPHRIVQDNFPKFKENCHIFTRLITAVPSLREHFENVKKAIGIFVSTSIEEV | |
| | FSFPFYNQLLTQTQIDLYNQLLGGISREAGTEKIKGLNEVLNLAIQKNDETAHIIASLPH | |
| | RFIPLFKQILSDRNTLSFILEEFKSDEEVIQSFCKYKTLLRNENVLETAEALFNELNSID | |
| | LTHIFISHKKLETISSALCDHWDTLRNALYERRISELTGKITKSAKEKVQRSLKHEDINL | |
| | QEIISAAGKELSEAFKQKTSEILSHAHAALDQPLPTTLKKQEEKEILKSQLDSLLGLYHL | |
| | LDWFAVDESNEVDPEFSARLTGIKLEMEPSLSFYNKARNYATKKPYSVEKFKLNFQMPTL | |
| | ASGWDVNKEKNNGAILFVKNGLYYLGIMPKQKGRYKALSFEPTEKTSEGFDKMYYDYFPD | |
| | AAKMIPKCSTQLKAVTAHFQTHTTPILLSNNFIEPLEITKEIYDLNNPEKEPKKFQTAYA | |
| | KKTGDQKGYREALCKWIDFTRDFLSKYTKTTSIDLSSLRPSSQYKDLGEYYAELNPLLYH | |
| | ISFQRIAEKEIMDAVETGKLYLFQIYNKDFAKGHHGKPNLHTLYWTGLFSPENLAKTSIK | |
| | LNGQAELFYRPKSRMKRMAHRLGEKMLNKKLKDQKTPIPDTLYQELYDYVNHRLSHDLSD | |
| | EARALLPNVITKEVSHEIIKDRRFTSDKFFFHVPITLNYQAANSPSKFNQRVNAYLKEHP | |
| | ETPIIGIDRGERNLIYITVIDSTGKILEQRSLNTIQQFDYQKKLDNREKERVAARQAWSV | |
| | VGTIKDLKQGYLSQVIHEIVDLMIHYQAVVVLENLNFGFKSKRTGIAEKAVYQQFEKMLI | |
| | DKLNCLVLKDYPAEKVGGVLNPYQLTDQFTSFAKMGTQSGFLFYVPAPYTSKIDPLTGFV | |
| | DPFVWKTIKNHESRKHFLEGFDFLHYDVKTGDFILHFKMNRNLSFQRGLPGFMPAWDIVF | |
| | EKNETQFDAKGTPFIAGKRIVPVIENHRFTGRYRDLYPANELIALLEEKGIVFRDGSNIL | |
| | PKLLENDDSHAIDTMVALIRSVLQMANSNAATGEDYINSPVRDLNGVCFDSRFQNPEWPM | |
| | DADANGAYHIALKGQLLLNHLKESKDLKLQNGISNQDWLAYIQELRN (SEQ ID NO: 384) | |
| LbCas12a | 1 | MNYKTGLEDF IGKESLSKTL RNALIPTEST KIHMEEMGVI RDDELRAEKQ QELKEIMDDY |
| (previously known | 61 | YRTFIEEKLG QIQGIQWNSL FQKMEETMED ISVRKDLDKI QNEKRKEICC YFTSDKRFKD |
| as Cpf1) | 121 | LFNAKLITDI LPNFIKDNKE YTEEEKAEKE QTRVLFQRFA TAFTNYFNQR RNNFSEDNIS |
| Lachnospiraceae | 181 | TAISFRIVNE NSEIHLQNMR AFQRIEQQYP EEVCGMEEEY KDMLQEWQMK HIYSVDFYDR |
| bacterium GAM79 | 241 | ELTQPGIEYY NGICGKINEH MNQFCQKNRI NKNDFRMKKL HKQILCKKSS YYEIPFRFES |
| Ref Seq. | 301 | DQEVYDALNE FIKTMKKKEI IRRCVHLGQE CDDYDLGKIY ISSNKYEQIS NALYGSWDTI |
| WP_119623382.1 | 361 | RKCIKEEYMD ALPGKGEKKE EKAEAAAKKE EYRSIADIDK IISLYGSEMD RTISAKKCIT |
| | 421 | EICDMAGQIS IDPLVCNSDI KLLQNKEKTT EIKTILDSFL HVYQWGQTFI VSDIIEKDSY |
| | 481 | FYSELEDVLE DFEGITTLYN HVRSYVTQKP YSTVKFKLHF GSPTLANGWS QSKEYDNNAI |
| | 541 | LLMRDQKFYL GIFNVRNKPD KQIIKGHEKE EKGDYKKMIY NLLPGPSKML PKVFITSRSG |
| | 601 | QETYKPSKHI LDGYNEKRHI KSSPKFDLGY CWDLIDYYKE CIHKHPDWKN YDFHFSDTKD |
| | 661 | YEDISGFYRE VEMQGYQIKW TYISADEIQK LDEKGQIFLF QIYNKDFSVH STGKDNLHTM |
| | 721 | YLKNLFSEEN LKDIVLKLNG EAELFFRKAS IKTPIVHKKG SVLVNRSYTQ TVGNKEIRVS |
| | 781 | IPEEYYTEIY NYLNHIGKGK LSSEAQRYLD EGKIKSFTAT KDIVKNYRYC CDHYFLHLPI |
| | 841 | TINFKAKSDV AVNERTLAYI AKKEDIHIIG IDRGERNLLY ISVVDVHGNI REQRSFNIVN |
| | 901 | GYDYQQKLKD REKSRDAARK NWEEIEKIKE LKEGYLSMVI HYIAQLVVKY NAVVAMEDLN |
| | 961 | YGFKTGRFKV ERQVYQKFET MLIEKLHYLV FKDREVCEEG GVLRGYQLTY IPESLKKVGK |
| | 1021 | QCGFIFYVPA GYTSKIDPTT GFVNLFSFKN LTNRESRQDF VGKFDEIRYD RDKKMFEFSF |
| | 1081 | DYNNYIKKGT ILASTKWKVY TNGTRLKRIV VNGKYTSQSM EVELTDAMEK MLQRAGIEYH |
| | 1141 | DGKDLKGQIV EKGIEAEIID IFRLTVQMRN SRSESEDREY DRLISPVLND KGEFFDTATA |
| | 1201 | DKTLPQDADA NGAYCIALKG LYEVKQIKEN WKENEQFPRN KLVQDNKTWF DFMQKKRYL |
| | | (SEQ ID NO: 385) |
| PcCas12a | 1 | MAKNFEDFKR LYSLSKTLRF EAKPIGATLD NIVKSGLLDE DEHRAASYVK VKKLIDEYHK |
| previously known | 61 | VFIDRVLDDG CLPLENKGNN NSLAEYYESY VSRAQDEDAK KKFKEIQQNL RSVIAKKLTE |
| at Cpf1 | 121 | DKAYANLFGN KLIESYKDKE DKKKIIDSDL IQFINTAEST QLDSMSQDEA KELVKEFWGF |
| Prevotella copri | 181 | VTYFYGFFDN RKNMYTAEEK STGIAYRLVN ENLPKFIDNI EAFNRAITRP EIQENMGVLY |
| Ref Seq. | 241 | SDFSEYLNVE SIQEMFQLDY YNMLLTQKQI DVYNAIIGGK TDDEHDVKIK GINEYINLYN |
| WP_119227726.1 | 301 | QQHKDDKLPK LKALFKQILS DRNAISWLPE EFNSDQEVLN AIKDCYERLA ENVLGDKVLK |
| | 361 | SLLGSLADYS LDGIFIRNDL QLTDISQKMF GNWGVIQNAI MQNIKRVAPA RKHKESEEDY |
| | 421 | EKRIAGIFKK ADSFSISYIN DCLNEADPNN AYFVENYFAT FGAVNTPTMQ RENLFALVQN |
| | 481 | AYTEVAALLH SDYPTVKHLA QDKANVSKIK ALLDAIKSLQ HFVKPLLGKG DESDKDERFY |
| | 541 | GELASLWAEL DTVTPLYNMI RNYMTRKPYS QKKIKLNFEN PQLLGGWDAN KEKDYATIIL |
| | 601 | RRNGLYYLAI MDKDSRKLLG KAMPSDGECY EKMVYKFFKD VTTMIPKCST QLKDVQAYFK |
| | 661 | VNTDDYVLNS KAFNKPLTIT KEVFDLNNVL YGKYKKFQKG YLTATGDNVG YTHAVNVWIK |
| | 721 | FCMDFLNSYD STCIYDFSSL KPESYLSLDA FYQDANLLLY KLSFARASVS YINQLVEEGK |
| | 781 | MYLFQIYNKD FSEYSKGTPN MHTLYWKALF DERNLADVVY KLNGQAEMFY RKKSIENTHP |
| | 841 | THPANHPILN KNKDNKKKES LFDYDLIKDR RYTVDKFMFH VPITMNFKSV GSENINQDVK |
| | 901 | AYLRHADDMH IIGIDRGERH LLYLVVIDLQ GNIKEQYSLN EIVNEYNGNT YHTNYHDLLD |
| | 961 | VREEERLKAR QSWQTIENIK ELKEGYLSQV IHKITQLMVR YHAIVVLEDL SKGFMRSRQK |
| | 1021 | VEKQVYQKFE KMLIDKLNYL VDKKTDVSTP GGLLNAYQLT CKSDSSQKLG KQSGFLFYIP |
| | 1081 | AWNTSKIDPV TGFVNLLDTH SLNSKEKIKA FFSKFDAIRY NKDKKWFEFN LDYDKFGKKA |
| | 1141 | EDTRTKWTLC TRGMRIDTFR NKEKNSQWDN QEVDLTTEMK SLLEHYYIDI HGNLKDAISA |
| | 1201 | QTDKAFFTGL LHILKLTLQM RNSITGTETD YLVSPVADEN GIFYDSRSCG NQLPENADAN |
| | 1261 | GAYNIARKGL MLIEQIKNAE DLNNVKFDIS NKAWLNFAQQ KPYKNG |
| | | (SEQ ID NO: 386) |
| ErCas12a- | 1 | MFSAKLISDI LPEFVIHNNN YSASEKEEKT QVIKLESRFA TSFKDYFKNR ANCESANDIS |
| previously known | 61 | SSSCHRIVND NAEIFFSNAL VYRRIVKNLS NDDINKISGD MKDSLKEMSL EEIYSYEKYG |
| at Cpf1 | 121 | EFITQEGISF YNDICGKVNL FMNLYCQKNK ENKNLYKLRK LHKQILCIAD TSYEVPYKFE |
| Eubacterium | 181 | SDEEVYQSVN GELDNISSKH IVERLRKIGE NYNGYNLDKI YIVSKFYESV SQKTYRDWET |
| rectale | 241 | INTALEIHYN NILPGNGKSK ADKVKKAVKN DLQKSITEIN ELVSNYKLCP DDNIKAETYI |
| Ref Seq. | 301 | HEISHILNNF EAQELKYNPE IHLVESELKA SELKNVLDVI MNAFHWCSVF MTEELVDKDN |
| WP_119223642.1 | 361 | NFYAELEEIY DEIYPVISLY NLVRNYVTQK PYSTKKIKLN FGIPTLADGW SKSKEYSNNA |
| | 421 | IILMRDNLYY LGIFNAKNKP DKKIIEGNTS ENKGDYKKMI YNLLPGPNKM IPKVFLSSKT |
| | 481 | GVETYKPSAY ILEGYKQNKH LKSSKDFDIT FCHDLIDYFK NCIAIHPEWK NFGFDFSDTS |
| | 541 | TYEDISGFYR EVELQGYKID WTYISEKDID LLQEKGQLYL FQIYNKDFSK KSSGNDNLHT |
| | 601 | MYLKNLFSEE NLKDIVLKLN GEAEIFFRKS SIKNPIIHKK GSILVNRTYE AEEKDQFGNI |
| | 661 | QIVRKTIPEN IYQELYKYFN DKSDKELSDE AAKLKNVVGH HEAATNIVKD YRYTYDKYFL |
| | 721 | HMPITINFKA NKTSFINDRI LQYIAKEKDL HVIGIDRGER NLIYVSVIDT CGNIVEQKSF |
| | 781 | NIVNGYDYQI KLKQQEGARQ IARKEWKEIG KIKEIKEGYL SLVIHEISKM VIKYNAIIAM |
| | 841 | EDLSYGFKKG RFKVERQVYQ KFETMLINKL NYLVFKDISI TENGGLLKGY QLTYIPDKLK |
| | 901 | NVGHQCGCIF YVPAAYTSKI DPTTGFVNIF KFKDLTVDAK REFIKKFDSI RYDSDKNLFC |
| | 961 | FTFDYNNFIT QNTVMSKSSW SVYTYGVRIK RRFVNGRFSN ESDTIDITKD MEKTLEMTDI |
| | 1021 | NWRDGHDLRQ DIIDYEIVQH IFEIFKLTVQ MRNSLSELED RDYDRLISPV LNENNIFYDS |
| | 1081 | AKAGDALPKD ADANGAYCIA LKGLYEIKQI TENWKEDGKF SRDKLKISNK DWFDFIQNKR |
| | 1141 | YL (SEQ ID NO: 387) |
| CsCas12a- | 1 | MNYKTGLEDF IGKESLSKTL RNALIPTEST KIHMEEMGVI RDDELRAEKQ QELKEIMDDY |
| previously known | 61 | YRAFIEEKLG QIQGIQWNSL FQKMEETMED ISVRKDLDKI QNEKRKEICC YFTSDKRFKD |
| at Cpf1 | 121 | LFNAKLITDI LPNFIKDNKE YTEEEKAEKE QTRVLFQRFA TAFTNYFNQR RNNFSEDNIS |
| Clostridium sp. | 181 | TAISFRIVNE NSEIHLQNMR AFQRIEQQYP EEVCGMEEEY KDMLQEWQMK HIYLVDFYDR |
| AF34-10BH | 241 | VLTQPGIEYY NGICGKINEH MNQFCQKNRI NKNDFRMKKL HKQILCKKSS YYEIPFRFES |
| Ref Seq. | 301 | DQEVYDALNE FIKTMKEKEI ICRCVHLGQK CDDYDLGKIY ISSNKYEQIS NALYGSWDTI |
| WP_118538418.1 | 361 | RKCIKEEYMD ALPGKGEKKE EKAEAAAKKE EYRSIADIDK IISLYGSEMD RTISAKKCIT |
| | 421 | EICDMAGQIS TDPLVCNSDI KLLQNKEKTT EIKTILDSFL HVYQWGQTFI VSDIIEKDSY |
| | 481 | FYSELEDVLE DFEGITTLYN HVRSYVTQKP YSTVKFKLHF GSPTLANGWS QSKEYDNNAI |
| | 541 | LLMRDQKFYL GIFNVRNKPD KQIIKGHEKE EKGDYKKMIY NLLPGPSKML PKVFITSRSG |
| | 601 | QETYKPSKHI LDGYNEKRHI KSSPKFDLGY CWDLIDYYKE CIHKHPDWKN YDFHFSDTKD |
| | 661 | YEDISGFYRE VEMQGYQIKW TYISADEIQK LDEKGQIFLF QIYNKDFSVH STGKDNLHTM |
| | 721 | YLKNLFSEEN LKDIVLKLNG EAELFFRKAS IKTPVVHKKG SVLVNRSYTQ TVGDKEIRVS |
| | 781 | IPEEYYTEIY NYLNHIGRGK LSTEAQRYLE ERKIKSFTAT KDIVKNYRYC CDHYFLHLPI |
| | 841 | TINFKAKSDI AVNERTLAYI AKKEDIHIIG IDRGERNLLY ISVVDVHGNI REQRSFNIVN |
| | 901 | GYDYQQKLKD REKSRDAARK NWEEIEKIKE LKEGYLSMVI HYIAQLVVKY NAVVAMEDLN |
| | 961 | YGFKTGRFKV ERQVYQKFET MLIEKLHYLV FKDREVCEEG GVLRGYQLTY IPESLKKVGK |
| | 1021 | QCGFIFYVPA GYTSKIDPTT GFVNLFSFKN LTNRESRQDF VGKFDEIRYD RDKKMFEFSF |
| | 1081 | DYNNYIKKGT MLASTKWKVY TNGTRLKRIV VNGKYTSQSM EVELTDAMEK MLQRAGIEYH |
| | 1141 | DGKDLKGQIV EKGIEAEIID IFRLTVQMRN SRSESEDREY DRLISPVLND KGEFFDTATA |
| | 1201 | DKTLPQDADA NGAYCIALKG LYEVKQIKEN WKENEQFPRN KLVQDNKTWF DFMQKKRYL |
| | | (SEQ ID NO: 388) |
| BhCas12b | 1 | MATRSFILKI EPNEEVKKGL WKTHEVLNHG IAYYMNILKL IRQEAIYEHH EQDPKNPKKV |
| Bacillus hisashii | 61 | SKAEIQAELW DFVLKMQKCN SFTHEVDKDE VFNILRELYE ELVPSSVEKK GEANQLSNKF |
| Ref Seq. | 121 | LYPLVDPNSQ SGKGTASSGR KPRWYNLKIA GDPSWEEEKK KWEEDKKKDP LAKILGKLAE |
| WP_095142515.1 | 181 | YGLIPLFIPY TDSNEPIVKE IKWMEKSRNQ SVRRLDKDMF IQALERFLSW ESWNLKVKEE |
| | 241 | YEKVEKEYKT LEERIKEDIQ ALKALEQYEK ERQEQLLRDT LNTNEYRLSK RGLRGWREII |
| | 301 | QKWLKMDENE PSEKYLEVFK DYQRKHPREA GDYSVYEFLS KKENHFIWRN HPEYPYLYAT |
| | 361 | FCEIDKKKKD AKQQATFTLA DPINHPLWVR FEERSGSNLN KYRILTEQLH TEKLKKKLTV |
| | 421 | QLDRLIYPTE SGGWEEKGKV DIVLLPSRQF YNQIFLDIEE KGKHAFTYKD ESIKFPLKGT |
| | 481 | LGGARVQFDR DHLRRYPHKV ESGNVGRIYF NMTVNIEPTE SPVSKSLKIH RDDFPKVVNF |
| | 541 | KPKELTEWIK DSKGKKLKSG IESLEIGLRV MSIDLGQRQA AAASIFEVVD QKPDIEGKLF |
| | 601 | FPIKGTELYA VHRASFNIKL PGETLVKSRE VLRKAREDNL KLMNQKLNFL RNVLHFQQFE |
| | 661 | DITEREKRVT KWISRQENSD VPLVYQDELI QIRELMYKPY KDWVAFLKQL HKRLEVEIGK |
| | 721 | EVKHWRKSLS DGRKGLYGIS LKNIDEIDRT RKFLLRWSLR PTEPGEVRRL EPGQRFAIDQ |
| | 781 | LNHLNALKED RLKKMANTII MHALGYCYDV RKKKWQAKNP ACQIILFEDL SNYNPYEERS |
| | 841 | RFENSKLMKW SRREIPRQVA LQGEIYGLQV GEVGAQFSSR FHAKTGSPGI RCSVVTKEKL |
| | 901 | QDNRFFKNLQ REGRLTLDKI AVLKEGDLYP DKGGEKFISL SKDRKCVTTH ADINAAQNLQ |
| | 961 | KRFWTRTHGF YKVYCKAYQV DGQTVYIPES KDQKQKIIEE FGEGYFILKD GVYEWVNAGK |
| | 1021 | LKIKKGSSKQ SSSELVDSDI LKDSFDLASE LKGEKLMLYR DPSGNVFPSD KWMAAGVFFG |
| | 1081 | KLERILISKL TNQYSISTIE DDSSKQSM (SEQ ID NO: 389) |
| ThCas 12b | 1 | MSEKTTQRAY TLRLNRASGE CAVCQNNSCD CWHDALWATH KAVNRGAKAF GDWLLTLRGG |
| Thermomonas | 61 | LCHTLVEMEV PAKGNNPPQR PTDQERRDRR VLLALSWLSV EDEHGAPKEF IVATGRDSAD |
| hydrothermalis | 121 | DRAKKVEEKL REILEKRDFQ EHEIDAWLQD CGPSLKAHIR EDAVWVNRRA LFDAAVERIK |
| Ref Seq. | 181 | TLTWEEAWDF LEPFFGTQYF AGIGDGKDKD DAEGPARQGE KAKDLVQKAG QWLSARFGIG |
| WP_072754838 | 241 | TGADFMSMAE AYEKIAKWAS QAQNGDNGKA TIEKLACALR PSEPPTLDTV LKCISGPGHK |
| | 301 | SATREYLKTL DKKSTVTQED LNQLRKLADE DARNCRKKVG KKGKKPWADE VLKDVENSCE |
| | 361 | LTYLQDNSPA RHREFSVMLD HAARRVSMAH SWIKKAEQRR RQFESDAQKL KNLQERAPSA |
| | 421 | VEWLDRFCES RSMTTGANTG SGYRIRKRAI EGWSYVVQAW AEASCDTEDK RIAAARKVQA |
| | 481 | DPEIEKFGDI QLFEALAADE AICVWRDQEG TQNPSILIDY VTGKTAEHNQ KRFKVPAYRH |
| | 541 | PDELRHPVFC DFGNSRWSIQ FAIHKEIRDR DKGAKQDTRQ LQNRHGLKMR LWNGRSMTDV |
| | 601 | NLHWSSKRLT ADLALDQNPN PNPTEVTRAD RLGRAASSAF DHVKIKNVFN EKEWNGRLQA |
| | 661 | PRAELDRIAK LEEQGKTEQA EKLRKRLRWY VSFSPCLSPS GPFIVYAGQH NIQPKRSGQY |
| | 721 | APHAQANKGR ARLAQLILSR LPDLRILSVD LGHRFAAACA VWETLSSDAF RREIQGLNVL |
| | 781 | AGGSGEGDLF LHVEMTGDDG KRRTVVYRRI GPDQLLDNTP HPAPWARLDR QFLIKLQGED |
| | 841 | EGVREASNEE LWTVHKLEVE VGRTVPLIDR MVRSGFGKTE KQKERLKKLR ELGWISAMPN |
| | 901 | EPSAETDEKE GEIRSISRSV DELMSSALGT LRLALKRHGN RARIAFAMTA DYKPMPGGQK |
| | 961 | YYFHEAKEAS KNDDETKRRD NQIEFLQDAL SLWHDLFSSP DWEDNEAKKL WQNHIATLPN |
| | 1021 | YQTPEEISAE LKRVERNKKR KENRDKLRTA AKALAENDQL RQHLHDTWKE RWESDDQQWK |
| | 1081 | ERLRSLKDWI FPRGKAEDNP SIRHVGGLSI TRINTISGLY QILKAFKMRP EPDDLRKNIP |
| | 1141 | QKGDDELENF NRRLLEARDR LREQRVKQLA SRIIEAALGV GRIKIPKNGK LPKRPRTTVD |
| | 1201 | TPCHAVVIES LKTYRPDDLR TRRENRQLMQ WSSAKVRKYL KEGCELYGLH FLEVPANYTS |
| | 1261 | RQCSRTGLPG IRCDDVPTGD FLKAPWWRRA INTAREKNGG DAKDRFLVDL YDHLNNLQSK |
| | 1321 | GEALPATVRV PRQGGNLFIA GAQLDDTNKE RRAIQADLNA AANIGLRALL DPDWRGRWWY |
| | 1381 | VPCKDGTSEP ALDRIEGSTA FNDVRSLPTG DNSSRRAPRE IENLWRDPSG DSLESGTWSP |
| | 1441 | TRAYWDTVQS RVIELLRRHA GLPTS (SEQ ID NO: 390) |
| LsCas12b | 1 | MSIRSFKLKL KTKSGVNAEQ LRRGLWRTHQ LINDGIAYYM NWLVLLRQED LFIRNKETNE |
| Laceyella sacchari | 61 | IEKRSKEEIQ AVLLERVHKQ QQRNQWSGEV DEQTLLQALR QLYEEIVPSV IGKSGNASLK |
| WP_132221894.1 | 121 | ARFFLGPLVD PNNKTTKDVS KSGPTPKWKK MKDAGDPNWV QEYEKYMAER QTLVRLEEMG |
| | 181 | LIPLFPMYTD EVGDIHWLPQ ASGYTRTWDR DMFQQAIERL LSWESWNRRV RERRAQFEKK |
| | 241 | THDFASRFSE SDVQWMNKLR EYEAQQEKSL EENAFAPNEP YALTKKALRG WERVYHSWMR |
| | 301 | LDSAASEEAY WQEVATCQTA MRGEFGDPAI YQFLAQKENH DIWRGYPERV IDFAELNHLQ |
| | 361 | RELRRAKEDA TFTLPDSVDH PLWVRYEAPG GTNIHGYDLV QDTKRNLTLI LDKFILPDEN |
| | 421 | GSWHEVKKVP FSLAKSKQFH RQVWLQEEQK QKKREVVFYD YSTNLPHLGT LAGAKLQWDR |
| | 481 | NFLNKRTQQQ IEETGEIGKV FFNISVDVRP AVEVKNGRLQ NGLGKALTVL THPDGTKIVT |
| | 541 | GWKAEQLEKW VGESGRVSSL GLDSLSEGLR VMSIDLGQRT SATVSVFEIT KEAPDNPYKF |
| | 601 | FYQLEGTEMF AVHQRSFLLA LPGENPPQKI KQMREIRWKE RNRIKQQVDQ LSAILRLHKK |
| | 661 | VNEDERIQAI DKLLQKVASW QLNEEIATAW NQALSQLYSK AKENDLQWNQ AIKNAHHQLE |
| | 721 | PVVGKQISLW RKDLSTGRQG IAGLSLWSIE ELEATKKLLT RWSKRSREPG VVKRIERFET |
| | 781 | FAKQIQHHIN QVKENRLKQL ANLIVMTALG YKYDQEQKKW IEVYPACQVV LFENLRSYRF |
| | 841 | SFERSRRENK KLMEWSHRSI PKLVQMQGEL FGLQVADVYA AYSSRYHGRT GAPGIRCHAL |
| | 901 | TEADLRNETN IIHELIEAGF IKEEHRPYLQ QGDLVPWSGG ELFATLQKPY DNPRILTLHA |
| | 961 | DINAAQNIQK RFWHPSMWFR VNCESVMEGE IVTYVPKNKT VHKKQGKTFR FVKVEGSDVY |
| | 1021 | EWAKWSKNRN KNTFSSITER KPPSSMILFR DPSGTFFKEQ EWVEQKTFWG KVQSMIQAYM |
| | 1081 | KKTIVQRMEE (SEQ ID NO: 391) |
| DtCas12b | 1 | MVLGRKDDTA ELRRALWTTH EHVNLAVAEV ERVLLRCRGR SYWTLDRRGD PVHVPESQVA |
| Dsulfonatronum | 61 | EDALAMAREA QRRNGWPVVG EDEEILLALR YLYEQIVPSC LLDDLGKPLK GDAQKIGTNY |
| thiodismutans | 121 | AGPLFDSDTC RRDEGKDVAC CGPFHEVAGK YLGALPEWAT PISKQEFDGK DASHLRFKAT |
| WP_031386437 | 181 | GGDDAFFRVS IEKANAWYED PANQDALKNK AYNKDDWKKE KDKGISSWAV KYIQKQLQLG |
| | 241 | QDPRTEVRRK LWLELGLLPL FIPVFDKTMV GNLWNRLAVR LALAHLLSWE SWNHRAVQDQ |
| | 301 | ALARAKRDEL AALFLGMEDG FAGLREYELR RNESIKQHAF EPVDRPYVVS GRALRSWTRV |
| | 361 | REEWLRHGDT QESRKNICNR LQDRLRGKFG DPDVFHWLAE DGQEALWKER DCVTSFSLLN |
| | 421 | DADGLLEKRK GYALMTFADA RLHPRWAMYE APGGSNLRTY QIRKTENGLW ADVVLLSPRN |
| | 481 | ESAAVEEKTF NVRLAPSGQL SNVSFDQIQK GSKMVGRCRY QSANQQFEGL LGGAEILFDR |
| | 541 | KRIANEQHGA TDLASKPGHV WFKLTLDVRP QAPQGWLDGK GRPALPPEAK HFKTALSNKS |
| | 601 | KFADQVRPGL RVLSVDLGVR SFAACSVFEL VRGGPDQGTY FPAADGRTVD DPEKLWAKHE |
| | 661 | RSFKITLPGE NPSRKEEIAR RAAMEELRSL NGDIRRLKAI LRLSVLQEDD PRTEHLRLFM |
| | 721 | EAIVDDPAKS ALNAELFKGF GDDRFRSTPD LWKQHCHFFH DKAEKVVAER FSRWRTETRP |
| | 781 | KSSSWQDWRE RRGYAGGKSY WAVTYLEAVR GLILRWNMRG RTYGEVNRQD KKQFGTVASA |
| | 841 | LLHHINQLKE DRIKTGADMI IQAARGFVPR KNGAGWVQVH EPCRLILFED LARYRFRTDR |
| | 901 | SRRENSRLMR WSHREIVNEV GMQGELYGLH VDTTEAGFSS RYLASSGAPG VRCRHLVEED |
| | 961 | FHDGLPGMHL VGELDWLLPK DKDRTANEAR RLLGGMVRPG MLVPWDGGEL FATLNAASQL |
| | 1021 | HVIHADINAA QNLQRRFWGR CGEAIRIVCN QLSVDGSTRY EMAKAPKARL LGALQQLKNG |
| | 1081 | DAPFHLTSIP NSQKPENSYV MTPTNAGKKY RAGPGEKSSG EEDELALDIV EQAEELAQGR |
| | 1141 | KTFFRDPSGV FFAPDRWLPS EIYWSRIRRR IWQVTLERNS SGRQERAEMD EMPY |
| | | (SEQ ID NO: 392) |

The base editors described herein may also comprise Cas12a/Cpf1 (dCpf1) variants that may be used as a guide nucleotide sequence-programmable DNA-binding protein domain. The Cas12a/Cpf1 protein has a RuvC-like endonuclease domain that is similar to the RuvC domain of Cas9 but does not have a HNH endonuclease domain, and the N-terminal of Cpf1 does not have the alfa-helical recognition lobe of Cas9. It was shown in Zetsche et al., Cell, 163, 759-771, 2015 (which is incorporated herein by reference) that, the RuvC-like domain of Cpf1 is responsible for cleaving both DNA strands and inactivation of the RuvC-like domain inactivates Cpf1 nuclease activity.

### Cytidine Deaminase Domains

In some embodiments, the base editor comprises a deaminase that is a cytosine deaminase. In some embodiments, the cytosine deaminase domain is fused to the N-terminus of the napDNAbp domain.

In some embodiments, the deaminase is an apolipoprotein B mRNA-editing complex (APOBEC) family deaminase. In some embodiments, the deaminase is an APOBEC1 deaminase, an APOBEC2 deaminase, an APOBEC3A deaminase, an APOBEC3B deaminase, an APOBEC3C deaminase, an APOBEC3D deaminase, an APOBEC3F deaminase, an APOBEC3G deaminase, an APOBEC3H deaminase, or an APOBEC4 deaminase. In some embodiments, the deaminase is an activation-induced deaminase (AID). In some embodiments, the deaminase is a Lamprey CDA1 (pmCDA1) deaminase. In some embodiments, the deaminase is from a human, chimpanzee, gorilla, monkey, cow, dog, rat, or mouse. In some embodiments, the deaminase is from a human. In some embodiments the deaminase is from a rat. In some embodiments, the deaminase is a human APOBEC1 deaminase. In some embodiments, the deaminase is pmCDA1. In some embodiments, the deaminase is human APOBEC3G. In some embodiments, the deaminase is a human APOBEC3G variant. In some embodiments, the deaminase is rat APOBEC1.

In certain embodiments, the cytidine deaminase domain is a “FERNY” polypeptide having an amino acid sequence according to SEQ ID NO: 393 or an amino acid sequence that is at least 80%, 85%, 90%, 95, 98%, 99%, or 99.5% identical to SEQ ID NO: 393, as follows:

(SEQ ID NO: 393)
MFERNYDPRELRKETYLLYEIKWGKSGKLWRHWCQNNRTQHAEVYFLEN
IFNARRFNPSTHCSITWYLSWSPCAECSQKIVDFLKEHPNVNLEIYVAR
LYYHEDERNRQGLRDLVNSGVTIRIMDLPDYNYCWKTFVSDQGGDEDYW
PGHFAPWIKQYSLKL

In certain other embodiment, the cytidine deaminase domain is a domain evolved from a wild-type domain, e.g., evolved through phage-assisted continuous evolution (PACE). In some embodiments, the cytidine deaminase domain is an “evoFERNY” polypeptide having an amino acid sequence according to SEQ ID NO: 394 or an amino acid sequence that is at least 85%, 90%, 95, 98%, 99%, or 99.5% identical to SEQ ID NO: 394, which contains H102P and D104N substitutions relative to SEQ ID NO: 393, as follows: MFERNYDPRELRKETYLLYEIKWGKSGKLWRHWCQNNRTQHAEVYFLENIFNARRF NPSTHCSITWYLSWSPCAECSQKIVDFLKEHPNVNLEIYVARLYYPENERNRQGLRD LVNSGVTIRIMDLPDYNYCWKTFVSDQGGDEDYWPGHFAPWIKQYSLKL (SEQ ID NO: 394). The FERNY and evoFERNY deaminase domains are 162 amino acids in length. These domains are thus shorter than those of any one of SEQ ID NOs: 276-277, 281, 133-134, 292-295, and 487.

In exemplary embodiments, the disclosed CBEs comprise a FERNY or evoFERNY deaminase domain. In some embodiments, the disclosed CBEs comprise a deaminase domain that comprises the amino acid sequence of SEQ ID NO: 393 or 394.

The state-of-the-art cytosine base editor BE3.9 contains a rat APOBEC1 (rAPOBEC1) cytidine deaminase domain. They have high overall activity, severely compromised activity editing GC targets, and high editing on TC targets. Alternative deaminases have been demonstrated as base editors. AID and CDA both work well on GC targets but have lower activity than APOBEC1 generally. APOBEC3G works less well than all of these (see Komor, A. C. et al. Improved base excision repair inhibition and bacteriophage Mu Gam protein yields C:G-to-T:A base editors with higher efficiency and product purity. Sci Adv 3, caao4774 (2017), which is incorporated herein by reference). The TARGET-AID base editing implementation uses CDA (Nishida, K. et al. Targeted nucleotide editing using hybrid prokaryotic and vertebrate adaptive immune systems. Science 353, aaf8729-aaf8729 (2016), which is incorporated herein by reference). “FERNY” is an N- and

C-terminally truncated ancestral sequence reconstruction based on an APOBEC family phylogenetic tree. rAPOBEC1: 229 aa; FERNY: 161 aa. The sequence similarity to rAPOBEC1 is 55%. The evolved FERNY genotype also has high GC activity and is comparably active to APOBEC despite being a shorter protein. Evolved FERNY is described in greater detail in PCT Publication No. WO 2019/023680, published Jan. 31, 2019, which is incorporated herein by reference. Additional exemplary cytidine deaminases are disclosed in PCT Publication No. WO 2021/108717, published Jun. 3, 2021.

Non-limiting examples of suitable cytosine deaminase domains are provided below, as SEQ ID NOs: 276-277, 281, 133-134, 292-295, and 487. In some embodiments, the deaminase is at least 80%, at least 85%, at least 90%, at least 92%, at least 95%, at least 96%, at least 97%, at least 98%, at least 99%, or at least 99.5% identical to any one of the amino acid sequences set forth below.

Human AID
(SEQ ID NO: 276)
MDSLLMNRRKFLYQFKNVRWAKGRRETYLCYVVKRRDSATSFSLDFGYL
RNKNGCHVELLFLRYISDWDLDPGRCYRVTWFTSWSPCYDCARHVADFL
RGNPNLSLRIFTARLYFCEDRKAEPEGLRRLHRAGVQIAIMTFKDYFYC
WNTFVENHERTFKAWEGLHENSVRLSRQLRRILLPLYEVDDLRDAFRTL
GL
Mouse AID
(SEQ ID NO: 277)
MDSLLMKQKKFLYHFKNVRWAKGRHETYLCYVVKRRDSATSCSLDFGHL
RNKSGCHVELLFLRYISDWDLDPGRCYRVTWFTSWSPCYDCARHVAEFL
RWNPNLSLRIFTARLYFCEDRKAEPEGLRRLHRAGVQIGIMTFKDYFYC
WNTFVENRERTFKAWEGLHENSVRLTRQLRRILLPLYEVDDLRDAFRML
GF
Rat APOBEC-3 (rAPOBEC3)
(SEQ ID NO: 281)
MGPFCLGCSHRKCYSPIRNLISQETFKFHFKNLRYAIDRKDTFLCYEVT
RKDCDSPVSLHHGVFKNKDNIHAEICFLYWFHDKVLKVLSPREEFKITW
YMSWSPCFECAEQVLRFLATHHNLSLDIFSSRLYNIRDPENQQNLCRLV
QEGAQVAAMDLYEFKKCWKKFVDNGGRRFRPWKKLLTNFRYQDSKLQEI
LRPCYIPVPSSSSSTLSNICLTKGLPETRFCVERRRVHLLSEEEFYSQF
YNQRVKHLCYYHGVKPYLCYQLEQFNGQAPLKGCLLSEKGKQHAEILFL
DKIRSMELSQVIITCYLTWSPCPNCAWQLAAFKRDRPDLILHIYTSRLY
FHWKRPFQKGLCSLWQSGILVDVMDLPQFTDCWTNFVNPKRPFWPWKGL
EIISRRTQRRLHRIKESWGLQDLVNDFGNLQLGPPMS
Human APOBEC-3G
(SEQ ID NO: 133)
MKPHFRNTVERMYRDTFSYNFYNRPILSRRNTVWLCYEVKTKGPSRPPL
DAKIFRGQVYSELKYHPEMRFFHWFSKWRKLHRDQEYEVTWYISWSPCT
KCTRDMATFLAEDPKVTLTIFVARLYYFWDPDYQEALRSLCQKRDGPRA
TMKIMNYDEFQHCWSKFVYSQRELFEPWNNLPKYYILLHIMLGEILRHS
MDPPTFTFNFNNEPWVRGRHETYLCYEVERMHNDTWVLLNQRRGFLCNQ
APHKHGFLEGRHAELCFLDVIPFWKLDLDQDYRVTCFTSWSPCFSCAQE
MAKFISKNKHVSLCIFTARIYDDQGRCQEGLRTLAEAGAKISIMTYSEF
KHCWDTFVDHQGCPFQPWDGLDEHSQDLSGRLRAILQNQEN
Human APOBEC-3F
(SEQ ID NO: 134)
MKPHFRNTVERMYRDTFSYNFYNRPILSRRNTVWLCYEVKTKGPSRPRL
DAKIFRGQVYSQPEHHAEMCFLSWFCGNQLPAYKCFQITWFVSWTPCPD
CVAKLAEFLAEHPNVTLTISAARLYYYWERDYRRALCRLSQAGARVKIM
DDEEFAYCWENFVYSEGQPFMPWYKFDDNYAFLHRTLKEILRNPMEAMY
PHIFYFHFKNLRKAYGRNESWLCFTMEVVKHHSPVSWKRGVFRNQVDPE
THCHAERCFLSWFCDDILSPNTNYEVTWYTSWSPCPECAGEVAEFLARH
SNVNLTIFTARLYYFWDTDYQEGLRSLSQEGASVEIMGYKDFKYCWENF
VYNDDEPFKPWKGLKYNFLFLDSKLQEILE
Human APOBEC-1
(SEQ ID NO: 292)
MTSEKGPSTGDPTLRRRIEPWEFDVFYDPRELRKEACLLYEIKWGMSRK
IWRSSGKNTTNHVEVNFIKKFTSERDFHPSMSCSITWFLSWSPCWECSQ
AIREFLSRHPGVTLVIYVARLFWHMDQQNRQGLRDLVNSGVTIQIMRAS
EYYHCWRNFVNYPPGDEAHWPQYPPLWMMLYALELHCIILSLPPCLKIS
RRWQNHLTFFRLHLQNCHYQTIPPHILLATGLIHPSVAWR
Mouse APOBEC-1
(SEQ ID NO: 293)
MSSETGPVAVDPTLRRRIEPHEFEVFFDPRELRKETCLLYEINWGGRHS
VWRHTSQNTSNHVEVNFLEKFTTERYFRPNTRCSITWFLSWSPCGECSR
AITEFLSRHPYVTLFIYIARLYHHTDQRNRQGLRDLISSGVTIQIMTEQ
EYCYCWRNFVNYPPSNEAYWPRYPHLWVKLYVLELYCIILGLPPCLKIL
RRKQPQLTFFTITLQTCHYQRIPPHLLWATGLK
Rat APOBEC-1 (rAPOBEC1)
(SEQ ID NO: 294)
MSSETGPVAVDPTLRRRIEPHEFEVFFDPRELRKETCLLYEINWGGRHS
IWRHTSQNTNKHVEVNFIEKFTTERYFCPNTRCSITWFLSWSPCGECSR
AITEFLSRYPHVTLFIYIARLYHHADPRNRQGLRDLISSGVTIQIMTEQ
ESGYCWRNFVNYSPSNEAHWPRYPHLWVRLYVLELYCIILGLPPCLNIL
RRKQPQLTFFTIALQSCHYQRLPPHILWATGLK
Petromyzon marinus CDA1 (pmCDA1)
(SEQ ID NO: 295)
MTDAEYVRIHEKLDIYTFKKQFFNNKKSVSHRCYVLFELKRRGERRACF
WGYAVNKPQSGTERGIHAEIFSIRKVEEYLRDNPGQFTINWYSSWSPCA
DCAEKILEWYNQELRGNGHTLKIWACKLYYEKNARNQIGLWNLRDNGVG
LNVMVSEHYQCCRKIFIQSSHNQLNENRWLEKTLKRAEKRRSELSIMIQ
VKILHTTKSPAV
Evolved pmCDA1 (evoCDA1)
(SEQ ID NO: 487)
MTDAEYVRIHEKLDIYTFKKQFSNNKKSVSHRCYVLFELKRRGERRACF
WGYAVNKPQSGTERGIHAEIFSIRKVEEYLRDNPGQFTINWYSSWSPCA
DCAEKILEWYNQELRGNGHTLKIWVCKLYYEKNARNQIGLWNLRDNGVG
LNVMVSEHYQCCRKIFIQSSHNQLNENRWLEKTLKRAEKRRSELSIMFQ
VKILHTTKSPAV

### Adenosine Deaminase Domains

The disclosure provides adenosine deaminase variants that have activity on deoxyadenosine nucleosides in DNA. As such, the variants provided herein are deoxyadenosine deaminases. In some embodiments, the disclosed adenosine deaminases are variants of known adenosine deaminase TadA7.10, which comprises the following mutations as compared to wild-type ecTadA (SEQ ID NO: 325): W23R, H36L, P48A, R51L, L84F, A106V, D108N, H123Y, S146C, D147Y, R152P, E155V, 1156F, and K157N. In some embodiments, the disclosed adenosine deaminases are variants of a TadA derived from a species other than E. coli, such as Staphylococcus aureus, Salmonella typhi, Shewanella putrefaciens, Haemophilus influenzae, Caulobacter crescentus, or Bacillus subtilis.

In some embodiments, the disclosed adenosine deaminase domain comprises TadA-8e, a variant of E. coli TadA 7.10. TadA-8e (set forth as SEQ ID NO: 433) contains the following substitutions: T111, D119, F149, R26, V88, A109, H122, T166, and D167, relative to TadA7.10 (SEQ ID NO: 315). TadA-8e is disclosed in PCT Publication No. WO 2021/158921, published Aug. 12, 2021, which is incorporated herein by reference. In some embodiments, the adenosine deaminase domain comprises TadA-8e (V106W), which contains a V106W substitution relative to TadA-8e.

In various embodiments, the disclosed adenosine deaminases hydrolytically deaminate a targeted adenosine in a nucleic acid of interest to an inosine, which is read as a guanosine (G) by DNA polymerase enzymes.

These variants may comprise a domain of any of the disclosed base editors (i.e., an adenosine deaminase domain of an adenine base editor). In some embodiments, any of the disclosed adenine base editors are capable of deaminating adenosine in a nucleic acid sequence (e.g., DNA or RNA). The disclosed adenine base editors are further capable of deaminating adenine in DNA.

Exemplary, non-limiting, embodiments of adenosine deaminases are provided herein. In some embodiments, the adenosine deaminase domain of any of the disclosed base editors comprises a single adenosine deaminase, or a monomer. In some embodiments, the adenosine deaminase domain comprises 2, 3, 4 or 5 adenosine deaminases. In some embodiments, the adenosine deaminase domain comprises two adenosine deaminases, or a dimer. In some embodiments, the deaminase domain comprises a dimer of an engineered (or evolved) deaminase and a wild-type deaminase, such as a wild-type E. coli-derived deaminase. It should be appreciated that the mutations provided herein (e.g., mutations in ecTadA) may be applied to adenosine deaminases in other adenine base editors, for example, those provided in PCT Publication No. WO 2018/027078, published Aug. 2, 2018; PCT Publication No. WO 2019/079347 on Apr. 25, 2019; International Application No PCT/US2019/033848, filed May 23, 2019, which published as PCT Publication No. WO 2019/226593 on Nov. 28, 2019; U.S. Patent Publication No. 2018/0073012, published Mar. 15, 2018, which issued as U.S. Pat. No. 10,113,163, on Oct. 30, 2018; U.S. Patent Publication No. 2017/0121693, published May 4, 2017, which issued as U.S. Pat. No. 10,167,457 on Jan. 1, 2019; PCT Publication No. WO 2017/070633, published Apr. 27, 2017; U.S. Patent Publication No. 2015/0166980, published Jun. 18, 2015; U.S. Pat. No. 9,840,699, issued Dec. 12, 2017; and U.S. Pat. No. 10,077,453, issued Sep. 18, 2018; PCT Application No. PCT/US2020/28568, filed Apr. 16, 2020, and PCT Publication No. WO 2021/158921, published Aug. 12, 2021; all of which are incorporated herein by reference in their entireties.

In some embodiments, any of the adenosine deaminases provided herein are capable of deaminating adenine, e.g., deaminating adenine in a deoxyadenosine nucleoside of DNA. The adenosine deaminase may be derived from any suitable organism (e.g., E. coli). In some embodiments, the adenosine deaminase is a naturally-occurring adenosine deaminase that includes one or more mutations corresponding to any of the mutations provided herein (e.g., mutations in ecTadA). One of skill in the art will be able to identify the corresponding residue in any homologous protein and in the respective encoding nucleic acid by methods well known in the art, e.g., by sequence alignment and determination of homologous residues. Exemplary TadA deaminases derived from Bacillus subtilis (set forth in full as SEQ ID NO: 318), S. aureus (SEQ ID NO: 317), and S. pyogenes (SEQ ID NO: 448) are provided. The amino acid substitutions in E. coli TadA-8c, and the homologous mutations in the B. subtilis, S. aureus, and S. pyogenes TadA deaminases, are shown. Accordingly, one of skill in the art would be able to generate mutations in any naturally-occurring adenosine deaminase (e.g., having homology to ccTadA) that corresponds to any of the mutations described herein, e.g., any of the mutations identified in ecTadA. In some embodiments, the adenosine deaminase is derived from a prokaryote. In some embodiments, the adenosine deaminase is from a bacterium. In some embodiments, the adenosine deaminase is from Escherichia coli, Staphylococcus aureus, Salmonella typhi, Shewanella putrefaciens, Haemophilus influenzae, Caulobacter crescentus, or Bacillus subtilis. In some embodiments, the adenosine deaminase is from E. coli.

In some embodiments, the adenosine deaminase comprises TadA9, or a variant thereof. TadA9 contains V82S and Q154R substitutions relative to TadA-8c. (Stated differently, TadA9 contains Y147R, Q154R and I76Y mutations relative to TadA7.10.) In some embodiments, the adenosine deaminase comprises an amino acid sequence that is at least 80%, at least 85%, at least 90%, at least 95%, at least 96%, at least 97%, at least 98%, at least 99%, or at least 99.5% identical to the amino acid sequence of TadA9 (SEQ ID NO: 33). TadA9 may be referred to in the art as TadA*8.9. An ABE containing the TadA9 deaminase is referred to herein as ABE9. TadA9 is described in additional detail in Gaudelli et al., Nat Biotechnol. 2020 July; 38 (7): 892-900 and PCT Publication No. WO 2021/050571, published Mar. 18, 2021, each of which are incorporated herein by reference.

In some embodiments, the adenosine deaminase comprises TadA20, or a variant thereof. TadA20 contains 176Y, V82S, Y123H, Y147R and Q154R substitutions relative to TadA7.10. TadA20 is described in additional detail in Gaudelli et al., Nat Biotechnol. 2020 July; 38 (7): 892-900 and WO 2021/050571, published Mar. 18, 2021. TadA20 may be referred to in the art as TadA*8.20. In some embodiments, the adenosine deaminase comprises an amino acid sequence that is at least 80%, at least 85%, at least 90%, at least 95%, at least 96%, at least 97%, at least 98%, at least 99%, or at least 99.5% identical to the amino acid sequence of TadA20 (SEQ ID NO: 326). An ABE containing the TadA20 deaminase is referred to herein as ABE20. It may be referred to in the art as ABE8.20, ABE8.20-d, or ABE8.20-m.

In some embodiments, the adenosine deaminase comprises an amino acid sequence that is at least 80%, at least 85%, at least 90%, at least 95%, at least 96%, at least 97%, at least 98%, at least 99%, or at least 99.5% identical to any of the amino acid sequences of SEQ ID NOs: 33, 315, 317-326, 433 or 448-449.

It should be appreciated that adenosine deaminases provided herein may include one or more mutations (e.g., any of the mutations provided herein). The disclosure provides adenosine deaminases with a certain percent identity plus any of the mutations or combinations thereof described herein. Any of the adenosine deaminases described herein may be a truncated variant of any of the other adenosine deaminases described herein, e.g., any of the adenosine deaminases of SEQ ID NOs: 33, 315, 317-326, 433 or 448-449.

Exemplary truncated adenosine deaminases may comprise truncations of 1, 2, 3, 4, 5, 6, 7, 8, 9, 10, 11, 12, 13, 14, 15, or more than 15 amino acids from the N-terminus. Other exemplary truncated adenosine deaminases may comprise truncations of 1, 2, 3, 4, 5, 6, 7, 8, 9, 10, 11, 12, 13, 14, 15, or more than 15 amino acids from the C-terminus. In some embodiments, the adenosine deaminase domain comprises a trunacted version of the wild-type ecTadA, as set forth in SEQ ID NO: 324. Any of the adenosine deaminases described herein may include an N-terminal methionine (M) amino acid residue.

It should be appreciated that any of the mutations provided herein (e.g., based on the ecTadA amino acid sequence of SEQ ID NO: 315) may be introduced into other adenosine deaminases, such as S. aureus TadA (saTadA), A. aeolicus TadA (AaTadA), or another adenosine deaminase (e.g., another bacterial adenosine deaminase), such as those sequences provided below. It would be apparent to the skilled artisan how to identify amino acid residues from other adenosine deaminases that are homologous to the mutated residues in ecTadA. Thus, any of the mutations identified in ecTadA may be made in other adenosine deaminases that have homologous amino acid residues. Any of the mutations provided herein may be made individually or in any combination in ecTadA or another adenosine deaminase. Any of the mutated deaminases provided herein may be used in the context of adenine base editor. Any of the deaminases provided herein may have a sequence that begins with a methionine (“M”) before the first amino acid shown in the sequences below.

Exemplary adenosine deaminase variants of the disclosure are described below. In certain embodiments, the adenosine deaminase domain comprises an adenosine deaminase that has a sequence with at least 80%, at least 85%, at least 90%, at least 95%, at least 98%, at least 99%, or at least 99.5% sequence identity to one of the following:

TadA 7.10 (E. coli)
(SEQ ID NO: 315)
SEVEFSHEYWMRHALTLAKRARDEREVPVGAVLVLNNRVIGEGWNRAIG
LHDPTAHAEIMALRQGGLVMQNYRLIDATLYVTFEPCVMCAGAMIHSRI
GRVVFGVRNAKTGAAGSLMDVLHYPGMNHRVEITEGILADECAALLCYF
FRMPRQVFNAQKKAQSSTD
TadA-8e (E. coli)
(SEQ ID NO: 433)
SEVEFSHEYWMRHALTLAKRARDEREVPVGAVLVLNNRVIGEGWNRAIG
LHDPTAHAEIMALRQGGLVMQNYRLIDATLYVTFEPCVMCAGAMIHSRI
GRVVFGVRNSKRGAAGSLMNVLNYPGMNHRVEITEGILADECAALLCDF
YRMPRQVFNAQKKAQSSIN
TadA9
(SEQ ID NO: 33)
SEVEFSHEYWMRHALTLAKRARDEGEVPVGAVLVLNNRVIGEGWNRAIG
LYDPTAHAEIMALRQGGLVMQNYRLIDATLYVTFEPCVMCAGAMIHSRI
GRVVFGVRNSKRGAAGSLMNVLNYPGMDHRVEITEGILANECAALLCDF
YRMPRQVFNAQKKAQSSIN
TadA20
(SEQ ID NO: 326)
SEVEFSHEYWMRHALTLAKRARDEREVPVGAVLVLNNRVIGEGWNRAIG
LHDPTAHAEIMALRQGGLVMQNYRLYDATLYSTFEPCVMCAGAMIHSRI
GRVVFGVRNAKTGAAGSLMDVLHHPGMNHRVEITEGILADECAALLCRF
FRMPRRVFNAQKKAQSSTD
Staphylococcus aureus TadA:
(SEQ ID NO: 317)
MGSHMTNDIYFMTLAIEEAKKAAQLGEVPIGAIITKDDEVIARAHNLRE
TLQQPTAHAEHIAIERAAKVLGSWRLEGCTLYVTLEPCVMCAGTIVMSR
IPRVVYGADDPKGGCSGSLMNLLQQSNFNHRAIVDKGVLKEACSTLLTT
FFKNLRANKKSTN
Bacillus subtilis TadA:
(SEQ ID NO: 318)
MTQDELYMKEAIKEAKKAEEKGEVPIGAVLVINGEIIARAHNLRETEQR
SIAHAEMLVIDEACKALGTWRLEGATLYVTLEPCPMCAGAVVLSRVEKV
VFGAFDPKGGCSGTLMNLLQEERFNHQAEVVSGVLEEECGGMLSAFFRE
LRKKKKAARKNLSE
Salmonella typhimurium (S. typhimurium) TadA:
(SEQ ID NO: 319)
MPPAFITGVTSLSDVELDHEYWMRHALTLAKRAWDEREVPVGAVLVHNH
RVIGEGWNRPIGRHDPTAHAEIMALRQGGLVLQNYRLLDTTLYVTLEPC
VMCAGAMVHSRIGRVVFGARDAKTGAAGSLIDVLHHPGMNHRVEIIEGV
LRDECATLLSDFFRMRRQEIKALKKADRAEGAGPAV
Shewanella putrefaciens (S. putrefaciens) TadA:
(SEQ ID NO: 320)
MDEYWMQVAMQMAEKAEAAGEVPVGAVLVKDGQQIATGYNLSISQHDPT
AHAEILCLRSAGKKLENYRLLDATLYITLEPCAMCAGAMVHSRIARVVY
GARDEKTGAAGTVVNLLQHPAFNHQVEVTSGVLAEACSAQLSRFFKRRR
DEKKALKLAQRAQQGIE
Haemophilus influenzae F3031 (H. influenzae) TadA:
(SEQ ID NO: 321)
MDAAKVRSEFDEKMMRYALELADKAEALGEIPVGAVLVDDARNIIGEGW
NLSIVQSDPTAHAEIIALRNGAKNIQNYRLLNSTLYVTLEPCTMCAGAI
LHSRIKRLVFGASDYKTGAIGSRFHFFDDYKMNHTLEITSGVLAEECSQ
KLSTFFQKRREEKKIEKALLKSLSDK
Caulobacter crescentus (C. crescentus) TadA:
(SEQ ID NO: 322)
MRTDESEDQDHRMMRLALDAARAAAEAGETPVGAVILDPSTGEVIATAG
NGPIAAHDPTAHAEIAAMRAAAAKLGNYRLTDLTLVVTLEPCAMCAGAI
SHARIGRVVFGADDPKGGAVVHGPKFFAQPTCHWRPEVTGGVLADESAD
LLRGFFRARRKAKI
Geobacter sulfurreducens (G. sulfurreducens) TadA:
(SEQ ID NO: 323)
MSSLKKTPIRDDAYWMGKAIREAAKAAARDEVPIGAVIVRDGAVIGRGH
NLREGSNDPSAHAEMIAIRQAARRSANWRLTGATLYVTLEPCLMCMGAI
ILARLERVVFGCYDPKGAAGSLYDLSADPRLNHQVRLSPGVCQEECGTM
LSDFFRDLRRRKKAKATPALFIDERKVPPEP
Streptococcus pyogenes (S. pyogenes) TadA
(SEQ ID NO: 448)
MPYSLEEQTYFMQEALKEAEKSLQKAEIPIGCVIVKDGEIIGRGHNARE
ESNQAIMHAEIMAINEANAHEGNWRLLDTTLFVTIEPCVMCSGAIGLAR
IPHVIYGASNQKFGGADSLYQILTDERLNHRVQVERGLLAADCANIMQT
FFRQGRERKKIAKHLIKEQSDPFD
Aquifex aeolicus (A. aeolicus) TadA
(SEQ ID NO: 449)
MGKEYFLKVALREAKRAFEKGEVPVGAIIVKEGEIISKAHNSVEELKDP
TAHAEMLAIKEACRRLNTKYLEGCELYVTLEPCIMCSYALVLSRIEKVI
FSALDKKHGGVVSVENILDEPTLNHRVKWEYYPLEEASELLSEFFKKLR
NNII

In some embodiments, the adenosine deaminase domain comprises an N-terminal truncated E. coli TadA. In certain embodiments, the adenosine deaminase comprises the amino acid sequence:

(SEQ ID NO: 324)
MSEVEFSHEYWMRHALTLAKRAWDEREVPVGAVLVHNNRVIGEGWNRPIG
RHDPTAHAEIMALRQGGLVMQNYRLIDATLYVTLEPCVMCAGAMIHSRIG
RVVFGARDAKTGAAGSLMDVLHHPGMNHRVEITEGILADECAALLSDFFR
MRRQEIKAQKKAQSSTD

In some embodiments, the TadA deaminase is a full-length E. coli TadA deaminase (ecTadA). For example, in certain embodiments, the adenosine deaminase domain comprises a deaminase that comprises the amino acid sequence:

(SEQ ID NO: 325)
MRRAFITGVFFLSEVEFSHEYWMRHALTLAKRAWDEREVPVGAVLVHNN
RVIGEGWNRPIGRHDPTAHAEIMALRQGGLVMQNYRLIDATLYVTLEPC
VMCAGAMIHSRIGRVVFGARDAKTGAAGSLMDVLHHPGMNHRVEITEGI
LADECAALLSDFFRMRRQEIKAQKKAQSSTD

In some embodiments, the base editor comprises an adenosine deaminase monomer. In other aspects, the base editor comprises an adenosine deaminase dimer. The base editor may comprise a heterodimer of a first adenosine deaminase and a second adenosine deaminase. In some embodiments, the first adenosine deaminase is N-terminal to the second adenosine deaminase in the base editor. In some embodiments, the first adenosine deaminase is C-terminal to the second adenosine deaminase in the base editor. In some embodiments, the first adenosine deaminase and the second deaminase are fused directly to each other or via a linker. In some embodiments, the first adenosine deaminase is fused N-terminal to the napDNAbp via a linker, and the second deaminase is fused C-terminal to the napDNAbp domain via a linker. In other embodiments, the second adenosine deaminase is fused N-terminal to the napDNAbp domain via a linker, and the first deaminase is fused C-terminal to the napDNAbp via a linker.

### Exemplary Base Editors

### Adenine Base Editors

In some aspects, the base editing methods of the disclosure comprise the use of an adenine base editor. Exemplary adenine base editors of this disclosure comprise the monomer and dimer versions of the following editors: Sauri-ABE8e, SaKKH-ABE8e, SaABE8e, CjCas9-ABE8e and Nme2Cas9-ABE8e; SaKKH-ABE8e (V106W), SauriCas9-ABE8e (V106W), CjCas9-ABE8e (V106W), Nme2Cas9-ABE8e (V106W), and SaCas9-ABE8e (V106W); SaKKH-ABE9, SauriCas9-ABE9, CjCas9-ABE9, Nme2Cas9-ABE9, and SaCas9-ABE9; SaKKH-ABE20, SauriCas9-ABE20, CjCas9-ABE20, Nme2Cas9-ABE20, and SaCas9-ABE20; and SaKKH-ABE7.10, SauriCas9-ABE7.10, CjCas9-ABE7.10, Nme2Cas9-ABE7.10, and SaCas9-ABE7.10. In some embodiments, the ABE is Sauri-ABE8e, SaKKH-ABE8e, or SaABE8e. These base editors are 1298, 1291, and 1291 amino acids in length, respectively. Additional base editors are 1221 amino acids (CjABE8e) and 1319 amino acids (Nme2ABE8e) in length. In exemplary embodiments, the ABE is SaKKH-ABE8e. Exemplary ABEs contain an adenosine deaminase domain that comprises a TadA8e and does not comprise a second adenosine deaminase (i.e., the adenosine deaminase domain consists of a deaminase monomer).

Additional exemplary adenine base editors are disclosed in PCT Publication No. WO 2020/051360, published Mar. 12, 2020; PCT Publication No. WO 2021/050571; PCT Publication No, WO 2020/168132, published Aug. 20, 2020; and PCT Publication No. WO 2021/158921, published Aug. 12, 2021, each of which is incorporated herein by reference.

ABE8e may be referred to in the art as “ABE8” or “ABE8.0”. The ABE8e base editor and variants thereof may comprise an adenosine deaminase domain containing a TadA-8c adenosine deaminase monomer (monomer form) or a TadA-8e adenosine deaminase homodimer or heterodimer (dimer form). In some embodiments, the architecture of base editors comprising an adenosine deaminase domain and a napDNAbp is as follows: NH₂-[adenosine deaminase]-[napDNAbp domain]-COOH; or NH₂-[napDNAbp domain]-[adenosine deaminase]-COOH. In certain embodiments, the base editors comprise an ABE8e monomer architecture, which comprises NH₂-[NLS]-[adenosine deaminase]-[napDNAbp domain]-[NLS]-COOH, wherein “NLS” is a nuclear localization sequence.

In some aspects, the disclosure provides complexes of adenine base editors and guide RNAs. Exemplary disclosed complexes comprise any of the following ABEs in conjunction with a guide RNA (such as a single-guide RNA): Sauri-ABE8c, SaKKH-ABE8c, SaABE8c, CjCas9-ABE8e and Nme2Cas9-ABE8c; SaKKH-ABE8c (V106W), SauriCas9-ABE8c (V106W), CjCas9-ABE8c (V106W), Nme2Cas9-ABE8c (V106W), and SaCas9-ABE8c (V106W); SaKKH-ABE9, SauriCas9-ABE9, CjCas9-ABE9, Nme2Cas9-ABE9, and SaCas9-ABE9; SaKKH-ABE20, SauriCas9-ABE20, CjCas9-ABE20, Nme2Cas9-ABE20, and SaCas9-ABE20; and SaKKH-ABE7.10, SauriCas9-ABE7.10, CjCas9-ABE7.10, Nme2Cas9-ABE7.10, and SaCas9-ABE7.10. Other ABEs may be used to deaminate a nucleobase in accordance with the disclosed complexes.

In some embodiments, the ABE is CjCas9-ABE8c. During adenine editing evaluation, this editor exhibited higher context preference for pyrimidines in the nucleotide position 5′ of the target adenine base (YA>>RA). As used herein, “preference” and “context preference” refer to a product purity of above 40% with respect to the target adenosine. Accordingly, in some aspects, the disclosure provides ABEs having pyrimidine (“Y”) context preference, where “context” refers to the presence of a pyrimidine or a purine (“R”) immediately 5′ of the adenine base to be edited (or the target adenine base), such as CjABE8e editors and variants thereof. These ABEs may have a preference for editing an adenosine in a target nucleic acid sequence of 5′-YAN-3′, wherein Y is C or T; N is A, T, C, G, or U; and A is the target adenosine. Accordingly, in some embodiments, an ABE is provided having context preference for deaminating an adenosine in a target nucleic acid sequence of 5′-YAN-3′, wherein Y is C or T, and N is A, T, C, G, or U; and A is the target adenosine.

An exemplary AAV-encoded adenine base editor construct is shown in FIG. 2A. This construct contains an SaABE8e base editor operably controlled by an EFS promoter and a bGH polyA sequence. It also contains a guide RNA encoded in the reverse orientation, as indicated by the arrow pointing away from the 3′ terminus.

The disclosed complexes of ABEs may possess an on-target editing efficiency of more than 50% after being contacted with a nucleic acid molecule comprising a target sequence. Further exemplary ABE complexes possess an on-target editing efficiency of more than 60% after being contacted with a nucleic acid molecule comprising a target sequence. Further exemplary ABEs possess an on-target editing efficiency of more than 65%, more than 70%, more than 75%, more than 80%, more than 82.5%, or more than 85% after being contacted with a nucleic acid molecule comprising a target sequence. The disclosed ABE complexes may exhibit indel frequencies of less than 2.5%, less than 2.4%, less than 2.2%, less than 2.0%, less than 1.75%, less than 1.5%, less than 1.3%, less than 1.1%, or less than 1.0% after being contacted with a nucleic acid molecule containing a target sequence.

In some aspects, the disclosure provide base editors comprising a napDNAbp domain and an adenosine deaminase domain as described herein. The Cas9 domain may be any of the Cas9 domains or Cas9 proteins (e.g., a Cas9 nickase, or nCas9) provided herein. In some embodiments, any of the Cas9 domains or Cas9 proteins (e.g., nCas9) provided herein may be fused with any of the adenosine deaminase domains provided herein.

In some embodiments, the base editors comprising adenosine deaminases and a napDNAbp (e.g., Cas9 domain) do not include a linker sequence. In some embodiments, a linker is present between the adenosine deaminase domain and/or between an adenosine deaminase and the napDNAbp. In some embodiments, the “]-[” used in the general architecture above indicates the presence of an optional linker. In some embodiments, an adenosine deaminase domain and the napDNAbp domain are fused via any of the linkers provided herein. For example, in some embodiments the adenosine deaminase domain (which may include one or more adenosine deaminases) and the napDNAbp are fused via any of the linkers provided below in the section entitled “Linkers”.

In some embodiments, the adenine base editors comprise adenosine deaminases comprising comprises a sequence with at least 80%, 85%, 90%, 95%, 98%, 99%, or 99.5% sequence identity to SEQ ID NO: 433 (TadA-8e). In some embodiments, the adenine base editors comprise the sequence of SEQ ID NO: 433.

In some embodiments, the adenine base editor of the disclosure comprises the sequence of SEQ ID NO: 181. In some embodiments, the adenine base editor of the disclosure comprises the sequence of SEQ ID NO: 182. In other embodiments, the adenine base editor of the disclosure comprises the sequence of SEQ ID NO: 183. In other embodiments, the adenine base editors of the disclosure comprises the sequence of SEQ ID NOs: 171 or 172.

In some embodiments, any of the adenine base editors described herein may comprise an amino acid sequence having 1, 2, 3, 4, 5, 6, 7, 8, 9, 10, 11, 12, 13, 14, 15, 16, 17, 18, 19, 20, 25, 30, or more than 30 amino acids that differ relative to the amino acid sequence of any of SEQ ID NOs: 171-172 and 181-183. These differences may comprise amino acids that have been inserted, deleted, or substituted relative to the reference sequence. In some embodiments, the disclosed adenosine deaminase domains contain stretches of about 50, about 75, about 100, about 125, about 150, about 175, about 200, about 300, about 400, about 500, or more than 500 consecutive amino acids in common with either of SEQ ID NOs: 171-172 and 181-183.

Exemplary base editors comprise sequences that are at least 85%, at least 90%, at least 95%, at least 98%, at least 99%, or at least 99.5% identical to any of the following amino acid sequences (SEQ ID NOs: 171-172 and 181-183). In some embodiments, the disclosed base editors have a sequence comprising any of the following amino acid sequences:

SaABE8e: NLS, linker, TadA-8e, SaCas9
(SEQ ID NO: 171)
MKRTADGSEFESPKKKRKVSEVEFSHEYWMRHALTLAKRARDEREVPVGAVLVLNNRVI
GEGWNRAIGLHDPTAHAEIMALRQGGLVMQNYRLIDATLYVTFEPCVMCAGAMIHSRIGR
VVFGVRNSKRGAAGSLMNVLNYPGMNHRVEITEGILADECAALLCDFYRMPRQVFNAQK
KAQSSINSGGSSGGSSGSETPGTSESATPESSGGSSGGSGKRNYILGLAIGITSVGYGIIDYET
RDVIDAGVRLFKEANVENNEGRRSKRGARRLKRRRRHRIQRVKKLLFDYNLLTDHSELSGI
NPYEARVKGLSQKLSEEEFSAALLHLAKRRGVHNVNEVEEDTGNELSTKEQISRNSKALEE
KYVAELQLERLKKDGEVRGSINRFKTSDYVKEAKQLLKVQKAYHQLDQSFIDTYIDLLETR
RTYYEGPGEGSPFGWKDIKEWYEMLMGHCTYFPEELRSVKYAYNADLYNALNDLNNLVIT
RDENEKLEYYEKFQIIENVFKQKKKPTLKQIAKEILVNEEDIKGYRVTSTGKPEFTNLKVYH
DIKDITARKEIIENAELLDQIAKILTIYQSSEDIQEELTNLNSELTQEEIEQISNLKGYTGTHNL
SLKAINLILDELWHTNDNQIAIFNRLKLVPKKVDLSQQKEIPTTLVDDFILSPVVKRSFIQSIK
VINAIIKKYGLPNDIIIELAREKNSKDAQKMINEMQKRNRQTNERIEEIIRTTGKENAKYLIEK
IKLHDMQEGKCLYSLEAIPLEDLLNNPFNYEVDHIIPRSVSFDNSFNNKVLVKQEENSKKG
NRTPFQYLSSSDSKISYETFKKHILNLAKGKGRISKTKKEYLLEERDINRFSVQKDFINRNLV
DTRYATRGLMNLLRSYFRVNNLDVKVKSINGGFTSFLRRKWKFKKERNKGYKHHAEDALII
ANADFIFKEWKKLDKAKKVMENQMFEEKQAESMPEIETEQEYKEIFITPHQIKHIKDFKD
YKYSHRVDKKPNRELINDTLYSTRKDDKGNTLIVNNLNGLYDKDNDKLKKLINKSPEKLLM
YHHDPQTYQKLKLIMEQYGDEKNPLYKYYEETGNYLTKYSKKDNGPVIKKIKYYGNKLNA
HLDITDDYPNSRNKVVKLSLKPYRFDVYLDNGVYKFVTVKNLDVIKKENYYEVNSKCYEEA
KKLKKISNQAEFIASFYNNDLIKINGELYRVIGVNNDLLNRIEVNMIDITYREYLENMNDKRP
PRIIKTIASKTQSIKKYSTDILGNLYEVKSKKHPQIIKKGSGGSKRTADGSEFEPKKKRKV
SaKKH-ABE8e: NLS, linker, TadA-8e, SaKKH
(SEQ ID NO: 181)
MKRTADGSEFESPKKKRKVSEVEFSHEYWMRHALTLAKRARDEREVPVGAVLVLNNRVI
GEGWNRAIGLHDPTAHAEIMALRQGGLVMQNYRLIDATLYVTFEPCVMCAGAMIHSRIGR
VVFGVRNSKRGAAGSLMNVLNYPGMNHRVEITEGILADECAALLCDFYRMPRQVFNAQK
KAQSSINSGGSSGGSSGSETPGTSESATPESSGGSSGGSGKRNYILGLAIGITSVGYGIIDYE
TRDVIDAGVRLFKEANVENNEGRRSKRGARRLKRRRRHRIQRVKKLLFDYNLLTDHSELSG
INPYEARVKGLSQKLSEEEFSAALLHLAKRRGVHNVNEVEEDTGNELSTKEQISRNSKALEE
KYVAELQLERLKKDGEVRGSINRFKTSDYVKEAKQLLKVQKAYHQLDQSFIDTYIDLLETR
RTYYEGPGEGSPFGWKDIKEWYEMLMGHCTYFPEELRSVKYAYNADLYNALNDLNNLVIT
RDENEKLEYYEKFQIIENVFKQKKKPTLKQIAKEILVNEEDIKGYRVTSTGKPEFTNLKVYH
DIKDITARKEIIENAELLDQIAKILTIYQSSEDIQEELTNLNSELTQEEIEQISNLKGYTGTHNL
SLKAINLILDELWHTNDNQIAIFNRLKLVPKKVDLSQQKEIPTTLVDDFILSPVVKRSFIQSIK
VINAIIKKYGLPNDIIIELAREKNSKDAQKMINEMQKRNRQTNERIEEIIRTTGKENAKYLIEK
IKLHDMQEGKCLYSLEAIPLEDLLNNPFNYEVDHIIPRSVSFDNSENNKVLVKQEENSKKG
NRTPFQYLSSSDSKISYETFKKHILNLAKGKGRISKTKKEYLLEERDINRFSVQKDFINRNLV
DTRYATRGLMNLLRSYFRVNNLDVKVKSINGGFTSFLRRKWKFKKERNKGYKHHAEDALII
ANADFIFKEWKKLDKAKKVMENQMFEEKQAESMPEIETEQEYKEIFITPHQIKHIKDFKD
YKYSHRVDKKPNRKLINDTLYSTRKDDKGNTLIVNNLNGLYDKDNDKLKKLINKSPEKLL
MYHHDPQTYQKLKLIMEQYGDEKNPLYKYYEETGNYLTKYSKKDNGPVIKKIKYYGNKLN
AHLDITDDYPNSRNKVVKLSLKPYRFDVYLDNGVYKFVTVKNLDVIKKENYYEVNSKCYEE
AKKLKKISNQAEFIASFYKNDLIKINGELYRVIGVNNDLLNRIEVNMIDITYREYLENMNDKR
PPHIIKTIASKTQSIKKYSTDILGNLYEVKSKKHPQIIKKGSGGSKRTADGSEFEPKKKRKV
SauriABE8e: NLS, linker, TadA-8e, SauriCas9
(SEQ ID NO: 172)
MKRTADGSEFESPKKKRKVSEVEFSHEYWMRHALTLAKRARDEREVPVGAVLVLNNRVI
GEGWNRAIGLHDPTAHAEIMALRQGGLVMQNYRLIDATLYVTFEPCVMCAGAMIHSRIGR
VVFGVRNSKRGAAGSLMNVLNYPGMNHRVEITEGILADECAALLCDFYRMPRQVFNAQK
KAQSSINSGGSSGGSSGSETPGTSESATPESSGGSSGGSQENQQKQNYILGLAIGITSVGYGL
IDSKTREVIDAGVRLFPEADSENNSNRRSKRGARRLKRRRIHRLNRVKDLLADYQMIDLNN
VPKSTDPYTIRVKGLREPLTKEEFAIALLHIAKRRGLHNISVSMGDEEQDNELSTKQQLQKN
AQQLQDKYVCELQLERLTNINKVRGEKNRFKTEDFVKEVKQLCETQRQYHNIDDQFIQQ
YIDLVSTRREYFEGPGNGSPYGWDGDLLKWYEKLMGRCTYFPEELRSVKYAYSADLFNAL
NDLNNLVVTRDDNPKLEYYEKYHIIENVFKQKKNPTLKQIAKEIGVQDYDIRGYRITKSGKP
QFTSFKLYHDLKNIFEQAKYLEDVEMLDEIAKILTIYQDEISIKKALDQLPELLTESEKSQIA
QLTGYTGTHRLSLKCIHIVIDELWESPENQMEIFTRLNLKPKKVEMSEIDSIPTTLVDEFILS
PVVKRAFIQSIKVINAVINRFGLPEDIIIELAREKNSKDRRKFINKLQKQNEATRKKIEQLLAK
YGNTNAKYMIEKIKLHDMQEGKCLYSLEAIPLEDLLSNPTHYEVDHIIPRSVSFDNSLNNKV
LVKQSENSKKGNRTPYQYLSSNESKISYNQFKQHILNLSKAKDRISKKKRDMLLEERDINKF
EVQKEFINRNLVDTRYATRELSNLLKTYFSTHDYAVKVKTINGGFTNHLRKVWDFKKHRNH
GYKHHAEDALVIANADFLFKTHKALRRTDKILEQPGLEVNDTTVKVDTEEKYQELFETPK
QVKNIKQFRDFKYSHRVDKKPNRQLINDTLYSTREIDGETYVVQTLKDLYAKDNEKVKKLF
TERPQKILMYQHDPKTFEKLMTILNQYAEAKNPLAAYYEDKGEYVTKYAKKGNGPAIHKIK
YIDKKLGSYLDVSNKYPETQNKLVKLSLKSFRFDIYKCEQGYKMVSIGYLDVLKKDNYYYIP
KDKYEAEKQKKKIKESDLFVGSFYYNDLIMYEDELFRVIGVNSDINNLVELNMVDITYKDF
CEVNNVTGEKRIKKTIGKRVVLIEKYTTDILGNLYKTPLPKKPQLIFKRGELSGGSKRTADG
SEFEPKKKRKV
CjABE8e: NLS, linker, TadA-8e, CjCas9
(SEQ ID NO: 182)
MKRTADGSEFESPKKKRKVSEVEFSHEYWMRHALTLAKRARDEREVPVGAVLVLNNRVI
GEGWNRAIGLHDPTAHAEIMALRQGGLVMQNYRLIDATLYVTFEPCVMCAGAMIHSRIGR
VVFGVRNSKRGAAGSLMNVLNYPGMNHRVEITEGILADECAALLCDFYRMPRQVFNAQK
KAQSSINSGGSSGGSSGSETPGTSESATPESSGGSSGGSARILAFAIGISSIGWAFSENDELK
DCGVRIFTKVENPKTGESLALPRRLARSARKRLARRKARLNHLKHLIANEFKLNYEDYQSF
DESLAKAYKGSLISPYELRFRALNELLSKQDFARVILHIAKRRGYDDIKNSDDKEKGAILKAI
KQNEEKLANYQSVGEYLYKEYFQKFKENSKEFTNVRNKKESYERCIAQSFLKDELKLIFKK
QREFGFSFSKKFEEEVLSVAFYKRALKDFSHLVGNCSFFTDEKRAPKNSPLAFMFVALTRII
NLLNNLKNTEGILYTKDDLNALLNEVLKNGTLTYKQTKKLLGLSDDYEFKGEKGTYFIEFK
KYKEFIKALGEHNLSQDDLNEIAKDITLIKDEIKLKKALAKYDLNQNQIDSLSKLEFKDHLN
ISFKALKLVTPLMLEGKKYDEACNELNLKVAINEDKKDFLPAFNETYYKDEVTNPVVLRAIK
EYRKVLNALLKKYGKVHKINIELAREVGKNHSQRAKIEKEQNENYKAKKDAELECEKLGL
KINSKNILKLRLFKEQKEFCAYSGEKIKISDLQDEKMLEIDHIYPYSRSFDDSYMNKVLVFT
KQNQEKLNQTPFEAFGNDSAKWQKIEVLAKNLPTKKQKRILDKNYKDKEQKNFKDRNLN
DTRYIARLVLNYTKDYLDFLPLSDDENTKLNDTQKGSKVHVEAKSGMLTSALRHTWGFSA
KDRNNHLHHAIDAVIIAYANNSIVKAFSDFKKEQESNSAELYAKKISELDYKNKRKFFEPFS
GFRQKVLDKIDEIFVSKPERKKPSGALHEETFRKEEEFYQSYGGKEGVLKALELGKIRKVN
GKIVKNGDMFRVDIFKHKKTNKFYAVPIYTMDFALKVLPNKAVARSKKGEIKDWILMDEN
YEFCFSLYKDSLILIQTKDMQEPEFVYYNAFTSSTVSLIVSKHDNKFETLSKNQKILFKNANE
KEVIAKSIGIQNLKVFEKYIVSALGEVTKAEFRQREDFKKSGGSKRTADGSEFEPKKKRK
V
Nme2ABE8e: NLS, linker, TadA-8e, Nme2Cas9
(SEQ ID NO: 183)
MKRTADGSEFESPKKKRKVSEVEFSHEYWMRHALTLAKRARDEREVPVGAVLVLNNRVI
GEGWNRAIGLHDPTAHAEIMALRQGGLVMQNYRLIDATLYVTFEPCVMCAGAMIHSRIGR
VVFGVRNSKRGAAGSLMNVLNYPGMNHRVEITEGILADECAALLCDFYRMPRQVFNAQK
KAQSSINSGGSSGGSSGSETPGTSESATPESSGGSSGGSAAFKPNPINYILGLAIGIASVGW
AMVEIDEEENPIRLIDLGVRVFERAEVPKTGDSLAMARRLARSVRRLTRRRAHRLLRARRLL
KREGVLQAADFDENGLIKSLPNTPWQLRAAALDRKLTPLEWSAVLLHLIKHRGYLSQRKN
EGETADKELGALLKGVANNAHALQTGDFRTPAELALNKFEKESGHIRNQRGDYSHTFSRK
DLQAELILLFEKQKEFGNPHVSGGLKEGIETLLMTQRPALSGDAVQKMLGHCTFEPAEPK
AAKNTYTAERFIWLTKLNNLRILEQGSERPLTDTERATLMDEPYRKSKLTYAQARKLLGLED
TAFFKGLRYGKDNAEASTLMEMKAYHAISRALEKEGLKDKKSPLNLSSELQDEIGTAFSLF
KTDEDITGRLKDRVQPEILEALLKHISFDKFVQISLKALRRIVPLMEQGKRYDEACAEIYGD
HYGKKNTEEKIYLPPIPADEIRNPVVLRALSQARKVINGVVRRYGSPARIHIETAREVGKSFK
DRKEIEKRQEENRKDREKAAAKFREYFPNFVGEPKSKDILKLRLYEQQHGKCLYSGKEIN
LVRLNEKGYVEIDHALPFSRTWDDSFNNKVLVLGSENQNKGNQTPYEYFNGKDNSREWQ
EFKARVETSRFPRSKKQRILLQKFDEDGFKECNLNDTRYVNRFLCQFVADHILLTGKGKR
RVFASNGQITNLLRGFWGLRKVRAENDRHHALDAVVVACSTVAMQQKITRFVRYKEMNAF
DGKTIDKETGKVLHQKTHFPQPWEFFAQEVMIRVFGKPDGKPEFEEADTPEKLRTLLAE
KLSSRPEAVHEYVTPLFVSRAPNRKMSGAHKDTLRSAKRFVKHNEKISVKRVWLTEIKLAD
LENMVNYKNGREIELYEALKARLEAYGGNAKQAFDPKDNPFYKKGGQLVKAVRVEKTQES
GVLLNKKNAYTIADNGDMVRVDVFCKVDKKGKNQYFIVPIYAWQVAENILPDIDCKGYRID
DSYTFCFSLHKYDLIAFQKDEKSKVEFAYYINCDSSNGRFYLAWHDKGSKEQQFRISTQNL
VLIQKYQVNELGKEIRPCRLKKRPPVRSGGSKRTADGSEFEPKKKRKV

### Cytosine Base Editors

In some aspects, the disclosure provides cytosine base editors (CBEs). Examples of these CBEs include CjCas9-BE3.9, CjCas9-FERNY-BE3.9, and CjCas9-evoFERNY-BE3.9 The CBEs of the disclosure may be a variant of any of CjCas9-BE3.9, CjCas9-FERNY-BE3.9, and CjCas9-evoFERNY-BE3.9.

In some embodiments, the disclosed cytosine base editors (CBEs) may comprise a fusion protein comprising: (i) a nucleic acid programmable DNA binding protein (napDNAbp) domain; (ii) a cytidine deaminase domain; and (iii) a uracil glycosylase inhibitor domain (UGI). In various embodiments, the disclosed CBEs contain a single UGI domain. The disclosed CBEs can be arranged structurally in a variety of configurations, which include, but are not limited to:
NH₂-[cytidine deaminase domain]-[napDNAbp domain]-[UGI]-COOH;
NH₂-[cytidine deaminase domain]-[UGI]-[napDNAbp domain]-COOH;
NH₂-[napDNAbp domain]-[UGI]-[cytidine deaminase domain]-COOH;
NH₂-[napDNAbp domain]-[cytidine deaminase domain]-[UGI]-COOH;
NH₂-[UGI]-[cytidine deaminase domain]-[napDNAbp domain]-COOH; or
NH₂-[UGI]-[napDNAbp domain]-[cytidine deaminase domain]-COOH, wherein each instance of “]-[” comprises an optional linker.

An exemplary construct encoding a single AAV-CBE is shown in FIG. 14. This CBE contains a BE3.9 architecture, with the deaminase FERNY (or evoFERNY) as the cytidine deaminase domain positioned 5′ of the napDNAbp domain. In this construct, the cytosine base editor contains a CjCas9 napDNAbp domain. A human U6-controlled (“hU6”) sgRNA is positioned at the 3′ end, in the reverse orientation to the base editor. This construct contains an EFS promoter driving the base editor, and an SV40 late polyA sequence. The construct has a length of 5.012 kb. Where indicated, “BE3.9” refers to the BE3.9 CBE architecture, i.e., NH₂-[first nuclear localization sequence]-[cytosine deaminase domain]-[32aa linker]-[napDNAbp domain]-[9aa linker]-[first UGI domain]-[second nuclear localization sequence]-COOH. The disclosed CBEs may further comprise one or more nuclear localization signals (NLSs).

Additional exemplary CBEs are disclosed in PCT Publication No. WO 2019/023680, published Jan. 31, 2019, and PCT Publication No. WO 2021/108717, published Jun. 3, 2021, each of which are incorporated herein by reference. Additional CBEs are disclosed in Villiger, L. et al. Nature Medicine 24, 1519-1525 (2018), incorporated herein by reference. Villiger and colleagues developed an intein-split S. aureus CBE. The disclosed single-AAV CBEs demonstrate comparable if not improved activity relative to that shown by Villiger.

The disclosed CBEs may comprise modified (or evolved) cytosine deaminase domains, such as deaminase domains that recognize an expanded PAM sequence, have improved efficiency of deaminating 5′-GC targets, and/or make edits in a narrower target window, In some embodiments, the disclosed cytidine nucleobase editors comprise evolved nucleic acid programmable DNA binding proteins (napDNAbp), such as an evolved Cas9.

In some aspects, the disclosure provides complexes of cytosine base editors and guide RNAs, e.g., complexes of any of CjCas9-BE3.9, CjCas9-FERNY-BE3.9, and CjCas9-evoFERNY-BE3.9 and a sgRNA.

Non-limiting examples of CBEs are provided below, as SEQ ID NOs: 19 and 20, which are FERNY-BE3.9 editors. These editors contain SpCas9 as a napDNAbp domain. Exemplary AAV-encoded CBEs of the disclosure contain a CjCas9 domain (SEQ ID NO: 379) in place of the SpCas9 domain of the below-described FERNY-BE3.9 editors.

The following base editor (SEQ ID NO: 19) contains wild-type FERNY, which may be used as a reference base editor. This base editor was evolved to generate the evoFERNY base editor shown as SEQ ID NO: 20. These base editors contain a bpNLS and a single UGI domain.

Amino acid sequence of FERNY-BE3.9
(SEQ ID NO: 19)
MKRTADGSEFESPKKKRKVSFERNYDPRELRKETYLLYEIKWGKSGKLWRHWCQ
NNRTQHAEVYFLENIFNARRFNPSTHCSITWYLSWSPCAECSQKIVDFLKEHPNVNLE
IYVARLYYHEDERNRQGLRDLVNSGVTIRIMDLPDYNYCWKTFVSDQGGDEDYWP
GHFAPWIKQYSLKLSGGSSGGSSGSETPGTSESATPESSGGSSGGSDKKYSIGLAIGTNS
VGWAVITDEYKVPSKKFKVLGNTDRHSIKKNLIGALLFDSGETAEATRLKRTARRRY
TRRKNRICYLQEIFSNEMAKVDDSFFHRLEESFLVEEDKKHERHPIFGNIVDEVAYHE
KYPTIYHLRKKLVDSTDKADLRLIYLALAHMIKFRGHFLIEGDLNPDNSDVDKLFIQL
VQTYNQLFEENPINASGVDAKAILSARLSKSRRLENLIAQLPGEKKNGLFGNLIALSL
GLTPNFKSNFDLAEDAKLQLSKDTYDDDLDNLLAQIGDQYADLFLAAKNLSDAILLS
DILRVNTEITKAPLSASMIKRYDEHHQDLTLLKALVRQQLPEKYKEIFFDQSKNGYAG
YIDGGASQEEFYKFIKPILEKMDGTEELLVKLNREDLLRKQRTFDNGSIPHQIHLGELH
AILRRQEDFYPFLKDNREKIEKILTFRIPYYVGPLARGNSRFAWMTRKSEETITPWNFE
EVVDKGASAQSFIERMTNFDKNLPNEKVLPKHSLLYEYFTVYNELTKVKYVTEGMR
KPAFLSGEQKKAIVDLLFKTNRKVTVKQLKEDYFKKIECFDSVEISGVEDRFNASLGT
YHDLLKIIKDKDFLDNEENEDILEDIVLTLTLFEDREMIEERLKTYAHLFDDKVMKQL
KRRRYTGWGRLSRKLINGIRDKQSGKTILDFLKSDGFANRNFMQLIHDDSLTFKEDIQ
KAQVSGQGDSLHEHIANLAGSPAIKKGILQTVKVVDELVKVMGRHKPENIVIEMARE
NQTTQKGQKNSRERMKRIEEGIKELGSQILKEHPVENTQLQNEKLYLYYLQNGRDM
YVDQELDINRLSDYDVDHIVPQSFLKDDSIDNKVLTRSDKNRGKSDNVPSEEVVKK
MKNYWRQLLNAKLITQRKFDNLTKAERGGLSELDKAGFIKRQLVETRQITKHVAQIL
DSRMNTKYDENDKLIREVKVITLKSKLVSDFRKDFQFYKVREINNYHHAHDAYLNA
VVGTALIKKYPKLESEFVYGDYKVYDVRKMIAKSEQEIGKATAKYFFYSNIMNFFKT
EITLANGEIRKRPLIETNGETGEIVWDKGRDFATVRKVLSMPQVNIVKKTEVQTGGFS
KESILPKRNSDKLIARKKDWDPKKYGGFDSPTVAYSVLVVAKVEKGKSKKLKSVKE
LLGITIMERSSFEKNPIDFLEAKGYKEVKKDLIIKLPKYSLFELENGRKRMLASAGELQ
KGNELALPSKYVNFLYLASHYEKLKGSPEDNEQKQLFVEQHKHYLDEIIEQISEFSKR
VILADANLDKVLSAYNKHRDKPIREQAENIIHLFTLTNLGAPAAFKYFDTTIDRKRYT
STKEVLDATLIHQSITGLYETRIDLSQLGGDSGGSGGSGGSTNLSDIIEKETGKQLVIQE
SILMLPEEVEEVIGNKPESDILVHTAYDESTDENVMLLTSDAPEYKPWALVIQDSNGE
NKIKMLSGGSKRTADGSEFEPKKKRKV*

The following base editor contains evoFERNY, which was evolved based on the base editor provided above (SEQ ID NO: 19).

Amino acid sequence of evoFERNY-BE3.9
(SEQ ID NO: 20)
MKRTADGSEFESPKKKRKVSFERNYDPRELRKETYLLYEIKWGKSGKLWRHWCQ
NNRTQHAEVYFLENIFNARRENPSTHCSITWYLSWSPCAECSQKIVDFLKEHPNVNLE
IYVARLYYPENERNRQGLRDLVNSGVTIRIMDLPDYNYCWKTFVSDQGGDEDYWPG
HFAPWIKQYSLKLSGGSSGGSSGSETPGTSESATPESSGGSSGGSDKKYSIGLAIGTNSV
GWAVITDEYKVPSKKFKVLGNTDRHSIKKNLIGALLFDSGETAEATRLKRTARRRYT
RRKNRICYLQEIFSNEMAKVDDSFFHRLEESFLVEEDKKHERHPIFGNIVDEVAYHEK
YPTIYHLRKKLVDSTDKADLRLIYLALAHMIKFRGHFLIEGDLNPDNSDVDKLFIQLV
QTYNQLFEENPINASGVDAKAILSARLSKSRRLENLIAQLPGEKKNGLFGNLIALSLGL
TPNFKSNFDLAEDAKLQLSKDTYDDDLDNLLAQIGDQYADLFLAAKNLSDAILLSDI
LRVNTEITKAPLSASMIKRYDEHHQDLTLLKALVRQQLPEKYKEIFFDQSKNGYAGYI
DGGASQEEFYKFIKPILEKMDGTEELLVKLNREDLLRKQRTFDNGSIPHQIHLGELHAI
LRRQEDFYPFLKDNREKIEKILTFRIPYYVGPLARGNSRFAWMTRKSEETITPWNFEE
VVDKGASAQSFIERMTNFDKNLPNEKVLPKHSLLYEYFTVYNELTKVKYVTEGMRK
PAFLSGEQKKAIVDLLFKTNRKVTVKQLKEDYFKKIECFDSVEISGVEDRFNASLGTY
HDLLKIIKDKDFLDNEENEDILEDIVLTLTLFEDREMIEERLKTYAHLFDDKVMKQLK
RRRYTGWGRLSRKLINGIRDKQSGKTILDFLKSDGFANRNFMQLIHDDSLTFKEDIQK
AQVSGQGDSLHEHIANLAGSPAIKKGILQTVKVVDELVKVMGRHKPENIVIEMAREN
QTTQKGQKNSRERMKRIEEGIKELGSQILKEHPVENTQLQNEKLYLYYLQNGRDMY
VDQELDINRLSDYDVDHIVPQSFLKDDSIDNKVLTRSDKNRGKSDNVPSEEVVKKM
KNYWRQLLNAKLITQRKFDNLTKAERGGLSELDKAGFIKRQLVETRQITKHVAQILD
SRMNTKYDENDKLIREVKVITLKSKLVSDFRKDFQFYKVREINNYHHAHDAYLNAV
VGTALIKKYPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEVQTGGFSK
ESILPKRNSDKLIARKKDWDPKKYGGFDSPTVAYSVLVVAKVEKGKSKKLKSVKEL
LGITIMERSSFEKNPIDFLEAKGYKEVKKDLIIKLPKYSLFELENGRKRMLASAGELQK
GNELALPSKYVNFLYLASHYEKLKGSPEDNEQKQLFVEQHKHYLDEIIEQISEFSKRV
ILADANLDKVLSAYNKHRDKPIREQAENIIHLFTLTNLGAPAAFKYFDTTIDRKRYTS
TKEVLDATLIHQSITGLYETRIDLSQLGGDSGGSGGSGGSTNLSDIIEKETGKQLVIQES
ILMLPEEVEEVIGNKPESDILVHTAYDESTDENVMLLTSDAPEYKPWALVIQDSNGE
NKIKMLSGGSKRTADGSEFEPKKKRKV*

In some embodiments, the disclosed base editors comprise CjCas9-FERNY-BE3.9, which is provided below as SEQ ID NO: 21. In some embodiments, the disclosed base editors comprise CjCas9-evoFERNY-BE3.9, which is provided below as SEQ ID NO: 22. Any of the disclosed base editors may comprise a sequence having at least 80%, 85%, 90%, 92.5%, 95%, 97%, 98% or 99% identity to any of SEQ ID NOs: 21 and 22. The base editors may comprise the sequence of SEQ ID NO: 21 or 22. These base editors contain a bpNLS and a single UGI domain. The FERNY deaminase is italicized.

CjCas9-evoFERNY-BE3.9
(SEQ ID NO: 21)
MKRTADGSEFESPKKKRKVSFERNYDPRELRKETYLLYEIKWGKSGKLWRHWCQNNRT
QHAEVYFLENIFNARRFNPSTHCSITWYLSWSPCAECSQKIVDFLKEHPNVNLEIYVARLYY
PENERNRQGLRDLVNSGVTIRIMDLPDYNYCWKTFVSDQGGDEDYWPGHFAPWIKQYSL
KLSGGSSGGSSGSETPGTSESATPESSGGSSGGSARILAFAIGISSIGWAFSENDELKDC
GVRIFTKVENPKTGESLALPRRLARSARKRLARRKARLNHLKHLIANEFKLNYEDYQ
SFDESLAKAYKGSLISPYELRFRALNELLSKQDFARVILHIAKRRGYDDIKNSDDKEK
GAILKAIKQNEEKLANYQSVGEYLYKEYFQKFKENSKEFTNVRNKKESYERCIAQSF
LKDELKLIFKKQREFGFSFSKKFEEEVLSVAFYKRALKDFSHLVGNCSFFTDEKRAPK
NSPLAFMFVALTRIINLLNNLKNTEGILYTKDDLNALLNEVLKNGTLTYKQTKKLLG
LSDDYEFKGEKGTYFIEFKKYKEFIKALGEHNLSQDDLNEIAKDITLIKDEIKLKKALA
KYDLNQNQIDSLSKLEFKDHLNISFKALKLVTPLMLEGKKYDEACNELNLKVAINED
KKDFLPAFNETYYKDEVTNPVVLRAIKEYRKVLNALLKKYGKVHKINIELAREVGK
NHSQRAKIEKEQNENYKAKKDAELECEKLGLKINSKNILKLRLFKEQKEFCAYSGEKI
KISDLQDEKMLEIDHIYPYSRSFDDSYMNKVLVFTKQNQEKLNQTPFEAFGNDSAKW
QKIEVLAKNLPTKKQKRILDKNYKDKEQKNFKDRNLNDTRYIARLVLNYTKDYLDF
LPLSDDENTKLNDTQKGSKVHVEAKSGMLTSALRHTWGFSAKDRNNHLHHAIDAVI
IAYANNSIVKAFSDFKKEQESNSAELYAKKISELDYKNKRKFFEPFSGFRQKVLDKID
EIFVSKPERKKPSGALHEETFRKEEEFYQSYGGKEGVLKALELGKIRKVNGKIVKNG
DMFRVDIFKHKKTNKFYAVPIYTMDFALKVLPNKAVARSKKGEIKDWILMDENYEF
CFSLYKDSLILIQTKDMQEPEFVYYNAFTSSTVSLIVSKHDNKFETLSKNQKILFKNAN
EKEVIAKSIGIQNLKVFEKYIVSALGEVTKAEFRQREDFKKSGGSGGSGGSTNLSDIIE
KETGKQLVIQESILMLPEEVEEVIGNKPESDILVHTAYDESTDENVMLLTSDAPEYKP
WALVIQDSNGENKIKMLSGGSKRTADGSEFEPKKKRKV
CjCas9-FERNY-BE3.9
(SEQ ID NO: 22)
MKRTADGSEFESPKKKRKVSFERNYDPRELRKETYLLYEIKWGKSGKLWRHWCQNNRT
QHAEVYFLENIFNARRFNPSTHCSITWYLSWSPCAECSQKIVDFLKEHPNVNLEIYVARLYY
HEDERNRQGLRDLVNSGVTIRIMDLPDYNYCWKTFVSDQGGDEDYWPGHFAPWIKQYSL
KLSGGSSGGSSGSETPGTSESATPESSGGSSGGSARILAFAIGISSIGWAFSENDELKDC
GVRIFTKVENPKTGESLALPRRLARSARKRLARRKARLNHLKHLIANEFKLNYEDYQ
SFDESLAKAYKGSLISPYELRFRALNELLSKQDFARVILHIAKRRGYDDIKNSDDKEK
GAILKAIKQNEEKLANYQSVGEYLYKEYFQKFKENSKEFTNVRNKKESYERCIAQSF
LKDELKLIFKKQREFGFSFSKKFEEEVLSVAFYKRALKDFSHLVGNCSFFTDEKRAPK
NSPLAFMFVALTRIINLLNNLKNTEGILYTKDDLNALLNEVLKNGTLTYKQTKKLLG
LSDDYEFKGEKGTYFIEFKKYKEFIKALGEHNLSQDDLNEIAKDITLIKDEIKLKKALA
KYDLNQNQIDSLSKLEFKDHLNISFKALKLVTPLMLEGKKYDEACNELNLKVAINED
KKDFLPAFNETYYKDEVTNPVVLRAIKEYRKVLNALLKKYGKVHKINIELAREVGK
NHSQRAKIEKEQNENYKAKKDAELECEKLGLKINSKNILKLRLFKEQKEFCAYSGEKI
KISDLQDEKMLEIDHIYPYSRSFDDSYMNKVLVFTKQNQEKLNQTPFEAFGNDSAKW
QKIEVLAKNLPTKKQKRILDKNYKDKEQKNFKDRNLNDTRYIARLVLNYTKDYLDF
LPLSDDENTKLNDTQKGSKVHVEAKSGMLTSALRHTWGFSAKDRNNHLHHAIDAVI
IAYANNSIVKAFSDFKKEQESNSAELYAKKISELDYKNKRKFFEPFSGFRQKVLDKID
EIFVSKPERKKPSGALHEETFRKEEEFYQSYGGKEGVLKALELGKIRKVNGKIVKNG
DMFRVDIFKHKKTNKFYAVPIYTMDFALKVLPNKAVARSKKGEIKDWILMDENYEF
CFSLYKDSLILIQTKDMQEPEFVYYNAFTSSTVSLIVSKHDNKFETLSKNQKILFKNAN
EKEVIAKSIGIQNLKVFEKYIVSALGEVTKAEFRQREDFKKSGGSGGSGGSTNLSDIIE
KETGKQLVIQESILMLPEEVEEVIGNKPESDILVHTAYDESTDENVMLLTSDAPEYKP
WALVIQDSNGENKIKMLSGGSKRTADGSEFEPKKKRKV

Recombinant Adeno-Associated Viral (rAAV) Vectors

Aspects of the present disclosure relate to using recombinant adeno-associated virus vectors for the delivery of any of the disclosed nucleic acid molecules. The rAAV particles of the present disclosure comprise a rAAV vector (i.e., a recombinant genome of the rAAV) encapsidated in the viral capsid proteins. See U.S. Patent Publication No. 2018/0127780, published May 10, 2018, and PCT Publication No. WO 2020/236982, published Nov. 26, 2020, the disclosures of each of which are incorporated herein by reference.

In some embodiments, the AAV nucleic acid vector is single-stranded. In some embodiments, the AAV nucleic acid vector is self-complementary. In various embodiments, the rAAV vectors of the disclosure do not contain any inteins.

In some embodiments, viral sequences that facilitate integration comprise Inverted Terminal Repeat (ITR) sequences. In some embodiments, nucleic acid molecule is flanked on each side by an ITR sequence. In some embodiments, the nucleic acid vector further comprises a region encoding an AAV Rep protein as described herein, either contained within the region flanked by ITRs or outside the region. The ITR sequences can be derived from any AAV serotype (e.g., 1, 2, 3, 4, 5, 6, 7, 8, 9, or 10) or can be derived from more than one serotype. In some embodiments, the ITR sequences are derived from AAV8 or AAV9. In some embodiments, in methods of packaging any of the disclosed rAAV particles, a nucleic acid plasmid, such as a helper plasmid, that comprises a region encoding a Rep protein and/or a Cap (capsid) protein is provided.

Thus, in some embodiments, the rAAV particles disclosed herein comprise an rAAV2 particle, rAAV6 particle, rAAV8 particle, rPHP.B particle, rPHP.eB particle, or rAAV9 particle, or a variant thereof. In particular embodiments, the disclosed rAAV particles are rAAV8 or rAAV9 particles.

Exemplary rAAV particles provided herein include, but are not limited to, an rAAV8-Sauri-ABE8c, rAAV9-Sauri-ABE8c, rAAV8-SaKKH-ABE8c, rAAV9-SaKKH-ABE8c, rAAV8-CjCas9-ABE8c, rAAV9-CjCas9-ABE8e, rAAV8-Nme2Cas9-ABE8e, or an rAAV9-Nme2Cas9-ABE8e particle. In certain embodiments, the rAAV particle comprises an rAAV8-SaKKH-ABE8c particle. In some embodiments, the rAAV particle comprises a rAAV9-CjBE3.9 particle or an rAAV8-CjBE3.9 particle.

ITR sequences and plasmids containing ITR sequences are known in the art and commercially available (see, e.g., products and services available from Vector Biolabs, Philadelphia, PA; Cellbiolabs, San Diego, CA; Agilent Technologies, Santa Clara, Ca; and Addgene, Cambridge, MA; and Gene delivery to skeletal muscle results in sustained expression and systemic delivery of a therapeutic protein. Kessler P D, Podsakoff G M, Chen X, McQuiston S A, Colosi P C, Matelis L A, Kurtzman G J, Byrne B J. Proc Natl Acad Sci USA. 1996 Nov. 26; 93 (24):14082-7; and Curtis A. Machida. Methods in Molecular Medicine™. Viral Vectors for Gene Therapy Methods and Protocols. 10.1385/1-59259-304-6:201 @ Humana Press Inc. 2003. Chapter 10. Targeted Integration by Adeno-Associated Virus. Matthew D. Weitzman, Samuel M. Young Jr., Toni Cathomen and Richard Jude Samulski; U.S. Pat. Nos. 5,139,941 and 5,962,313, all of which are incorporated herein by reference).

In some embodiments, the rAAV vector of the present disclosure comprises one or more regulatory elements to control the expression of the heterologous nucleic acid region (e.g., promoters, transcriptional terminators, and/or other regulatory elements). In some embodiments, the first and/or second nucleotide sequence is operably linked to one or more (e.g., 1, 2, 3, 4, 5, or more) transcriptional terminators. Non-limiting examples of transcriptional terminators that may be used in accordance with the present disclosure include transcription terminators (or polyadenylation signals) of the bovine growth hormone gene (bGH), human growth hormone gene (hGH), SV40, CW3, φ, or combinations thereof. In exemplary embodiments, the transcriptional terminator is an SV40 polyadenylation signal. In exemplary embodiments, the transcriptional terminator does not contain a posttranscription response element, such as WPRE element.

In some aspects, provided herein are methods of making (or manufacturing, or packaging) any of the disclosed rAAV particles. rAAV particles may be manufactured according to any method known in the art. Methods of packaging are known in the art and reagents are commercially available (see, e.g., Zolotukhin et al. Production and purification of serotype 1, 2, and 5 recombinant adeno-associated viral vectors. Methods 28 (2002) 158-167; and U.S. Patent Publication Numbers US 2007-0015238 and US 2012-0322861, which are incorporated herein by reference; and plasmids and kits available from ATCC and Cell Biolabs, Inc.). For example, a plasmid comprising a gene of interest may be combined with one or more helper plasmids, e.g., that contain a rep gene (e.g., encoding Rep78, Rep68, Rep52 and Rep40) and a cap gene (encoding VP1, VP2, and VP3, including a modified VP2 region as described herein), and transfected into a recombinant cells such that the rAAV particle can be packaged and subsequently purified.

Packaging cells (or host cells) are typically used to form virus particles that are capable of infecting a host cell. Such cells include 293 cells, which package adenovirus, and ψ2 cells or PA317 cells, which package retrovirus. Viral vectors used in gene therapy are usually generated by producing a cell line that packages a nucleic acid vector into a viral particle. The vectors typically contain the minimal viral sequences required for packaging and subsequent integration into a host, other viral sequences being replaced by an expression cassette for the polynucleotide(s) to be expressed. The missing viral functions are typically supplied in trans by the packaging cell line. For example, AAV vectors used in gene therapy typically only possess ITR sequences from the AAV genome which are required for packaging and integration into the host genome. Viral DNA is packaged in a cell line, which contains a helper plasmid encoding the other AAV genes, namely rep and cap, but lacking ITR sequences. The cell line may also be infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV. Additional methods for the delivery of nucleic acids to cells are known to those skilled in the art, such as those disclosed in US 2003/0087817, published May 8, 2003, PCT Application No. WO 2016/205764, published Dec. 22, 2016, and PCT Application No. WO 2018/071868, published Apr. 19, 2018.

In various embodiments, the base editor constructs may be engineered for delivery in one or more rAAV vectors. An rAAV as related to any of the methods and compositions provided herein may be of any serotype including any derivative or pseudotype (e.g., 1, 2, 3, 4, 5, 6, 7, 8, 9, 10, 11, 12, 13, 2/1, 2/5, 2/8, 2/9, 3/1, 3/5, 3/8, or 3/9). An rAAV may comprise a genetic payload (i.e., a recombinant nucleic acid vector that expresses a transgene of interest, such as a whole base editor that is carried by the rAAV into a cell) that is to be delivered to a cell. An rAAV may be chimeric.

As used herein, the serotype of an rAAV refers to the serotype of the capsid proteins of the recombinant virus. Non-limiting examples of derivatives and pseudotypes include rAAV2/1, rAAV2/5, rAAV2/8, rAAV2/9, AAV2-AAV3 hybrid, AAVrh.10, AAVrh.74, AAVhu. 14, AAV3a/3b, AAVrh32.33, AAV-HSC15, AAV-HSC17, AAVhu.37, AAVrh.8, CHt-P6, AAV2.5, AAV6.2, AAV218, AAV-HSC15/17, AAVM41, AAV9.45, AAV6 (Y445F/Y731F), AAV2.5T, AAV-HAE1/2, AAV clone 32/83, AAVShH10, AAV2 (Y->F), AAV8 (Y733F), AAV2.15, AAV2.4, AAVM41, and AAVr3.45. A non-limiting example of derivatives and pseudotypes that have chimeric VP1 proteins is rAAV2/5-1VP1u, which has the genome of AAV2, capsid backbone of AAV5 and VP1u of AAV1. Other non-limiting example of derivatives and pseudotypes that have chimeric VP1 proteins are rAAV2/5-8VP1u, rAAV2/9-1VP1u, and rAAV2/9-8VP1u. In some embodiments, the capsid of the disclosed rAAV particles is AAV8 (serotype 8). In some embodiments, the capsid of the disclosed rAAV particles is AAV8 (serotype 9). In some embodiments, the capsid is of serotype 2, 6, PHP.B, or PHP.cB.

Compositions comprising a plurality of any of the disclosed rAAV particles are provided herein. Exemplary compositions contain a plurality of any of the disclosed rAAV8 particles, rAAV9 particles, rAAV2 particles, rAAV6 particles, rAAVPHP.B particles, and rAAVPHP.cB particles.

In some embodiments, disclosed are methods of administering compositions of rAAV8 particles or rAAV9 particles by intravenous administration. In other embodiments, tissues not well-transduced by intravenous AAV9 injections may be transduced by other existing AAV variants, such as AAV4 transduction of the lung, or by different delivery routes, such as AAV9 transduction of kidney cells by retrograde ureteral infusion.

AAV derivatives/pseudotypes, and methods of producing such derivatives/pseudotypes are known in the art (see, e.g., Mol. Ther. 2012 April; 20 (4): 699-708. doi: 10.1038/mt.2011.287. Epub 2012 Jan. 24. The AAV vector toolkit: poised at the clinical crossroads. Asokan Al, Schaffer D V, Samulski R J.). Methods for producing and using pseudotyped rAAV vectors are known in the art (see, e.g., Duan et al., J. Virol., 75:7662-7671, 2001; Halbert et al., J. Virol., 74:1524-1532, 2000; Zolotukhin et al., Methods, 28:158-167, 2002; and Auricchio et al., Hum. Molec. Genet., 10:3075-3081, 2001).

In some aspects, the disclosure provides compositions containing a plurality of any of the disclosed rAAV particles. In some aspects, the disclosure provides host cells containing a plurality of any of the disclosed rAAV particles. In some embodiments, the host cells are mammalian cells, such as human cells. In other embodiments, the host cells are yeast cells, plant cells, or bacterial cells.

Methods of delivery to a target cell or target tissue of any of the disclosed rAAV particles and compositions and host cells comprising rAAV particles are known in the art. In some embodiments, any of the disclosed rAAV particles, host cells, or compositions are delivered to a subject, such as a mammalian subject. In some embodiments, the rAAV particles are delivered to a human subject.

In some embodiments, the disclosed rAAV particles and compositions are administered to a subject in a single injection, such as a single systemic injection. In some embodiments, the disclosed rAAV particles and compositions are administered to a subject in multiple injections. rAAV particles are known to transduce target tissues within days, but are typically allowed three to four weeks to complete transduction, genome integration, and clearance, from the cell. Accordingly, in some aspects, any of the disclosed rAAV particles or compositions are administered to a subject for a period of three weeks. in some aspects, any of the disclosed rAAV particles or compositions are administered to a subject for a period of between three and four weeks.

In some embodiments, any of the disclosed rAAV particles or compositions is administered to a subject or a target tissue in a therapeutically effective amount of about 10¹⁵, about 10¹⁴, about 10¹³, about 10¹², about 10¹¹, or less than about 10¹¹ vector genomes (vg) per kg weight of the subject. In some embodiments, the rAAV particles are administered in an amount of between 10¹⁵ and 10¹⁴, between 10¹⁴ and 10¹³, between 10¹³ and 10¹², between 10¹² and 10¹¹, or between 10¹² and 10¹¹ vgs per kg. In some embodiments, the rAAV particles are administered in an amount of between 10¹⁴ and 10¹¹ vgs per kg. In some embodiments, any of the disclosed rAAV particles or compositions is administered to a target tissue of a subject in a lower dose than is convention for dual AAV particle delivery, such as that described in PCT Publication No. WO 2020/236982, published Nov. 26, 2020 and Levy, J. M., et al. Nat Biomed Eng 4, 97-110 (2020).

In some aspects, the present disclosure provides single AAV vector delivery of base editors to target tissues, such as liver, neuronal, heart, muscular, or ocular tissue. In some embodiments, any of the disclosed rAAV particles or compositions is administered to the liver (hepatic) tissue of a subject. In some embodiments, any of the disclosed rAAV particles or compositions is administered to the cardiac tissue of a subject. In some embodiments, any of the disclosed rAAV particles or compositions is administered to the neuronal tissue of a subject. In some embodiments, any of the disclosed rAAV particles or compositions is administered to the muscular, or neuromuscular, tissue of a subject. In some embodiments, any of the disclosed rAAV particles or compositions is administered to the ocular tissue of a subject.

In some embodiments, the disclosed rAAV particles provide for transduction of the target tissue to achieve expression and translation of the payload or transgene, e.g., a base editor in accordance with the present disclosure, for a sufficient duration to install desired mutations in the genome of a target cell. In some embodiments, the desired mutation is an A to G mutation. In some embodiments, the desired mutation is a C to T mutation. In some embodiments, the disclosed rAAV particles provide for sufficient expression and translation of the base editor transgene for a sufficient duration to install desired (on-target) mutations in the genome with a tolerable degree of off-target effects, such as bystander edits. In some embodiments, the disclosed rAAV particles provide for sufficient expression and translation of the base editor transgene for a sufficient duration to install desired mutations in the genome without appreciable off-target editing. In some embodiments, the disclosed rAAV particles provide for sufficient expression and translation of the base editor transgene for a sufficient duration to install desired mutations in the genome without appreciable bystander editing.

Suitable routes of administrating the disclosed compositions of rAAV particles include, without limitation: topical, subcutaneous, transdermal, intradermal, intralesional, intraarticular, intraperitoneal, intravesical, transmucosal, gingival, intradental, intracochlear, transtympanic, intraorgan, epidural, intrathecal, intramuscular, intravenous, systemic, intravascular, intraosseus, periocular, intratumoral, intracerebral, parenteral, and intracerebroventricular administration. In some embodiments, the route of administration is systemic (intravenous). In some embodiments, the pharmaceutical composition described herein is administered locally to a diseased site.

In some aspects, pharmaceutical compositions comprising any of the disclosed compositions and a pharmaceutically acceptable carrier are provided herein. In some embodiments, the pharmaceutical composition is formulated for delivery to a subject, e.g., for base editing in a genome. In some embodiments, the disclosed compositions are formulated in accordance with routine procedures as a composition adapted for intravenous or subcutaneous administration to a subject, e.g., a human. In some embodiments, compositions provided herein are formulated for delivery to a subject, for example, to a human subject, in order to effect a targeted genomic modification within the subject. In some embodiments, cells are obtained from the subject and contacted with a any of the pharmaceutical compositions provided herein. In some embodiments, cells removed from a subject and contacted ex vivo with a pharmaceutical composition are re-introduced into the subject, optionally after the desired genomic modification has been effected or detected in the cells. Subjects to which administration of the pharmaceutical compositions is contemplated include, but are not limited to, humans and/or other primates; mammals, domesticated animals, pets, and commercially relevant mammals such as cattle, pigs, horses, sheep, cats, dogs, mice, and/or rats; and/or birds, including commercially relevant birds such as chickens, ducks, geese, and/or turkeys.

Formulations of the pharmaceutical compositions described herein may be prepared by any method known or hereafter developed in the art of pharmacology. In general, such preparatory methods include the step of bringing the active ingredient(s) into association with an excipient and/or one or more other accessory ingredients, and then, if necessary and/or desirable, shaping and/or packaging the product into a desired single- or multi-dose unit. Pharmaceutical formulations may additionally comprise a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired.

In some embodiments, pharmaceutical compositions of rAAV particles for administration by injection are solutions in sterile isotonic aqueous buffer. Where necessary, the pharmaceutical can also include a solubilizing agent and a local anesthetic such as lignocaine to case pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the pharmaceutical is to be administered by infusion, it can be dispensed with an infusion bottle containing sterile pharmaceutical grade water or saline. Where the pharmaceutical composition is administered by injection, an ampoule of sterile water for injection or saline can be provided so that the ingredients can be mixed prior to administration.

As used here, the term “pharmaceutically acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the compound from one site (e.g., the delivery site) of the body, to another site (e.g., organ, tissue or portion of the body). A pharmaceutically acceptable carrier is “acceptable” in the sense of being compatible with the other ingredients of the formulation and not injurious to the tissue of the subject (e.g., physiologically compatible, sterile, physiologic pH, etc.). Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C2-C12 alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein.

The rAAV particle pharmaceutical compositions of this disclosure may be administered or packaged as a unit dose, for example. The term “unit dose” when used in reference to a pharmaceutical composition of the present disclosure refers to physically discrete units suitable as unitary dosage for the subject, each unit containing a predetermined quantity of active material calculated to produce the desired therapeutic effect in association with the required diluent, i.e., a carrier or vehicle.

rAAV Vector Sequences

In some aspects, the disclosure provides the rAAV vector nucleic acid sequences set forth below. In some embodiments, the disclosed vectors comprise a nucleic acid sequence having at least 80%, at least 85%, at least 90%, at least 95%, at least 98%, at least 99%, or at least 99.5% sequence identity to any of SEQ ID NOs: 100-102. In certain embodiments, the disclosed vectors the nucleic acid sequence of any of SEQ ID NOs: 100-102. The components of each sequence, along with the ITR-to-ITR length, is indicated.

In some embodiments, any of the vectors described herein may comprise a nucleic acid sequence having 1-5, 5-10, 10-15, 15-20, 20-25, 25-30, 30-35, 35-40, 40-45, 45-50, or more than 50 nucleotides that differ relative to the sequence of any of SEQ ID NOs: 100-102. These differences may comprise nucleotides that have been inserted, deleted, or substituted relative to any of SEQ ID NOs: 100-102. In some embodiments, the disclosed vectors contain stretches of about 50, about 75, about 100, about 125, about 150, about 175, about 200, about 300, about 400, about 500, or more than 500 consecutive nucleotides in common with any of SEQ ID NOs: 100-102.

Single AAV SaABE8e
ITR-EFS [symbol] -SaABE8e (start codon-BPNLS-TadA-SaCas9 D10A-BPNLS-stop

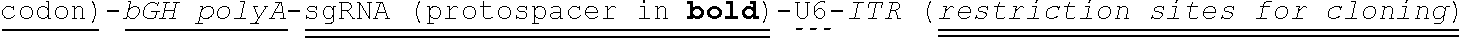

[4804 bp is the ITR-to-ITR length]
CTGCGCGCTCGCTCGCTCACTGAGGCCGCCCGGGCAAAGCCCGGGCGTCGGGCGACCTTTGGTCGCC
CGGCCTCAGTGAGCGAGCGAGCGCGCAGAGAGGGAGTGGCCAACTCCATCACTAGGGGTTCCTGCGGC
CTCTAGAATTCGCTAGCTAGGTCTTGAAAGGAGTGGGAATTGGCTCCGGTGCCCGTCAGTGG
GCAGAGCGCACATCGCCCACAGTCCCCGAGAAGTTGGGGGGAGGGGTCGGCAATTGATCCG
GTGCCTAGAGAAGGTGGCGCGGGGTAAACTGGGAAAGTGATGTCGTGTACTGGCTCCGCCT
TTTTCCCGAGGGTGGGGGAGAACCGTATATAAGTGCAGTAGTCGCCGTGAACGTTCTTTTTC
GCAACGGGTTTGCCGCCAGAACACAGGACCGGTGCCACCATGAAACGGACAGCCGACGGAAG
CGAGTTCGAGTCACCAAAGAAGAAGCGGAAAGTCTCTGAGGTGGAGTTTTCCCACGAGTACTGGA
TGAGACATGCCCTGACCCTGGCCAAGAGGGCACGGGATGAGAGGGAGGTGCCTGTGGGAGCCGTG
CTGGTGCTGAACAATAGAGTGATCGGCGAGGGCTGGAACAGAGCCATCGGCCTGCACGACCCAAC
AGCCCATGCCGAAATTATGGCCCTGAGACAGGGCGGCCTGGTCATGCAGAACTACAGACTGATTG
ACGCCACCCTGTACGTGACATTCGAGCCTTGCGTGATGTGCGCCGGCGCCATGATCCACTCTAGGA
TCGGCCGCGTGGTGTTTGGCGTGAGGAACTCAAAAAGAGGCGCCGCAGGCTCCCTGATGAACGTG
CTGAACTACCCCGGCATGAATCACCGCGTCGAAATTACCGAGGGAATCCTGGCAGATGAATGTGC
CGCCCTGCTGTGCGATTTCTATCGGATGCCTAGACAGGTGTTCAATGCTCAGAAGAAGGCCCAGAG
CTCCATCAACTCCGGAGGATCTAGCGGAGGCTCCTCTGGCTCTGAGACACCTGGCACAAGCGAGA
GCGCAACACCTGAAAGCAGCGGGGGCAGCAGCGGGGGGTCAGGGAAGCGAAATTACATTCTGGG
GCTGGCCATTGGCATTACATCAGTGGGCTATGGCATCATTGACTACGAGACAAGGGACGTGATCG
ACGCCGGCGTGAGACTGTTCAAGGAGGCCAACGTGGAGAACAATGAGGGCCGGAGATCCAAGAG
GGGAGCAAGGCGCCTGAAGCGGAGAAGGCGCCACAGAATCCAGAGAGTGAAGAAGCTGCTGTTC
GATTACAACCTGCTGACCGACCACTCCGAGCTGTCTGGCATCAATCCTTATGAGGCCAGAGTGAAG
GGCCTGTCCCAGAAGCTGTCTGAGGAGGAGTTTAGCGCCGCCCTGCTGCACCTGGCAAAGAGGAG
AGGCGTGCACAACGTGAATGAGGTGGAGGAGGACACCGGCAACGAGCTGTCCACAAAGGAGCAG
ATCAGCCGCAATTCCAAGGCCCTGGAGGAGAAGTATGTGGCCGAGCTGCAGCTGGAGCGGCTGAA
GAAGGATGGCGAGGTGAGGGGCTCCATCAATCGCTTCAAGACCTCTGACTACGTGAAGGAGGCCA
AGCAGCTGCTGAAGGTGCAGAAGGCCTACCACCAGCTGGATCAGTCCTTTATCGATACATATATCG
ACCTGCTGGAGACAAGGCGCACATACTATGAGGGACCAGGAGAGGGCTCTCCCTTCGGCTGGAAG
GACATCAAGGAGTGGTACGAGATGCTGATGGGCCACTGCACCTATTTTCCAGAGGAGCTGAGAAG
CGTGAAGTACGCCTATAACGCCGATCTGTACAACGCCCTGAATGACCTGAACAACCTGGTCATCAC
CAGGGATGAGAACGAGAAGCTGGAGTACTATGAGAAGTTCCAGATCATCGAGAACGTGTTCAAGC
AGAAGAAGAAGCCTACACTGAAGCAGATCGCCAAGGAGATCCTGGTGAACGAGGAGGACATCAA
GGGCTACCGCGTGACCTCCACAGGCAAGCCAGAGTTCACCAATCTGAAGGTGTATCACGATATCA
AGGACATCACAGCCCGGAAGGAGATCATCGAGAACGCCGAGCTGCTGGATCAGATCGCCAAGATC
CTGACCATCTATCAGAGCTCCGAGGACATCCAGGAGGAGCTGACCAACCTGAATAGCGAGCTGAC
ACAGGAGGAGATCGAGCAGATCAGCAATCTGAAGGGCTACACCGGCACACACAACCTGAGCCTG
AAGGCCATCAATCTGATCCTGGATGAGCTGTGGCACACAAACGACAATCAGATCGCCATCTTTAAC
CGGCTGAAGCTGGTGCCAAAGAAGGTGGACCTGTCCCAGCAGAAGGAGATCCCAACCACACTGGT
GGACGATTTCATCCTGTCTCCCGTGGTGAAGCGGAGCTTCATCCAGAGCATCAAAGTGATCAACGC
CATCATCAAGAAGTACGGCCTGCCCAATGATATCATCATCGAGCTGGCCAGGGAGAAGAACTCCA
AGGACGCCCAGAAGATGATCAATGAGATGCAGAAGAGGAACCGCCAGACCAATGAGCGGATCGA
GGAGATCATCAGAACCACAGGCAAGGAGAACGCCAAGTACCTGATCGAGAAGATCAAGCTGCAC
GATATGCAGGAGGGCAAGTGTCTGTATTCTCTGGAGGCCATCCCTCTGGAGGACCTGCTGAACAAT
CCATTCAACTACGAGGTGGATCACATCATCCCCCGGAGCGTGAGCTTCGACAATTCTTTTAACAAT
AAGGTGCTGGTGAAGCAGGAGGAGAACAGCAAGAAGGGCAATAGGACCCCTTTCCAGTACCTGTC
TAGCTCCGATTCTAAGATCAGCTACGAGACATTCAAGAAGCACATCCTGAATCTGGCCAAGGGCA
AGGGCCGCATCAGCAAGACCAAGAAGGAGTACCTGCTGGAGGAGCGGGACATCAACAGATTCTCC
GTGCAGAAGGACTTCATCAACCGGAATCTGGTGGACACCAGATACGCCACACGCGGCCTGATGAA
TCTGCTGCGGTCTTATTTCAGAGTGAACAATCTGGATGTGAAGGTGAAGAGCATCAACGGCGGCTT
CACCTCCTTTCTGCGGAGAAAGTGGAAGTTTAAGAAGGAGCGCAACAAGGGCTATAAGCACCACG
CCGAGGATGCCCTGATCATCGCCAATGCCGACTTCATCTTTAAGGAGTGGAAGAAGCTGGACAAG
GCCAAGAAAGTGATGGAGAACCAGATGTTCGAGGAGAAGCAGGCCGAGAGCATGCCCGAGATCG
AGACAGAGCAGGAGTACAAGGAGATTTTCATCACACCTCACCAGATCAAGCACATCAAGGACTTC
AAGGACTACAAGTATTCTCACAGGGTGGATAAGAAGCCCAACCGCGAGCTGATCAATGACACCCT
GTATAGCACACGGAAGGACGATAAGGGCAATACCCTGATCGTGAACAATCTGAACGGCCTGTACG
ACAAGGATAATGACAAGCTGAAGAAGCTGATCAACAAGTCTCCCGAGAAGCTGCTGATGTACCAC
CACGATCCTCAGACATATCAGAAGCTGAAGCTGATCATGGAGCAGTACGGCGACGAGAAGAACCC
ACTGTATAAGTACTATGAGGAGACAGGCAACTACCTGACAAAGTATAGCAAGAAGGATAATGGCC
CCGTGATCAAGAAGATCAAGTACTATGGCAACAAGCTGAATGCCCACCTGGACATCACCGACGAT
TACCCTAACTCTCGCAATAAGGTGGTGAAGCTGAGCCTGAAGCCATACCGGTTCGACGTGTACCTG
GACAACGGCGTGTATAAGTTTGTGACAGTGAAGAATCTGGATGTGATCAAGAAGGAGAACTACTA
TGAGGTGAACAGCAAGTGCTACGAGGAGGCCAAGAAGCTGAAGAAGATCAGCAACCAGGCCGAG
TTCATCGCCTCTTTTTACAACAATGACCTGATCAAGATCAATGGCGAGCTGTATAGAGTGATCGGC
GTGAACAATGATCTGCTGAACAGAATCGAAGTGAATATGATCGACATCACCTACAGGGAGTATCT
GGAGAACATGAATGATAAGAGGCCCCCTCGCATCATCAAGACCATCGCCTCTAAGACACAGAGCA
TCAAGAAGTACAGCACAGACATCCTGGGGAACCTGTATGAAGTCAAGAGCAAGAAACATCCTCAG
ATTATCAAGAAAGGCTCTGGCGGCTCAAAAAGAACCGCCGACGGCAGCGAATTCGAGCCCAAGAA

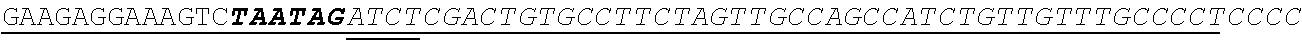

CGTGCCTTCCTTGACCCTGGAAGGTGCCACTCCCACTGTCCTTTCCTAATAAAATGAGGAAATTGCATCGC
ATTGTCTGAGTAGGTGTCATTCTATTCTGGGGGGGGGGTGGGGCAGGACAGCAAGGGGGAGGATTGG
GAAGACAATAGCAGGCATGCTGGGGATGCGGTGGGCTCTATGGCTCGAGCGGCCCAAGCTTAAAAAAA
TCTCGCCAACAAGTTGACGAGATAAACACGGCATTTTGCCTTGTTTTAGTAGATTCTGTAATTTTCA

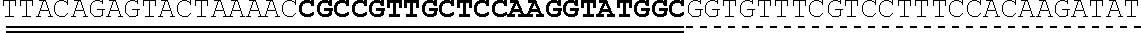

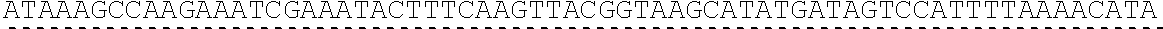

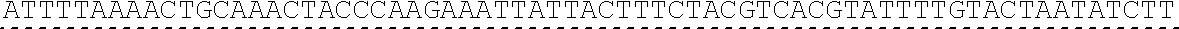

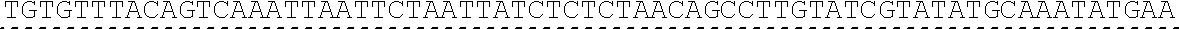

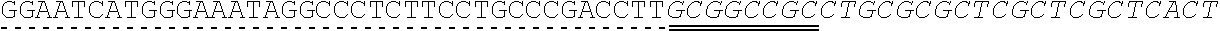

GAGGCCGCCCGGGCAAAGCCCGGGCGTCGGGCGACCTTTGGTCGCCCGGCCTCAGTGAGCGAGCGAG
CGCGCAGAGAGGGAGTGGCCAACTCCATCACTAGGGGTTCCT (SEQ ID NO: 100)
Single AAV SaKKH-ABE8e
ITR-EFS [symbol] -SaKKHABE8e (start codon-BPNLS-TadA-SaKKHCas9 D10A-BPNLS-

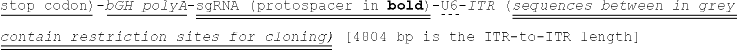

CTGCGCGCTCGCTCGCTCACTGAGGCCGCCCGGGCAAAGCCCGGGCGTCGGGCGACCTTTGGTCGCC
CGGCCTCAGTGAGCGAGCGAGCGCGCAGAGAGGGAGTGGCCAACTCCATCACTAGGGGTTCCTGCGGC
CTCTAGAATTCGCTAGCTAGGTCTTGAAAGGAGTGGGAATTGGCTCCGGTGCCCGTCAGTGG
GCAGAGCGCACATCGCCCACAGTCCCCGAGAAGTTGGGGGGAGGGGTCGGCAATTGATCCG
GTGCCTAGAGAAGGTGGCGCGGGGTAAACTGGGAAAGTGATGTCGTGTACTGGCTCCGCCT
TTTTCCCGAGGGTGGGGGAGAACCGTATATAAGTGCAGTAGTCGCCGTGAACGTTCTTTTTC
GCAACGGGTTTGCCGCCAGAACACAGGACCGGTGCCACCATGAAACGGACAGCCGACGGAAG
CGAGTTCGAGTCACCAAAGAAGAAGCGGAAAGTCTCTGAGGTGGAGTTTTCCCACGAGTACTGGA
TGAGACATGCCCTGACCCTGGCCAAGAGGGCACGGGATGAGAGGGAGGTGCCTGTGGGAGCCGTG
CTGGTGCTGAACAATAGAGTGATCGGCGAGGGCTGGAACAGAGCCATCGGCCTGCACGACCCAAC
AGCCCATGCCGAAATTATGGCCCTGAGACAGGGCGGCCTGGTCATGCAGAACTACAGACTGATTG
ACGCCACCCTGTACGTGACATTCGAGCCTTGCGTGATGTGCGCCGGCGCCATGATCCACTCTAGGA
TCGGCCGCGTGGTGTTTGGCGTGAGGAACTCAAAAAGAGGCGCCGCAGGCTCCCTGATGAACGTG
CTGAACTACCCCGGCATGAATCACCGCGTCGAAATTACCGAGGGAATCCTGGCAGATGAATGTGC
CGCCCTGCTGTGCGATTTCTATCGGATGCCTAGACAGGTGTTCAATGCTCAGAAGAAGGCCCAGAG
CTCCATCAACTCCGGAGGATCTAGCGGAGGCTCCTCTGGCTCTGAGACACCTGGCACAAGCGAGA
GCGCAACACCTGAAAGCAGCGGGGGCAGCAGCGGGGGGTCAGGGAAGCGAAATTACATTCTGGG
GCTGGCCATTGGCATTACATCAGTGGGCTATGGCATCATTGACTACGAGACAAGGGACGTGATCG
ACGCCGGCGTGAGACTGTTCAAGGAGGCCAACGTGGAGAACAATGAGGGCCGGAGATCCAAGAG
GGGAGCAAGGCGCCTGAAGCGGAGAAGGCGCCACAGAATCCAGAGAGTGAAGAAGCTGCTGTTC
GATTACAACCTGCTGACCGACCACTCCGAGCTGTCTGGCATCAATCCTTATGAGGCCAGAGTGAAG
GGCCTGTCCCAGAAGCTGTCTGAGGAGGAGTTTAGCGCCGCCCTGCTGCACCTGGCAAAGAGGAG
AGGCGTGCACAACGTGAATGAGGTGGAGGAGGACACCGGCAACGAGCTGTCCACAAAGGAGCAG
ATCAGCCGCAATTCCAAGGCCCTGGAGGAGAAGTATGTGGCCGAGCTGCAGCTGGAGCGGCTGAA
GAAGGATGGCGAGGTGAGGGGCTCCATCAATCGCTTCAAGACCTCTGACTACGTGAAGGAGGCCA
AGCAGCTGCTGAAGGTGCAGAAGGCCTACCACCAGCTGGATCAGTCCTTTATCGATACATATATCG
ACCTGCTGGAGACAAGGCGCACATACTATGAGGGACCAGGAGAGGGCTCTCCCTTCGGCTGGAAG
GACATCAAGGAGTGGTACGAGATGCTGATGGGCCACTGCACCTATTTTCCAGAGGAGCTGAGAAG
CGTGAAGTACGCCTATAACGCCGATCTGTACAACGCCCTGAATGACCTGAACAACCTGGTCATCAC
CAGGGATGAGAACGAGAAGCTGGAGTACTATGAGAAGTTCCAGATCATCGAGAACGTGTTCAAGC
AGAAGAAGAAGCCTACACTGAAGCAGATCGCCAAGGAGATCCTGGTGAACGAGGAGGACATCAA
GGGCTACCGCGTGACCTCCACAGGCAAGCCAGAGTTCACCAATCTGAAGGTGTATCACGATATCA
AGGACATCACAGCCCGGAAGGAGATCATCGAGAACGCCGAGCTGCTGGATCAGATCGCCAAGATC
CTGACCATCTATCAGAGCTCCGAGGACATCCAGGAGGAGCTGACCAACCTGAATAGCGAGCTGAC
ACAGGAGGAGATCGAGCAGATCAGCAATCTGAAGGGCTACACCGGCACACACAACCTGAGCCTG
AAGGCCATCAATCTGATCCTGGATGAGCTGTGGCACACAAACGACAATCAGATCGCCATCTTTAAC
CGGCTGAAGCTGGTGCCAAAGAAGGTGGACCTGTCCCAGCAGAAGGAGATCCCAACCACACTGGT
GGACGATTTCATCCTGTCTCCCGTGGTGAAGCGGAGCTTCATCCAGAGCATCAAAGTGATCAACGC
CATCATCAAGAAGTACGGCCTGCCCAATGATATCATCATCGAGCTGGCCAGGGAGAAGAACTCCA
AGGACGCCCAGAAGATGATCAATGAGATGCAGAAGAGGAACCGCCAGACCAATGAGCGGATCGA
GGAGATCATCAGAACCACAGGCAAGGAGAACGCCAAGTACCTGATCGAGAAGATCAAGCTGCAC
GATATGCAGGAGGGCAAGTGTCTGTATTCTCTGGAGGCCATCCCTCTGGAGGACCTGCTGAACAAT
CCATTCAACTACGAGGTGGATCACATCATCCCCCGGAGCGTGAGCTTCGACAATTCTTTTAACAAT
AAGGTGCTGGTGAAGCAGGAGGAGAACAGCAAGAAGGGCAATAGGACCCCTTTCCAGTACCTGTC
TAGCTCCGATTCTAAGATCAGCTACGAGACATTCAAGAAGCACATCCTGAATCTGGCCAAGGGCA
AGGGCCGCATCAGCAAGACCAAGAAGGAGTACCTGCTGGAGGAGCGGGACATCAACAGATTCTCC
GTGCAGAAGGACTTCATCAACCGGAATCTGGTGGACACCAGATACGCCACACGCGGCCTGATGAA
TCTGCTGCGGTCTTATTTCAGAGTGAACAATCTGGATGTGAAGGTGAAGAGCATCAACGGCGGCTT
CACCTCCTTTCTGCGGAGAAAGTGGAAGTTTAAGAAGGAGCGCAACAAGGGCTATAAGCACCACG
CCGAGGATGCCCTGATCATCGCCAATGCCGACTTCATCTTTAAGGAGTGGAAGAAGCTGGACAAG
GCCAAGAAAGTGATGGAGAACCAGATGTTCGAGGAGAAGCAGGCCGAGAGCATGCCCGAGATCG
AGACAGAGCAGGAGTACAAGGAGATTTTCATCACACCTCACCAGATCAAGCACATCAAGGACTTC
AAGGACTACAAGTATTCTCACAGGGTGGATAAGAAGCCCAACCGCAAGCTGATCAATGACACCCT
GTATAGCACACGGAAGGACGATAAGGGCAATACCCTGATCGTGAACAATCTGAACGGCCTGTACG
ACAAGGATAATGACAAGCTGAAGAAGCTGATCAACAAGTCTCCCGAGAAGCTGCTGATGTACCAC
CACGATCCTCAGACATATCAGAAGCTGAAGCTGATCATGGAGCAGTACGGCGACGAGAAGAACCC
ACTGTATAAGTACTATGAGGAGACAGGCAACTACCTGACAAAGTATAGCAAGAAGGATAATGGCC
CCGTGATCAAGAAGATCAAGTACTATGGCAACAAGCTGAATGCCCACCTGGACATCACCGACGAT
TACCCTAACTCTCGCAATAAGGTGGTGAAGCTGAGCCTGAAGCCATACCGGTTCGACGTGTACCTG
GACAACGGCGTGTATAAGTTTGTGACAGTGAAGAATCTGGATGTGATCAAGAAGGAGAACTACTA
TGAGGTGAACAGCAAGTGCTACGAGGAGGCCAAGAAGCTGAAGAAGATCAGCAACCAGGCCGAG
TTCATCGCCTCTTTTTACAAGAATGACCTGATCAAGATCAATGGCGAGCTGTATAGAGTGATCGGC
GTGAACAATGATCTGCTGAACAGAATCGAAGTGAATATGATCGACATCACCTACAGGGAGTATCT
GGAGAACATGAATGATAAGAGGCCCCCTCATATCATCAAGACCATCGCCTCTAAGACACAGAGCA
TCAAGAAGTACAGCACAGACATCCTGGGGAACCTGTATGAAGTCAAGAGCAAGAAACATCCTCAG
ATTATCAAGAAAGGCTCTGGCGGCTCAAAAAGAACCGCCGACGGCAGCGAATTCGAGCCCAAGAA

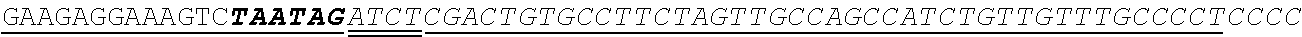

CGTGCCTTCCTTGACCCTGGAAGGTGCCACTCCCACTGTCCTTTCCTAATAAAATGAGGAAATTGCATCGC
ATTGTCTGAGTAGGTGTCATTCTATTCTGGGGGGTGGGGTGGGGCAGGACAGCAAGGGGGAGGATTGG
GAAGACAATAGCAGGCATGCTGGGGATGCGGTGGGCTCTATGGCTCGAGCGGCCCAAGCTTAAAAAAA
TCTCGCCAACAAGTTGACGAGATAAACACGGCATTTTGCCTTGTTTTAGTAGATTCTGTAATTTTCA

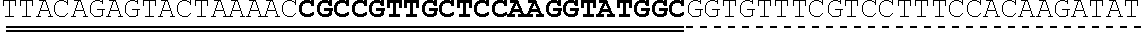

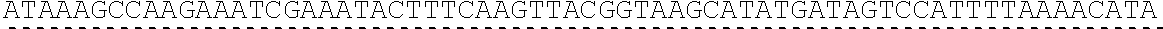

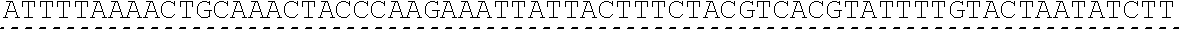

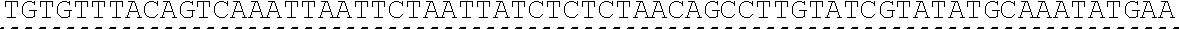

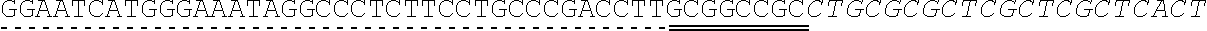

GAGGCCGCCCGGGCAAAGCCCGGGCGTCGGGCGACCTTTGGTCGCCCGGCCTCAGTGAGCGAGCGAG
CGCGCAGAGAGGGAGTGGCCAACTCCATCACTAGGGGTTCCT (SEQ ID NO: 101)
Single AAV SauriABE8e
ITR-EFS [symbol] -SauriABE8e (start codon-BPNLS-TadA-SauriCas9 D10A-BPNLS-stop

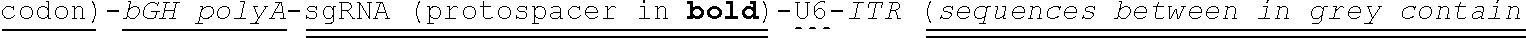

restriction sites for cloning)
[4828 bp is the ITR-to-ITR length]
CTGCGCGCTCGCTCGCTCACTGAGGCCGCCCGGGCAAAGCCCGGGCGTCGGGCGACCTTTGGTCGCC
CGGCCTCAGTGAGCGAGCGAGCGCGCAGAGAGGGAGTGGCCAACTCCATCACTAGGGGTTCCTGCGGC
CTCTAGAATTCGCTAGCTAGGTCTTGAAAGGAGTGGGAATTGGCTCCGGTGCCCGTCAGTGG
GCAGAGCGCACATCGCCCACAGTCCCCGAGAAGTTGGGGGGAGGGGTCGGCAATTGATCCG
GTGCCTAGAGAAGGTGGCGCGGGGTAAACTGGGAAAGTGATGTCGTGTACTGGCTCCGCCT
TTTTCCCGAGGGTGGGGGAGAACCGTATATAAGTGCAGTAGTCGCCGTGAACGTTCTTTTTC
GCAACGGGTTTGCCGCCAGAACACAGGACCGGTGCCACCATGAAACGGACAGCCGACGGAAG
CGAGTTCGAGTCACCAAAGAAGAAGCGGAAAGTCTCTGAGGTGGAGTTTTCCCACGAGTACTGGA
TGAGACATGCCCTGACCCTGGCCAAGAGGGCACGGGATGAGAGGGAGGTGCCTGTGGGAGCCGTG
CTGGTGCTGAACAATAGAGTGATCGGCGAGGGCTGGAACAGAGCCATCGGCCTGCACGACCCAAC
AGCCCATGCCGAAATTATGGCCCTGAGACAGGGCGGCCTGGTCATGCAGAACTACAGACTGATTG
ACGCCACCCTGTACGTGACATTCGAGCCTTGCGTGATGTGCGCCGGCGCCATGATCCACTCTAGGA
TCGGCCGCGTGGTGTTTGGCGTGAGGAACTCAAAAAGAGGCGCCGCAGGCTCCCTGATGAACGTG
CTGAACTACCCCGGCATGAATCACCGCGTCGAAATTACCGAGGGAATCCTGGCAGATGAATGTGC
CGCCCTGCTGTGCGATTTCTATCGGATGCCTAGACAGGTGTTCAATGCTCAGAAGAAGGCCCAGAG
CTCCATCAACTCCGGAGGATCTAGCGGAGGCTCCTCTGGCTCTGAGACACCTGGCACAAGCGAGA
GAACTACATCCTGGGCCTGGCCATCGGAATCACCAGCGTCGGCTACGGACTGATCGATAGCAAGA
CAAGAGAAGTGATCGACGCCGGCGTTAGACTCTTTCCAGAAGCTGATAGCGAGAACAACTCCAAC
CGCAGAAGCAAGCGGGGCGCCAGACGGTTAAAACGGAGAAGAATCCACCGGCTGAACCGGGTCA
AAGACCTGCTCGCTGATTACCAGATGATCGATCTTAACAATGTTCCTAAGAGCACCGACCCCTACA
CCATCAGAGTGAAGGGCCTCCGGGAGCCTCTGACAAAAGAAGAATTCGCCATCGCCCTCCTGCAT
ATCGCTAAGAGAAGAGGCCTGCACAACATCAGTGTGTCCATGGGCGACGAAGAGCAGGACAATG
AACTGAGCACCAAGCAGCAGCTGCAAAAGAATGCCCAGCAACTGCAGGACAAGTATGTGTGCGA
ACTGCAGTTAGAACGGCTGACCAACATCAACAAGGTCAGAGGCGAGAAGAACAGATTTAAGACA
GAGGACTTTGTGAAAGAAGTGAAACAGCTGTGCGAAACCCAGAGACAGTACCACAACATCGACG
ACCAATTCATCCAGCAGTACATCGACCTGGTGTCTACAAGACGGGAGTACTTCGAGGGCCCCGGC
AACGGCTCTCCATACGGCTGGGACGGCGACCTGCTGAAGTGGTACGAGAAGCTGATGGGCAGATG
CACCTATTTCCCCGAAGAACTGAGGTCCGTGAAGTACGCCTACAGCGCCGACCTCTTCAACGCCCT
GAACGACCTGAACAACCTCGTTGTGACCAGGGATGACAATCCAAAGCTTGAGTACTACGAGAAGT
ACCACATTATTGAGAACGTGTTCAAGCAAAAGAAGAATCCCACACTCAAACAAATCGCCAAAGAG
ATCGGCGTGCAAGATTACGACATCCGGGGCTATAGAATCACAAAGAGCGGCAAACCTCAGTTCAC
CTCTTTTAAGCTGTATCACGACCTGAAGAACATCTTCGAGCAGGCCAAATACCTGGAAGATGTGGA
AATGCTGGACGAGATCGCCAAGATCCTGACCATCTACCAGGATGAGATTAGCATCAAGAAAGCCC
TGGACCAGCTGCCCGAACTGCTGACAGAGAGCGAGAAATCTCAGATCGCACAGCTCACCGGCTAT
ACAGGCACCCACAGACTGAGCCTGAAGTGCATCCACATTGTGATCGACGAGCTGTGGGAGAGCCC
CGAGAACCAGATGGAAATCTTTACCAGACTGAATCTGAAACCTAAGAAGGTGGAAATGAGCGAGA
TCGACAGCATACCCACCACCCTGGTCGACGAGTTCATCCTCTCACCTGTGGTGAAGCGGGCCTTCA
TCCAGAGCATCAAGGTAATCAACGCAGTGATCAATCGGTTCGGCCTGCCAGAGGACATCATCATC
GAGCTGGCCAGAGAAAAGAATAGCAAGGATCGGAGAAAGTTCATTAACAAGCTGCAGAAACAAA
ATGAGGCCACAAGAAAGAAAATCGAACAGCTGCTGGCCAAGTACGGCAACACCAATGCCAAGTA
CATGATCGAGAAGATCAAGCTGCACGACATGCAGGAGGGCAAGTGCCTGTACAGCCTGGAGGCTA
TTCCTCTGGAAGACCTGCTGAGCAACCCGACACACTACGAAGTTGACCACATTATCCCCAGATCTG
TGAGCTTTGACAACAGCCTGAACAACAAAGTGCTGGTGAAACAAAGCGAAAACAGCAAGAAGGG
CAATCGCACCCCTTACCAGTACCTGAGCAGCAACGAGTCTAAGATTAGCTACAACCAGTTTAAGCA
GCACATCCTGAACCTGAGCAAGGCCAAGGACAGAATCAGCAAGAAAAAAAGAGATATGCTGCTG
GAAGAGAGAGATATCAACAAGTTCGAAGTGCAGAAGGAATTCATTAACCGGAACCTGGTGGATAC
ACGGTACGCCACCAGAGAACTGTCTAACCTGCTGAAGACCTACTTCAGCACCCATGACTACGCCGT
GAAGGTGAAGACCATCAACGGCGGCTTCACTAACCACCTGAGGAAGGTGTGGGATTTCAAGAAGC
ACAGAAACCACGGCTACAAGCACCACGCCGAAGATGCCCTGGTGATCGCCAACGCCGACTTCCTG
TTTAAGACACATAAGGCCCTGCGGAGAACCGATAAGATCCTGGAACAACCTGGCCTGGAAGTGAA
TGATACAACCGTGAAAGTGGACACCGAGGAAAAATACCAGGAGCTGTTCGAGACACCTAAGCAA
GTGAAGAACATCAAGCAGTTCCGGGACTTCAAGTACAGCCACCGAGTGGACAAGAAGCCTAACCG
GCAGCTTATCAACGACACACTGTACTCCACCAGAGAGATTGATGGCGAAACCTACGTGGTGCAGA
CCCTTAAGGATCTGTACGCCAAGGACAACGAGAAAGTGAAGAAGCTGTTCACCGAAAGACCTCAG
AAGATCCTGATGTACCAGCACGACCCTAAGACCTTCGAGAAACTGATGACAATCCTGAACCAGTA
CGCTGAGGCCAAGAACCCTCTGGCTGCTTATTACGAGGACAAAGGCGAGTACGTGACCAAGTACG
CCAAGAAAGGCAATGGACCTGCCATCCACAAGATCAAGTATATCGATAAGAAGCTTGGATCTTAC
CTGGATGTTAGCAACAAGTATCCTGAGACACAGAACAAGCTTGTGAAGCTGTCCCTGAAGAGCTTT
AGATTCGACATCTACAAGTGTGAACAGGGCTACAAGATGGTGTCCATCGGATACCTGGACGTGCT
GAAGAAAGATAACTACTACTACATCCCTAAGGACAAGTACGAGGCCGAGAAGCAGAAAAAGAAG
ATCAAGGAATCTGATCTTTTTGTGGGCAGCTTCTACTACAACGACCTCATCATGTACGAGGATGAA
CTGTTCAGAGTGATAGGAGTGAACAGCGACATCAACAATCTGGTTGAGCTAAACATGGTCGACAT
TACCTACAAGGACTTCTGCGAGGTGAACAACGTGACAGGCGAGAAAAGAATCAAAAAGACTATCG
GCAAGCGCGTGGTCCTGATCGAGAAGTACACCACAGATATTCTAGGCAACCTGTACAAGACTCCC
CTGCCTAAGAAGCCCCAGCTTATCTTCAAGCGGGGAGAACTGTCTGGCGGCTCAAAAAGAACCGC

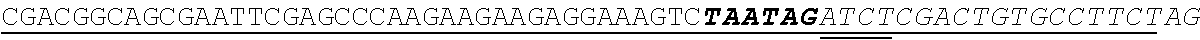

TTGCCAGCCATCTGTTGTTTGCCCCTCCCCCGTGCCTTCCTTGACCCTGGAAGGTGCCACTCCCACTGTC
CTTTCCTAATAAAATGAGGAAATTGCATCGCATTGTCTGAGTAGGTGTCATTCTATTCTGGGGGGTGGGGT
GGGGCAGGACAGCAAGGGGGAGGATTGGGAAGACAATAGCAGGCATGCTGGGGATGCGGTGGGCTCT
ATGGCTCGAGCGGCCCAAGCTTAAAAAAATCTCGCCAACAAGTTGACGAGATAAACACGGCATTTT
GCCTTGTTTTAGTAGATTCTGTAATTTTCATTACAGAGTACTAAAACCGTTGCTCCAAGGTATGG

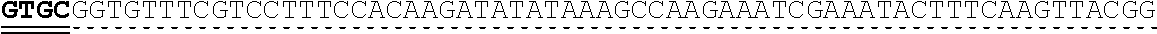

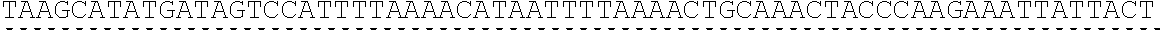

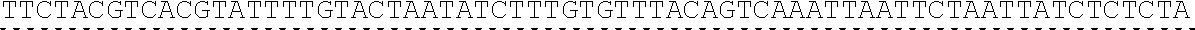

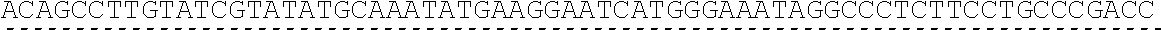

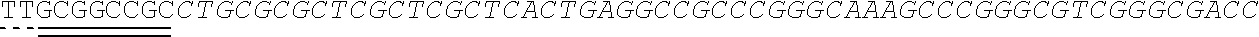

TTTGGTCGCCCGGCCTCAGTGAGCGAGCGAGCGCGCAGAGAGGGAGTGGCCAACTCCATCACTAGGGG
TTCCT (SEQ ID NO: 102)

### Methods of Editing a Target Nucleic Acid Molecule

In some aspects, provided herein are methods of contacting any of the disclosed AAV-encoded base editors with a nucleic acid molecule, e.g., a nucleic acid molecule (e.g., DNA) comprising a target sequence. In some embodiments, the nucleic acid molecule comprises a DNA, e.g., a single-stranded DNA or a double-stranded DNA. The target sequence of the nucleic acid molecule may comprise a target nucleobase pair containing an adenine (A). The target sequence of the nucleic acid molecule may comprise a target nucleobase pair containing a cytosine (C). The target sequence may be a genomic sequence, e.g., a human genomic sequence. The target sequence may comprise a sequence, e.g., a target sequence with point mutation, associated with a disease or disorder. The target sequence with a point mutation may be associated with cardiovascular disease.

In some embodiments, the target nucleotide sequence is a DNA sequence in a genome, e.g., a eukaryotic genome. In certain embodiments, the target nucleotide sequence is in a mammalian (e.g., a human) genome. In certain embodiments, the target nucleotide sequence is in a human genome. In other embodiments, the target nucleotide sequence is in the genome of a rodent, such as a mouse or a rat. In other embodiments, the target nucleotide sequence is in the genome of a domesticated animal, such as a horse, cat, dog, or rabbit. In some embodiments, the target nucleotide sequence is in the genome of a research animal. In some embodiments, the target nucleotide sequence is in the genome of a genetically engineered non-human subject. In some embodiments, the target nucleotide sequence is in the genome of a plant. In some embodiments, the target nucleotide sequence is in the genome of a microorganism, such as a bacteria.

In some embodiments, the disclosed AAV-encoded base editors exhibit low off-target effects, such as low off-target editing frequencies. In some embodiments, the disclosed AAV-encoded base editors exhibit low off-target editing frequencies while exhibiting high on-target editing efficiencies. In some embodiments, use of the TadA-8e deaminase or TadA-8c (V106W) deaminase in any for the disclosed AAV-encoded adenine base editors may exhibit off-target editing frequencies of 0.32% or less while maintaining on-target editing efficiencies of about 80% or more. See PCT Publication No. WO 2021/158921, published Aug. 12, 2021.

The disclosed base editors may provide (or yield) on-target editing efficiencies of greater than 50% or greater than 60% (such as greater than 70%, greater than 75%, greater than 80%, or greater than 85%) at the target nucleobase pair for one or more base editors under evaluation. Any of the disclosed methods of editing may yield an on-target editing efficiency of at least about 50%, at least about 55%, at least about 60%, at least about 65%, at least about 70%, at least about 75%, at least about 80%, or at least about 85%.

In some embodiments, the disclosed BEs and editing methods comprising the step of contacting a cell comprising a target DNA sequence with any of the disclosed BEs result in an actual or average off-target DNA editing frequency of about 2.0% or less, 1.75% or less, 1.5% or less, 1.2% or less, 1% or less, 0.9% or less, 0.8% or less, 0.75% or less, 0.7% or less, 0.65% or less, or 0.6% or less. These off-target editing frequencies may be obtained in sequences having any level of sequence identity to the target sequence. As used herein to refer to off-target DNA editing frequencies, the modifier “average” refers to a mean value over all editing events detected at sites other than a given target nucleobase pair (e.g., as detected by high-throughput sequencing).

In various embodiments, the disclosed editing methods result in an on-target DNA base editing efficiency of at least about 35%, 40%, 50%, 60%, 70%, 80%, 85%, 90%, 95%, 98%, or 99% at the target nucleobase pair. The step of contacting may result in in a DNA base editing efficiency of at least about 60%, 61%, 62%, 63%, 64%, 65%, 66%, 67%, 68%, 69%, 70%, 71%, 72%, 73%, 74%, or 75%. In particular, the step of contacting results in on-target base editing efficiencies of greater than 75%. The step of contacting may result in in a DNA base editing efficiencies of between 60 and 85%. In certain embodiments, base editing efficiencies of greater than 85% may be realized.

These editing efficiencies may be realized following administration of any of the disclosed AAV-encoded base editors in any target tissue, such as liver tissue, cardiac tissue, muscle tissue, neuronal tissue, or ocular tissue.

Upon administration of any of the disclosed rAAV vectors or particle to cardiac tissue or muscle tissue (e.g., skeletal muscle tissue), editing efficiencies of at least about 20%, at least about 22%, at least about 24%, at least about 27%, at least about 30%, at least about 33%, or at least about 36%, may be realized. These editing efficiencies represent between 2- and 2.5-fold increases relative to the editing efficiencies in cardiac and muscle tissues reported for dual AAV vectors. Upon administration of any of the disclosed rAAV vectors or particle to cardiac tissue or muscle tissue (e.g., cardiac tissue), indel rates of 2.5% or less, 2.0% or less, 1.5% or less, 1.2% or less, or 1.0% or less in the cardiac cell or muscle cell may be realized.

In various embodiments, the disclosed editing methods result in a ratio of on-target:off-target editing of about 25:1, 50:1, 65:1, 75:1, 80:1, 85:1, 90:1, 95:1, 100:1, 110:1, 125:1, or more than 125:1. In various embodiments, the disclosed editing methods result in a ratio of on-target:off-target editing of about 150:1, 200:1, 300:1, 400:1, 500:1, 600:1, 700:1, 800:1, 900:1, 1000:1, 1100:1, 1200:1, 1250:1, 1275:1, 1300:1, 1325:1, 1350:1, 1400:1, 1500:1, or more than 1500:1. As used herein, a ratio of on-target:off-target editing is equivalent to a ratio of sequencing reads reflecting on-target deaminations relative to deaminations of known or predicted off-target sites, or candidate off-target sites. Candidate off-target sites may be identified, and hence the ratio of on-target:off-target editing may be measured, using an experimental assay or a computation algorithm (e.g., Cas-OFFinder). For example, candidate off-target sites may be identified using an experimental assay such as EndoV-Seq, GUIDE-Seq, or CIRCLE-Seq. In some embodiments, the ratios of on-target editing:off-target editing relies on the use of EndoV-Seq.

In some embodiments, the disclosed editing methods result in, and the disclosed base editors generate, a minimal degree of bystander edits (i.e., synonymous off-target point mutations at nucleobases that are near the target base and do not change the outcome of the intended editing method). In some embodiments, the disclosed editing methods result in less than 10, less than 9, less than 8, less than 7, less than 6, less than 5, less than 4, less than 3, less than 2, less than 1, or zero non-silent bystander edits. For example, editing methods using the disclosed single-AAV encoded SaKKH-ABE8e editor in liver tissue may result in few (i.e., minimal) non-silent bystander edits.

Some aspects of the disclosure are based on the recognition that any of the adenine base editors provided herein are capable of modifying a specific DNA base without generating a significant proportion of indels. An “indel”, as used herein, refers to the insertion or deletion of a nucleotide base within a DNA substrate. Such insertions or deletions can lead to frame shift mutations within a coding region of a gene. In some embodiments, it is desirable to generate adenine base editors that efficiently modify (e.g. mutate or deaminate) a specific nucleotide within a DNA, without generating a large number of insertions or deletions (i.e., indels) in the nucleic acid (while at the same time having lower RNA editing effects than existing adenine base editors).

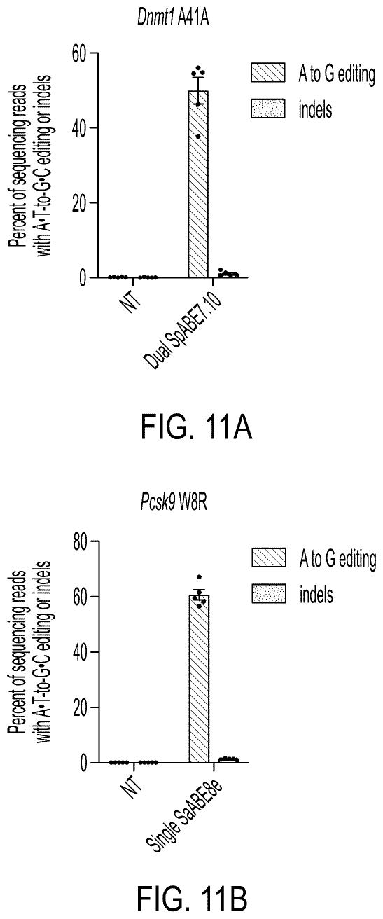

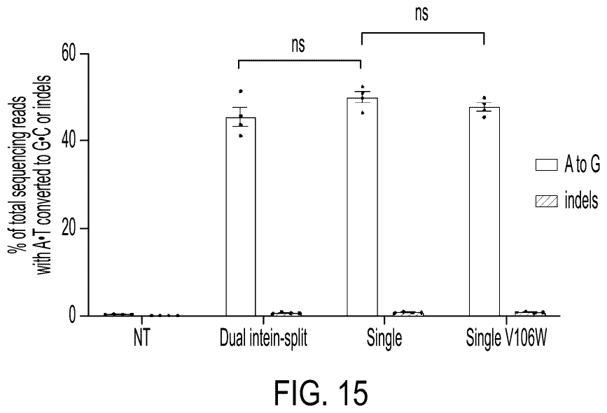

In some embodiments, the disclosed editing methods that use the disclosed BEs may result in less than 20%, 19%, 18%, 16%, 14%, 12%, 10%, 8%, 6%, 4%, 2%, 1.5%, 1%, 0.5%, 0.2%, or 0.1% indel formation in a nucleic acid (e.g., a DNA) comprising a target sequence. In some embodiments, the disclosed editing methods result in an indel rate of 2.5% or less, 2.0% or less, 1.5% or less, 1.2% or less, or 1.0% or less. See FIGS. 11A, 11B, and 15.

In some embodiments, the disclosed editing methods result in a base edit:indel ratio of at least about 5:1, 7:1, 8:1, 9:1, 10:1, 11:1, 12:1, 13:1 or greater than about 15:1.

Some aspects of the disclosure are based on the recognition that any of the base editors provided herein are capable of efficiently generating an intended mutation, such as a point mutation, in DNA (e.g. DNA within a genome of a subject) without generating a significant number of unintended mutations, such as unintended point mutations. In some embodiments, an intended mutation is a mutation that is generated by a specific base editor bound to a gRNA, specifically designed to generate the intended mutation (e.g. deamination).

In some embodiments, the intended mutation is a mutation associated with a disease or disorder, such as sickle cell disease. In some embodiments, the intended mutation is an adenine (A) to guanine (G) point mutation associated with a disease or disorder. In some embodiments, the intended mutation is a thymine (T) to cytosine (C) point mutation associated with a disease or disorder. In some embodiments, the intended mutation is an adenine (A) to guanine (G) point mutation within the coding region of a gene. In some embodiments, the intended mutation is a thymine (T) to cytosine (C) point mutation within the coding region of a gene.

In some embodiments, the intended mutation is a deamination that generates a stop codon, for example, a premature stop codon within the coding region of a gene. In some embodiments, the intended mutation is a mutation that eliminates a stop codon. In some embodiments, the intended mutation eliminates a stop codon comprising the nucleic acid sequence 5′-TAG-3′, 5′-TAA-3′, or 5′-TGA-3′.

In some embodiments, the intended mutation is a deamination that alters the regulatory sequence of a gene (e.g., a gene promoter or gene repressor). In some embodiments, the intended mutation is a deamination introduced into the gene promoter. In particular embodiments, the deamination introduced into the gene promoter leads to a decrease in the transcription of a gene operably linked to the gene promoter. In other embodiments, the deamination leads to an increase in the transcription of a gene operably linked to the gene promoter.

In some embodiments, the intended mutation is a deamination that alters the splicing of a genetic sequence, or gene. Accordingly, in some embodiments, the intended deamination results in the introduction of a splice site in a gene. In other embodiments, the intended deamination results in the removal of a splice site. In some embodiments, the intended deamination results in the introduction of a stop codon in a gene. In other embodiments, the intended deamination results in the removal of a stop codon.

In some embodiments, any of the base editors provided herein are capable of generating a ratio of intended mutations to unintended mutations (e.g., intended point mutations:unintended point mutations) that is greater than 1:1. In some embodiments, any of the base editors provided herein are capable of generating a ratio of intended mutations to unintended mutations (e.g., intended point mutations:unintended point mutations) that is at least 1.5:1, at least 2:1, at least 2.5:1, at least 3:1, at least 3.5:1, at least 4:1, at least 4.5:1, at least 5:1, at least 5.5:1, at least 6:1, at least 6.5:1, at least 7:1, at least 7.5:1, at least 8:1, at least 10:1, at least 12:1, at least 15:1, at least 20:1, at least 25:1, at least 30:1, at least 40:1, at least 50:1, at least 100:1, at least 150:1, at least 200:1, at least 250:1, at least 500:1, or at least 1000:1, or more. It should be appreciated that the characteristics of the base editors described in this section and the following section of the disclosure may be applied to any of the base editors, or methods of using the base editors provided herein.

The disclosed AAV-encoded base editors disclosed herein have reduced and/or low RNA editing effects. In some embodiments, the base editors are evolved or engineered to have reduced RNA editing effects. The term “RNA editing effects,” as used herein, refers to the introduction of modifications (e.g. deaminations) of nucleotides within cellular RNA, e.g., messenger RNA (mRNA). An important goal of DNA base editing efficiency is the modification (e.g. deamination) of a specific nucleotide within DNA, without introducing modifications of similar nucleotides within RNA. RNA editing effects are “low” or “reduced” when a detected mutation is introduced into RNA molecules at a frequency of 0.3% or less.

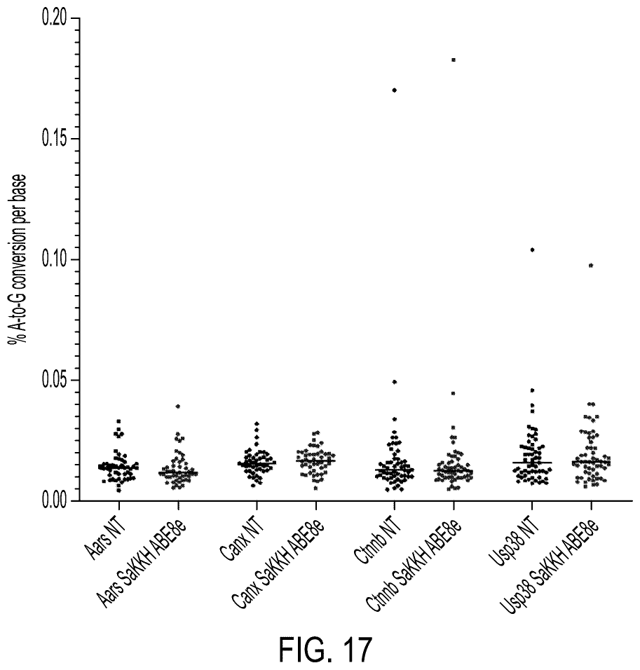

The present disclosure further provides methods of administering the disclosed base editors wherein the method yields reduced and/or low RNA editing effects. The present disclosure further provides base editors (such as the disclosed ABEs) that induce (or yield, provide or cause) low and/or undetectable RNA editing effects (see FIG. 17). In some embodiments, the base editors provide an average adenosine (A) to inosine (I) (A-to-I) editing frequency in cellular mRNA transcripts of 0.3% or less. In some embodiments, the base editors provide an average adenosine (A) to inosine (I) (A-to-I) actual and/or consistent editing frequencies in RNA of about 0.3% or less. The base editors may provide actual or average A-to-I editing frequencies in RNA of about 0.5% or less, 0.4% or less, 0.35% or less, 0.25% or less, 0.2% or less, 0.15% or less, 0.12% or less, 0.1% or less, 0.08% or less, or 0.075% or less.

### Guide Sequences (e.g., Guide RNAs)

The present disclosure further provides guide RNAs for use in accordance with the disclosed methods of editing. The disclosure provides guide RNAs that are designed to recognize target sequences. Such gRNAs may be designed to have guide sequences (or “spacers”) having complementarity to a protospacer within the target sequence.

Guide RNAs are also provided for use with one or more of the disclosed base editors, e.g., in the disclosed methods of editing a nucleic acid molecule. Such gRNAs may be designed to have guide sequences having complementarity to a protospacer within a target sequence to be edited, and to have backbone sequences that interact specifically with the napDNAbp domains of any of the disclosed base editors, such as Cas9 nickase domains of the disclosed base editors. Guide RNAs in accordance with the disclosed methods of editing may have complementarity to any of the protospacer sequences listed in Table 1 (SEQ ID NOs: 430-565).

In various embodiments, the base editors may be complexed, bound, or otherwise associated with (e.g., via any type of covalent or non-covalent bond) one or more guide sequences. The guide sequence becomes associated or bound to the base editor and directs its localization to a specific target sequence having complementarity to the guide sequence or a portion thereof. The particular design embodiments of a guide sequence will depend upon the nucleotide sequence of a genomic target sequence (i.e., the desired site to be edited) and the type of napDNAbp (e.g., type of Cas9 protein) present in the base editor, among other factors, such as PAM sequence locations, percent G/C content in the target sequence, the degree of microhomology regions, secondary structures, etc.

In general, a guide sequence is any polynucleotide sequence having sufficient complementarity with a target polynucleotide sequence to hybridize with the target sequence and direct sequence-specific binding of the napDNAbp (e.g., a Cas9 or Cas9 variant) to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies, ELAND (Illumina, San Diego, Calif.), SOAP (available at soap.genomics.org.cn), and Maq (available at maq.sourceforge.net).

In some embodiments, a guide sequence is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a guide sequence is less than about 75, 50, 45, 40, 35, 30, 25, 20, 15, 12, or fewer nucleotides in length. In some embodiments, the guide RNA is 15-300, 25-300, 50-300, 25-250, 25-200, 15-200, or 15-100 nucleotides in length. In some embodiments, the guide RNA is between about 25 and 200 nucleotides in length. In some embodiments, the guide RNA is between about 15 and 200, or between 15 and 100, nucleotides in length.

The ability of a guide sequence to direct sequence-specific binding of a base editor to a target sequence may be assessed by any suitable assay. For example, the components of a base editor, including the guide sequence to be tested, may be provided to a host cell having the corresponding target sequence, such as by transfection with vectors encoding the components of a base editor disclosed herein, followed by an assessment of preferential cleavage within the target sequence. Similarly, cleavage of a target polynucleotide sequence may be evaluated in situ by providing the target sequence, components of a base editor, including the guide sequence to be tested and a control guide sequence different from the test guide sequence, and comparing binding or rate of cleavage at the target sequence between the test and control guide sequence reactions. Other assays are possible, and will occur to those skilled in the art.

A guide sequence may be selected to target any target sequence. In some embodiments, the target sequence is a sequence within a genome of a cell. Exemplary target sequences include those that are unique in the target genome.

In some embodiments, a guide sequence is selected to reduce the degree of secondary structure within the guide sequence. Secondary structure may be determined by any suitable polynucleotide folding algorithm. Some programs are based on calculating the minimal Gibbs free energy. An example of one such algorithm is mFold, as described by Zuker & Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see, e.g., A. R. Gruber et al., 2008, Cell 106 (1): 23-24; and PA Carr & GM Church, 2009, Nature Biotechnology 27 (12): 1151-62). Additional algorithms may be found in Chuai, G. et al., DeepCRISPR: optimized CRISPR guide RNA design by deep learning, Genome Biol. 19:80 (2018), and U.S. Application Ser. No. 61/836,080 and U.S. Pat. No. 8,871,445, issued Oct. 28, 2014, the entireties of each of which are incorporated herein by reference.

The guide sequence of the gRNA is linked to a tracr mate (also known as a “backbone”) sequence which in turn hybridizes to a traer sequence. A traer mate sequence includes any sequence that has sufficient complementarity with a traer sequence to promote one or more of: (1) excision of a guide sequence flanked by tracr mate sequences in a cell containing the corresponding tracr sequence; and (2) formation of a complex at a target sequence, wherein the complex comprises the tracr mate sequence hybridized to the tracr sequence. In general, degree of complementarity is with reference to the optimal alignment of the tracr mate sequence and tracr sequence, along the length of the shorter of the two sequences. Optimal alignment may be determined by any suitable alignment algorithm, and may further account for secondary structures, such as self-complementarity within either the tracr sequence or tracr mate sequence. In some embodiments, the degree of complementarity between the tracr sequence and tracr mate sequence along the length of the shorter of the two when optimally aligned is about or more than about 25%, 30%, 40%, 50%, 60%, 70%, 80%, 90%, 95%, 97.5%, 99%, or higher. In some embodiments, the tracr sequence is about or more than about 5, 6, 7, 8, 9, 10, 11, 12, 13, 14, 15, 16, 17, 18, 19, 20, 25, 30, 40, 50, or more nucleotides in length. In some embodiments, the traer sequence and traer mate sequence are contained within a single transcript, such that hybridization between the two produces a transcript having a secondary structure, such as a hairpin. Preferred loop forming sequences for use in hairpin structures are four nucleotides in length, and most preferably have the sequence GAAA. However, longer or shorter loop sequences may be used, as may alternative sequences. The sequences preferably include a nucleotide triplet (for example, AAA), and an additional nucleotide (for example C or G). Examples of loop forming sequences include CAAA and AAAG. In an embodiment of the invention, the transcript or transcribed polynucleotide sequence has at least two or more hairpins. In certain embodiments, the transcript has two, three, four or five hairpins. In a further embodiment of the invention, the transcript has at most five hairpins. In some embodiments, the single transcript further includes a transcription termination sequence; preferably this is a polyT sequence, for example six T nucleotides.

In some embodiments, the guide RNAs for use in accordance with the disclosed methods of editing comprise synthetic single guide RNAs (sgRNAs) containing modified ribonucleotides. In some embodiments, the guide RNAs contain modifications such as 2′-O-methylated nucleotides and phosphorothioate linkages. In some embodiments, the guide RNAs contain 2′-O-methyl modifications in the first three and last three nucleotides, and phosphorothioate bonds between the first three and last three nucleotides. Exemplary modified synthetic sgRNAs are disclosed in Hendel A. et al., Nat. Biotechnol. 33, 985-989 (2015), incorporated herein by reference.

In some embodiments, the guide RNAs for use in accordance with the disclosed methods of editing comprise a backbone structure that is recognized by an N. meningitis Cas9 protein or domain, such as an Nme2Cas9 domain. The backbone structure (or scaffold) recognized by an Nme2Cas9 protein may comprise the sequence provided below: 5′-[guide sequence]-gttgtagctccctttctcatttcggaaacgaaatgagaaccgttgctacaataaggccgtctgaaaagatgtgccgcaacgctctgcccc ttaaagcttctgctttaaggggcatcgttta-3′ (SEQ ID NO: 719). This scaffold sequence is recognized by the NmeCas9, NmelCas9, Nme2Cas9, and Nme3Cas9 proteins. Exemplary guide RNAs for editing with base editors containing Nme2Cas9 domains and variants thereof are described in Edraki et al., Molecular Cell 73, 714-726, incorporated herein by reference.

In other embodiments, the guide RNAs for use in accordance with the disclosed methods of editing comprise a backbone structure that is recognized by an S. pyogenes Cas9 protein or domain, such as an SpCas9 domain of the disclosed base editors. The backbone structure recognized by an SpCas9 protein may comprise the sequence 5′-[guide sequence]-guuuuagagcuagaaauagcaaguuaaaauaaggcuaguccguuaucaacuugaaaaaguggcaccgagucggugcuuu uu-3′ (SEQ ID NO: 339), wherein the guide sequence comprises a sequence that is complementary to the protospacer of the target sequence. See U.S. Publication No. 2015/0166981, published Jun. 18, 2015, the disclosure of which is incorporated herein by reference. The guide sequence is typically 20 nucleotides long.

In other embodiments, the guide RNAs for use in accordance with the disclosed methods of editing comprise a backbone structure that is recognized by an C. jejuni Cas9 protein or domain, such as a CjCas9 domain of the disclosed base editors. The backbone structure recognized by a CjCas9 protein may comprise the sequence 5′-[guide sequence]-gttttagtccctgaaaagggactaaaataaagagtttgcgggactctgcggggttacaatcccctaaaaccgcttttttt-3′ (SEQ ID NO: 340), wherein the guide sequence comprises a sequence that is complementary to the protospacer of the target sequence.

In other embodiments, the guide RNAs for use in accordance with the disclosed methods of editing comprise a backbone structure that is recognized by an S. aureus Cas9 protein. The backbone structure recognized by an SaCas9 protein may comprise the sequence 5′-[guide sequence]-guuuuaguacucuguaaugaaaauuacagaaucuacuaaaacaaggcaaaaugccguguuuaucucgucaacuuguugg cgagauuuuuuu-3′ (SEQ ID NO: 78). This is also the backbone structure recognized by SaKKH-Cas9 and the SaCas9 ortholog, SauriCas9.

The guide RNAs for use in accordance with the disclosed methods of editing may comprise a backbone structure as listed in Table 2 (SEQ ID NOs: 566-571).

The sequences of suitable guide RNAs for targeting the disclosed BEs to specific genomic target sites will be apparent to those of skill in the art based on the present disclosure. Such suitable guide RNA sequences typically comprise guide sequences that are complementary to a nucleic sequence within 50 nucleotides upstream or downstream of the target nucleobase pair to be edited. Some exemplary guide RNA sequences suitable for targeting any of the provided BEs to specific target sequences are provided herein. Additional guide sequences are well known in the art and may be used with the base editors described herein. Additional exemplary guide sequences are disclosed in, for example, Jinek M., et al., Science 337:816-821 (2012); Mali P, Esvelt K M & Church G M (2013) Cas9 as a versatile tool for engineering biology, Nature Methods, 10, 957-963; Li J F et al., (2013) Multiplex and homologous recombination-mediated genome editing in Arabidopsis and Nicotiana benthamiana using guide RNA and Cas9, Nature Biotechnology, 31, 688-691; Hwang, W. Y. et al., Efficient genome editing in zebrafish using a CRISPR-Cas system, Nature Biotechnology 31, 227-229 (2013); Cong L et al., (2013) Multiplex genome engineering using CRIPSR/Cas systems, Science, 339, 819-823; Cho S W et al., (2013) Targeted genome engineering in human cells with the Cas9 RNA-guided endonuclease, Nature Biotechnology, 31, 230-232; Jinek, M. et al., RNA-programmed genome editing in human cells, eLife 2, c00471 (2013); Dicarlo, J. E. et al., Genome engineering in Saccharomyces cerevisiae using CRISPR-Cas systems. Nucleic Acid Res. (2013); Briner A E et al., (2014) Guide RNA functional modules direct Cas9 activity and orthogonality, Mol Cell, 56, 333-339, the entire contents of each of which are incorporated herein by reference.

### Methods for Generating Cas Variants and Base Editors

The invention further relates in various aspects to methods of making the disclosed improved base editors by various modes of manipulation that include, but are not limited to, codon optimization to achieve greater expression levels in a cell, and the use of nuclear localization sequences (NLSs), preferably at least two NLSs, e.g., two bipartite NLSs, to increase the localization of the expressed base editors into a cell nucleus.

### Preparation of Base Editors for Increased Expression in Cells

The base editors contemplated herein can include modifications that result in increased expression, for example, through codon optimization.

In some embodiments, the base editors (or a component thereof) is codon optimized for expression in particular cells, such as eukaryotic cells. The eukaryotic cells may be those of or derived from a particular organism, such as a mammal, including, but not limited to, human, mouse, rat, rabbit, dog, or non-human primate. In general, codon optimization refers to a process of modifying a nucleic acid sequence for enhanced expression in the host cells of interest by replacing at least one codon (e.g. about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database”, and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, Pa.), are also available. In some embodiments, one or more codons (e.g. 1, 2, 3, 4, 5, 10, 15, 20, 25, 50, or more, or all codons) in a sequence encoding a CRISPR enzyme correspond to the most frequently used codon for a particular amino acid.

### Nuclear Localization Sequences and Additional Base Editor Components

In some embodiments, the base editors provided herein further comprise one or more nuclear targeting sequences, for example, a nuclear localization sequence (NLS). In some embodiments, a NLS comprises an amino acid sequence that facilitates the importation of a protein, that comprises an NLS, into the cell nucleus (e.g., by nuclear transport). In some embodiments, any of the base editors provided herein further comprise one or more nuclear localization sequences (NLSs). In certain embodiments, any of the base editors comprise two NLSs. In some embodiments, one or more of the NLSs are bipartite NLSs (“bpNLS”). In certain embodiments, the disclosed base editors comprise two bipartite NLSs. In some embodiments, the disclosed base editors comprise more than two bipartite NLSs.

In some embodiments, the NLS is fused to the N-terminus of the base editor. In some embodiments, the NLS is fused to the C-terminus of the base editor. In some embodiments, the NLS is fused to the C-terminus of the napDNAbp. In some embodiments, the NLS is fused to the N-terminus of the adenosine deaminase. In some embodiments, the NLS is fused to the C-terminus of the adenosine deaminase. In some embodiments, the NLS is fused to the base editor via one or more linkers. In some embodiments, the NLS is fused to the base editor without a linker.

In some embodiments, the NLS comprises an amino acid sequence of any one of the NLS sequences provided or referenced herein. In some embodiments, the NLS comprises an amino acid sequence as set forth in SEQ ID NO: 408 or SEQ ID NO: 409. Additional nuclear localization sequences are known in the art and would be apparent to the skilled artisan. For example, NLS sequences are described in Plank et al., PCT/EP2000/011690, the contents of which are incorporated herein by reference for their disclosure of exemplary nuclear localization sequences. In some embodiments, a NLS comprises the amino acid sequence PKKKRKV (SEQ ID NO: 408), MDSLLMNRRKFLYQFKNVRWAKGRRETYLC (SEQ ID NO: 409), KRTADGSEFESPKKKRKV (SEQ ID NO: 410), or KRTADGSEFEPKKKRKV (SEQ ID NO: 411). In other embodiments, the NLS comprises the amino acid sequence:

(SEQ ID NO: 482)
NLSKRPAAIKKAGQAKKKK,
(SEQ ID NO: 483)
PAAKRVKLD,
(SEQ ID NO: 484)
RQRRNELKRSF,
or
(SEQ ID NO: 485)
NQSSNFGPMKGGNFGGRSSGPYGGGGQYFAKPRNQGGY.

In some embodiments, the base editor comprises a bpNLS. The bpNLS may comprise an amino acid sequence selected from the group consisting of: KRTADGSEFEPKKKRKV (SEQ ID NO: 398), KRPAATKKAGQAKKKK (SEQ ID NO: 344), KKTELQTTNAENKTKKL (SEQ ID NO: 345), KRGINDRNFWRGENGRKTR (SEQ ID NO: 346), and RKSGKIAAIVVKRPRK (SEQ ID NO: 347). In certain embodiments, the bpNLA comprises the amino acid sequence set forth in SEQ ID NO: 344 or 398.

In some embodiments, the base editors provided herein do not comprise a linker. In some embodiments, a linker is present between one or more of the domains or proteins (e.g., deaminase, napDNAbp, and/or NLS). In some embodiments, the “]-[” used in the general architecture above indicates the presence of an optional linker.

In some embodiments, the general architecture of exemplary base editors with a first adenosine deaminase, a second adenosine deaminase, and a napDNAbp domain comprises any one of the following structures, where NLS is a nuclear localization sequence (e.g., any NLS provided herein), NH₂ is the N-terminus of the base editor, and COOH is the C-terminus of the base editor.

Exemplary base editors comprising a deaminase, a napDNAbp domain, and an NLS (e.g., any NLS provided herein) may have the following architecture: NH₂-[deaminase domain]-[napDNAbp domain]-[NLS]-COOH; NH₂-[napDNAbp domain]-[deaminase domain]-[NLS]-COOH; NH₂-[NLS]-[deaminase domain]-[napDNAbp domain]-COOH; or NH₂-[NLS]-[napDNAbp domain]-[deaminase domain]-COOH.

In particular embodiments, the disclosed base editors comprise the ABE architecture that follows, where TadA-8e is the adenosine deaminase domain: NH₂-[bpNLS]-[TadA-8c]-[napDNAbp domain]-[bpNLS]-COOH; NH₂-[bpNLS]-[napDNAbp domain]-[TadA-8c]-[bpNLS]-COOH; NH₂-[bpNLS]-[TadA-8c]-[napDNAbp domain]-[bpNLS]-COOH; or NH₂-[bpNLS]-[napDNAbp domain]-[TadA-8c]-[bpNLS]-COOH.

Exemplary base editors comprising a cytidine deaminase, a napDNAbp domain, a UGI domain, and an NLS (e.g., any NLS provided herein) may have the following architecture: NH₂-[napDNAbp domain]-[cytidine deaminase domain]-[UGI domain]-[bpNLS]-COOH; or NH₂-[bpNLS]-[napDNAbp domain]-[cytidine deaminase domain]-[UGI domain]-COOH; NH₂-[cytidine deaminase domain]-[napDNAbp domain]-[UGI domain]-[bpNLS]-COOH; or NH₂-[bpNLS]-[cytidine deaminase domain]-[napDNAbp domain]-[UGI domain]-COOH.

A representative nuclear localization signal is a peptide sequence that directs the protein to the nucleus of the cell in which the sequence is expressed. A nuclear localization signal is predominantly basic, can be positioned almost anywhere in a protein's amino acid sequence, generally comprises a short sequence of four amino acids (Autieri & Agrawal, (1998) J. Biol. Chem. 273:14731-37, incorporated herein by reference) to eight amino acids, and is typically rich in lysine and arginine residues (Magin et al., (2000) Virology 274:11-16, incorporated herein by reference). Nuclear localization signals often comprise proline residues. A variety of nuclear localization signals have been identified and have been used to effect transport of biological molecules from the cytoplasm to the nucleus of a cell. See, e.g., Tinland et al., (1992) Proc. Natl. Acad. Sci. U.S.A. 89:7442-46; Moede et al., (1999) FEBS Lett. 461:229-34, which is incorporated herein by reference. Translocation is currently thought to involve nuclear pore proteins.

Most NLSs can be classified in three general groups: (i) a monopartite NLS exemplified by the SV40 large T antigen NLS (PKKKRKV (SEQ ID NO: 408)); (ii) a bipartite motif consisting of two basic domains separated by a variable number of spacer amino acids and exemplified by the Xenopus nucleoplasmin NLS (KRXXXXXXXXXXKKKL (SEQ ID NO: 486)); and (iii) noncanonical sequences such as M9 of the hnRNP A1 protein, the influenza virus nucleoprotein NLS, and the yeast Gal4 protein NLS (Dingwall and Laskey, Trends Biochem Sci. 1991 December; 16 (12): 478-81).

Nuclear localization signals appear at various points in the amino acid sequences of proteins. NLSs have been identified at the N-terminus, the C-terminus, and in the central region of proteins. Thus, the specification provides base editors that may be modified with one or more NLSs at the C-terminus, the N-terminus, as well as at in internal region of the base editor. The residues of a longer sequence that do not function as component NLS residues should be selected so as not to interfere, for example tonically or sterically, with the nuclear localization signal itself. Therefore, although there are no strict limits on the composition of an NLS-comprising sequence, in practice, such a sequence can be functionally limited in length and composition.

The present disclosure contemplates any suitable means by which to modify a fusion protein (or base editor) to include one or more NLSs. In one aspect, the base editors can be engineered to express a fusion protein that is translationally fused at its N-terminus or its C-terminus (or both) to one or more NLSs, i.e., to form a fusion protein-NLS fusion construct. In other embodiments, the fusion protein-encoding nucleotide sequence can be genetically modified to incorporate a reading frame that encodes one or more NLSs in an internal region of the encoded fusion protein. In addition, the NLSs may include various amino acid linkers or spacer regions encoded between the fusion protein and the N-terminally, C-terminally, or internally-attached NLS amino acid sequence. Thus, the present disclosure also provides for nucleotide constructs, vectors, and host cells for expressing base editors that comprise a fusion protein and one or more NLSs.

The base editors described herein may also comprise nuclear localization signals which are linked to a fusion protein through one or more linkers, e.g., polymeric, amino acid, polysaccharide, chemical, or nucleic acid linker element. In certain embodiments, the NLS is linked to a fusion protein using an XTEN linker, as set forth in SEQ ID NO: 412. The linkers within the contemplated scope of the disclosure are not intended to have any limitations and can be any suitable type of molecule (e.g., polymer, amino acid, polysaccharide, nucleic acid, lipid, or any synthetic chemical linker domain) and be joined to the fusion protein by any suitable strategy that effectuates forming a bond (e.g., covalent linkage, hydrogen bonding) between the fusion protein and the one or more NLSs.

The base editors described herein also may include one or more additional elements. In certain embodiments, an additional element may comprise an effector of base repair, such as an inhibitor of base repair.

In some embodiments, the base editors described herein may comprise one or more heterologous protein domains (e.g., about or more than about 1, 2, 3, 4, 5, 6, 7, 8, 9, 10, or more domains in addition to the base editors components). A base editor may comprise any additional protein sequence, and optionally a linker sequence between any two domains. Other exemplary features that may be present are localization sequences, such as cytoplasmic localization sequences, export sequences, such as nuclear export sequences, or other localization sequences, as well as sequence tags.

Examples of heterologous protein domains that may be fused to a base editor or component thereof (e.g., the napDNAbp domain, the nucleotide modification domain, or the NLS domain) include, without limitation, epitope tags and reporter gene sequences. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-5-transferase (GST), horseradish peroxidase (HRP), chloramphenicol acetyltransferase (CAT), beta-galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP). A base editor may be fused to a gene sequence encoding a protein or a fragment of a protein that binds DNA molecules or binds other cellular molecules, including, but not limited to, maltose binding protein (MBP), S-tag, Lex A DNA binding domain (DBD) fusions, GAL4 DNA binding domain fusions, and herpes simplex virus (HSV) BP16 protein fusions. Additional domains that may form part of a base editor are described in US Patent Publication No. 2011/0059502, published Mar. 10, 2011, and incorporated herein by reference in its entirety.

In an aspect of the disclosure, a reporter gene which includes, but is not limited to, glutathione-5-transferase (GST), horseradish peroxidase (HRP), chloramphenicol acetyltransferase (CAT) beta-galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP), may be introduced into a cell to encode a gene product which serves as a marker by which to measure the alteration or modification of expression of the gene product. In certain embodiments of the disclosure the gene product is luciferase. In a further embodiment of the disclosure the expression of the gene product is decreased.

Other exemplary features that may be present are tags that are useful for solubilization, purification, or detection of the base editor. Suitable protein tags provided herein include, but are not limited to, biotin carboxylase carrier protein (BCCP) tags, myc-tags, calmodulin-tags, FLAG-tags, hemagglutinin (HA)-tags, bgh-PolyA tags, polyhistidine tags, and also referred to as histidine tags or His-tags, maltose binding protein (MBP)-tags, nus-tags, glutathione-S-transferase (GST)-tags, green fluorescent protein (GFP)-tags, thioredoxin-tags, S-tags, Softags (e.g., Softag 1, Softag 3), strep-tags, biotin ligase tags, FlAsH tags, V5 tags, and SBP-tags. Additional suitable sequences will be apparent to those of skill in the art. In some embodiments, the base editor comprises one or more His tags.

### Linkers

In certain embodiments, linkers may be used to link any of the peptides or peptide domains or domains of the base editor (e.g., a napDNAbp domain covalently linked to an adenosine deaminase domain which is covalently linked to an NLS domain). The base editors described herein may comprise linkers of 32 amino acids in length.

The linker may be as simple as a covalent bond, or it may be a polymeric linker many atoms in length. In certain embodiments, the linker is a polypeptide or based on amino acids. In other embodiments, the linker is not peptide-like. In certain embodiments, the linker is a covalent bond (e.g., a carbon-carbon bond, disulfide bond, carbon-heteroatom bond, etc.). In certain embodiments, the linker is a carbon-nitrogen bond of an amide linkage. In certain embodiments, the linker is a cyclic or acyclic, substituted or unsubstituted, branched or unbranched aliphatic or heteroaliphatic linker. In certain embodiments, the linker is polymeric (e.g., polyethylene, polyethylene glycol, polyamide, polyester, etc.). In certain embodiments, the linker comprises a monomer, dimer, or polymer of aminoalkanoic acid. In certain embodiments, the linker comprises an aminoalkanoic acid (e.g., glycine, ethanoic acid, alanine, beta-alanine, 3-aminopropanoic acid, 4-aminobutanoic acid, 5-pentanoic acid, etc.). In certain embodiments, the linker comprises a monomer, dimer, or polymer of aminohexanoic acid (Ahx). In certain embodiments, the linker is based on a carbocyclic moiety (e.g., cyclopentane, cyclohexane). In other embodiments, the linker comprises a polyethylene glycol moiety (PEG). In other embodiments, the linker comprises amino acids. In certain embodiments, the linker comprises a peptide. In certain embodiments, the linker comprises an aryl or heteroaryl moiety. In certain embodiments, the linker is based on a phenyl ring. The linker may include functionalized moieties to facilitate attachment of a nucleophile (e.g., thiol, amino) from the peptide to the linker. Any electrophile may be used as part of the linker. Exemplary electrophiles include, but are not limited to, activated esters, activated amides, Michael acceptors, alkyl halides, aryl halides, acyl halides, and isothiocyanates.

In some embodiments, the linker is 5-100 amino acids in length, for example, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35-40, 40-45, 45-50, 50-60, 60-70, 70-80, 80-90, 90-100, 100-110, 110-120, 120-130, 130-140, 140-150, or 150-200 amino acids in length. Longer or shorter linkers are also contemplated. In some embodiments, the linker is 32 amino acids in length. In exemplary embodiments, the linker comprises the 32-amino acid sequence SGGSSGGSSGSETPGTSESATPESSGGSSGGS (SEQ ID NO: 412), also known as an XTEN linker. In some embodiments, the linker comprises the 9-amino acid sequence SGGSGGSGGS (SEQ ID NO: 413). In some embodiments, the linker comprises the 4-amino acid sequence SGGS (SEQ ID NO: 414).

In some embodiments, the linker comprises the amino acid sequence (GGGGS), (SEQ ID NO: 415), (G)ₙ (SEQ ID NO: 416), (EAAAK)ₙ (SEQ ID NO: 417), (GGS), (SEQ ID NO: 418), (SGGS)ₙ (SEQ ID NO: 419), (XP)ₙ (SEQ ID NO: 420), or any combination thereof, wherein n is independently an integer between 1 and 30, and wherein X is any amino acid. In some embodiments, the linker comprises the amino acid sequence (GGS), (SEQ ID NO: 421), wherein n is 1, 3, or 7. In some embodiments, the linker comprises the amino acid sequence SGSETPGTSESATPES (SEQ ID NO: 422).

In some embodiments, a linker comprises SGSETPGTSESATPES (SEQ ID NO: 422), and SGGS (SEQ ID NO: 414). In some embodiments, a linker comprises SGGSSGSETPGTSESATPESSGGS (SEQ ID NO: 423). In some embodiments, a linker comprises SGGSSGGSSGSETPGTSESATPESSGGSSGGS (SEQ ID NO: 412). In some embodiments, a linker comprises GGSGGSPGSPAGSPTSTEEGTSESATPESGPGTSTEPSEGSAPGSPAGSPTSTEEGTSTE PSEGSAPGTSTEPSEGSAPGTSESATPESGPGSEPATSGGSGGS (SEQ ID NO: 424). In some embodiments, the linker is 24 amino acids in length. In some embodiments, the linker comprises the amino acid sequence SGGSSGGSSGSETPGTSESATPES (SEQ ID NO: 425). In some embodiments, the linker is 40 amino acids in length. In some embodiments, the linker comprises the amino acid sequence SGGSSGGSSGSETPGTSESATPESSGGSSGGSSGGSSGGS (SEQ ID NO: 426). In some embodiments, the linker is 64 amino acids in length. In some embodiments, the linker comprises the amino acid sequence SGGSSGGSSGSETPGTSESATPESSGGSSGGSSGGSSGGSSGSETPGTSESATPESSGGS SGGS (SEQ ID NO: 427). In some embodiments, the linker is 92 amino acids in length. In some embodiments, the linker comprises the amino acid sequence PGSPAGSPTSTEEGTSESATPESGPGTSTEPSEGSAPGSPAGSPTSTEEGTSTEPSEGSAP GTSTEPSEGSAPGTSESATPESGPGSEPATS (SEQ ID NO: 428). It should be appreciated that any of the linkers provided herein may be used to link a first adenosine deaminase and a second adenosine deaminase; an adenosine deaminase domain (comprising, e.g., a first and/or a second adenosine deaminase) and a napDNAbp; a napDNAbp and an NLS; or an adenosine deaminase domain and an NLS.

In some embodiments, any of the base editors provided herein, comprise an adenosine deaminase and a napDNAbp that are fused to each other via a linker. In some embodiments, any of the base editors provided herein, comprise a first adenosine deaminase and a second adenosine deaminase that are fused to each other via a linker. In some embodiments, any of the base editors provided herein, comprise an NLS, which may be fused to an adenosine deaminase (e.g., a first and/or a second adenosine deaminase) and a nucleic acid programmable DNA binding protein (napDNAbp). Various linker lengths and flexibilities between an adenosine deaminase (e.g., an engineered ecTadA) and a napDNAbp (e.g., a Cas9 domain), and/or between a first adenosine deaminase and a second adenosine deaminase may be employed (e.g., ranging from very flexible linkers of the form of SEQ ID NOs: 119, 121-124 (see, e.g., Guilinger J P, Thompson D B, Liu D R. Fusion of catalytically inactive Cas9 to FokI nuclease improves the specificity of genome modification. Nat. Biotechnol. 2014; 32 (6): 577-82; the entire contents are incorporated herein by reference) and (XP), (SEQ ID NO: 420)) in order to achieve the optimal length for deaminase activity for the specific application. In some embodiments, n is 1, 2, 3, 4, 5, 6, 7, 8, 9, 10, 11, 12, 13, 14, or 15. In some embodiments, the linker comprises a (GGS), (SEQ ID NO: 421) motif, wherein n is 1, 3, or 7. In some embodiments, the adenosine deaminase and the napDNAbp, and/or the first adenosine deaminase and the second adenosine deaminase of any of the base editors provided herein are fused via a linker comprising an amino acid sequence selected from SEQ ID NOs: 119-132. In some embodiments, the linker is 24 amino acids in length. In some embodiments, the linker comprises the amino acid sequence (SGGS) 2-SGSETPGTSESATPES-(SGGS)₂ (SEQ ID NO: 412), which may also be referred to as (SGGS) 2-XTEN-(SGGS)₂ (SEQ ID NO: 429). In some embodiments, the linker comprises the amino acid sequence, wherein n is 0, 1, 2, 3, 4, 5, 6, 7, 8, 9, or 10. In some embodiments, the linker is 40 amino acids in length. In some embodiments, the linker is 64 amino acids in length. In some embodiments, the linker is 92 amino acids in length.

The above description is meant to be non-limiting with regard to making base editors having increased expression, and thereby increase editing efficiencies.

### Methods of Treatment and Uses

Other aspects of the present disclosure provide methods of delivering the base editor into a cell to form a complete and functional Cas9 protein or nucleobase editor. For example, in some embodiments, a cell is contacted with a composition described herein (e.g., compositions comprising nucleotide sequences encoding the base editor or AAV particles containing nucleic acid vectors comprising such nucleotide sequences). In some embodiments, the contacting results in the delivery of such nucleotide sequences into a cell, wherein the N-terminal portion of the Cas9 protein or the nucleobase editor and the C-terminal portion of the Cas9 protein or the nucleobase editor are expressed in the cell and are joined to form a complete Cas9 protein or a complete nucleobase editor.

It should be appreciated that any rAAV particle, nucleic acid molecule or composition provided herein may be introduced into the cell in any suitable way, either stably or transiently. In some embodiments, the disclosed proteins may be transfected into the cell. In some embodiments, the cell may be transduced or transfected with a nucleic acid molecule. For example, a cell may be transduced (e.g., with a virus encoding a protein), or transfected (e.g., with a plasmid encoding a protein) with a nucleic acid molecule that encodes a protein, or an rAAV particle containing a viral genome encoding one or more nucleic acid molecules. Such transduction may be a stable or transient transduction. In some embodiments, cells expressing a protein or containing a protein may be transduced or transfected with one or more guide RNA sequences, for example in delivery of a base editor. In some embodiments, a plasmid expressing a protein may be introduced into cells through electroporation, transient (e.g., lipofection) and stable genome integration (e.g., nucleofection or piggyback) and viral transduction or other methods known to those of skill in the art.

In some aspects, the invention provides methods comprising delivering one or more base editor-encoding polynucleotides, one or more transcripts thereof, and/or one or proteins transcribed therefrom, to a cell using a non-viral delivery method. Methods of non-viral delivery of nucleic acids include lipofection, nucleofection, microinjection, biolistics, virosomes, liposomes, immunoliposomes, polycation or lipid: nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Feigner, WO 1991/17424; WO 1991/16024. Delivery can be to cells (e.g. in vitro or ex vivo administration) or target tissues (e.g. in vivo administration).

In certain embodiments, the compositions provided herein comprise a lipid and/or polymer. In certain embodiments, the lipid and/or polymer is cationic. The preparation of such lipid particles is well known. Sec, e.g. U.S. Pat. Nos. 4,880,635; 4,906,477; 4,911,928; 4,917,951; 4,920,016; 4,921,757; and 9,737,604, each of which is incorporated herein by reference.

In some embodiments, the target nucleotide sequence is a DNA sequence in a genome, e.g. a eukaryotic genome. In certain embodiments, the target nucleotide sequence is in a mammalian (e.g. a human) genome.

The target nucleotide sequence may comprise a target sequence (e.g., a point mutation) associated with a disease, disorder, or condition. The target sequence may comprise a T to C (or A to G) point mutation associated with a disease, disorder, or condition, and wherein the deamination of the mutant C base results in mismatch repair-mediated correction to a sequence that is not associated with a disease, disorder, or condition. The target sequence may otherwise comprise a G to A (or C to T) point mutation associated with a disease, disorder, or condition, and wherein the deamination of the mutant A base results in mismatch repair-mediated correction to a sequence that is not associated with a disease, disorder, or condition. The target sequence may encode a protein, and where the point mutation is in a codon and results in a change in the amino acid encoded by the mutant codon as compared to a wild-type codon. The target sequence may also be at a splice site, and the point mutation results in a change in the splicing of an mRNA transcript as compared to a wild-type transcript. In addition, the target may be at a non-coding sequence of a gene, such as a promoter, and the point mutation results in increased or decreased expression of the gene.

Thus, in some aspects, the deamination of a mutant C results in a change of the amino acid encoded by the mutant codon, which in some cases can result in the expression of a wild-type amino acid. In other aspects, the deamination of a mutant A results in a change of the amino acid encoded by the mutant codon, which in some cases can result in the expression of a wild-type amino acid.

The methods described herein involving contacting a cell with a composition or rAAV particle can occur in vitro, ex vivo, or in vivo. In certain embodiments, the step of contacting a cell occurs in a subject. In certain embodiments, the subject has been diagnosed with a disease, disorder, or condition. In some embodiments, the step of contacting a cell occurs ex vivo, or outside of a subject.

In some embodiments, the methods disclosed herein involve contacting a mammalian cell with a composition or rAAV particle. In particular embodiments, the methods involve contacting a retinal cell, cortical cell or cerebellar cell.

The compositions described herein may be administered to a subject in need thereof in a therapeutically effective amount to treat and/or prevent a disease or disorder the subject is suffering from. Any disease or disorder that may be treated and/or prevented using CRISPR/Cas9-based genome-editing technology may be treated by the base editor described herein. It is to be understood that, if the nucleotide sequences encoding the base editor does not further encode a gRNA, a separate nucleic acid vector encoding the gRNA may be administered together with the compositions described herein.

Exemplary suitable diseases, disorders or conditions include, without limitation, cardiovascular disease, cystic fibrosis, phenylketonuria, epidermolytic hyperkeratosis (EHK), chronic obstructive pulmonary disease (COPD), Charcot-Marie-Toot disease type 4J, neuroblastoma (NB), von Willebrand disease (vWD), myotonia congenital, hereditary renal amyloidosis, dilated cardiomyopathy, hereditary lymphedema, familial Alzheimer's disease, prion disease, chronic infantile neurologic cutaneous articular syndrome (CINCA), congenital deafness, Niemann-Pick disease type C (NPC) disease, and desmin-related myopathy (DRM).

In some embodiments, the disease or condition is cardiovascular disease. In some embodiments, the disease or condition is Niemann-Pick disease type C (NPC) disease.

In some embodiments, the disease, disorder or condition is associated with a point mutation that introduces a stop codon, for example, a premature stop codon within the coding region of a gene. In some embodiments, the desired base edit removes a stop codon within the coding region of a gene. In some embodiments, the desired base edit disrupts a splice acceptor site or a splice donor site.

In some embodiments, the desired base edit is associated with disruption of a splice acceptor site or a splice donor site in a PCSK9 gene, or an Angpil3 gene. In certain embodiments, the desired base edit is associated with the disruption of a splice acceptor site at W8 in a PCKS9 gene. In some embodiments, the desired base edit is an A to G edit that disrupts the splice acceptor site at residue 8, generating a W8R substitution.

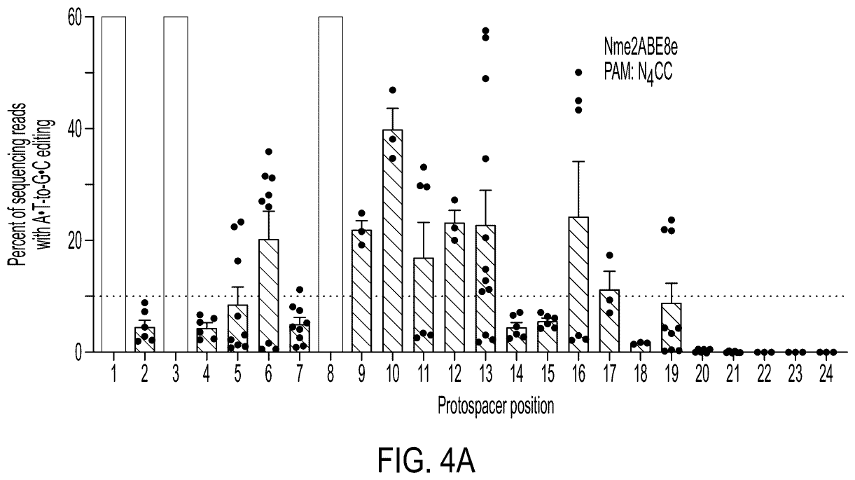

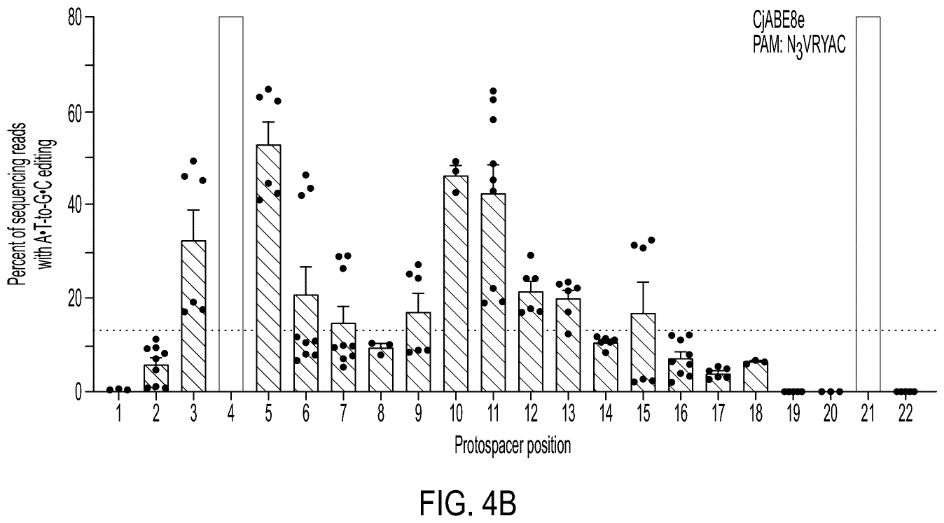

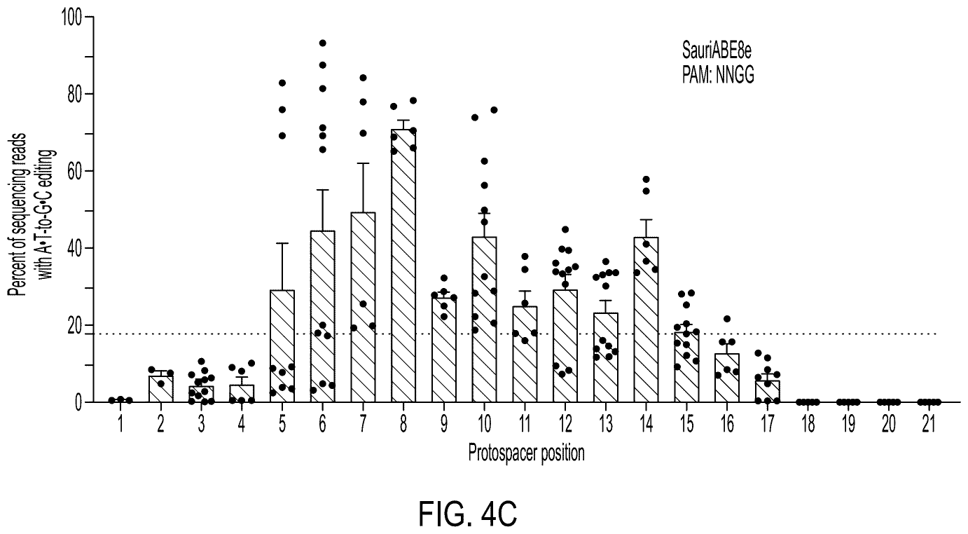

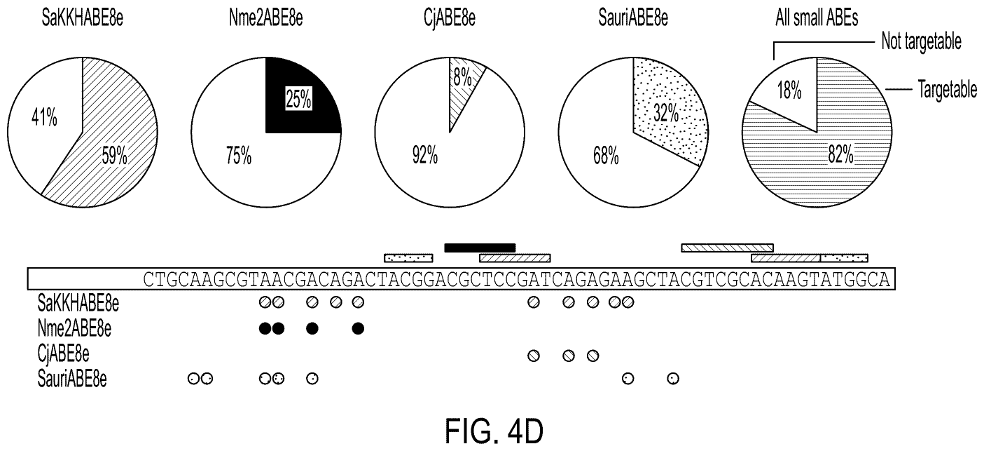

Additional exemplary diseases, disorders and conditions include cystic fibrosis (see, e.g., Schwank et al., Functional repair of CFTR by CRISPR/Cas9 in intestinal stem cell organoids of cystic fibrosis patients. Cell stem cell. 2013; 13:653-658; and Wu et. al., Correction of a genetic disease in mouse via use of CRISPR-Cas9. Cell stem cell. 2013; 13:659-662, neither of which uses a deaminase fusion protein to correct the genetic defect); phenylketonuria—e.g., phenylalanine to serine mutation at position 835 (mouse) or 240 (human) or a homologous residue in phenylalanine hydroxylase gene (T>C mutation)—see, e.g., McDonald et al., Genomics. 1997; 39:402-405; Bernard-Soulier syndrome (BSS)—e.g., phenylalanine to serine mutation at position 55 or a homologous residue, or cysteine to arginine at residue 24 or a homologous residue in the platelet membrane glycoprotein IX (T>C mutation)—see, e.g., Noris et al., British Journal of Haematology. 1997; 97:312-320, and Ali et al., Hematol. 2014; 93:381-384; epidermolytic hyperkeratosis (EHK)—e.g., leucine to proline mutation at position 160 or 161 (if counting the initiator methionine) or a homologous residue in keratin 1 (T>C mutation)—see, e.g., Chipev et al., Cell. 1992; 70:821-828, see also accession number P04264 in the UNIPROT database at www[dot]uniprot[dot]org; chronic obstructive pulmonary disease (COPD)—e.g., leucine to proline mutation at position 54 or 55 (if counting the initiator methionine) or a homologous residue in the processed form of α₁-antitrypsin or residue 78 in the unprocessed form or a homologous residue (T>C mutation)—see, e.g., Poller et al., Genomics. 1993; 17:740-743, see also accession number P01011 in the UNIPROT database; Charcot-Marie-Toot disease type 4J—e.g., isoleucine to threonine mutation at position 41 or a homologous residue in FIG. 4 (T>C mutation)—see, e.g., Lenk et al., PLoS Genetics. 2011; 7: e1002104; neuroblastoma (NB)—e.g., leucine to proline mutation at position 197 or a homologous residue in Caspase-9 (T>C mutation)—see, e.g., Kundu et al., 3 Biotech. 2013, 3:225-234; von Willebrand disease (vWD)—e.g., cysteine to arginine mutation at position 509 or a homologous residue in the processed form of von Willebrand factor, or at position 1272 or a homologous residue in the unprocessed form of von Willebrand factor (T>C mutation)—see, e.g., Lavergne et al., Br. J. Haematol. 1992, see also accession number P04275 in the UNIPROT database; 82:66-72; myotonia congenital—e.g., cysteine to arginine mutation at position 277 or a homologous residue in the muscle chloride channel gene CLCN1 (T>C mutation)—see, e.g., Weinberger et al., The J. of Physiology. 2012; 590:3449-3464; hereditary renal amyloidosis—e.g., stop codon to arginine mutation at position 78 or a homologous residue in the processed form of apolipoprotein All or at position 101 or a homologous residue in the unprocessed form (T>C mutation)—see, e.g., Yazaki et al., Kidney Int. 2003; 64:11-16; dilated cardiomyopathy (DCM)—e.g., tryptophan to Arginine mutation at position 148 or a homologous residue in the FOXD4 gene (T>C mutation), see, e.g., Minoretti et. al., Int. J. of Mol. Med. 2007; 19:369-372; hereditary lymphedema—e.g., histidine to arginine mutation at position 1035 or a homologous residue in VEGFR3 tyrosine kinasc (A>G mutation), see, e.g., Irrthum et al., Am. J. Hum. Genet. 2000; 67:295-301; familial Alzheimer's disease—e.g., isoleucine to valine mutation at position 143 or a homologous residue in presenilin1 (A>G mutation), see, e.g., Gallo ct. al., J. Alzheimer's disease. 2011; 25:425-431; Prion disease—e.g., methionine to valine mutation at position 129 or a homologous residue in prion protein (A>G mutation)—see, e.g., Lewis et. al., J. of General Virology. 2006; 87:2443-2449; chronic infantile neurologic cutaneous articular syndrome (CINCA)—e.g., Tyrosine to Cysteine mutation at position 570 or a homologous residue in cryopyrin (A>G mutation)—see, e.g., Fujisawa et. al. Blood. 2007; 109:2903-2911; and desmin-related myopathy (DRM)—e.g., arginine to glycine mutation at position 120 or a homologous residue in αβ crystallin (A>G mutation)—see, e.g., Kumar et al., J. Biol. Chem. 1999; 274:24137-24141. The entire contents of all references and database entries is incorporated herein by reference.

Treatment of a disease or disorder includes delaying the development or progression of the disease, or reducing disease severity. Treating the disease does not necessarily require curative results.

As used therein, “delaying” the development of a disease means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset.

As used herein “onset” or “occurrence” of a disease includes initial onset and/or recurrence. Conventional methods, known to those of ordinary skill in the art of medicine, can be used to administer the isolated polypeptide or pharmaceutical composition to the subject, depending upon the type of disease to be treated or the site of the disease.

In some aspects, the present disclosure provides uses of any one of the disclosed base editors described herein and a guide RNA targeting this nucleobase editor to a target in the manufacture of a medicament. In some aspects, uses of any one of the nucleobase editors and guide RNAs described herein are provided in the manufacture of a kit for base editing, wherein the base editing comprises contacting the nucleic acid molecule with the base editor and guide RNA under conditions suitable for the substitution of the adenine (A) of a A:T nucleobase pair in the target with a guanine (G), or for the substitution of the cytosine (C) of a C:T nucleobase pair in the target with a thymine (T). In some embodiments, the step of contacting of induces separation of the double-stranded DNA at a target region. In some embodiments, the step of contacting further comprises nicking one strand of the double-stranded DNA, wherein the one strand comprises an unmutated strand.

In some embodiments of the described uses, the step of contacting is performed in vitro. In other embodiments, the step of contacting is performed in vivo. In some embodiments, the step of contacting is performed in a subject (e.g., a human subject or a non-human animal subject). In some embodiments, the step of contacting is performed in a human or non-human animal cell. In some embodiments, the step of contacting is performed in a plant cell.

The present disclosure also provides uses of any one of the nucleobase editors or any one of the complexes of nucleobase editors and guide RNAs described herein as a medicament. The present disclosure also provides uses of the described pharmaceutical compositions or cells comprising, and vectors or rAAV particles encoding, any of the disclosed nucleobase editors or complexes herein as a medicament. In some embodiments, the medicament is for treatment of cardiovascular disease.

In some aspects, the present disclosure provides uses of any one of the base editors described herein and a guide RNA targeting this base editor to a target base pair in a nucleic acid molecule in the manufacture of a kit for nucleic acid editing, wherein the nucleic acid editing comprises contacting the nucleic acid molecule with the base editor and guide RNA under conditions suitable for the desired base edit. In some embodiments, the desired base edit is the substitution of the adenine (A) of a target A:T base pair with a guanine (G). In some embodiments of these uses, the nucleic acid molecule is a double-stranded DNA molecule. In some embodiments, the step of contacting induces separation of the double-stranded DNA at a target region. In some embodiments, the step of contacting thereby comprises nicking one strand of the double-stranded DNA, wherein the one strand comprises an unmutated strand that comprises the T of the target A:T nucleobase pair.

In some embodiments of the described uses, the step of contacting is performed in vitro. In other embodiments, the step of contacting is performed in vivo. In some embodiments, the step of contacting is performed in a subject (e.g., a human subject or a non-human animal subject). In some embodiments, the step of contacting is performed in a human or non-human animal cell. In some embodiments, the step of contacting is performed in a plant cell.

The present disclosure also provides uses of any one of the adenine base editors described herein as a medicament. The present disclosure also provides uses of any one of the complexes of adenine base editors and guide RNAs described herein as a medicament.

### Kits

The compositions of the present disclosure may be assembled into kits. In some embodiments, the kit comprises nucleic acid vectors for the expression of the nucleobase editors described herein. In some embodiments, the kit further comprises appropriate guide nucleotide sequences (e.g., gRNAs) or nucleic acid vectors for the expression of such guide nucleotide sequences, to target the Cas9 protein or nucleobase editor to the desired target sequence.

The kit described herein may include one or more containers housing components for performing the methods described herein and optionally instructions for use. Any of the kit described herein may further comprise components needed for performing the assay methods. Each component of the kits, where applicable, may be provided in liquid form (e.g., in solution) or in solid form, (e.g., a dry powder). In certain cases, some of the components may be reconstitutable or otherwise processible (e.g., to an active form), for example, by the addition of a suitable solvent or other species (for example, water), which may or may not be provided with the kit.

In some embodiments, the kits may optionally include instructions and/or promotion for use of the components provided. As used herein, “instructions” can define a component of instruction and/or promotion, and typically involve written instructions on or associated with packaging of the disclosure. Instructions also can include any oral or electronic instructions provided in any manner such that a user will clearly recognize that the instructions are to be associated with the kit, for example, audiovisual (e.g., videotape, DVD, etc.), Internet, and/or web-based communications, etc. The written instructions may be in a form prescribed by a governmental agency regulating the manufacture, use, or sale of pharmaceuticals or biological products, which can also reflect approval by the agency of manufacture, use or sale for animal administration. As used herein, “promoted” includes all methods of doing business including methods of education, hospital and other clinical instruction, scientific inquiry, drug discovery or development, academic research, pharmaceutical industry activity including pharmaceutical sales, and any advertising or other promotional activity including written, oral and electronic communication of any form, associated with the disclosure. Additionally, the kits may include other components depending on the specific application, as described herein.

The kits may contain any one or more of the components described herein in one or more containers. The components may be prepared sterilely, packaged in a syringe and shipped refrigerated. Alternatively it may be housed in a vial or other container for storage. A second container may have other components prepared sterilely. Alternatively the kits may include the active agents premixed and shipped in a vial, tube, or other container.

The kits may have a variety of forms, such as a blister pouch, a shrink wrapped pouch, a vacuum scalable pouch, a scalable thermoformed tray, or a similar pouch or tray form, with the accessories loosely packed within the pouch, one or more tubes, containers, a box or a bag. The kits may be sterilized after the accessories are added, thereby allowing the individual accessories in the container to be otherwise unwrapped. The kits can be sterilized using any appropriate sterilization techniques, such as radiation sterilization, heat sterilization, or other sterilization methods known in the art. The kits may also include other components, depending on the specific application, for example, containers, cell media, salts, buffers, reagents, syringes, needles, a fabric, such as gauze, for applying or removing a disinfecting agent, disposable gloves, a support for the agents prior to administration, etc.

### Host Cells

Cells that may contain any of the compositions described herein include prokaryotic cells and eukaryotic cells. The methods described herein are used to deliver a Cas9 protein or a nucleobase editor into a eukaryotic cell (e.g., a mammalian cell, such as a human cell). In some embodiments, the cell is in vitro (e.g., cultured cell. In some embodiments, the cell is in vivo (e.g., in a subject such as a human subject). In some embodiments, the cell is ex vivo (e.g., isolated from a subject and may be administered back to the same or a different subject).

Mammalian cells of the present disclosure include human cells, primate cells (e.g., vero cells), rat cells (e.g., GH3 cells, OC23 cells) or mouse cells (e.g., MC3T3 (“3T3”) cells or mouse neuroblastoma neuro-2A (“N2A”) cells). There are a variety of human cell lines, including, without limitation, human embryonic kidney (HEK, or HEK293T) cells, HeLa cells, cancer cells from the National Cancer Institute's 60 cancer cell lines (NCI60), DU145 (prostate cancer) cells, Lncap (prostate cancer) cells, MCF-7 (breast cancer) cells, MDA-MB-438 (breast cancer) cells, PC3 (prostate cancer) cells, T47D (breast cancer) cells, THP-1 (acute myeloid leukemia) cells, U87 (glioblastoma) cells, SHSY5Y human neuroblastoma cells (cloned from a myeloma) and Saos-2 (bone cancer) cells. In some embodiments, rAAV vectors are delivered into human embryonic kidney (HEK) cells (e.g., HEK 293 or HEK 293T cells). In some embodiments, rAAV vectors are delivered into stem cells (e.g., human stem cells) such as, for example, pluripotent stem cells (e.g., human pluripotent stem cells including human induced pluripotent stem cells (hiPSCs)). A stem cell refers to a cell with the ability to divide for indefinite periods in culture and to give rise to specialized cells. A pluripotent stem cell refers to a type of stem cell that is capable of differentiating into all tissues of an organism, but not alone capable of sustaining full organismal development. A human induced pluripotent stem cell refers to a somatic (e.g., mature or adult) cell that has been reprogrammed to an embryonic stem cell-like state by being forced to express genes and factors important for maintaining the defining properties of embryonic stem cells (see, e.g., Takahashi and Yamanaka, Cell 126 (4): 663-76, 2006, incorporated herein by reference). Human induced pluripotent stem cell cells express stem cell markers and are capable of generating cells characteristic of all three germ layers (ectoderm, endoderm, mesoderm).

Examples of cell lines that may be used in accordance with the present disclosure include 293-T, 293-T, 3T3, N2A, 4T1, 721, 9L, A-549, A172, A20, A253, A2780, A2780ADR, A2780cis, A431, ALC, B16, B35, BCP-1, BEAS-2B, bEnd.3, BHK-21, BR 293, BxPC3, C2C12, C3H-10T1/2, C6, C6/36, Cal-27, CGR8, CHO, CML T1, CMT, COR-L23, COR-L23/5010, COR-L23/CPR, COR-L23/R23, COS-7, COV-434, CT26, D17, DH82, DU145, DuCaP, E14Tg2a, EL4, EM2, EM3, EMT6/AR1, EMT6/AR10.0, FM3, H1299, H69, HB54, HB55, HCA2, Hepa1c1c7, High Five cells, HL-60, HMEC, HT-29, HUVEC, J558L cells, Jurkat, JY cells, K562 cells, KCL22, KG1, Ku812, KYO1, LNCap, Ma-Mel 1, 2, 3 . . . 48, MC-38, MCF-10A, MCF-7, MDA-MB-231, MDA-MB-435, MDA-MB-468, MDCK II, MG63, MONO-MAC 6, MOR/0.2R, MRC5, MTD-1A, MyEnd, NALM-1, NCI-H69/CPR, NCI-H69/LX10, NCI-H69/LX20, NCI-H69/LX4, NIH-3T3, NW-145, OPCN/OPCT Peer, PNT-1A/PNT 2, PTK2, Raji, RBL cells, RenCa, RIN-5F, RMA/RMAS, S2, Saos-2 cells, Sf21, Sf9, SiHa, SKBR3, SKOV-3, T-47D, T2, T84, THP1, U373, U87, U937, VCaP, WM39, WT-49, X63, YAC-1 and YAR cells.

Without further elaboration, it is believed that one skilled in the art can, based on the above description, utilize the present disclosure to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

### Example 1

In this study small, highly active ABE8e variants were constructed and the minimal necessary cis-acting components on the AAV genome were identified to develop highly efficient single-AAV vectors with broad in vivo targeting capability. ABE8e variants that use compact CjCas9, Nme2Cas9 and SauriCas9 domains were characterized to develop a suite of single-AAV, high-activity adenine base editors that collectively offered compatibility with a broad range of PAM sequences, including commonly occurring N₄CC and N2GG PAMs, enabling base editing of approximately 82% of adenines in the human genome in principle (see FIG. 4D). Finally, the performance of single-AAV ABEs was assessed in mice by using them to install base edits associated with decreased cardiovascular disease risk, resulting in efficient editing (averaging 50%) of human PCSK9, mouse Pesk9 and mouse Angptl3 in bulk liver at a range of clinically relevant doses with concomitant substantial reduction in circulating target protein, total cholesterol, and triglycerides. These findings illustrated advancements in the therapeutic potential of base editing, established the benefits of single-AAV base editor constructs, and provided a suite of single-AAV adenine base editors with broad collective targeting capability that supported efficient in vivo base editing. Development of a size-minimized AAV backbone for ABE delivery

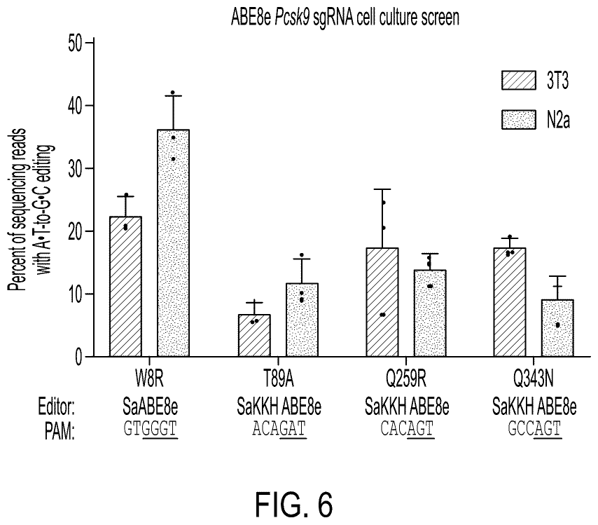

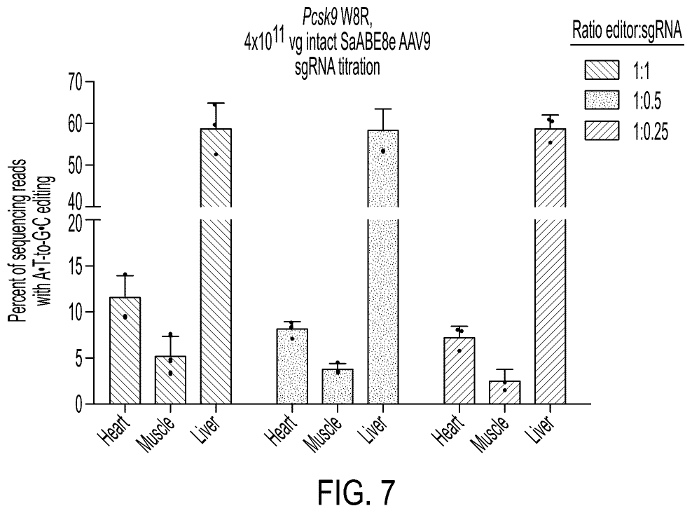

Starting with the small, robust editor SaABE8e (3.9 kb) 30 and its PAM-variant SaKKH-ABE8c^{30, 33}, the components of the AAV genome were optimized to yield size-minimized AAV-based delivery of adenine base editors in which the entire base editor, its guide RNA, and all necessary promoters and regulatory sequences were present in a single AAV (≤˜4.9 kb, not including ITRs). First, high-efficiency guide RNAs targeting Pcsk9 to allow evaluation of in vivo genome editing (irrespective of protein knockdown) in N2A and 3T3 cells were identified by transfecting plasmids encoding SaABE8e or SaKKH-ABE8c and corresponding sgRNAs with spacers that targeted the endogenous Pesk9 gene. The editing efficiency of each sgRNA was analyzed by targeted high-throughput DNA sequencing (HTS) (FIG. 6). The most efficient guide RNA installed a W8R coding mutation in Pesk9 using SaABE8c (FIG. 6).

To encode the full-length SaABE8e protein on a single AAV genome, the small ubiquitous promoter EFS (EF-1a short) and a terminator, that was previously validated, was used to yield efficient split-intein base editor delivery: the gamma portion of WPRE (W3) with bovine growth hormone (bGH) polyadenylation signal³⁴. To simplify production when testing multiple AAV architectures, sgRNA targeting Pcsk9 W8 was provided on a separate AAV.

First, the in vivo editing activity of intact SaABE8e delivered on a second AAV was compared to that of intein-split SaABE8e delivered on a second and third AAV using a previously validated intein split site²² (FIG. 1A). To assess editing across multiple tissues, a mixture of two or three AAV9 encoding (1) EGFP and sgRNA targeting Pesk9 and (2) either the intact AAV9 SaABE8e or the intein-split AAV9 SaABE8e (FIG. 1a) was systemically administered by retroorbital injection into 6 to 7-week-old wild-type C57BL/6J mice. Either a high total dose (8×10¹¹ vg, or 4×10¹³ vg/kg) or low total dose (8×10¹⁰ vg, or 4×10¹² vg/kg) of AAV consisting of a 1:1 mixture of sgRNA AAV to total base editor AAV was injected. Low doses of AAV were purposefully chosen to avoid saturating editing efficiency and to increase the likelihood of observing differences in editing outcomes between different ABE-AAV architectures. Three weeks post-injection, liver, heart, and muscle was harvested for analysis by HTS.

Both intact and intein-split SaABE8e AAVs resulted in dose-dependent and tissue-dependent editing activity consistent with the tropism of AAV9 across harvested tissues^{35, 36} with liver showing the highest editing efficiencies, followed by heart, then skeletal muscle. Intact SaABE8e AAV yielded robust editing at both doses administered, reaching 59%, 12%, and 5.3% editing of bulk liver, heart, and skeletal muscle, respectively, comparable or higher to the editing efficiencies achieved with intein-split SaABE8e in all tested tissues and doses (FIG. 1B). Decreasing the amount of sgRNA AAV to one half or one quarter the amount of base editor AAV did not affect editing efficiency in the liver, but decreased editing efficiency by ˜1,4-fold per 2-fold decrease in administered sgRNA AAV in both heart and muscle (FIG. 6), indicating that the dose of guide RNA partially limited editing efficiency in extrahepatic tissues under these conditions.

Next, improved editing efficiency and minimizing the size of ABE AAV was sought by identifying minimal necessary elements on the AAV genome. Five AAV genome architectures were designed and compared for delivery of SaABE8e to assess the impact of modifying the EFS promoter by adding a minimal minute virus of mice (MVM) intron³⁷, modifying the terminator by removing the truncated WPRE gamma subunit W3, or replacing the bGH polyadenylation signal with an SV40 late polyadenylation signal. The following AAV expression cassettes were designed and produced: (1) EFS-SaABE8c-W3bGH, (2) EFS-MVM-SaABE8c-W3bGH, (3) EFS-MVM-SaABE8c-bGH, (4) EFS-SaABE8c-bGH, and (5) EFS-SaABE8e-W3-SV40. Each of the five AAV candidates were administered to 6- to 7-week-old wild-type C57BL/6J mice by retro-orbital injection at a high dose (4×10¹¹ vg editor AAV plus 4×10¹¹ vg sgRNA AAV) or low dose (4×10¹⁰ vg editor AAV plus 4×10¹⁰ vg sgRNA AAV) (FIG. 2C).

Three weeks after AAV injection, liver, heart, and skeletal muscle tissues were harvested. Each tissue was analyzed by HTS. Editing efficiencies followed a consistent pattern among architectures, with the highest efficiency construct of EFS promoter driving SaABE8e expression and a bGH polyadenylation signal without W3 (EFS-SaABE8c-bGH) outperforming the other architectures across all assessed doses and tissues. These data demonstrated that the cis-acting W3 element was not necessary for sufficient expression of SaABE8e from the EFS promoter in these tissues and cell types and highlighted the importance of assessing AAV elements in the context of a specific editing application.

### Development of a Single-AAV Adenine Base Editing System

The space gained by removal of W3 (250 bp) allowed the addition of an sgRNA expression cassette on the AAV genome, thereby enabling a single AAV with both ABE and guide RNA expression cassettes (FIG. 2A). The U6 sgRNA cassette was inserted proximal to the 3′ ITR, as this orientation was previously found to enhance base editing activity in intein-split BE AAVs³⁴. This single-AAV9 SaABE8e was injected retro-orbitally into 6-8-week-old C57BL/6J mice at a dose of 4×10¹¹ vg or 4×10¹⁰ vg, matching the dose of base editor AAV used in previous experiments, and corresponding to half the previously used total AAV dose since the sgRNA was now expressed from the same AAV as the base editor. Single-AAV SaABE8e performed similarly at half the total AAV dose to intact SaABE8e and sgRNA expressed from two different AAVs in the liver at both the high and low doses. Single-AAV SaABE8e yielded 64% and 55% editing of bulk liver at high and low dose, respectively (FIG. 2B), similar to editing achieved with dual-AAV SaABE8c at high and low doses. Single-AAV SaABE8e also resulted in 23% and 13% editing of bulk heart tissue at the high and low dose, respectively, corresponding to 1.4-fold and 4.8-fold higher editing efficiency compared to dual-AAV ABE (FIG. 2B, P=0.038 and P=0.0012, respectively, by unpaired t-test). Single-AAV SaABE8e yielded comparable editing to dual-AAV SaABE8e at the high dose and low dose in skeletal muscle compared to dual AAV treatment, yielding 7.8% and 5.5% at high and low dose, respectively (FIG. 2B).

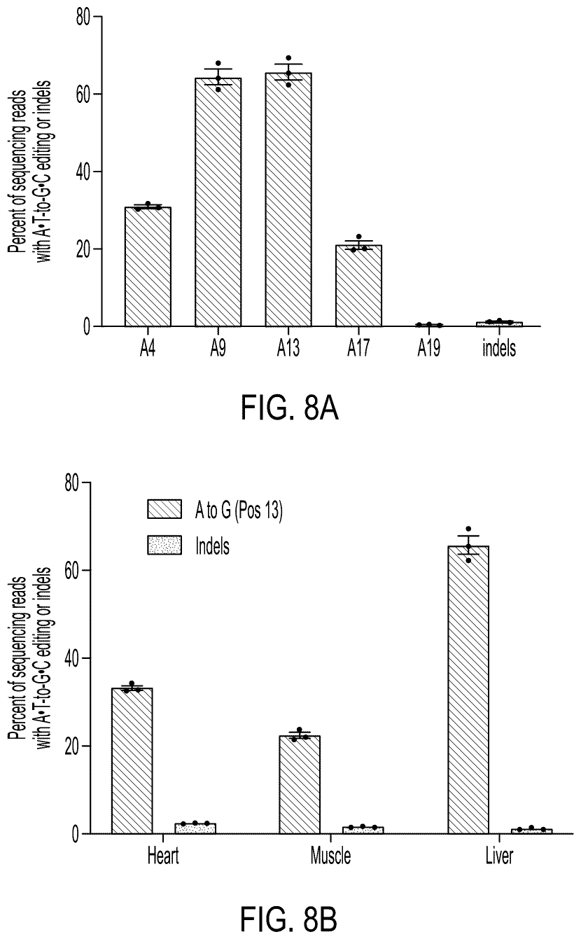

Next, the single-AAV ABE construct was assessed at a dose of 8×10¹¹ vg per mouse, equal in total AAV dose per mouse to that of the high-dose experiments described above requiring two AAVs. Further improvements in editing were observed compared to the lower doses, especially in heart and muscle, which were edited with 33% and 22% average efficiency, respectively (FIG. 2C, FIGS. 8A and 8B). This level of editing corresponded to a 2.1-fold and 2.5-fold increase in editing in heart and muscle, respectively, compared to the highest observed level of base editing from dual-AAV SaABE8e with editor and sgRNA delivered on separate AAVs (FIG. 2C, P-0.00048 and P=0.0020, respectively, by unpaired t-test) at the same total dose of AAV. The relatively wide editing window of SaABE8e is maintained in vivo, and indels remain low in each tissue (FIGS. 8A and 8B).

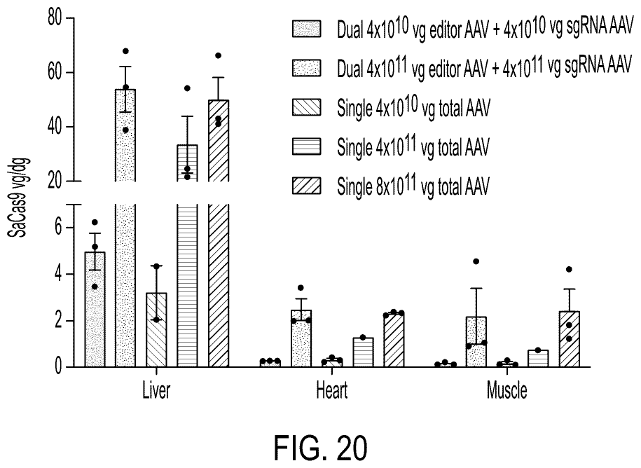

Next, the degree of transduction of AAV genomes in tissues were quantified by digital droplet PCR (ddPCR). It was observed that editing efficiencies correlated with the quantity of delivered genomes in each tissue (FIG. 20), with liver being much more amenable to transduction with AAV9 than heart and muscle. Tissues with the largest differences in editing between the single and dual-AAV strategies were less efficiently transduced than liver, consistent with previous analysis of biodistribution of AAV975. Heart and muscle tissues were transduced to a similar degree, which may indicate that the EFS promoter is more active in heart or may reflect effects of tissue heterogeneity.

The levels of base editing achieved in the liver, heart, and muscle tissue from a single-AAV ABE injection would be sufficient to offer therapeutic benefit for many genetic disorders^{21, 38-40}. These data represented some of the highest reported somatic cell in vivo base editing in these tissues at clinically relevant doses^{21, 26, 34, 41} of ≤10¹⁴ vg/kg. Together, these results demonstrated that this engineered single-AAV ABE architecture mediated robust genome editing in vivo and highlighted the benefits of single-AAV systems over dual-AAV methods, especially in less well transduced tissues or when lower total doses of AAV were used.

### Example 2

Development of a Suite of Size-Minimized ABEs with Broad Collective PAM Compatibility

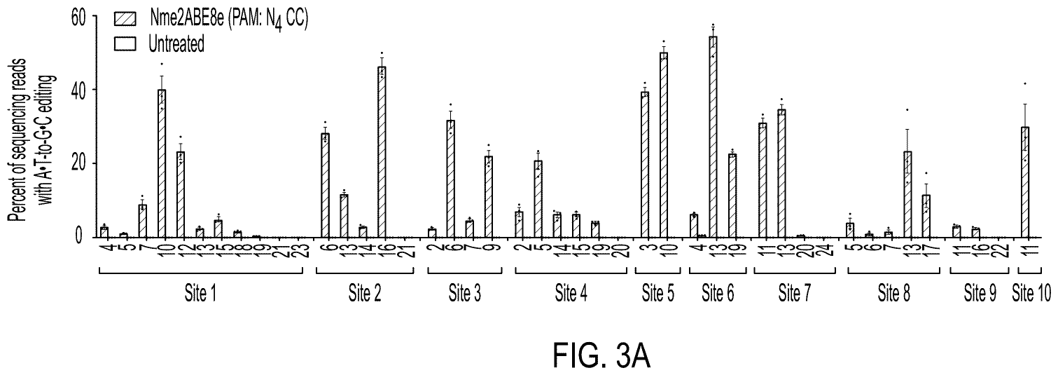

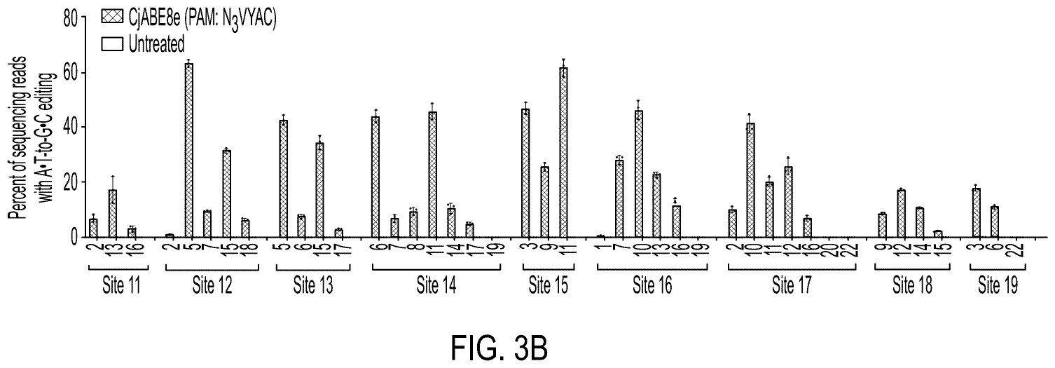

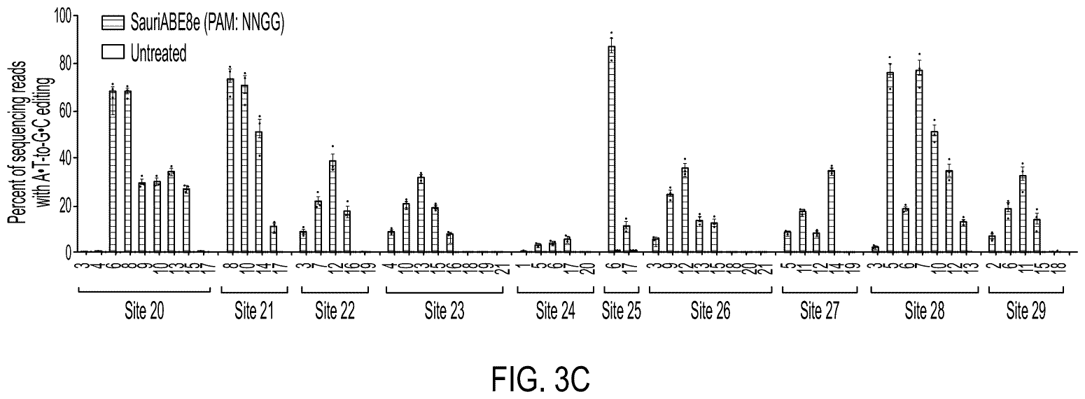

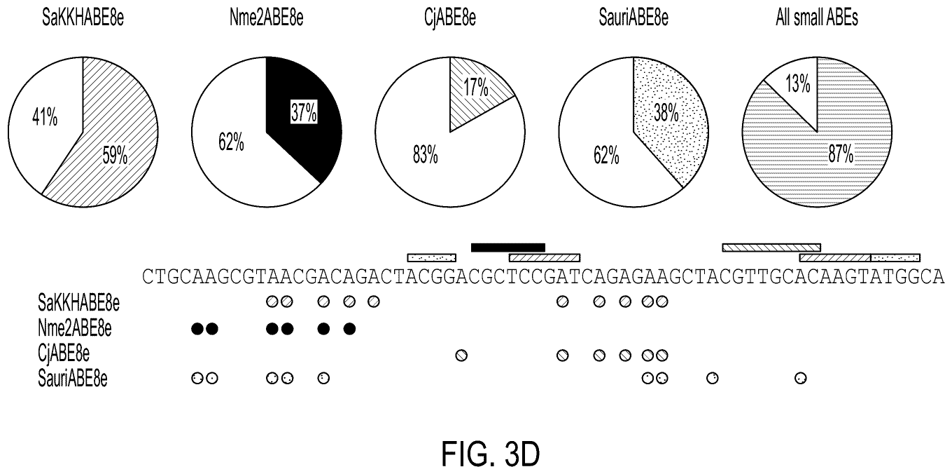

To broaden the targeting scope of single-AAV ABEs beyond that of SaCas9 (3.16 kb, PAM=NNGRRT) or engineered variants such as SaKKH³³ (3.16 kb, PAM=NNNRRT), the editing activity of ABE8c that used the nickase forms of the small Cas orthologs Nme2Cas9^{42, 43} (3.24 kb, PAM=N4CC), CjCas9^{44, 45} (2.95 kb, PAM=N4RYAC), and SauriCas9⁴⁶ (3.18 kb, PAM=N2GG) was profiled. To profile the activity of these size-reduced ABEs across multiple loci, plasmids encoding each editor and a corresponding sgRNA targeting a PAM-matched site were transfected into HEK293T cells. Three days later, the cells were analyzed by targeted high-throughput DNA sequencing (FIGS. 3A-3C).

All three of the tested small ABEs supported efficient base editing in HEK293T cells, with peak efficiencies at each target site generally ranging from 40-70%. Consistent with prior studies on base editors comprised of smaller Cas variants^{30, 47, 48}, Nme2ABE8c, CjABE8c, and SauriABE8c all exhibited broader base editing windows compared to SpCas9-based ABE8c. For Nme2ABE8e, the editing windows spanned much of the distal half of the 24-nt protospacer (position 2 to 19, counting the PAM as positions 25-30), with improved editing occurring between positions 6 and 17 (FIGS. 3A and 4A). For CjABE8, the smallest of the three small-Cas variants tested, the window was even larger, spanning positions 2 to 18, counting the PAM as positions 24-31, with optimal editing occurring between positions 3 and 15 (FIGS. 3B and 4B). CjABE8e also appeared to be more sensitive to the context preferences of the fused deaminase than the other tested ABEs. The editing efficiency varied substantially depending on the nucleobase 5′ of the target adenine (YA>>RA). The editing window of SauriCas9-ABE8e typically ranged from protospacer positions 3-16 (counting the PAM as positions 22-25) with improved editing occurring between positions 5-15 (FIGS. 3C and 4C), which resembled the wide editing window enabled by the related SaCas9. SauriCas9's broad PAM compatibility (3′ NNGG) allowed access to previously characterized SpCas9 targets (3′ NGG PAM) with single-AAV base editors.

The collective targeting scope of four small ABE8c variants (SaKKHABE8c, Nme2ABE8c, CjABE8c, and SauriABE8c) were assessed by determining the number of adenines in the entire hg38 human reference genome that were targetable by at least one of these variants. The sequence context surrounding each adenine was analyzed for the presence of a small ABE8e-targetable PAM that would place each adenine within an appropriate base editing window. This analysis revealed that 82% of all adenines in the human genome were targetable in principle by at least one of these four small ABESe variants. This data suggested that the single AAV ABE platform could potentially target the vast majority of adenines across the genome, although bystander editing in some cases could result in additional mutations, approximately half of which would be non-silent⁴⁹.

Adenine Base Editing of Pcsk9 and Angptl3 with Small-Cas ABEs in Cultured Cells

To test the in vivo therapeutic potential of the single-AAV ABE system, mutations were installed in mice that were associated with decreased cardiovascular disease risk in humans⁵⁰⁻⁵² by knocking down Pcsk9 or Angpt13 protein levels. Knockdown of these proteins reduced levels of serum biomarkers including circulating protein and total cholesterol. Additionally, levels of triglycerides were reduced when Angpt13 was knocked down, facilitating robust functional assessment of editing efficiency. SaABE8c, SaKKH-ABE8c, and newly designed SauriABE8e were used to disrupt start codons, splice donors, and splice acceptors⁵³ to block production of the targeted protein without relying on double-strand breaks or indel formation.

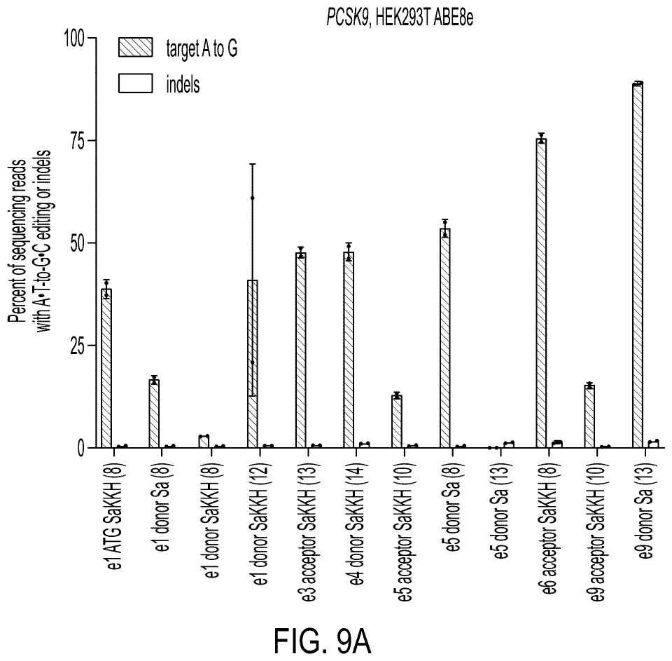

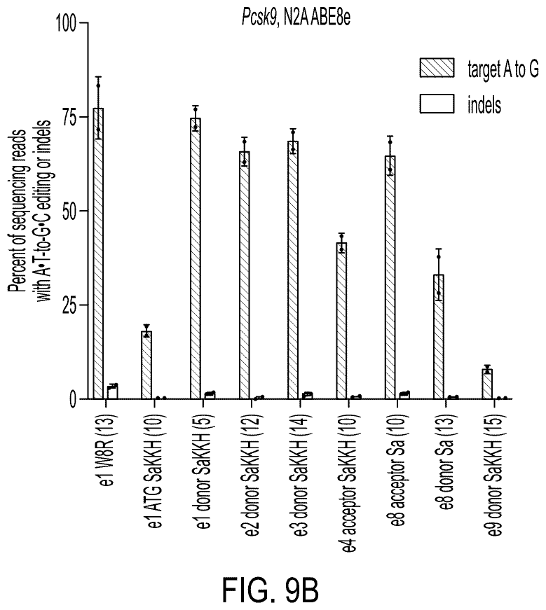

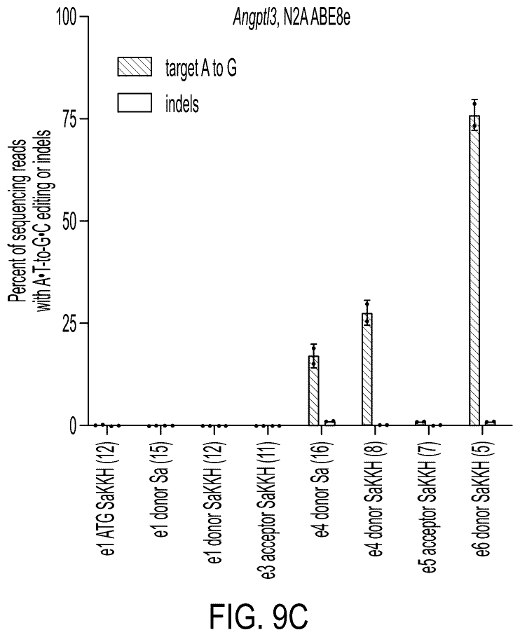

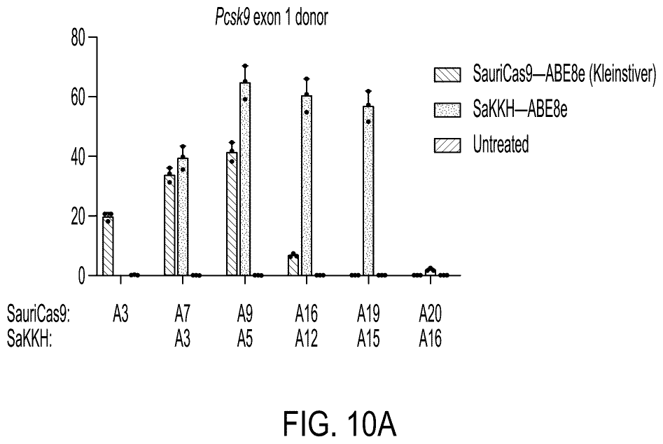

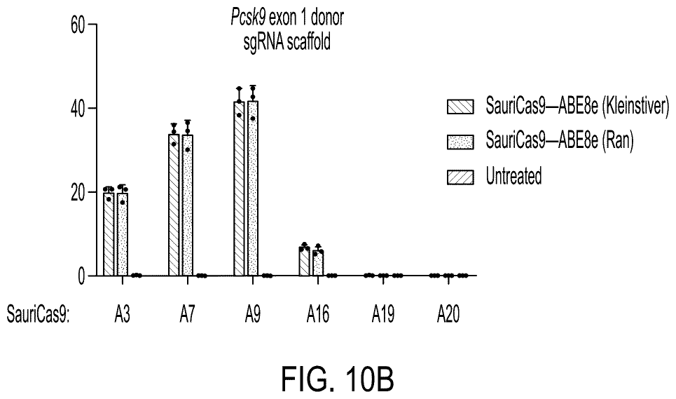

First, editing activity was measured of guide RNAs designed to disrupt production of Pcsk9 or Angpt13 by transfection of plasmids encoding a size-reduced ABE8e and sgRNA targeting sites throughout human PCSK9 in HEK293T cells (FIG. 9A) or mouse Pcsk9 and Angptl3 in Neuro-2a cells (FIGS. 9B and 9C, 10A and 10B). Base editing efficiencies varied from undetectable to 89% as measured by deep sequencing of genomic DNA at the targeted loci. Three highly efficient SaKKH-ABE8e guide RNAs that targeted exon 1 splice donor site of human PCSK9 and mouse Pesk9 and exon 6 splice donor of mouse Angptl3, as well as an SauriABE8e sgRNA targeting the exon 1 splice donor site of mouse Pesk9, were advanced to in vivo experiments.

### Single-AAV Adenine Base Editing of Pcsk9 and Angpil3 in Mice

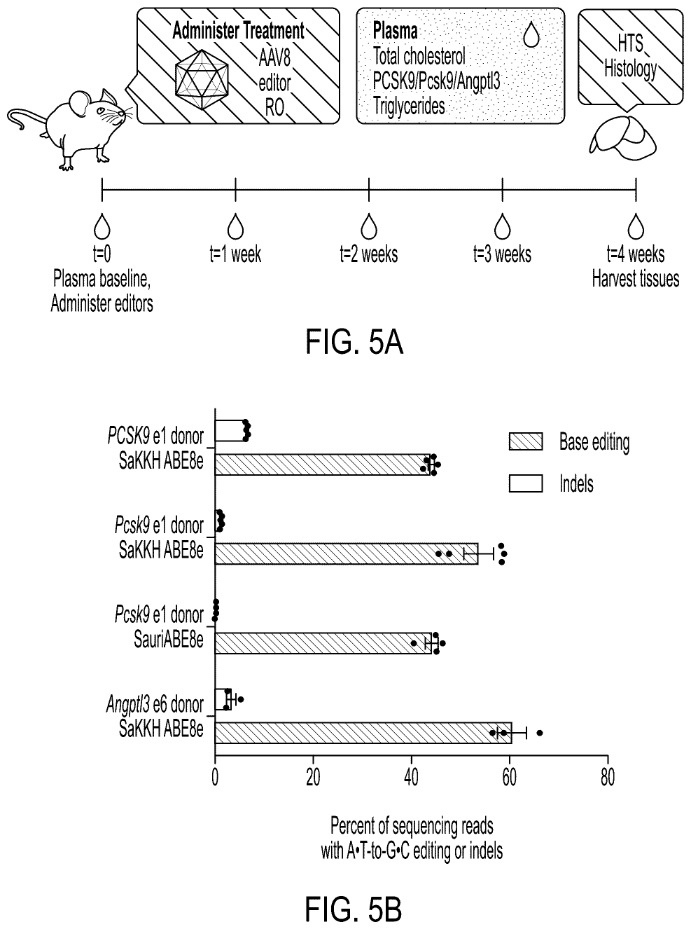

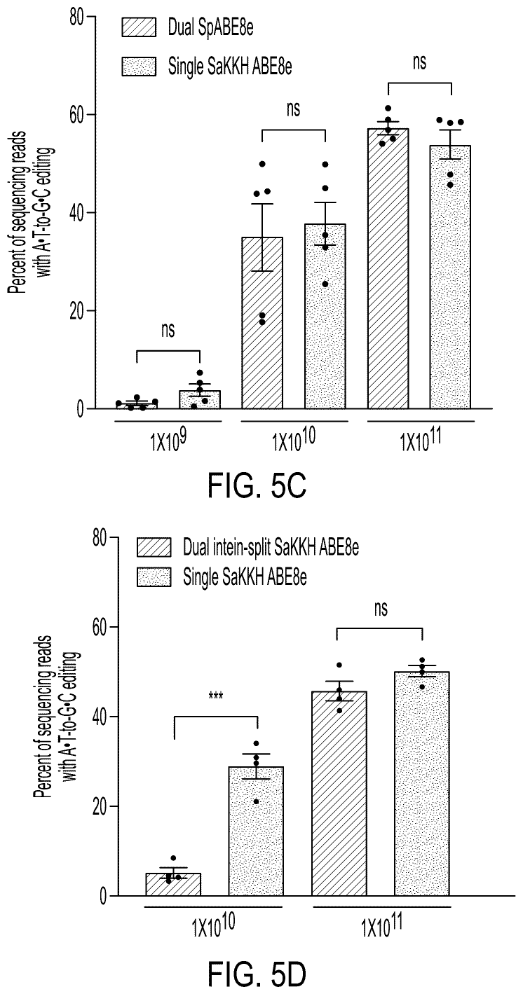

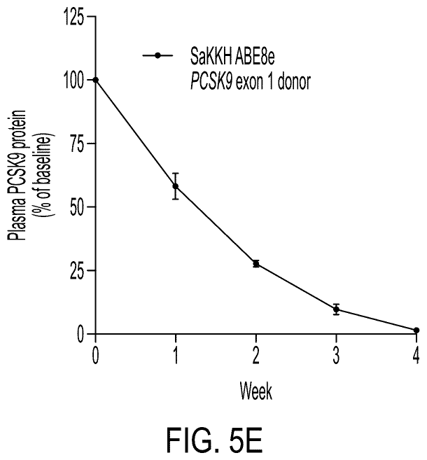

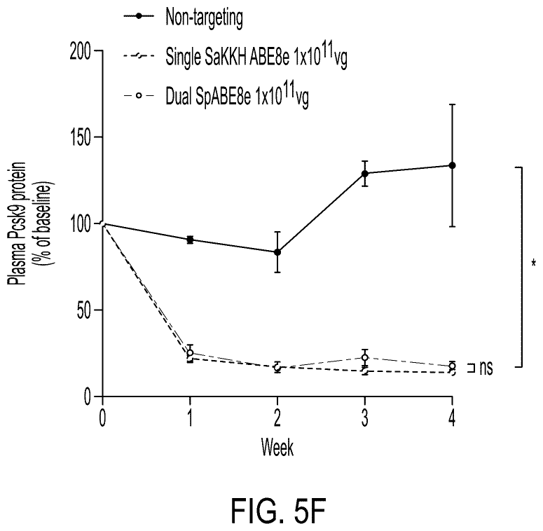

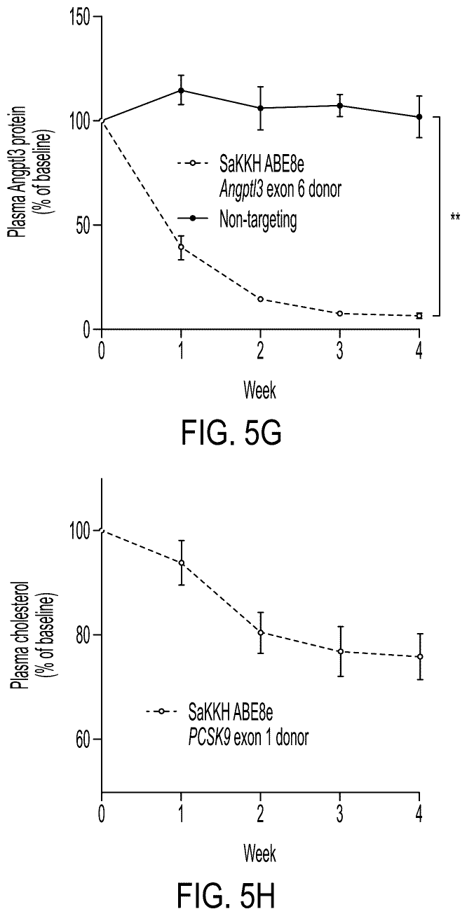

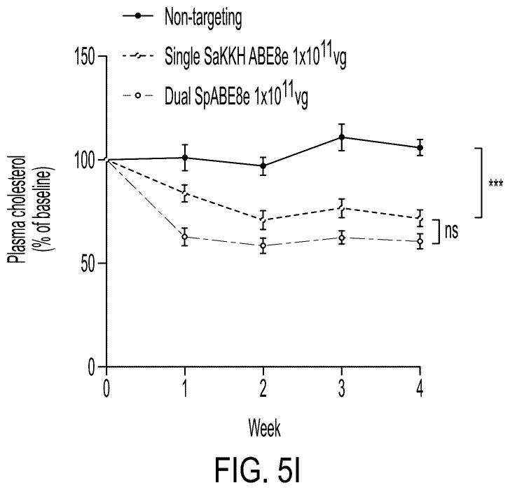

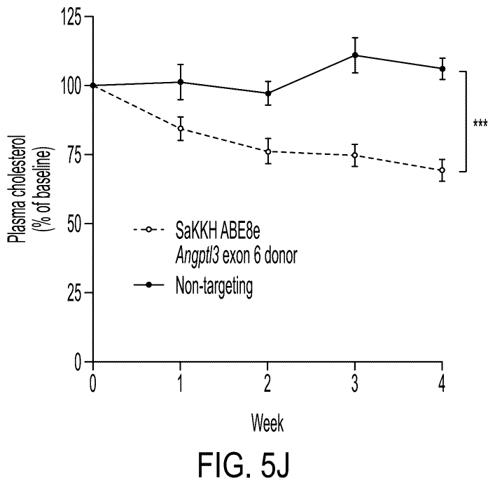

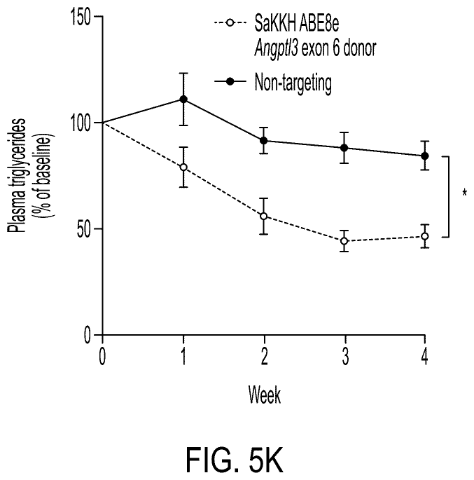

To assess in vivo editing activity with sgRNAs targeting PCSK9, Pcsk9, and Angptl3 together with the corresponding size-minimized ABE8e variant, single-AAV ABEs were prepared in AAV8, a serotype that efficiently transduced murine hepatocytes⁵⁴, and administered to 6- to 8-week old mice systemically via retroorbital injection at a dose of 1×10¹¹ vg per mouse (5×10¹² vg/kg). AAV8 encoding SaKKH-ABE8e and sgRNA targeting the exon 1 splice donor of human PCSK9 were injected into humanized mice containing the human PCSK9 sequence⁵⁵. Similarly, AAV8 encoding SaKKH-ABE8c, SaKKH-ABE8c (V106W), or SauriABE8c and editor-matched sgRNA targeting exon 1 splice donor of mouse Pesk9 or SaKKH-ABE8e and sgRNA targeting the exon 6 splice donor of mouse Angptl3 were injected to wild-type C57BL/6J mice. After four weeks bulk liver tissue was analyzed by HTS. These treatments achieved 44%, 54%, 46%, and 61% base editing of bulk liver tissue for human PCSK9 using SaKKH-ABE8c, mouse Pcsk9 using SaKKH-ABE8c, mouse Pcsk9 using SauriABE8c, and mouse Angptl3 using SaKKH-ABE8e, respectively (FIG. 5B). The relative editing efficiency of each target in cultured HEK293T and N2A cells (FIGS. 9A-9C) paralleled relative editing efficiencies in vivo (FIG. 5B). SaKKH-ABE8c (V106W), which uses a mutant of evolved TadA-8e deaminase that reduces guide-independent DNA and mRNA off-target editing⁷⁶, maintained high editing efficiency in vivo. These single-AAV in vivo base editing efficiencies approached those of reported LNP-mediated ABE mRNA liver delivery methods targeting Pcsk9 and Angptl3 reported in pre-clinical studies in mice^{41, 58}.

The editing activity of single-AAV8 SaKKH-ABE8c (1×10¹¹ vg) targeting the Pcsk9 exon 1 splice donor was compared to the previously optimized³⁴ dual AAV split-intein SpABE8e architecture paired with an SpCas9 sgRNA validated to efficiently edit and knockdown Pcsk9 in vivo^{41, 59} by targeting the same splice donor. Single-AAV8 SaKKH-ABE8c and dual-AAV8 SpABE8e were administered at the same total dose per mouse to 6- to 8-week-old C57BL/6J mice at three doses (1×10¹¹ vg, 1×10¹⁰ vg, or 1×10⁹ vg total AAV per mouse). The disruption of Pesk9 exon 1 spice donor was measured in liver four weeks after administration by HTS. The maximum vg/kg dose used (5×10¹² vg/kg for a 20-g mouse) was comparable to or lower than those used in gene therapy non-human primate studies and human clinical trials^{6, 60}. It was observed that the single- and dual-AAV ABE systems performed similarly at each dose, with the single-AAV yielding 54%, 38%, and 3.7% average editing in liver at a dose of 1×10¹¹ vg, 1×10¹⁰ vg, and 1×10⁹ vg, respectively (FIG. 4C), and editing via dual-AAV SpABE8e in liver at the same dose of AAV averaging 57%, 35%, and 1.0%, respectively. There were no significant difference at any dose by unpaired t-test. These results showed that single-AAV ABE performed comparably to the highly active previously optimized dual-AAV SpABE8e at a range of doses at a therapeutically relevant locus³⁴. Dual SpABE8e editing data has also been reported in Banskota et al. Engineered virus-like particles for efficient in vivo delivery of therapeutic proteins. Cell 185 (2): 250-265 (2022), which is incorporated herein by reference.

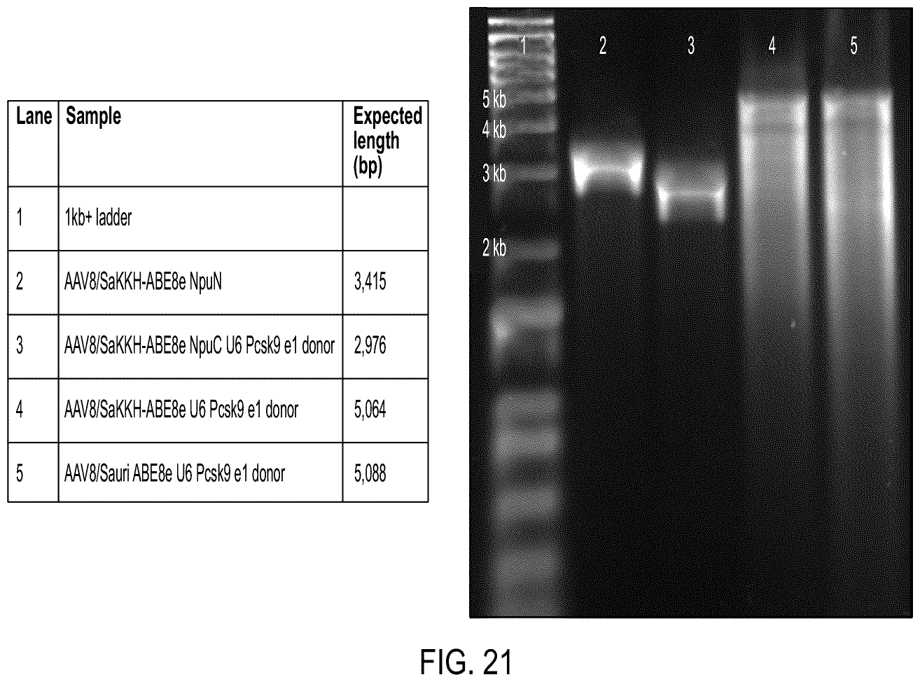

To investigate the modest apparent editing efficiency improvement of the single-AAV platform relative to split SpABE8e in liver, editing by single-AAV8 SaKKH-ABE8c and dual-AAV8 intein-split SaKKH-ABE8e with matched promoter and polyA signal by systemic retro-orbital delivery to 6-8 week old C57BL/6 mice were compared at two doses (FIG. 5D). Single and dual-AAV intein-split SaKKH-ABE8e showed similar editing frequencies at the high dose of 1×10¹¹ vg (50% and 46%, respectively, not significant by unpaired t-test); however, the dual-AAV intein-split SaKKH-ABE8e showed markedly reduced activity compared to single-AAV SaKKH-ABE8e at a lower dose of 1×10¹⁰ vg (5.2% versus 29%, respectively, P=0.0004). These data indicate that single-AAV SaKKH-ABE8e may perform similarly to optimized dual-AAV SpABE8e under conditions in which AAV transduction is already at or near saturation. This observation may be due to the higher activity of SpABE8e and/or increased activity afforded by cis-regulatory elements that may be included with the extra space on two AAV genomes overcoming the limitations of intein-splitting and two transduction events. For applications in which AAV transduction is well below saturation, however, single-AAV delivery can result in substantial editing efficiency improvements. The integrity of AAV genomes packaged in single-AAV ABEs was also assessed. The results of alkaline gel electrophoresis of single-AAV ABEs and smaller intein-split dual-AAV ABEs (FIG. 21) indicated that single-AAV ABEs can efficiently package full-length and truncated genomic species, consistent with their size being at or near the AAV packaging limit¹⁹.

### Example 3

### Reduction in Circulating Protein and Lipids Upon Editing of Pcsk9 and Angptl3

To assess whether the efficient editing observed in the liver translated into efficient target gene knockdown and concomitant reduction in circulating lipid levels, editor treated mice were serially bled and plasma levels of the targeted protein and total cholesterol were measured. A non-targeting control of dual AAV ABE7.10 targeting Dnmt1, an edit not expected to affect cholesterol or lipid metabolism, was included for comparison (FIGS. 11A and 11B). Complete protein knockdown in all experimental conditions at a dose of 1×10¹¹ vg (5×10¹² vg/kg) single-AAV SaABE8e or SaKKH-ABE8e was observed by four weeks, with most knockdown evident by two weeks (FIGS. 5D-5F). Dnmt1 encodes DNA methyltransferase 1. On average, single-AAV ABE treatment at this dose resulted in 99%, 91%, and 94% knockdown of human PCSK9, mouse Pcsk9, and mouse Angpt13 protein levels, respectively, compared to control animals treated with AAV encoding the Dnmt1-targeting guide RNA. The knockdown of these proteins matched the results achieved by reported LNP-mediated ABE mRNA delivery to the liver^{41, 58}. This high level of protein knockdown is also consistent with observed editing levels (FIG. 4B) since the hepatocytic tropism of AAV858 and the fact that hepatocytes constitute roughly 70% of the murine liver⁵⁷ imply that 44-61% editing in bulk liver tissue corresponds to about 60-85% base editing in hepatocyte cells.

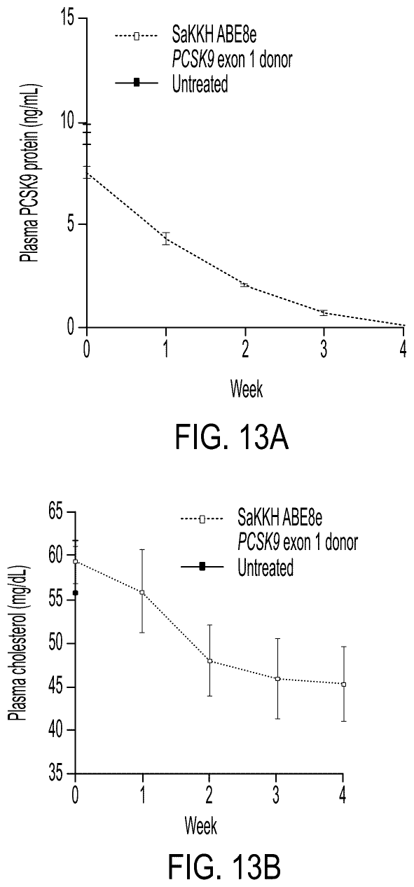

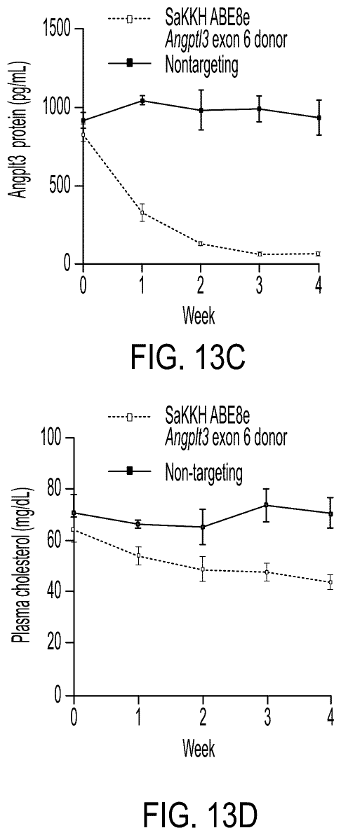

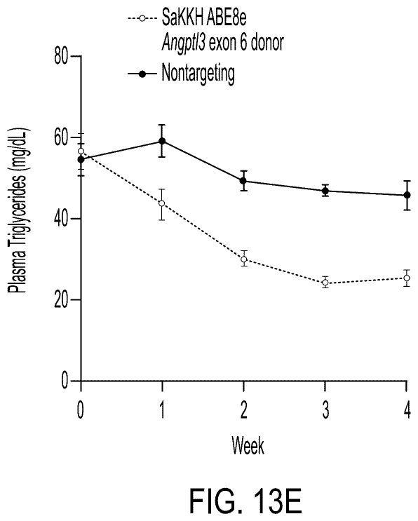

Protein knockdown resulted in decreased circulating cholesterol in all ABE treated mice (FIGS. 5G-5I). Plasma total cholesterol in human PCSK9-targeted mice decreased by 24% from baseline levels to 45 mg/dL after 4 weeks. At the highest AAV dose in Pesk9-targeted mice, plasma cholesterol was lowered by an average of 25% compared to age-matched nontargeting controls to 53 mg/dL after four weeks (FIGS. 5H and 13E) nearing the degree of cholesterol lowering observed in liver-specific Pcsk9 knockout mice⁶¹. For Angpt13 targeted mice, a 38% decrease in plasma cholesterol was observed compared to age-matched nontargeting controls to 44 mg/dL. These results demonstrated substantial lowering of cholesterol using single-AAV ABEs.

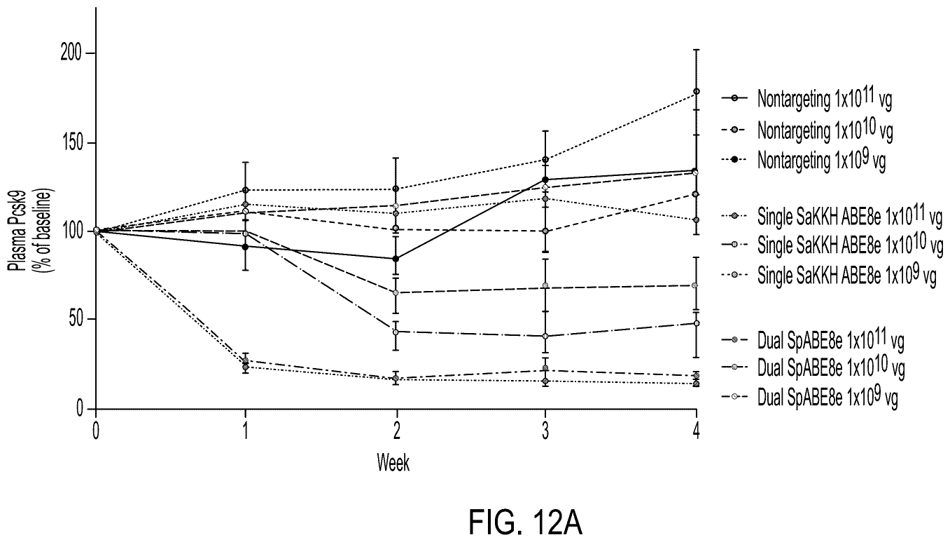

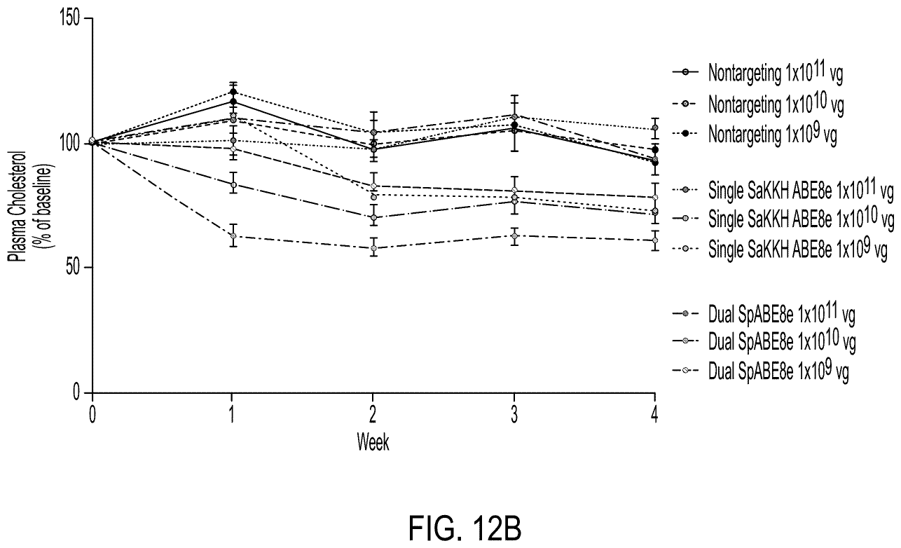

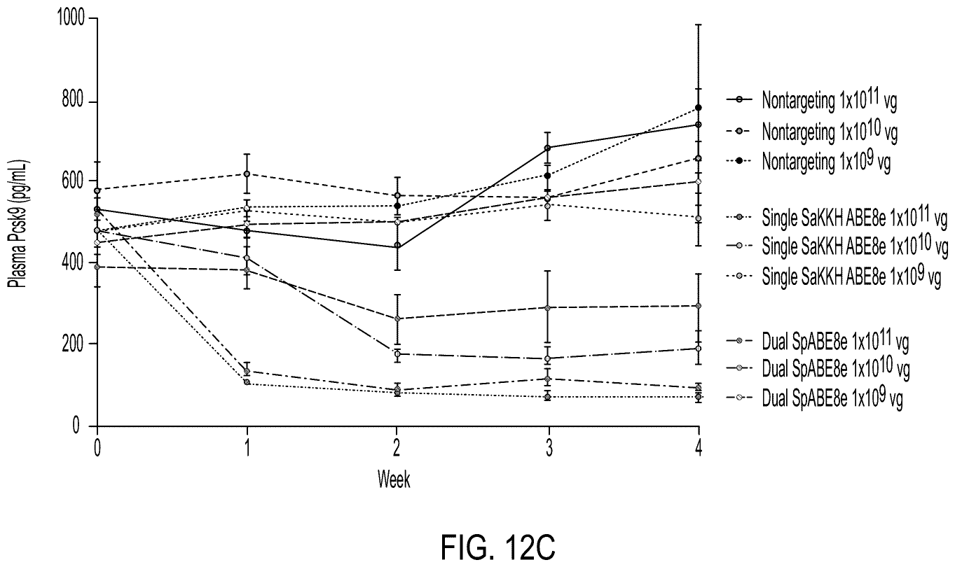

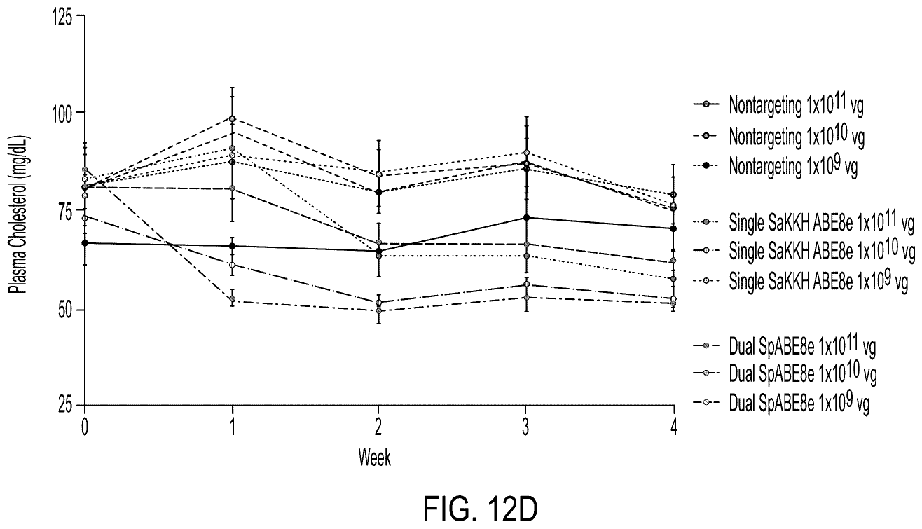

The dose-dependence of AAV dose and editing was assessed on circulating Pcsk9 and total cholesterol. Knockdown of mouse Pcsk9 and the decrease in total plasma cholesterol was dose-dependent and closely reflected the level of editing observed at each dose (FIGS. 12A-12B, 13A-13E). Dual-SpABE8e, which effected editing levels similar to single AAV ABE8e, also resulted in decreased plasma cholesterol in a dose-dependent manner (FIGS. 12C-12D). For both single- and dual-AAV ABEs targeting mouse Pcsk9, cholesterol and protein knockdown correlated closely with editing percentage, regardless of editor type administered.

Plasma triglycerides were measured in Angptl3-targeted mice, as loss-of-function alleles of Angptl3 are known to reduce levels of both cholesterol and triglycerides⁶². In the Angptl3-targeted mice, a 45% decrease in circulating triglycerides was observed compared to nontargeting control to 25 mg/dL after four weeks (FIG. 5K). The editing and reduction of circulating Angpt13, cholesterol, and triglycerides achieved here with single AAV ABE is likely the highest reported upon targeted genome editing to knockdown Angptl3^{58, 63}. Together, these results demonstrate robust base editing at multiple therapeutically relevant loci achieved with single-AAV ABEs, resulting in strong effects on target protein level and metabolic changes in adult mice.

### Additional Editing Experiments: Off-Target Effects

Lastly, liver morphology and off-target editing in mice treated with single-AAV ABEs was assessed. Histology performed on livers from mice treated with single-AAV8 SaKKH-ABE8e and guide targeting PCSK9 exon 1 donor at 1×10¹¹ vg four weeks after administration did not indicate morphological changes relative to untreated mice (FIGS. 19A and 19B).

As shown in FIGS. 15-17, on-target editing of single-AAV SaKKH-ABE8e was comparable to, or better than, on-target editing of dual-AAV SaKKH-ABE8e, at the Pcsk9 exon 1 splice donor site. The results of the on-target editing comparison is shown in FIG. 14. Indel rates approached 0% for this experiment.

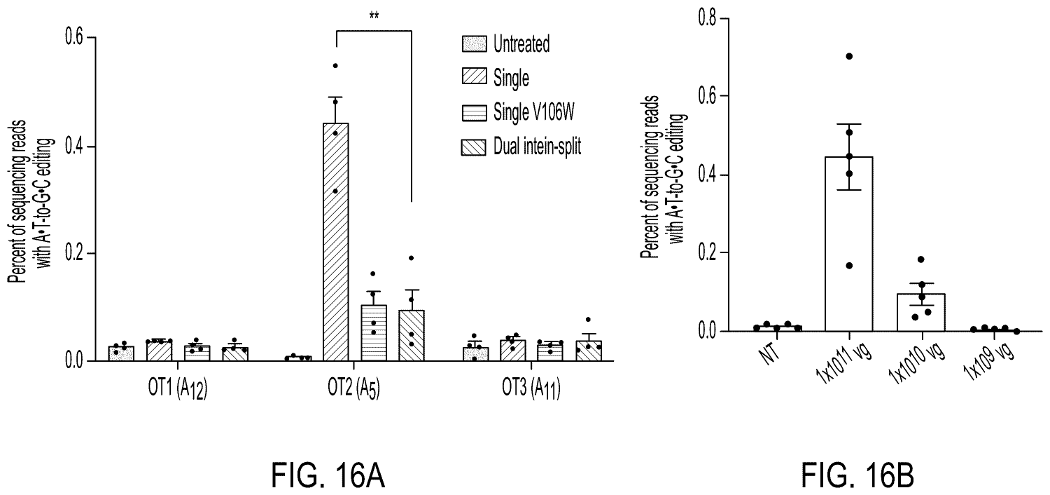

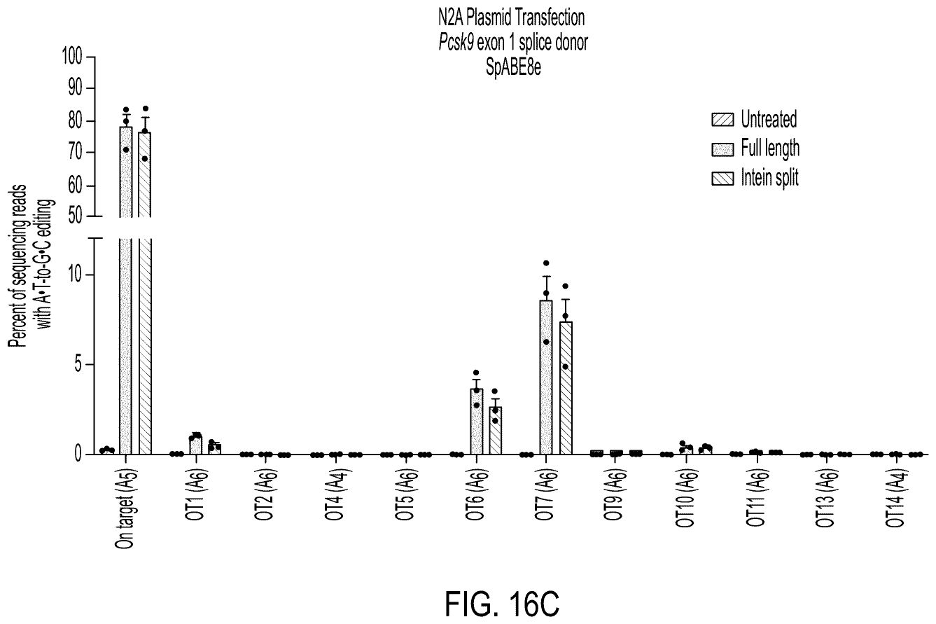

Although off-target editing in cell culture has been characterized for SaCas9-based genome editing agents, no off-target editing in tissues treated with SaCas9-based editing agents in vivo has been reported^{22, 74}. To assess single-AAV ABEs in vivo off-target editing, the liver tissue of 6- to 8-week-old C57BL/6J mice treated with 1×10¹¹ vg single-AAV8 SaKKH-ABE8c, 1×10¹¹ vg single-AAV8 SaKKH-ABE8c (V106W), or 5×10¹⁰ vg of each half of dual-AAV8 intein-split SaKKH-ABE8e targeting mouse Pesk9 exon 1 four weeks after administration of these three editors. The top three computationally predicted sites^{77, 78} from these livers were sequenced. A low but detectable (up to 0.45%) dose-dependent frequency of editing was at one off-target site in vivo for single-AAV SaKKH-ABE8e was observed (FIGS. 16A and 16B). This suggests the importance of considering off-target editing outcomes when using single-AAV ABEs, even though observed off-target editing was relatively rare at the sites examined.

This in vivo off-target editing was ameliorated by administration of a single-AAV SaKKH-ABE8c (V106W) variant, which has been reported to lower guide-independent DNA off-target editing and mRNA off-target editing^{30, 76}. It was also ameliorated by administration of dual-AAV intein-split SaKKH-ABE8e, which may be due to inherent lower overall activity of intein-linked ABE8c, or the lower dose of complete base editors, although no significant difference between full-length or intein-split SpABE8e off-target editing in N2A cells by plasmid transfection in vitro was observed (FIG. 16B). As shown in FIG. 16C, HEK293T cells were transfected with plasmids encoding full-length SpABE8e or intein-split SpABE8e and sgRNA targeting the human PCSK9 exon 1 splice donor site. CIRCLE-seq predicted off-targets were analyzed three days after transfection by high-throughput DNA sequencing (HTS).

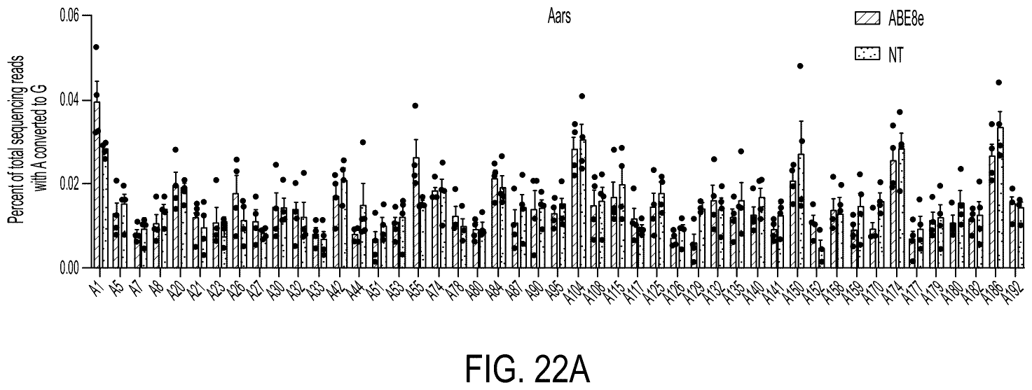

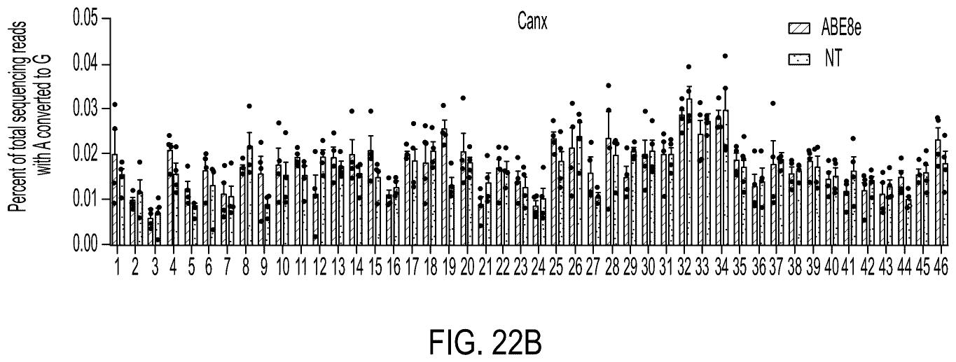

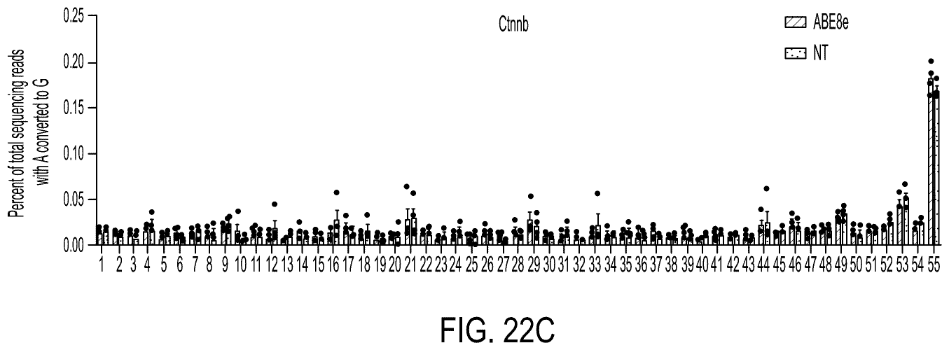

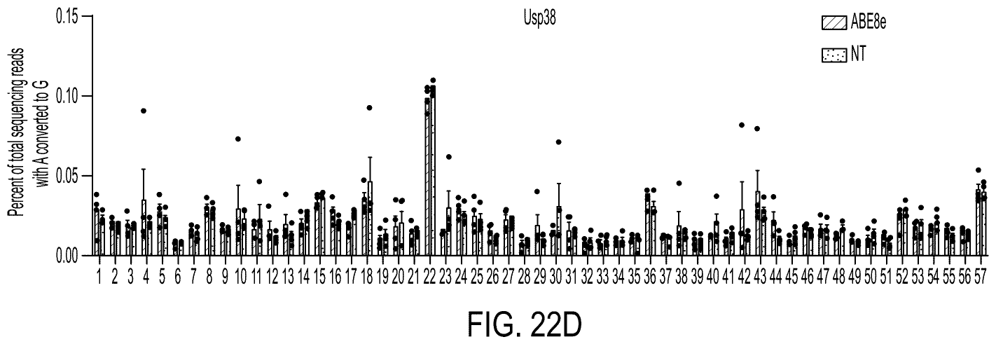

Next, we assessed in vivo off-target mRNA editing resulting from treatment of mice with single AAV8-encoded SaKKH-ABE8e editors by analyzing cDNA amplicons of mouse homologs of validated ABE mRNA off-target human amplicons⁷⁶, some containing partial TadA recognition sequences. In particular, adult C57BL/6J mice were injected retro-orbitally with 1×10¹¹ vg single-AAV8 SaKKH-ABE8e with sgRNA targeting the Pesk9 exon 1 splice donor site or saline. At four weeks after injection, RNA was isolated from liver tissue and reverse transcribed. cDNA amplicons Aars, Canx/IP90, Ctnnb, and Usp38 were analyzed by HTS. No off-target mRNA editing was observed compared to untreated mice (FIGS. 22A-22D). In particular, no significant increases in mRNA off-target editing in livers from single-AAV ABE treated-mice relative those from untreated mice were observed, across the four measured cDNAs. These results suggest that single-AAV ABEs maintain low levels of off-target DNA and RNA editing in vivo for the guide RNA tested, and that deaminase mutations can further minimize off-target editing in vivo.

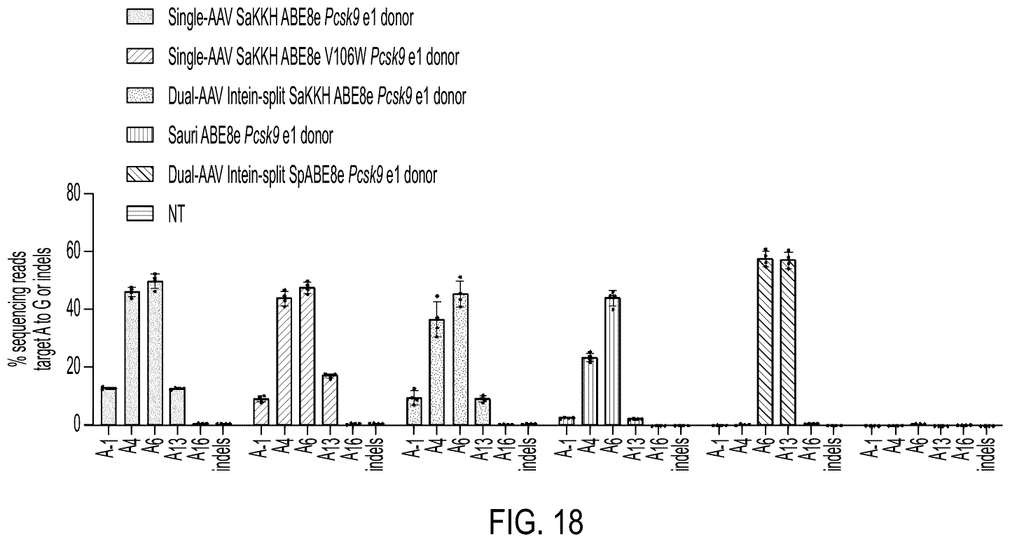

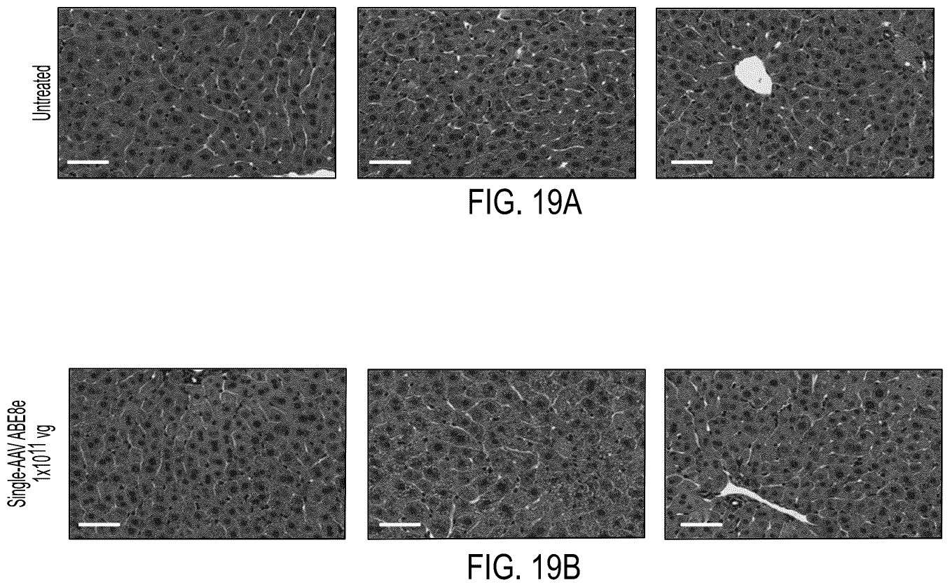

Finally, an analysis of the width of the editing windows of the disclosed AAV-encoded base editors was performed. ABEs were delivered by AAV8 at a total dose of 1×10¹¹ vg by retro-orbital injection and liver was harvested at 4 weeks post injection for HTS As shown in FIG. 18, the editing windows of SaKKH-ABE8e and SauriABE8e are as large as 13 nucleotides, which constitutes wide editing windows.

Overall, these results suggest that on-target editing is comparable for single-AAV8 SaKKH-ABE8c, single-AAV8 SaKKH-ABE8c (V106W), and dual-AAV8 intein-split SaKKH-ABE8c.

### DISCUSSION

By minimizing the size of adenine base editors and AAV components, a suite of single-AAV adenine base editor systems was developed that support robust editing in vivo and have broad targeting capability due to their collective PAM compatibility. Single-AAV ABE supported base editing efficiencies of up to 66%, 33%, and 22% editing in liver, heart, and muscle, respectively, and outperformed dual-AAV approaches especially when tissue type or AAV dose prevented saturating levels of transduction. The largest editing efficiency increases compared to dose-matched dual-AAV were 2.1-fold in heart and 2.5-fold in skeletal muscle, potentially due to the relatively lower transduction efficiency in these tissues. These findings suggested that a single AAV platform may be especially preferable when targeting non-liver tissues such as heart and skeletal muscle, or when toxicity limits AAV dosage (FIG. 2C).

Single-AAV ABEs having serotypes AAV8 and AAV9 were packaged in multiple serotypes which facilitated editing in a variety of tissues and cell types outside the liver or with alternate administration routes. Even for base editing in the liver, the organ for which LNP-mediated mRNA delivery is the most potent, single-AAV ABEs resulted in editing efficiencies, target protein knockdown, and desired phenotypic changes comparable to reported preclinical LNP-mediated mRNA delivery efforts^{41, 59}. In organs such as the heart for which LNP-mediated delivery is not particularly efficient, the single-AAV systems described herein may prove especially useful.

Single-AAV base editor delivery is currently limited to ABEs that use small Cas enzymes ≤˜3.2 kb in gene size. The activity of a variety of size-reduced ABEs that together cover a targetable genome similar to that targetable by SpCas9-ABE has been demonstrated. It is estimated that roughly 82% of genomic adenines can be edited using the suite of size-minimized ABEs described in this disclosure. While a small fraction can be targeted without any bystander edits, for many applications, bystander editing may be acceptable, for example, because it results in silent or benign mutations, because the target is in a non-coding regulatory sequence, or because the application seeks to disrupt the function of a sequence. Further work to analyze single-AAV base editors that exhibit sequence context preferences or altered activity window locations^{48, 64} would further broaden the applicability of single-AAV in vivo base editing and is ongoing.

The single-AAV ABE systems described herein yielded robust editing efficiencies in vivo, facilitating therapeutically relevant levels of editing in liver, heart, and muscle tissue at moderate doses of AAV. While AAV allow targeting of tissues inaccessible with technologies such as LNPs, toxicity associated with AAV was recognized in NHPs and in clinical trials at high doses^{65, 66}. Animal studies have also indicated that AAV genomic integration may lead to hepatocellular carcinoma⁶⁷, although a causal link between liver tumors and AAV has not been established in humans treated with recombinant AAV vectors^{68, 69}. While the therapeutic landscape of AAV continues to be explored, these limitations suggest the potential safety advantages of highly potent editing agents that limit the amount of AAV required to achieve therapeutic target editing levels. Immune responses to gene editing agents delivered via AAV have not been thoroughly characterized in large animal models. Early data indicated that stable genome editing using non-native nucleases expressed from AAV was achievable in non-human primates without major adverse effects, although, some loss of edited helpatytes was observed⁷⁰. Systems for inducible expression of editing agents⁷¹ and alternative delivery vectors such as engineered virus-like particles (cVLPs)^{2, 72, 73} could also be incorporated with administration of size-reduced base editors.

### Methods

### Molecular Biology

Expression vectors for tissue culture were cloned using KLD, Gibson, or USER assembly. sgRNA expression plasmids were cloned via KLD or Goldengate assembly to install the protospacers as indicated in Table 1. Base editor plasmids were cloned via USER assembly or Gibson assembly of PCR-amplified fragments. Plasmids encoding rAAV genomes were cloned by Gibson assembly of plasmid restriction fragments and PCR amplicons with Gibson-compatible overhangs. All plasmids for mammalian tissue culture experiments were purified using Plasmid Plus Maxiprep or Midiprep kits (Qiagen), ZymoPURE II Midiprep kit (Zymo Research) or Pure Yield plasmid miniprep kits (Promega).

### Culture and Transfection of HEK293T and N2A Cells

HEK293T cells (ATCC CRL-3216) and Neuro-2A cells (ATCC CCL-131) were maintained in Dulbecco's Modified Eagle's Medium plus GlutaMax (Thermo Fisher Scientific) supplemented with 10% (v/v) FBS at 37° C. with 5% CO₂. 16-24 hours before transfection, HEK293T cells or N2A cells were seeded on 96-well plates (Corning) at 1.4×10⁴-2.0×10⁴ cells/well at >90% viability, or for SauriABE transfections, HEK293T cells were seeded in 48-well plates (Corning) at 4.0×10⁴ ells/well, >90% viability. Cells in 96-well plates were transfected at approximately 70-85% confluency with 0.5 μL of Lipofectamine 2000 (Thermo Fisher Scientific) and 187.5 ng of base editor plasmid, 37.5 ng of sgRNA plasmid per well (180 ng editor and 60 ng sgRNA for SauriABE8c). Cells in 48-well plates were transfected with 1.5 μL Lipofectamine 2000 with 750 ng editor and 250 μL sgRNA. Cells were cultured for 72 hours after transfection. Next, the media was removed, cells were washed with 1× PBS (Thermo Fisher Scientific), and genomic DNA was extracted by addition of 30-60 μL lysis buffer [10 mM Tris-HCl, pH 7.5-8.0, 0.05% SDS, 20 μg/mL Proteinase K (New England Biolabs)] per well for 96 well plates and 150 μL per well for 48 well plates. Genomic DNA was stored temporarily at 4° C. or longer term at −20° C. until further use.

### High Throughput Sequencing and Data Analysis

Genomic DNA was amplified by PCR using Phusion Hot Start II DNA polymerase or Phusion U Hot Start DNA polymerase with 0%-3% DMSO added. Barcodes for Illumina sequencing were added via a second PCR step, using 1 μL of the first PCR as a template. Total PCR cycles were kept to a minimum to avoid PCR bias. Barcoded PCR products were pooled according to amplicon. The gel was extracted (MinElute; Qiagen) and quantified by qPCR (KAPA; KK4824) or Qubit dsDNA HS Assay Kit (Thermo Fisher Scientific).

Sequencing of pooled libraries was performed using Illumina MiSeq according to the manufacturer's instructions. Primers for amplification of each locus from genomic DNA are compiled in Table 3.

Sequencing reads were demultiplexed using MiSeq Reporter (Illumina). Alignment of amplicon sequences to reference sequence was performed using CRISPResso2⁷⁸ with “discard_indel_reads” on. For quantification of base editing, efficiency was calculated as percentage of (reads containing an A to G edit at given position without indels)/(number of total reads). Indels were calculated explicitly as (discarded reads)/(total aligned reads)×100. Base editing at a given position was calculated explicitly as: (frequency of specified point mutation in non-discarded reads)×100×(100−(indel reads))/100).

### AAV Production

AAV was produced as previously described³⁵. HEK293T clone 17 cells (ATCC CRL-11268) were maintained in Dulbecco's Modified Eagle's Medium plus GlutaMax (Thermo Fisher Scientific) supplemented with 10% (v/v) FBS without antibiotic in 150-mm dishes (Thermo Fisher Scientific; 157150) at 37° C. with 5% CO₂ and passaged every 2-3 days. Cells were split 1:3 the day before polyethyleneimine transfection with 5.7 μg AAV genome plasmid, 11.4 μg pHelper (Chlontech) and 22.8 μg rep-cap plasmid per plate. Media was exchanged for DMEM 5% FBS the day after transfection. Four days after transfection, cells and media were collected using a rubber cell scraper (Corning), pelleted by centrifugation at 2000 g for 10 minutes, resuspended in 500 μL hypertonic lysis buffer per plate [40 mM Tris base, 500 mM NaCl, 2 mM MgCl2 and 100 U/mL salt active nuclease (ArcticZymes; 70910-202)] and incubated at 37° C. for 1 hour to lyse the cells. The media was decanted and combined with a 5× solution of 40% poly(ethylene glycol) 8,000 (PEG 8k, Sigma-Aldrich 89510) in 2.5 M NaCl for a final concentration of 8% PEG/500 mM NaCl, incubated on ice for 2 hours, and then centrifuged at 3200 g for 30 minutes. The pellet was resuspended in 500 μL of hypertonic lysis buffer per plate and added to the cell lysate. Crude lysates were either incubated at 4° C. overnight or taken immediately to ultracentrifugation.

Cell lysates were clarified by centrifugation at 2,000 g for 10 minutes and added to Beckman Quick-Seal tubes via 16-gauge 5 inch disposable needles (Air-Tite N165). A discontinuous iodixanol gradient was formed by sequentially floating layers: 9 ml 15% iodixanol in 500 mM NaCl and 1× PBS-MK (1× PBS plus 1 mM MgCl2 and 2.5 mM KCl), 6 ml 25% iodixanol in 1× PBS-MK, and 5 mL each of 40% and 60% iodixanol in 1× PBS-MK. Phenol red at a final concentration of 1 μg/mL was added to the 15, 25 and 60% layers to facilitate identification. Ultracentrifugation was performed using a Ti 70 rotor in a Sorvall wX+ Ultracentrifuge (Thermo Scientific) at 58,000 rpm for 2 hours 15 minutes at 18° C. Immediately following centrifugation, 3 mL of solution was withdrawn from the 40-60% iodixanol interface via an 18-gauge needle. The solution was exchanged into cold PBS containing 0.001% F-68 using PES 100 kD MWCO columns (Thermo Scientific, Pierce 88533) and concentrated. The concentrated AAV solution was sterile filtered using a 0.22 μm filter, quantified by qPCR (AAVpro Titration Kit version 2; Clontech), and stored at 4° C. until use.

### Animals

All experiments in live animals were approved by the Broad Institute and University of Pennsylvania Institutional Animal Care and Use Committees and were consistent with local, state, and federal regulations as applicable, including the National Institutes of Health Guide for the Care and Use of Laboratory Animals. C57BL/6J mice (stock no. 000664) for use in experiments were purchased from The Jackson Laboratory. Humanized PCSK9 mice were reported previously⁵⁷. All mice were housed in a room maintained on a 12-hour light and dark cycle with ad libitum access to standard rodent diet and water except for 4-hour fasts just prior to bleeds for plasma analysis.

### Retro-Orbital Injections

AAV was diluted into 100 μL of sterile 0.9% NaCl USP (Fresenius Kabi; 918610) before injection. Anesthesia was induced with 2-4% isoflurane. Following induction, as measured by unresponsiveness to bilateral toe pinch, the right eye was protruded by gentle pressure on the skin, and an insulin syringe was advanced, with the bevel facing away from the eye, into the retrobulbar sinus where AAV solution was slowly injected. One drop of Proparacaine Hydrochloride Ophthalmic Solution (Patterson Veterinary; 07-885-9765) was then applied to the eye as an analgesic. At harvest, mice were euthanized by carbon dioxide asphyxiation. Genomic DNA was purified from minced tissue using gDNAdvance kit (Beckman Coulter A48705) according to the manufacturer's instructions and used as template for high throughput sequencing. RNA was purified from 30 mg of snap frozen liver tissue with RNeasy Plus Mini kit (Qiagen 74134) according to the manufacturer's instructions, then reverse transcribed to cDNA using SuperScript III first-strand synthesis supermix (Invitrogen 18080-450) with an oligo dT primer, which was used as template for high-throughput sequencing.

### Digital Droplet PCR

Genomic DNA was purified from tissue using Beckman gDNAdvance kit (Beckman Coulter A48705) according to the manufacturer's instructions and used as template for digital droplet PCR. ddPCR was carried out using ddPCR Supermix for Probes (BioRad 1863026) with 10 ng of genomic DNA as template and 3 units NEB EcoRI-HF (R3101S) per reaction. Droplets were autogenerated and PCR was performed at an annealing and extension temperature of 61° C. for 2 minutes for a total of 60 cycles. Droplets were analyzed on a QX200 droplet analyzer and droplet fluorescence was quantified using QuantaSoft (BioRad).

### Calculation of Targetable Genomic Adenosines

A custom Python script, shown in Example 5, was used to analyze the targetability of all adenosines in the hg38 human reference genome. An adenosine was counted as targetable if the surrounding genomic sequence context contained a small ABE8e-targetable PAM that would place that adenosine within an appropriate base editing window. The PAM sequences, protospacer lengths, and base editing windows associated with each small ABE8e variant are provided in in Table 4A (Example 4). The percentage of calculated genomic adenines on each chromosome is shown in Table 4B.

### Blood Collection and Plasma Analysis

Initial blood samples were collected following a 4-hour fast. Age-matched littermates/colonymates were randomly assigned to experimental groups and administered AAV particles (n=5) via retro-orbital injection. Blood samples were collected following a 4-hour fast at 1-week intervals via the tail tip. After 4 weeks, all mice were euthanized by carbon dioxide asphyxiation after a 4-hour fast. Whole livers were harvested for genomic DNA isolation and analysis and for hematoxylin/eosin staining, and terminal blood samples were collected.

Pre-treatment and post-treatment plasma human PCSK9, mouse Pcsk9, or mouse ANGPTL3 was measured using the Human Proprotein Convertase 9/PCSK9 Quantikine ELISA Kit, Mouse Proprotein Convertase 9/PCSK9 Quantikine ELISA Kit, or Human Angiopoietin-like 3 Quantikine ELISA Kit, respectively, according to the manufacturer's instructions (R&D Systems). Total cholesterol or triglyceride levels were measured using the Infinity Cholesterol Reagent or Infinity Triglycerides Reagent, respectively, according to the manufacturer's instructions (Thermo Fisher Scientific).

### Liver Tissue Fixation & Histology

A portion of the left medial lobe was fixed in 4% paraformaldehyde at 4° C. overnight, washed with PBS, then dehydrated gradually by serial substitution of PBS for 30%, 50%, 70%, then 100% ethanol. Samples were kept at −20° C. until analysis, when they were paraffinized by the Rodent Histopathology Core of Harvard Medical School. Liver paraffin block was then cut into 5 μm sections followed by hematoxylin and eosin staining for histopathological examination.

### Alkaline Gel Electrophoresis of AAV Genomes

1% alkaline agarose gel (1% agarose in water with 50 mM NaOH and 1 mM EDTA) was prepared by dissolving agarose in water, allowing to cool but not solidify, then adding a 50×solution of NaOH and EDTA. The formed gel was submerged in 1× alkaline running buffer (50 mM NaOH, 1 mM EDTA) in a submarine style gel electrophoresis setup at 4° C. 5×10¹⁰ vg of AAV was treated with DNAse I (NEB M0303S) then lysed in 1× alkaline lysis buffer (50 mM NaOH, 1 mM EDTA, 0.3% SDS, 5% glycerol, 0.0025% xylene cyanol) for 3 minutes at 95° C., then cooled on ice. Samples were loaded into the gel then electrophoresed at 20 V for 15 hours. The gel was neutralized in 0.1 M Tris pH 8 for 1 hour at 4° C. with rocking. The gel was stained in 4× SYBR Gold in 0.1 M NaCl at 4° C. with rocking protected from light. The gel was briefly washed with deionized water then imaged on a UV transilluminator.

### Statistical Analysis

Data are presented as mean and SEM unless otherwise noted. The number of independent replicates and statistical tests are described in the brief description of the drawings. All statistical tests were calculated using GraphPad Prism 9.

### Example 4

TABLE 1
sgRNA protospacer and PAM sequences used in the Examples

| Figure | Site ID | Protospacer sequence | PAM |
|---|---|---|---|
| FIG. | Sa-W8R | GCCACCGCAGCCACGCAGAGCA | GTGGGT |
| 1 | | (SEQ ID NO: 430) | |
| 3a | Site 1 TS90-GAPDH | GCAAGAGCACAAGAGGAAGAGAG | AGACCC |
| | | (SEQ ID NO: 432) | |
| 3a | Site 2 TS89-SEC61B | GCCCTCATCTCCAATATGGTATGG | CGGCCC |
| | | (SEQ ID NO: 434) | ( |
| 3a | Site 3 TS88-FANCF | GAGGCAAGAGGGCGGCTTTGGGCG | GGGTCC |
| | | (SEQ ID NO: 436) | |
| 3a | Site 4 TS72-LINC01588 | GACCAGCCCCTCGAAGGCAAGGCC | AGGACC |
| | | (SEQ ID NO: 720) | |
| 3a | Site 5 TS81-LSP1 | TATGTTCCAGCTTCCTGGGTCTGC | AGGTCC |
| | | (SEQ ID NO: 440) | |
| 3a | Site 6 Nme10-EMX1 | GGACCCTCTCCATTTCTACCCCT | GGGTCC |
| | | (SEQ ID NO: 442) | |
| 3a | Site 7 Nme91-SpRY- | GTTTGTCCCCACAGTCCCCAGGGA | AAAGCC |
| | NCAC-1 | (SEQ ID NO: 444) | |
| 3a | Site 8 TS71 | GCGCAAAGCTGCATCCACCCCCCG | AGGACC |
| | | (SEQ ID NO: 446) | |
| 3a | Site 9 Nme99 | GCTGGCCGCCAGCCCAGTTGTAGC | ACCGCC |
| | | (SEQ ID NO: 721) | |
| 3a | Site 10 TS75 | GTTCCCCTTCATTGCGGCGGGCTG | CGGGCC |
| | | (SEQ ID NO: 450) | |
| 3b | Site 11 Cj63-AAVS1 | GATTGTGCTGTCAGGAGCTCGG | GGGAGT |
| | | (SEQ ID NO: 452) | AC |
| 3b | Sote 12 Cj56-AAVS1 | GAGTAGAGGCGGCCACGACCTG | GTGAAC |
| | | (SEQ ID NO: 454) | AC |
| 3b | Site 13 Cj672-RNF2 | TCCTAATCCCTTGCACAGGGTC | TGGCAT |
| | | (SEQ ID NO: 456) | AC |
| 3b | Site 14 CJ725-HEK4 | GGTCCAAAGCAGGATGACAGGC | AGGGGG |
| | | (SEQ ID NO: 458) | CAC |
| 3b | Site 15 Cj57-AAVS1 | GGAGTGTGACAGCCTGGGGCCC | AGGCAC |
| | | (SEQ ID NO: 460) | AC |
| 3b | Site 16 Cj61-AAVS1 | AGCTGCAGCACCAGGATCAGTG | AAACGC |
| | | (SEQ ID NO: 462) | AC |
| 3b | Site 17 Cj62-AAVS1 | GAGGTGGCTAAAGCCAGGGAGA | CGGGGT |
| | | (SEQ ID NO: 464) | AC |
| 3b | Site 18 Cj58 | GTGGGGTGAGGAGAAGGCTGGG | AGGGAT |
| | | (SEQ ID NO: 466) | AC |
| 3b | Site 19 Cj59 | GCAGGAGGTTTTCCTTGTGGCA | GGAGAT |
| | | (SEQ ID NO: 468) | AC |
| 3c | Site 20 Sauri-HEK2 | GGAACACAAAGCATAGACTGC (SEQ | GGGG |
| | | ID NO: 470) | |
| 3c | Site 21 Sauri-HEK3 | GGGGCCCAGACTGAGCACGTG (SEQ | ATGG |
| | | ID NO: 472) | |
| 3c | Siete 22 Sauri-RNF2 | GCAGTCATCTTAGTCATTACCT (SEQ | GAGG |
| | | ID NO: 474) | |
| 3c | Site 23 Sauri-EMX1 | GCTGAGTCCGAGCAGAAGAAGA | AGGG |
| | | (SEQ ID NO: 476) | |
| 3c | Site 24 Sauri-FANCF | GATGGAATCCCTTCTGCAGCAC | CTGG |
| | | (SEQ ID NO: 722) | |
| 3c | Site 25 HEK4 | GTGGCACTGCGGCTGGAGGTG (SEQ | GGGG |
| | | ID NO: 480) | |
| 3c | Site 26 GAPDH | CCAGCCCCAGCAAGAGCACAA (SEQ | GAGG |
| | | ID NO: 733) | |
| 3c | Site 27 SEC61B | CCTCATCTCCAATATGGTATG (SEQ | GCGG |
| | | ID NO: 734) | |
| 3c | Site 28 ABE8e Site 1 | GGACAAACCAGAAGCCGCTCC (SEQ | TGGG |
| | | ID NO: 735) | |
| 3c | Site 29 ABE8e Site 7 | CAGTCATCTTAGTCATTACCT (SEQ | GAGG |
| | | ID NO: 488) | |
| 4b | PCSK9 exon 1 donor | GCCTACACCCGCACCTTGGCGC | AGCGGT |
| | SaKKH | (SEQ ID NO: 490) | |
| 4b | Pcsk9 exon 1 donor | GCCATACCTTGGAGCAACGGCG | GAAGGT |
| | SaKKH | (SEQ ID NO: 492) | |
| 4b | Pcsk9 exon 1 donor Sauri | GCACCCATACCTTGGAGCAACG | GCGG |
| | | (SEQ ID NO: 494) | |
| 4b | Angpt13 exon 6 donor | GAGATACCTGAGTAACTTTCTG | GACAGT |
| | SaKKH | (SEQ ID NO: 496) | |
| 4c | Pcsk9 exon 1 donor Sp | GCCCATACCTTGGAGCAACGG (SEQ | CGG |
| | | ID NO: 498) | |
| 6 | Sa-W8R | GCCACCGCAGCCACGCAGAGCA | GTGGGT |
| | | (SEQ ID NO: 500) | |
| 6 | SaKKH-T89A | ATGGAGGAGACCCAGAGGCT (SEQ | ACAGAT |
| | | ID NO: 502) | |
| 6 | SaKKH-Q259R | GCTCAACTGTCAAGGGAAGGG (SEQ | CACAGT |
| | | ID NO: 504) | |
| 6 | SaKKH-Q343N | GCCACGAATGCCCAGGACCA (SEQ | GCCAGT |
| | | ID NO: 506) | |
| 9a | PCSK9_exon1_start_ | GGTGCCCATGAGGGCCAGGGG (SEQ | AGAGGT |
| | SaKKH | ID NO: 508) | |
| 9a | PCSK9_exon1_don_ | GCACCCGCACCTTGGCGCAGCG | GTGGAA |
| | SaSaKKH | (SEQ ID NO: 510) | |
| 9a | PCSK9_exon1_don_ | GCCTACACCCGCACCTTGGCGC | AGCGGT |
| | SaKKH | (SEQ ID NO: 512) | |
| 9a | PCSK9_exon3_acc_ | GTTGCATGGGGCCAGGATCCGT | GGAGGT |
| | SaKKH | (SEQ ID NO: 514) | |
| 9a | PCSK9_exon4_don_ | GAGATGGGGGTCTTACCGGGGG | GCTGGT |
| | SaKKH | (SEQ ID NO: 516) | |
| 9a | PCSK9_exon5_acc_ | GCCTTGGAAAGACGGAGGCAG | CCTGGT |
| | SaKKH | (SEQ ID NO: 518) | |
| 9a | PCSK9_exon5_don_ | GCGTGCTTACCTGTCTGTGGAA | GCGGGT |
| | Sa | (SEQ ID NO: 520) | |
| 9a | PCSK9_exon5_don_Sa_ | GACGGCCGTGCTTACCTGTCTG | TGGAAG |
| | SaKKH | (SEQ ID NO: 522) | |
| 9a | PCSK9_exon6_acc_SaK | GTCGAGCAGGCCAGCAAGTGT (SEQ | GACAGT |
| | KH | ID NO: 524) | |
| 9a | PCSK9_exon9_acc_SaK | GCCCTGCACCAGGCATTGCAGC | CATGAT |
| | KH | (SEQ ID NO: 526) | |
| 9a | PCSK9_exon9_don_Sa | GCCATCCTGCTTACCTGCCCC (SEQ | ATGGGT |
| | | ID NO: 528) | |
| 5b | Pcsk9_exon1_start_ | GTGGGTGCCCATCGGGGCGAGG | AGAGGT |
| | SaKKH | (SEQ ID NO: 530) | |
| 9b | Pcsk9_exon1_don_ | GCCATACCTTGGAGCAACGGCG | GAAGGT |
| | SaKKH | (SEQ ID NO: 532) | |
| 9b | Pcsk9_exon2_don_ | GAAGATGGCTCACCAGGCCCA (SEQ | ACAGGT |
| | SaKKH | ID NO: 534) | |
| 9b | Pcsk9_exon3_don_ | GTACTGGGGACCTTACCAGGGG | AGCGGT |
| | SaKKH | (SEQ ID NO: 536) | |
| 9b | Pcsk9_exon4_acc_ | GCCATGGGAAGATGGAAGCAG | CCAGGT |
| | SaKKH | (SEQ ID NO: 538) | |
| 9b | Pcsk9_exon8_acc_Sa | GTGTACCTCCAGGCATTGTGGC | TCGGAT |
| | | (SEQ ID NO: 540) | |
| 9b | Pcsk9_exon8_don_Sa | GCCATCCTGCTCACCTGTCTC (SEQ | ATGGGT |
| | | ID NO: 542) | |
| 9b | Pcsk9_exon9_don_ | GAGACAGGGTCGGGCACCTCAA | TCCAAT |
| | SaKKH | (SEQ ID NO: 544) | |
| 9c | Angptl3_exon1_start_ | GTGAGACAAAAAATGCACACAA | TTAAAT |
| | SaKKH | (SEQ ID NO: 546) | |
| 9c | Angptl3_exon1_don_Sa | GTGCTGTGATTTCTACTTACT (SEQ | TTGAGT |
| | | ID NO: 548) | |
| 9c | Angptl3_exon1_don_ | GCTGTGATTTCTACTTACTTTG (SEQ | AGTGAT |
| | SaKKH | ID NO: 550) | |
| 9c | Angptl3_exon3_acc_ | GTTCTTTTATCAGCTCAGAAAG | ACTGGT |
| | SaKKH | (SEQ ID NO: 552) | |
| 9c | Angptl3_exon4_don_Sa | GTGTTTCCATGGGTTTACCTGA (SEQ | TTGGGT |
| | | ID NO: 554) | |
| 9c | Angptl3_exon4_don_ | GTGGGTTTACCTGATTGGGTAT | CACAGT |
| | SaKKH | (SEQ ID NO: 556) | |
| 9c | Angptl3_exon5_acc_ | GCTCTCCAGGCAGTCCATGGAC | ATTAAT |
| | SaKKH | (SEQ ID NO: 558) | |
| 9c | Angptl3_exon6_don_ | GAGATACCTGAGTAACTTTCTG | GACAGT |
| | SaKKH | (SEQ ID NO: 560) | |
| 10B | Sauri_Pcsk9_exon1_don_ | GCACCCATACCTTGGAGCAACG | GCGG |
| | Klienstiver | (SEQ ID NO: 562) | |
| 10B | Sauri_Pcsk9_exon1_don_ | GCACCCATACCTTGGAGCAACG | GCGG |
| | Ran | (SEQ ID NO: 564) | |

TABLE 2
sgRNA scaffolds

| Editor | Scaffold sequence |
|---|---|
| SaABE8e | GTTTTAGTACTCTGTAATGAAAATTACAGAATCTACTAAAACAAG |
| | GCAAAATGCCGTGTTTATCTCGTCAACTTGTTGGCGAGATTTTTTT |
| | (SEQ ID NO: 566) |
| SaKKH- | GTTTTAGTACTCTGTAATGAAAATTACAGAATCTACTAAAACAAG |
| ABE8e | GCAAAATGCCGTGTTTATCTCGTCAACTTGTTGGCGAGATTTTTTT |
| | (SEQ ID NO: 567) |
| Nme2 | GTTGTAGCTCCCTTTCTCATTTCGGAAACGAAATGAGAACCGTTGC |
| ABE8e | TACAATAAGGCCGTCTGAAAAGATGTGCCGCAACGCTCTGCCCCT |
| | TAAAGCTTCTGCTTTAAGGGGCATCGTTTATTTTTTT (SEQ ID NO: |
| | 568) |
| CjABE8e | GTTTTAGTCCCTGAAAAGGGACTAAAATAAAGAGTTTGCGGGACT |
| | CTGCGGGGTTACAATCCCCTAAAACCGCTTTTTTT (SEQ ID NO: 569) |
| SauriABE8e | GTTTTAGTACTCTGGAAACAGAATCTACTAAAACAAGGCAAAATG |
| in vitro2 | CCGTGTTTATCTCGTCAACTTGTTGGCGAGATTTTTTT (SEQ ID NO: |
| | 570) |
| SauriABE8e | GTTTTAGTACTCTGTAATGAAAATTACAGAATCTACTAAAACAAG |
| in vivo1 | GCAAAATGCCGTGTTTATCTCGTCAACTTGTTGGCGAGATTTTTTT |
| | (SEQ ID NO: 571) |

TABLE 3
Primer sequences used to amplify genomic DNA and cDNA for
high throughput sequencing

| Figure | Site ID | HTS bind Forward | HTS bind Reverse |
|---|---|---|---|
| FIG. | Pcsk9-W8R | GGCGTCCATGTCCTTCCC | ACATGTGCGGCCTCATCAGC |
| 1 | | GA (SEQ ID NO: 572) | CA (SEQ ID NO: 573) |
| 3a | Site 1 Nme2- | GCTCAGAAAAAGGGCCC | GAGATTCAGTGTGGTGGGGG |
| | TS90-GAPDH | TGA (SEQ ID NO: 574) | (SEQ ID NO: 575) |
| 3a | Site 2 Nme2- | AACTTTCTATCCGTCCGC | GGCTGTAGAGGGAGACAAG |
| | TS89- | GT (SEQ ID NO: 576) | C (SEQ ID NO: 577) |
| | SEC61B | | |
| 3a | Site 3 Nme2- | GGGCCTGGAAGTTCGCT | TGGATCGCTTTTCCGAGCTT |
| | TS88-FANCF | AAT (SEQ ID NO: 578) | (SEQ ID NO: 579) |
| 3a | Site 4 Nme2- | ATTGCTCTTTTCTCCGCC | TTCACAAAACAGGGGTGGCT |
| | TS72- | CA (SEQ ID NO: 580) | (SEQ ID NO: 581) |
| | LINC01588 | | |
| 3a | Site 5 Nme2- | GACGTCTTCTCCTGTGGT | GGGTGTCTGGCTGGAATCTC |
| | TS81-LSP1 | GG (SEQ ID NO: 582) | (SEQ ID NO: 583) |
| 3a | Site 6 Nme10- | CCCCCGCACTCCTTCTTC | AATCTACCTCCGCGGACCT |
| | EMX1 | (SEQ ID NO: 584) | (SEQ ID NO: 585) |
| 3a | Site 7 Nme91- | GGCTCCCTCTCCCAGTTA | CACCACCATCCGCTCTGCCC |
| | SpRY-NCAC- | CCG (SEQ ID NO: 586) | (SEQ ID NO: 587) |
| | 1 | | |
| 3a | Site 8 TS71 | TTCAGGCTGTGAACCTTG | CCGCTACCAGCCGACTTTTA |
| | | GT (SEQ ID NO: 588) | A (SEQ ID NO: 589) |
| 3a | Site 9 Nme99 | GGTGGAAGGTCCCTCCA | CCTTCAACCTGACCTGGGAC |
| | | GA (SEQ ID NO: 590) | (SEQ ID NO: 591) |
| 3a | Site 10 TS75 | ATTGCTCTTTTCTCCGCC | TTCACAAAACAGGGGTGGCT |
| | | CA (SEQ ID NO: 592) | (SEQ ID NO: 593) |
| 3b | Site 11 Cj63- | AAAGAGGAAGCTGTCTC | AGTCTCAGCCAGCCACTTTC |
| | AAVS1 | CGC (SEQ ID NO: 594) | (SEQ ID NO: 595) |
| 3b | Site 12 Cj56- | GCTCTGGGCGGAGGAAT | TCCGTGCGTCAGTTTTACCT |
| | AAVS1 | ATG (SEQ ID NO: 596) | (SEQ ID NO: 597) |
| 3b | Site 13 Cj672- | GATCTGGACGAATAGTG | GCAAGTTGGTTGCTGTCTTTT |
| | RNF2 | TAGGTTAAG (SEQ ID NO: | GG (SEQ ID NO: 599) |
| | | 598) | |
| 3b | Site 14 CJ725- | GAACCCAGGTAGCCAGA | TCCTTTCAACCCGAACGGAG |
| | HEK4 | GAC (SEQ ID NO: 600) | (SEQ ID NO: 601) |
| 3b | Site 15 Cj57- | GCTATGCAGGGTGGAGG | TGGACTTCGGCTTTTGTCCC |
| | AAVS1 | AAG (SEQ ID NO: 602) | (SEQ ID NO: 736) |
| 3b | Site 16 Cj61- | GGGACTAGAAAGGTGAA | GAGGATCCTGGGAGGGAGA |
| | AAVS1 | GAGCC (SEQ ID NO: 603) | G (SEQ ID NO: 604) |
| 3b | Site 17 Cj62- | GGGACTAGAAAGGTGAA | GAGGATCCTGGGAGGGAGA |
| | AAVS1 | GAGCC (SEQ ID NO: 737) | G (SEQ ID NO: 605) |
| 3b | Site 18 Cj58 | GGTCTTCGTTCCTGGCTG | CTTTTCCCTTGGACCCCAGG |
| | | AT (SEQ ID NO: 606) | (SEQ ID NO: 607) |
| 3b | Site 19 Cj59 | ATCAAGGTCAACACAGG | TCACCATGTTGACCAGGCTG |
| | | GGG (SEQ ID NO: 608) | (SEQ ID NO: 609) |
| 3c | Site 20 Sauri- | CCAGCCCCATCTGTCAA | CCGATCTTGAATGGATTCCTT |
| | HEK2 | ACT (SEQ ID NO: 610) | GGAAACAATGA (SEQ ID NO: |
| | | | 611) |
| 3c | Site 21 Sauri- | ATGTGGGCTGCCTAGAA | CCCAGCCAAACTTGTCAACC |
| | HEK3 | AGG (SEQ ID NO: 612) | (SEQ ID NO: 613) |
| 3c | Site 22 Sauri- | ACGTCTCATATGCCCCTT | ACGTAGGAATTTTGGTGGGA |
| | RNF2 | GG (SEQ ID NO: 614) | CA (SEQ ID NO: 615) |
| 3c | Site 23 Sauri- | CAGCTCAGCCTGAGTGTT | CCGATCTCTCGTGGGTTTGTG |
| | EMX1 | GA (SEQ ID NO: 616) | GTTGC (SEQ ID NO: 617) |
| 3c | Site 24 Sauri- | CATTGCAGAGAGGCGTA | GGGGTCCCAGGTGCTGAC |
| | FANCF | TCA (SEQ ID NO: 618) | (SEQ ID NO: 619) |
| 3c | Site 25 HEK4 | GAACCCAGGTAGCCAGA | TCCTTTCAACCCGAACGGAG |
| | | GAC (SEQ ID NO: 620) | (SEQ ID NO: 621) |
| 3c | Site 26 | GCTCAGAAAAAGGGCCC | GAGATTCAGTGTGGTGGGGG |
| | GAPDH | TGA (SEQ ID NO: 622) | (SEQ ID NO: 623) |
| 3c | Site 27 | AACTTTCTATCCGTCCGC | GGCTGTAGAGGGAGACAAG |
| | SEC61B | GT (SEQ ID NO: 624) | C (SEQ ID NO: 625) |
| 3c | Site 28 ABE8e | AGCCCTCTTTTTATTGGA | CCGACTGGTCCACTTACCTA |
| | Site 1 | ACTGTG (SEQ ID NO: 626) | (SEQ ID NO: 627) |
| 3c | Site 29 ABE8e | AACGGAACTCAACCATT | CCAACATACAGAAGTCAGGA |
| | Site 7 | AAGCA (SEQ ID NO: 628) | ATGC (SEQ ID NO: 629) |
| 4b | PCSK9 exon 1 | GCGCACGGCCTCTAGGT | GAGGAAACAGCACCGCACC |
| | donor | CT (SEQ ID NO: 630) | G (SEQ ID NO: 631) |
| 4b | Pcsk9 exon 1 | GCGTCCATGTCCTTCCCG | CCAACCCCAAAGCAACGCCG |
| | donor | AG (SEQ ID NO: 632) | (SEQ ID NO: 633) |
| 4b | Angpt13 exon | TCACCTGGGCAGTCACG | ACACCATCAACATAGTGAGG |
| | 6 donor | AAA (SEQ ID NO: 634) | AGAA (SEQ ID NO: 635) |
| 6 | Pcsk9-W8R | GGCGTCCATGTCCTTCCC | ACATGTGCGGCCTCATCAGC |
| | | GA (SEQ ID NO: 636) | CA (SEQ ID NO: 637) |
| 6 | Pcsk9-T89A | GATGAGGTCATGCTGGG | GTAGAACCTTGATGACATAG |
| | | GTCCT (SEQ ID NO: 638) | CCCCG (SEQ ID NO: 639) |
| 6 | Pcsk9-Q259R | AGGAACTGTTCTCCTCAC | GCCTGACAGTGAGACCTTGC |
| | | TCCCA (SEQ ID NO: 640) | TTC (SEQ ID NO: 641) |
| 6 | Pcsk9-Q343N | TGTGGGAACTGGAGTTG | CTGGACGCTCCGATGATGTC |
| | | GCTAGT (SEQ ID NO: 642) | CTT (SEQ ID NO: 643) |
| 9a | PCSK9_exon1 | GAGGAAACAGCACCGCA | GAGGAAACAGCACCGCACC |
| | | CCG (SEQ ID NO: 644) | G (SEQ ID NO: 645) |
| 9a | PCSK9_exon3 | ACACCTAGGGTTTGCTG | TATGCAGTGGCCCAGCCCTA |
| | acc | GGTTTCTT (SEQ ID NO: | TCA (SEQ ID NO: 647) |
| | | 646) | |
| 9a | PCSK9_exon4_ | AGGCTGTGGCTGTGTTTG | GGAAACCACCAGCAGGGAG |
| | don | CT (SEQ ID NO: 648) | G (SEQ ID NO: 649) |
| 9a | PCSK9_exon5 | CCAGTGCCTGGGATGTG | CAGGAGTACAGCTGCAACGC |
| | | CTCTG (SEQ ID NO: 650) | TC (SEQ ID NO: 651) |
| 9a | PCSK9_exon6_ | TCCAGCCACCTGCTGATT | CCTCCCAGGCCAGGTCCAG |
| | acc | TGT (SEQ ID NO: 652) | (SEQ ID NO: 738) |
| 9a | PCSK9_exon9 | GCCGGGCCATCACCATC | CACACAGACCTCCCAAGCCC |
| | | TTT (SEQ ID NO: 653) | (SEQ ID NO: 654) |
| 9b | Pcsk9_exon1 | GCGTCCATGTCCTTCCCG | CCAACCCCAAAGCAACGCCG |
| | | AG (SEQ ID NO: 655) | (SEQ ID NO: 656) |
| 9b | Pcsk9_exon2_ | GATTGAACAAACTGCCC | CAAGAGCCACAGGCGCTTAC |
| | don | ACCGC (SEQ ID NO: 657) | TG (SEQ ID NO: 658) |
| 9b | Pcsk9_exon3_ | TTGGGAAGAGGACGGGC | TGGGGAGGTGGACAGTCAGG |
| | don | AGA (SEQ ID NO: 659) | (SEQ ID NO: 660) |
| 9b | Pcsk9_exon4_ | CCGTCTAATGCGTGGGG | GGCACGCTGTTGAAGTCGGT |
| | acc | TGG (SEQ ID NO: 661) | (SEQ ID NO: 662) |
| 9b | Pcsk9_exon8 | CCCTATATTTTGGGAAGC | AGAAAACCTTCCTGTGAGGC |
| | | AGGGC (SEQ ID NO: 663) | CA (SEQ ID NO: 664) |
| 9b | Pcsk9_exon9_ | GGGTGCTCCCTCCACCCT | AGGTGCTGCTCTCCAGCCAA |
| | don | AA (SEQ ID NO: 665) | (SEQ ID NO: 666) |
| 9c | Angptl3_exon | GGAGGGAGAAGTTCCAA | CAGCTGCAGGAGGCCATTCG |
| | 1 | ATTGCTTA (SEQ ID NO: | (SEQ ID NO: 668) |
| | | 667) | |
| 9c | Angptl3_exon | TGACACACTTACCCAGC | ATTTTGTAGAGAAAACTGCC |
| | 3_acc | ATTCCCT (SEQ ID NO: | CGGAT (SEQ ID NO: 670) |
| | | 669) | |
| 9c | Angptl3_exon | GCCGACTGCTCTGCCGTT | CCCAGATTTGCGTGAATAAC |
| | 4_don | T (SEQ ID NO: 671) | CATTT (SEQ ID NO: 672) |
| 9c | Angptl3_exon | GCTAAAACCAAACTCAA | CCCATGTCAGTGCAACCAGC |
| | 5_acc | AACGGGT (SEQ ID NO: | TA (SEQ ID NO: 674) |
| | | 673) | |
| 9c | Angptl3_exon | TCACCTGGGCAGTCACG | ACACCATCAACATAGTGAGG |
| | 6_don | AAA (SEQ ID NO: 675) | AGAA (SEQ ID NO: 676) |
| 10A- | Pcsk9_exon1 | GCGTCCATGTCCTTCCCG | CCAACCCCAAAGCAACGCCG |
| 10B | | AG (SEQ ID NO: 677) | (SEQ ID NO: 678) |
| 12A- | OT1 | AACCCCACCACCTTGGC | ACAGTCCACAGTCTAAGTGC |
| 12E | | (SEQ ID NO: 679) | AAA (SEQ ID NO: 680) |
| 12A- | OT2 | TTTGGAGTGGCATATTTG | TGTGGCACAGTGGGCCTTTA |
| 12E | | ACAGCA (SEQ ID NO: 681) | C (SEQ ID NO: 682) |
| 12A- | OT3 | TCATGCTCTGCCCTGTAG | ATGGCATTCATTTAAGCCTA |
| 12E | | GTA (SEQ ID NO: 683) | ACTTC (SEQ ID NO: 684) |
| 16C | OT1 | AAGTATGTTGGGACCCTT | TGGCCTGTTCTACTGACTATG |
| | | GGCTGG (SEQ ID NO: 685) | GGG (SEQ ID NO: 686) |
| 16C | OT2 | GACAGACACAGGGAAGC | AACCTTCCAGGAGAGAGAAA |
| | | CTTGGG (SEQ ID NO: 687) | CCTGT (SEQ ID NO: 688) |
| 16C | OT4 | TTCAAGCAATCACGAGA | CCCACCACCCAGCAGCTTTA |
| | | CACTCAG (SEQ ID NO: | TTG (SEQ ID NO: 690) |
| | | 689) | |
| 16C | OT5 | TCTCAGGCGACCTGGTTT | TCTGCCAGATGCGTCCGATC |
| | | CTGC (SEQ ID NO: 691) | A (SEQ ID NO: 692) |
| 16C | OT6 | GCCAGCCCTGCCTGGAA | TGACCTCCGGGATTCTCAGC |
| | | GTTAG (SEQ ID NO: 693) | CC (SEQ ID NO: 694) |
| 16C | OT7 | GCTTCCTGTCTGCAATTG | AGTAGGTTGCGGGGCTCAGG |
| | | GGGTCT (SEQ ID NO: 695) | A (SEQ ID NO: 696) |
| 16C | OT9 | AACCTCCACGGGGGTAT | ACCTGGCAAGTGGGGTACTG |
| | | CTGAGG (SEQ ID NO: 697) | G (SEQ ID NO: 698) |
| 16C | OT10 | GTCTAAATGGGCAAGCA | CCAGGATCCCACAGGGTCCT |
| | | ATCCCCT (SEQ ID NO: | TCT (SEQ ID NO: 700) |
| | | 699) | |
| 16C | OT11 | TCCCCAGAGCCCAGGGA | TGTTGCTCCGATGGAAGGAT |
| | | ATATCA (SEQ ID NO: 701) | GGG (SEQ ID NO: 702) |
| 16C | OT13 | CCACCAGAAGCGCCCCA | GGGGAATCGCCTCCACTGCC |
| | | GAA (SEQ ID NO: 703) | (SEQ ID NO: 704) |
| 16C | OT14 | GTTCTTATTGGCCAGGGA | CTCCCCAAGTGACAGGAACC |
| | | GCCTT (SEQ ID NO: 705) | ACG (SEQ ID NO: 706) |
| 22A | cDNA_Canx/ | GGGTCTGGAATATACTC | TGACCAGTCTGTTGTGAACA |
| | Ip90 | AGGTTCGT (SEQ ID NO: | GTG (SEQ ID NO: 708) |
| | | 707) | |
| 22B | cDNA_Ctnnb1 | TGACCTGATGGAGTTGG | ACTTGCTCTTGCGTGAAGGA |
| | | ACATGG (SEQ ID NO: 709) | CT (SEQ ID NO: 710) |
| 22C | cDNA_Usp38 | ACAGGTCTTCCACAGAA | ACTTGTTTACAAGAGGTGAC |
| | | GGGC (SEQ ID NO: 711) | CAGT (SEQ ID NO: 712) |
| 22D | cDNA_Aars | AGCCAGAATCCTCCCTG | CAGAACACCATCCGACTCCC |
| | | GCAA (SEQ ID NO: 713) | TGT (SEQ ID NO: 714) |

TABLE 4A
Summary of the base editing activity windows of size-minimized ABEs
developed in this manuscript and the percentages of targetable genomic
adenines.

| | Protospacer | | Window |
|---|---|---|---|
| Variant | length (nt) | PAM | (25% max) |
| SauriABE | 21 | NNGG (SEQ ID NO: 715) | 5-15 |
| SaKKHABE | 21 | NNNRRT (SEQ ID NO: 716) | 2-13 |
| CjABE | 22 | NNNV RYAC (SEQ ID NO: | 3-7, 9-13, 15 |
| | | 717) | |
| Nme2ABE | 24 | NNNNCC (SEQ ID NO: 718) | 6, 9-13, 16-17 |

TABLE 4B
Window widths used for the calculation of targetable genomic adenines. Window
widths are shown with respect to the standard protospacer lengths of each
editor, with position 1 being defined as the 5′ end of the protospacer.

| | | | | | percent |
|---|---|---|---|---|---|
| | total_A | targetable_A | total_T | targetable_T | targetable |
| chr1 | 32546775 | 26685188 | 32599119 | 26731923 | 81.99612857 |
| chr10 | 19549602 | 16072176 | 19566475 | 16079439 | 82.19539756 |
| chr11 | 18530910 | 15213431 | 18548654 | 15231448 | 82.10689586 |
| chr12 | 18869776 | 15364041 | 18905105 | 15390534 | 81.415412 |
| chr13 | 15663586 | 12722581 | 15718153 | 12773277 | 81.24424845 |
| chr14 | 13041618 | 10670545 | 13118537 | 10736532 | 81.83084924 |
| chr15 | 11901023 | 9791465 | 11881736 | 9779219 | 82.2893761 |
| chr16 | 10761831 | 8880058 | 10847090 | 8959068 | 82.55445054 |
| chr17 | 10711334 | 8892175 | 10719812 | 8897966 | 83.01068454 |
| chr18 | 11963020 | 9779925 | 12016805 | 9824978 | 81.75582182 |
| chr19 | 5808203 | 4848031 | 5816556 | 4854410 | 83.46358836 |
| chr2 | 36905447 | 30200054 | 36965508 | 30246198 | 81.82681813 |
| chr20 | 8190084 | 6756662 | 8272621 | 6829290 | 82.52563598 |
| chr21 | 5754013 | 4719119 | 5767220 | 4730454 | 82.01876483 |
| chr22 | 4583339 | 3855516 | 4582999 | 3855167 | 84.11955789 |
| chr3 | 29608996 | 24099572 | 29636045 | 24126868 | 81.40164845 |
| chr4 | 29167021 | 23603218 | 29177885 | 23608189 | 80.91778741 |
| chr5 | 27049804 | 22032634 | 27155675 | 22123573 | 81.46078185 |
| chr6 | 26137405 | 21311082 | 26196421 | 21367050 | 81.54980299 |
| chr7 | 23255299 | 19020621 | 23243204 | 19007154 | 81.78279417 |
| chr8 | 21395653 | 17440637 | 21361906 | 17416402 | 81.52251863 |
| chr9 | 17364609 | 14209902 | 17335711 | 14192377 | 81.85019331 |
| chrX | 17917313 | 14524778 | 17926781 | 14532656 | 81.06616951 |
| chrY | 2994088 | 2426016 | 3002884 | 2431968 | 81.00728167 |
| all | 419670749 | 343119427 | 420362902 | 343726140 | 81.76405388 |

The percentage of genomic adenincs targetable with one or more size-minimized ABEs developed in this study using the activity window definitions in (a).

Custom python script for calculating the targetable adenincs in the human genome with small ABE8c targetable PAMs.

```
import re
from Bio import SeqIO
import Bio
from Bio.Seq import Seq
import pandas as pd
def is_targetable(sequence, A_position, window, PAM_seq, protospacer_length):
  #convert PAM to regex PAM
  regex_PAM =
PAM_seq.replace(‘N’,‘[ATGC]’).replace(‘R’,‘[AG]’).replace(‘Y’,‘[CT]’).replace(
‘V’,‘[AGC]’)
  is_targetable = 0
  for coords in window:
    test_for_PAM = sequence[A_position + (protospacer_length − coords[1])
+ 1:A_position + (protospacer_length − coords[0]) + 1 + len(PAM_seq)]
    if [m.start( ) for m in re.finditer(regex_PAM, test_for_PAM)]:
      is_targetable = 1
  return is_targetable
genome_fa = ‘/Volumes/Storage/AR/ /genome_builds/hg38/hg38.fa’
records = SeqIO.to_dict(SeqIO.parse(genome_fa,‘fasta’))
keys = records.keys( )
#store genome data as dict = {‘chrN’: (seq, reverse complement seq)}
sequences = {key: (str(records[key].seq), str(records[key].seq.reverse_complement( ))) for
key in records.keys( )}
#output targetable counts as dict = {‘chrN’: {‘targetable_A’: int}, {‘total_A’: int}, {‘total_A’:
int}, {‘targetable_T’: int}, {‘total_T’}: int}
output = {key: { } for key in records.keys( )}
#free up some memory
del records
#iterate over chromosomes and populate output dict
for chromosome in keys:
  print(‘Tabulating ’ + str(chromosome) + ‘ ... ’)
  #do forward (sense) seq first; findall As
  A_positions = [m.start( ) for m in re.finditer(‘A’, sequences[chromosome][0])]
  #update total_A value
  output[chromosome][‘total_A’] = len(A_positions)
  #calculate targetable As
  targetable_As = [ ]
  for A_position in A_positions:
    targetable_As.append(0)
    if is_targetable(sequences[chromosome][0], A_position, [1,12]],
‘NNNRRT’, 20):
      targetable_As[−1] = 1
    elif is_targetable(sequences[chromosome][0], A_position, [[4,14]],
‘NNGG’, 20):
      targetable_As[−1] = 1
    elif is_targetable(sequences[chromosome][0], A_position, [[2,2], [5,9],
[12,13], ‘NNNNCC’, 23):
      targetable_As[−1] = 1
    elif is_targetable(sequences[chromosome][0], A_position, [[1,5], [7,11],
[13,13]], ‘NNNNRYAC’, 22):
      targetable_As[−1] = 1
  #update targetable_A value
  output[chromosome][‘targetable_A’] = sum(targetable_As)
  #do reverse complement seq next
  T_positions = [m.start( ) for m in re.finditer(‘A’, sequences[chromosome][1])]
  output[chromosome][‘total_T’] = len(T_positions)
  targetable_Ts = [ ]
  for T_position in T_positions:
    targetable_Ts.append(0)
    if is_targetable(sequences[chromosome][1], T_position, [1,12]],
‘NNNRRT’, 20):
      targetable_Ts[−1] = 1
    elif is_targetable(sequences[chromosome][1], T_position, [4,14]],
‘NNGG’, 20):
      targetable_Ts[−1] = 1
    elif is_targetable(sequences[chromosome][1], T_position,
[[2,2],
[5,9], [12,13]], ‘NNNNCC’, 20):
      targetable_Ts[−1] = 1
    elif is_targetable(sequences[chromosome][1], T_position,
[[1,5],
[7,11], [13,13], ‘NNNNRYAC’, 20):
      targetable_Ts[−1] = 1
  output[chromosome][‘targetable_T’] = sum(targetable_Ts)
#calculate combined totals for all chromosomes
all_total_A = 0
all_total_T = 0
all_targetable_A = 0
all_targetable_T = 0
for chromosome in output.keys( ):
  all_total_A += output[chromosome][‘total_A’]
  all_targetable_A += output[chromosome][‘targetable_A’]
  all_total_T += output[chromosome][‘total_T’]
  all_targetable_T += output[chromosome][‘targetable_T’]
output[‘all’] = {‘total_A’: all_total_A, ‘targetable_A’: all_targetable_A, ‘total_T’: all_total_T,
‘targetable_T’: all_targetable_T}
output_df = pd.DataFrame.from_dict(output, orient=‘index’)
output_df[‘percent targetable’] = (output_df[‘targetable_A’] +
output_df[‘targetable_T’])/(output_df[‘total_A’] + output_df[‘total_T’])*100
  output_df.to_csv(‘2022-06-03/genome-wide_BE_search_Sauri-SaKKH-Nme2-
Cj.csv’)
```

### REFERENCES

1. Newby, G. A. & Liu, D. R. In vivo somatic cell base editing and prime editing. Molecular Therapy 29, 3107-3124 (2021).
2. Banskota, S., et al. Engineered virus-like particles for efficient in vivo delivery of therapeutic proteins. Cell 185 (2): 250-265 (2022).
3. Palaschak, B., Herzog, R. W. & Markusic, D. M. AAV-Mediated Gene Delivery to the Liver: Overview of Current Technologies and Methods. in Adeno-Associated Virus Vectors: Design and Delivery (ed. Castle, M. J.) 333-360 (Springer New York, New York, NY, 2019).
4. Deverman, B. E., Ravina, B. M., Bankiewicz, K. S., Paul, S. M. & Sah, D. W. Y. Gene therapy for neurological disorders: progress and prospects. Nature Reviews Drug Discovery 17, 641-659 (2018).
5. Mendell, J. R., et al. Current Clinical Applications of In Vivo Gene Therapy with AAVs. Molecular Therapy 29, 464-488 (2021).
6. Mendell, J. R., et al. Single-Dose Gene-Replacement Therapy for Spinal Muscular Atrophy. New England Journal of Medicine 377, 1713-1722 (2017).
7. Russell, S., et al. Efficacy and safety of voretigene neparvovec (AAV2-hRPE65v2) in patients with <em>RPE65</em>-mediated inherited retinal dystrophy: a randomised, controlled, open-label, phase 3 trial. The Lancet 390, 849-860 (2017).
8. Komor, A. C., Kim, Y. B., Packer, M. S., Zuris, J. A. & Liu, D. R. Programmable editing of a target base in genomic DNA without double-stranded DNA cleavage. Nature 533, 420-424 (2016).
9. Gaudelli, N. M., et al. Programmable base editing of A•T to G•C in genomic DNA without DNA cleavage. Nature 551, 464 (2017).
10. Anzalone, A. V., Koblan, L. W. & Liu, D. R. Genome editing with CRISPR-Cas nucleases, base editors, transposases and prime editors. Nature Biotechnology 38, 824-844 (2020).
11. Giannoukos, G., et al. UDiTaS™, a genome editing detection method for indels and genome rearrangements. BMC Genomics 19, 212 (2018).
12. Zuccaro, M. V., et al. Allele-Specific Chromosome Removal after Cas9 Cleavage in Human Embryos. Cell 183, 1650-1664.e1615 (2020).
13. Kosicki, M., Tomberg, K. & Bradley, A. Repair of double-strand breaks induced by CRISPR-Cas9 leads to large deletions and complex rearrangements. Nature Biotechnology 36, 765-771 (2018).
14. Boutin, J., et al. CRISPR-Cas9 globin editing can induce megabase-scale copy-neutral losses of heterozygosity in hematopoietic cells. Nat Commun 12, 4922 (2021).
15. Ihry, R. J., et al. p53 inhibits CRISPR-Cas9 engineering in human pluripotent stem cells. Nature Medicine 24, 939-946 (2018).
16. Haapaniemi, E., Botla, S., Persson, J., Schmierer, B. & Taipale, J. CRISPR-Cas9 genome editing induces a p53-mediated DNA damage response. Nature Medicine 24, 927-930 (2018).
17. Leibowitz, M. L., et al. Chromothripsis as an on-target consequence of CRISPR-Cas9 genome editing. Nature Genetics 53, 895-905 (2021).
18. Dong, J. Y., Fan, P. D. & Frizzell, R. A. Quantitative analysis of the packaging capacity of recombinant adeno-associated virus. Hum Gene Ther 7, 2101-2112 (1996).
19. Wu, Z., Yang, H. & Colosi, P. Effect of genome size on AAV vector packaging. Molecular therapy: the journal of the American Society of Gene Therapy 18, 80-86 (2010).
20. Anzalone, A. V., et al. Search-and-replace genome editing without double-strand breaks or donor DNA. Nature 576, 149-157 (2019).
21. Koblan, L. W., et al. In vivo base editing rescues Hutchinson-Gilford progeria syndrome in mice. Nature 589, 608-614 (2021).
22. Villiger, L., et al. Treatment of a metabolic liver disease by in vivo genome base editing in adult mice. Nat Med 24, 1519-1525 (2018).
23. Chen, Y., et al. Development of Highly Efficient Dual-AAV Split Adenosine Base Editor for In Vivo Gene Therapy. Small Methods 4, 2000309 (2020).
24. Lim, C. K. W., et al. Treatment of a Mouse Model of ALS by In Vivo Base Editing. Molecular Therapy 28, 1177-1189 (2020).
25. Chemello, F., et al. Precise correction of Duchenne muscular dystrophy exon deletion mutations by base and prime editing. Science advances 7, eabg4910 (2021).
26. Xu, L., et al. Efficient precise in vivo base editing in adult dystrophic mice. Nat Commun 12, 3719 (2021).
27. Zettler, J., Schutz, V. & Mootz, H. D. The naturally split Npu DnaE intein exhibits an extraordinarily high rate in the protein trans-splicing reaction. FEBS letters 583, 909-914 (2009).
28. Ryu, S.-M., et al. Adenine base editing in mouse embryos and an adult mouse model of Duchenne muscular dystrophy. Nature Biotechnology 36, 536-539 (2018).
29. Kuzmin, D. A., et al. The clinical landscape for AAV gene therapies. Nat Rev Drug Discov 20, 173-174 (2021).
30. Richter, M. F., et al. Phage-assisted evolution of an adenine base editor with improved Cas domain compatibility and activity. Nature Biotechnology 38, 883-891 (2020).
31. Grünewald, J., et al. CRISPR DNA base editors with reduced RNA off-target and self-editing activities. Nature biotechnology 37, 1041-1048 (2019).
32. Nguyen Tran, M. T., et al. Engineering domain-inlaid SaCas9 adenine base editors with reduced RNA off-targets and increased on-target DNA editing. Nat Commun 11, 4871 (2020).
33. Kleinstiver, B. P., et al. Broadening the targeting range of Staphylococcus aureus CRISPR-Cas9 by modifying PAM recognition. Nature Biotechnology 33, 1293-1298 (2015).
34. Levy, J. M., et al. Cytosine and adenine base editing of the brain, liver, retina, heart and skeletal muscle of mice via adeno-associated viruses. Nat Biomed Eng 4, 97-110 (2020).
35. Inagaki, K., et al. Robust systemic transduction with AAV9 vectors in mice: efficient global cardiac gene transfer superior to that of AAV8. Molecular Therapy 14, 45-53 (2006).
36. Wu, Z., Asokan, A. & Samulski, R. J. Adeno-associated Virus Serotypes: Vector Toolkit for Human Gene Therapy. Molecular Therapy 14, 316-327 (2006).
37. Wu, Z., et al. Optimization of Self-complementary AAV Vectors for Liver-directed Expression Results in Sustained Correction of Hemophilia B at Low Vector Dose. Molecular Therapy 16, 280-289 (2008).
38. Long, C., et al. Prevention of muscular dystrophy in mice by CRISPR/Cas9& #x2013; mediated editing of germline DNA. Science 345, 1184-1188 (2014).
39. Song, C.-Q., et al. In Vivo Genome Editing Partially Restores Alphal-Antitrypsin in a Murine Model of AAT Deficiency. Human Gene Therapy 29, 853-860 (2018).
40. Shen, S., et al. Amelioration of Alpha-1 Antitrypsin Deficiency Diseases with Genome Editing in Transgenic Mice. Human Gene Therapy 29, 861-873 (2018).
41. Rothgangl, T., et al. In vivo adenine base editing of PCSK9 in macaques reduces LDL cholesterol levels. Nature Biotechnology 39, 949-957 (2021).
42. Edraki, A., et al. A Compact, High-Accuracy Cas9 with a Dinucleotide PAM for In Vivo Genome Editing. Molecular Cell 73, 714-726.e714 (2019).
43. Liu, Z., et al. Efficient and high-fidelity base editor with expanded PAM compatibility for cytidine dinucleotide. Science China Life Sciences 64, 1355-1367 (2021).
44. Kim, E., et al. In vivo genome editing with a small Cas9 orthologue derived from Campylobacter jejuni. Nat Commun 8, 14500 (2017).
45. Li, X., et al. Programmable base editing of mutated TERT promoter inhibits brain tumour growth. Nature Cell Biology 22, 282-288 (2020).
46. Hu, Z., et al. A compact Cas9 ortholog from Staphylococcus Auricularis (SauriCas9) expands the DNA targeting scope. PLoS Biology 18, e3000686 (2020).
47. Kim, Y. B., et al. Increasing the genome-targeting scope and precision of base editing with engineered Cas9-cytidine deaminase fusions. Nature biotechnology 35, 371-376 (2017).
48. Huang, T. P., et al. Circularly permuted and PAM-modified Cas9 variants broaden the targeting scope of base editors. Nature Biotechnology 37, 626-631 (2019).
49. Rees, H. A. & Liu, D. R. Base editing: precision chemistry on the genome and transcriptome of living cells. Nature Reviews Genetics 19, 770-788 (2018).
50. Cohen, J. C., Boerwinkle, E., Mosley, T. H. & Hobbs, H. H. Sequence Variations in PCSK9, Low LDL, and Protection against Coronary Heart Disease. New England Journal of Medicine 354, 1264-1272 (2006).
51. Dewey, F. E., et al. Genetic and Pharmacologic Inactivation of ANGPTL3 and Cardiovascular Disease. New England Journal of Medicine 377, 211-221 (2017).
52. Stitziel, N. O., et al. ANGPTL3 Deficiency and Protection Against Coronary Artery Disease. Journal of the American College of Cardiology 69, 2054-2063 (2017).
53. Kluesner, M. G., et al. CRISPR-Cas9 cytidine and adenosine base editing of splice-sites mediates highly-efficient disruption of proteins in primary and immortalized cells. Nat Commun 12, 2437 (2021).
54. Gao, G.-P., et al. Novel adeno-associated viruses from rhesus monkeys as vectors for human gene therapy. Proceedings of the National Academy of Sciences of the United States of America 99, 11854-11859 (2002).
55. Essalmani, R., et al. A single domain antibody against the Cys- and His-rich domain of PCSK9 and evolocumab exhibit different inhibition mechanisms in humanized PCSK9 mice. Biological Chemistry 399, 1363-1374 (2018).
56. Nakai, H., et al. Unrestricted hepatocyte transduction with adeno-associated virus serotype 8 vectors in mice. Journal of virology 79, 214-224 (2005).
57. Racanelli, V. & Rehermann, B. The liver as an immunological organ. Hepatology 43, S54-S62 (2006).
58. Qiu, M., et al. Lipid nanoparticle-mediated codelivery of Cas9 mRNA and single-guide RNA achieves liver-specific in vivo genome editing of <em>Angpt13</em>. Proceedings of the National Academy of Sciences 118, e2020401118 (2021).
59. Musunuru, K., et al. In vivo CRISPR base editing of PCSK9 durably lowers cholesterol in primates. Nature 593, 429-434 (2021).
60. Meyer, K., et al. Improving single injection CSF delivery of AAV9-mediated gene therapy for SMA: a dose-response study in mice and nonhuman primates. Mol Ther 23, 477-487 (2015).
61. Rashid, S., et al. Decreased plasma cholesterol and hypersensitivity to statins in mice lacking <em>Pcsk9</em>. Proceedings of the National Academy of Sciences of the United States of America 102, 5374-5379 (2005).
62. Koishi, R., et al. Angpt13 regulates lipid metabolism in mice. Nature Genetics 30, 151-157 (2002).
63. Chadwick, A. C., Evitt, N. H., Lv, W. & Musunuru, K. Reduced Blood Lipid Levels With In Vivo CRISPR-Cas9 Base Editing of ANGPTL3. Circulation 137, 975-977 (2018).
64. Chu, S. H., et al. Rationally Designed Base Editors for Precise Editing of the Sickle Cell Disease Mutation. The CRISPR Journal 4, 169-177 (2021).
65. High-dose AAV gene therapy deaths. Nature Biotechnology 38, 910-910 (2020).
66. Hinderer, C., et al. Severe Toxicity in Nonhuman Primates and Piglets Following High-Dose Intravenous Administration of an Adeno-Associated Virus Vector Expressing Human SMN. Human Gene Therapy 29, 285-298 (2018).
67. Chandler, R. J., Sands, M. S. & Venditti, C. P. Recombinant Adeno-Associated Viral Integration and Genotoxicity: Insights from Animal Models. Hum Gene Ther 28, 314-322 (2017).
68. Schmidt, M., Gil-Farina, I. & Büning, H. Reply to “Wild-type AAV insertions in hepatocellular carcinoma do not inform debate over genotoxicity risk of vectorized AAV”. Molecular Therapy 24, 661-662 (2016).
69. Mullard, A. Gene therapy community grapples with toxicity issues, as pipeline matures. Nature Reviews Drug Discovery, 804-805 (2021).
70. Wang, L., et al. Long-term stable reduction of low-density lipoprotein in nonhuman primates following in vivo genome editing of PCSK9. Molecular Therapy 29, 2019-2029 (2021).
71. Monteys, A. M., et al. Regulated control of gene therapies by drug-induced splicing. Nature 596, 291-295 (2021).
72. Dahlman, J. E., et al. Barcoded nanoparticles for high throughput in vivo discovery of targeted therapeutics. Proceedings of the National Academy of Sciences 114, 2060-2065 (2017).
73. Piotrowski-Daspit, A. S., Glaze, P. M. & Saltzman, W. M. Debugging the genetic code: non-viral in vivo delivery of therapeutic genome editing technologies. Curr Opin Biomed Eng 7, 24-32 (2018).
74. Ran, F. A., et al. (2015). In vivo genome editing using Staphylococcus aureus Cas9. Nature 520 (7546): 186-191.
75. Zincarelli, C., Soltys, S., Rengo, G. & Rabinowitz, J. E. Analysis of AAV Serotypes 1-9 Mediated Gene Expression and Tropism in Mice After Systemic Injection. Molecular Therapy 16, 1073-1080 (2008).
76. Rees, H. A., Wilson, C., Doman, J. L. & Liu, D. R. Analysis and minimization of cellular RNA editing by DNA adenine base editors. Science Advances 5, eaax5717 (2019).
77. Bac, S., Park, J. & Kim, J.-S. Cas-OFFinder: a fast and versatile algorithm that searches for potential off-target sites of Cas9 RNA-guided endonucleases. Bioinformatics 30, 1473-1475 (2014).
78. Concordet, J.-P. & Hacussler, M. CRISPOR: intuitive guide selection for CRISPR/Cas9 genome editing experiments and screens. Nucleic acids research 46, W242-W245 (2018).

### EQUIVALENTS AND SCOPE

Those skilled in the art will recognize, or be able to ascertain using no more than routine experimentation, many equivalents to the specific embodiments of the invention described herein. The scope of the present invention is not intended to be limited to the above description, but rather is as set forth in the appended claims.

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process.

Furthermore, it is to be understood that the invention encompasses all variations, combinations, and permutations in which one or more limitations, elements, clauses, descriptive terms, etc., from one or more of the claims or from relevant portions of the description is introduced into another claim. For example, any claim that is dependent on another claim can be modified to include one or more limitations found in any other claim that is dependent on the same base claim. Furthermore, where the claims recite a composition, it is to be understood that methods of using the composition for any of the purposes disclosed herein are included, and methods of making the composition according to any of the methods of making disclosed herein or other methods known in the art are included, unless otherwise indicated or unless it would be evident to one of ordinary skill in the art that a contradiction or inconsistency would arise.

Where elements are presented as lists, e.g., in Markush group format, it is to be understood that each subgroup of the elements is also disclosed, and any element(s) can be removed from the group. It is also noted that the term “comprising” is intended to be open and permits the inclusion of additional elements or steps. It should be understood that, in general, where the invention, or aspects of the invention, is/are referred to as comprising particular elements, features, steps, etc., certain embodiments of the invention or aspects of the invention consist, or consist essentially of, such elements, features, steps, etc. For purposes of simplicity those embodiments have not been specifically set forth in haec verba herein. Thus for each embodiment of the invention that comprises one or more elements, features, steps, etc., the invention also provides embodiments that consist or consist essentially of those elements, features, steps, etc.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in different embodiments of the invention, to the tenth of the unit of the lower limit of the range, unless the context clearly dictates otherwise. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

In addition, it is to be understood that any particular embodiment of the present invention may be explicitly excluded from any one or more of the claims. Where ranges are given, any value within the range may explicitly be excluded from any one or more of the claims. Any embodiment, element, feature, application, or aspect of the compositions and/or methods of the invention, can be excluded from any one or more claims. For purposes of brevity, all of the embodiments in which one or more elements, features, purposes, or aspects is excluded are not set forth explicitly herein.

All publications, patents and sequence database entries mentioned herein, including those items listed above, are hereby incorporated by reference in their entirety as if each individual publication or patent was specifically and individually indicated to be incorporated by reference. In case of conflict, the present application, including any definitions herein, will control.

What is claimed is:

1. A nucleic acid molecule comprising:
(i) a 5′ inverted terminal repeat (ITR);
(ii) a first nucleic acid segment comprising a sequence encoding a base editor operably linked to a first promoter, wherein the base editor comprises a nucleic acid programmable DNA binding protein (napDNAbp) domain and a deaminase domain; and a polyadenylation (polyA) signal;
(iii) a second nucleic acid segment encoding a guide RNA (gRNA) operably linked to a second promoter; and
(iv) a 3′ ITR,
wherein the length the length between the 5′ ITR and the 3′ ITR is less than about 4.90 kb.

2. The nucleic acid molecule of claim 1, wherein the first nucleic acid segment does not encode an intein.

3. The nucleic acid molecule of claim 1 or 2, wherein the nucleic acid molecule does not comprise a posttranscriptional response element.

4. The nucleic acid molecule of any one of claims 1-3, wherein the second promoter is a U6 promoter.

5. The nucleic acid molecule of any one of claims 1-4, wherein the first promoter has a length of less than 325 nucleotides, less than 315 nucleotides, less than 300 nucleotides, less than 285 nucleotides, less than 270 nucleotides, less than 265 nucleotides, or less than 250 nucleotides.

6. The nucleic acid molecule of any one of claims 1-5, wherein the first promoter has a length of approximately 280 nucleotides.

7. The nucleic acid molecule of any one of claims 1-5, wherein the first promoter is an EF-1a short (EFS) promoter, a MeCP2 promoter, a P3 promoter, or a U1A promoter.

8. The nucleic acid molecule of any one of claims 1-7, wherein the first promoter is an EF-1a short (EFS) promoter.

9. The nucleic acid molecule of any one of claims 1-8, wherein the first promoter is a tissue-specific promoter.

10. The nucleic acid molecule of claim 9, wherein the first promoter is a cardiac tissue-specific promoter, a muscle tissue-specific promoter, or a neuronal tissue-specific promoter.

11. The nucleic acid molecule of any one of claims 1-10, wherein the base editor comprises (i) a nucleic acid programmable DNA binding protein (napDNAbp) domain; and
(ii) a deaminase domain.

12. The nucleic acid molecule of any one of claims 1-11, wherein the napDNAbp domain is a Cas9 domain.

13. The nucleic acid molecule of any one of claims 1-12, wherein the napDNAbp domain is selected from a S. aureus Cas9 (SaCas9) domain, an N. meningitidis 2 Cas9 (Nme2Cas9) domain, a C. jejuni Cas9 (CjCas9) domain, an S. auricularis (SauriCas9) domain, and variants thereof.

14. The nucleic acid molecule of any one of claims 1-13, wherein the napDNAbp domain has nickase activity.

15. The nucleic acid molecule of any one of claims 1-14, wherein the napDNAbp domain is a compact variant of S. pyogenes Cas9 (SpCas9), Cpf1, CasX, CasY, C2c1, C2c2, C2c3, Cas12a, Cas12b, Cas12g, Cas12h, Cas12i, Cas13b, Cas13c, Cas13d, Cas14, Csn2, Cas3, or CasΦ.

16. The nucleic acid molecule of any one of claims 1-14, wherein the napDNAbp domain is an SaCas9 domain, SaCas9 nickase domain, SaKKH domain, or SaKKH nickase domain.

17. The nucleic acid molecule of any one of claims 1-14 and 16, wherein the napDNAbp domain is an SaKKH nickase domain.

18. The nucleic acid molecule of any one of claims 1-14 and 16, wherein the napDNAbp domain is a CjCas9 nickase domain.

19. The nucleic acid molecule of any one of claims 1-18, wherein the deaminase domain comprises an adenosine deaminase.

20. The nucleic acid molecule of any one of claims 1-19, wherein the deaminase domain consists of an adenosine deaminase monomer.

21. The nucleic acid molecule of any one of claims 1-20, wherein the deaminase domain is a TadA-8e, a TadA-8e (V106W), a TadA9, a TadA20, or a TadA7.10 deaminase.

22. The nucleic acid molecule of any one of claims 1-21, wherein the deaminase domain is a TadA-8e deaminase domain.

23. The nucleic acid molecule of any one of claims 1-22, wherein the base editor further comprises one or more nuclear localization sequences (NLS).

24. The nucleic acid molecule of any one of claims 1-23, wherein the base editor further comprises a bipartite NLS (bpNLS).

25. The nucleic acid molecule of claim 24, wherein the bipartite nuclear localization signal comprises an amino acid sequence selected from the group consisting of: KRTADGSEFEPKKKRKV (SEQ ID NO: 398), KRPAATKKAGQAKKKK (SEQ ID NO: 344), KKTELQTTNAENKTKKL (SEQ ID NO: 345), KRGINDRNFWRGENGRKTR (SEQ ID NO: 346), and RKSGKIAAIVVKRPRK (SEQ ID NO: 347).

26. The composition of claim 24 or 25, wherein the bipartite nuclear localization signal comprises the amino acid sequence set forth in SEQ ID NO: 344 or 398.

27. The nucleic acid molecule of any one of claims 1-26, wherein the base editor is ABE8e, ABE8e (V106W), ABE9, ABE20, ABE7.10, or a variant thereof.

28. The nucleic acid molecule of any one of claims 1-27, wherein the base editor is SaKKH-ABE8e, SauriCas9-ABE8e, CjCas9-ABE8e, Nme2Cas9-ABE8e, or SaCas9-ABE8e.

29. The nucleic acid molecule of any one of claims 1-28, wherein the base editor comprises the structure: NH₂-[adenosine deaminase]-[napDNAbp domain]-COOH; or NH₂-[napDNAbp domain]-[adenosine deaminase]-COOH, wherein each “]-[” in the structure indicates the presence of an optional linker sequence.

30. The nucleic acid molecule of any one of claims 1-18 and 23-26, wherein the deaminase domain comprises a cytidine deaminase.

31. The nucleic acid molecule of any one of claims 1-18, 23-26, and 30, wherein the deaminase domain is FERNY or evolved FERNY (evoFERNY).

32. The nucleic acid molecule of claims 1-18, 23-26, 30, and 31, wherein the base editor is BE3.9, FERNY-BE3.9, or a variant thereof.

33. The nucleic acid molecule of any one of claims 1-18, 23-26, and 30-32, wherein the base editor further comprises a uracil glycosylase inhibitor (UGI) domain.

34. The nucleic acid molecule of any one of claims 1-18 and 30-33, wherein the base editor comprises the structure:
NH₂-[cytidine deaminase domain]-[napDNAbp domain]-[UGI]-COOH;
NH₂-[cytidine deaminase domain]-[UGI]-[napDNAbp domain]-COOH;
NH₂-[napDNAbp domain]-[UGI]-[cytidine deaminase domain]-COOH;
NH₂-[napDNAbp domain]-[cytidine deaminase domain]-[UGI]-COOH;
NH₂-[UGI]-[cytidine deaminase domain]-[napDNAbp domain]-COOH; or
NH₂-[UGI]-[napDNAbp domain]-[cytidine deaminase domain]-COOH,
wherein each instance of “]-[” in the structure indicates the presence of an optional linker sequence.

35. The nucleic acid molecule of any one of claims 1-18, 23-26, and 30-33, wherein the base editor comprises the amino acid sequence of SEQ ID NO: 21 or 22.

36. The nucleic acid molecule of any one of claims 1-29, wherein the base editor comprises the amino acid sequence of any of SEQ ID NOs: 171-172 and 181-183.

37. The nucleic acid molecule of any one of claims 1-29 and 36, wherein the base editor comprises the amino acid sequence of SEQ ID NO: 171.

38. The nucleic acid molecule of any one of claims 1-37, wherein the polyA signal is a bovine growth hormone (bGH) signal, human growth hormone (hGH) signal, or SV40 signal.

39. The nucleic acid molecule of any one of claims 1-38, wherein the polyA signal is a bovine growth hormone (bGH) signal.

40. The nucleic acid molecule of any one of claims 1-39, wherein the first nucleic acid segment further comprises a minimal minute virus of mice (MVM) intron.

41. The nucleic acid molecule of any one of claims 1-40, wherein the length between the 5′ ITR and the 3′ ITR is between 4.7 kb and 4.9 kb.

42. The nucleic acid molecule of any one of claims 1-41, wherein the length between the 5′ ITR and the 3′ ITR is about 4.65 kb, about 4.70 kb, about 4.725 kb, about 4.75 kb, about 4.80 kb, about 4.825 kb, about 4.85 kb, or about 4.90 kb.

43. The nucleic acid molecule of any one of claims 1-42, wherein the length between the 5′ ITR and the 3′ ITR is about 4.80 kb.

44. The nucleic acid molecule of any one of claims 1-43, wherein the nucleic acid molecule consists essentially of:
(i) the 5′ inverted terminal repeat (ITR);
(ii) the sequence encoding a base editor operably linked to a first promoter;
(iii) the polyadenylation (polyA) signal;
(iv) the second nucleic acid segment encoding a guide RNA (gRNA) operably linked to a second promoter, wherein the direction of transcription of the second nucleic acid segment is reversed relative to the direction of transcription of the nucleic acid molecule; and
(v) the 3′ ITR,
wherein the length the length between the 5′ ITR and the 3′ ITR is less than about 4.90 kb.

45. The nucleic acid molecule of any one of claims 1-44, wherein the nucleic acid molecule is single-stranded or self-complementary.

46. The nucleic acid molecule of any one of claims 1-29 and 36-45, wherein the nucleic acid molecule comprises the sequence of any one of SEQ ID NOs: 100-102.

47. The nucleic acid molecule of any one of claims 1-29 and 36-46, wherein the nucleic acid molecule comprises the sequence of SEQ ID NO: 100.

48. The nucleic acid molecule of any one of claims 1-47, wherein the direction of transcription of the second nucleic acid segment is reversed relative to the direction of transcription of the first nucleic acid segment.

49. The nucleic acid molecule of any one of claims 1-29 and 36-48, wherein the nucleic acid molecule comprises, from 5′ to 3′:
(i) a 5′ inverted terminal repeat (ITR);
(ii) a first nucleic acid segment comprising a sequence encoding a SaKKH-ABE8e, a SauriCas9-ABE8e, a CjCas9-ABE8e base editor, or a Nme2Cas9 base editor operably linked to a first promoter, wherein the first promoter is selected from the EFS, MeCP2, P3, and U1A promoters; and a bGH polyadenylation (polyA) signal;
(iii) a second nucleic acid segment encoding a guide RNA (gRNA) operably linked to a U6 promoter, wherein the direction of transcription of the second nucleic acid segment is reversed relative to the direction of transcription of the nucleic acid molecule; and
(iv) a 3′ ITR,
wherein the length between the 5′ ITR and the 3′ ITR is less than about 4.90 kb.

50. The nucleic acid vector of claim 49, wherein the first promoter is EFS.

51. The nucleic acid vector of claim 49 or 50, wherein the base editor is SaKKH-ABE8e.

52. The nucleic acid molecule of any one of claims 1-18, 23-26, 30-35, and 38-43, wherein the nucleic acid molecule comprises, from 5′ to 3′:
(i) a 5′ inverted terminal repeat (ITR);
(ii) a first nucleic acid segment comprising a sequence encoding a CjCas9-FERNY-BE3.9 or CjCas9-evoFERNY-BE3.9 base editor operably linked to a first promoter, wherein the first promoter is selected from the EFS, MeCP2, P3, and UlA promoters; and a bGH polyadenylation (polyA) signal;
(iii) a second nucleic acid segment encoding a guide RNA (gRNA) operably linked to a U6 promoter, wherein the direction of transcription of the second nucleic acid segment is reversed relative to the direction of transcription of the nucleic acid molecule; and
(iv) a 3′ ITR,
wherein the length between the 5′ ITR and the 3′ ITR is less than about 4.90 kb.

53. The nucleic acid vector of claim 52, wherein the first promoter is EFS.

54. The nucleic acid vector of claim 52 or 53, wherein the base editor is CjCas9-evoFERNY-BE3.9.

55. An AAV nucleic acid molecule that comprises, in 5′ to 3′ order:
(i) a 5′ inverted terminal repeat (ITR);
(ii) a first nucleic acid segment comprising a transgene operably linked to a first promoter, wherein the first promoter has a length of less than 300 nucleotides; and a transcriptional terminator that does not contain a posttranscriptional response element;
(iii) a second nucleic acid segment operably linked to a second promoter, wherein the direction of transcription of the second nucleic acid segment is reversed relative to the direction of transcription of the first nucleic acid segment; and
(iv) a 3′ ITR,
wherein the length the length between the 5′ ITR and the 3′ ITR is less than about 4.90 kb.

56. The AAV nucleic acid molecule of claim 55, wherein the first nucleic acid segment encodes a base editor, and the second nucleic acid segment encodes a gRNA.

57. The AAV nucleic acid molecule of claim 56, wherein the base editor contains a napDNAbp domain that is a compact Cas9 protein.

58. The AAV nucleic acid molecule of claim 57, wherein the napDNAbp domain is selected from a S. aureus Cas9 (SaCas9) domain, an N. meningitidis 2 Cas9 (Nme2Cas9) domain, a C. jejuni Cas9 (CjCas9) domain, an S. auricularis (SauriCas9) domain, and variants thereof.

59. The nucleic acid molecule of claim 55, wherein the napDNAbp domain is a compact variant of S. pyogenes Cas9 (SpCas9), Cpf1, CasX, CasY, C2c1, C2c2, C2c3, Cas12a, Cas12b, Cas12g, Cas12h, Cas12i, Cas13b, Cas13c, Cas13d, Cas14, Csn2, Cas3, or CasΦ.

60. The nucleic acid molecule of any one of claims 55-59, wherein the napDNAbp domain has nickase activity.

61. A recombinant AAV (rAAV) particle comprising the nucleic acid molecule of any one of claims 1-60 encapsidated in a capsid.

62. The rAAV particle of claim 61, wherein the capsid is of serotype 2, 6, 8, 9, PHP.B, or PHP.eB.

63. The rAAV particle of claim 61 or 62, wherein the capsid is of serotype 8 or serotype 9.

64. The rAAV particle of any one of claims 61-63, wherein the rAAV particle is an rAAV8-Sauri-ABE8e, rAAV9-Sauri-ABE8e, rAAV8-SaKKH-ABE8e, rAAV9-SaKKH-ABE8e, rAAV8-CjCas9-ABE8e, rAAV9-CjCas9-ABE8e, rAAV8-Nme2Cas9-ABE8e, or rAAV9-Nme2Cas9-ABE8e particle.

65. The rAAV particle of any one of claims 61-64, wherein the rAAV particle is an rAAV8-SaKKH-ABE8e particle.

66. A composition comprising a plurality of the rAAV particles of any one of claims 61-65.

67. A composition comprising the nucleic acid molecule of any one of claims 1-60.

68. A pharmaceutical composition comprising the composition of claim 66 or 67 and a pharmaceutically acceptable carrier.

69. A cell comprising the nucleic acid molecule of any one of claims 1-60, the rAAV particle of any one of claims 61-65, or the composition of any one of claims 66-68.

70. The cell of claim 69, wherein the cell is a bacterial cell.

71. The cell of claim 69, wherein the cell is a eukaryotic cell.

72. The cell of claim 69 or 71, wherein the cell is a yeast cell, a plant cell, or a mammalian cell.

73. The cell of claim 69, wherein the cell is a human cell.

74. A kit comprising the nucleic acid molecule of any one of claims 1-60, the rAAV particle of any one of claims 61-65, or the composition of any one of claims 66-68, and instructions for delivery to a cell.

75. A method for editing a target nucleic acid molecule comprising contacting a cell comprising the target nucleic acid molecule with the nucleic acid molecule of any one of claims 1-60, the rAAV particle of any one of claims 61-65, or the composition of any one of claims 66-68.

76. The method of claim 75, wherein the contacting is performed in vitro.

77. The method of claim 75, wherein the contacting is performed in vivo.

78. The method of claim 75, wherein the contacting is performed in a subject.

79. The method of claim 78, wherein the subject has been diagnosed with a disease or disorder.

80. The method of any one of claims 75-79, wherein the guide RNA comprises a guide sequence of at least 10 contiguous nucleotides that is complementary to a target sequence in the target nucleic acid molecule.

81. The method of any one of claims 75-80, wherein the guide RNA is between about 25 and 200 nucleotides in length.

82. The method of any one of claims 75-81, wherein the target sequence comprises a point mutation associated with a disease or disorder.

83. The method of claim 82, wherein the point mutation comprises a T→C point mutation associated with the disease or disorder.

84. The method of claim 82, wherein the point mutation comprises an A→G point mutation associated with the disease or disorder.

85. The method of any one of claims 82-84, wherein the step of editing the target nucleic acid results in correction of the point mutation.

86. The method of any one of claims 75-85, wherein the step of contacting results in the introduction of a splice site.

87. The method of any one of claims 75-85, wherein the step of contacting results in the removal of a splice site.

88. The method of any one of claims 75-85, wherein the step of contacting results in the removal of a stop codon.

89. The method of any one of claims 75-85, wherein the step of contacting results in the introduction of a stop codon.

90. The method of claim 89, wherein the stop codon comprises the nucleic acid sequence 5′-TAG-3′, 5′-TAA-3′, or 5′-TGA-3′.

91. The method of any one of claims 75-90, wherein the target sequence is within a PCSK9 gene or an ANGPTL3 gene.

92. The method of any one of claims 75-91, wherein the step of contacting results in an editing efficiency of at least about 50%, at least about 55%, at least about 60%, at least about 65%, at least about 70%, at least about 75%, at least about 80%, or at least about 85%.

93. The method of any one of claims 75-92, wherein the step of contacting results in a base edit: indel ratio of at least about 5:1, 7:1, 8:1, 9:1, 10:1, 11:1, 12:1, 13:1, or greater than about 15:1.

94. The method of any one of claims 75-93, wherein the step of contacting results in an indel rate of 2.5% or less, 2.0% or less, 1.5% or less, 1.2% or less, or 1.0% or less.

95. The method of any one of claims 75-94, wherein the step of contacting results in minimal bystander editing.

96. A method comprising contacting a cell with the rAAV particle of any one of claims 61-65, or the composition of any one of claims 66-68, wherein the contacting results in the delivery of the nucleic acid molecule into the cell.

97. The method of claim 96, wherein the cell is a human cell.

98. The method of claim 96 or 97, wherein the cell is a cardiac cell or muscle cell.

99. The method of claim 98, wherein the step of contacting results in an editing efficiency of at least about 20%, at least about 22%, at least about 24%, at least about 27%, at least about 30%, at least about 33%, or at least about 36% in the cardiac cell or muscle cell.

100. The method of any one of claims 96-99, wherein the step of contacting results in an indel rate of 2.5% or less, 2.0% or less, 1.5% or less, 1.2% or less, or 1.0% or less in the cardiac cell or muscle cell.

101. A method comprising administering to a subject in need thereof a therapeutically effective amount of the rAAV particle of any one of claims 61-65, the pharmaceutical composition of claim 68, or the cell of any one of claims 69-73.

102. The method of claim 101, wherein the subject is human.

103. The method of claim 101 or 102, wherein the subject has a disease or disorder.

104. The method of any one of claims 101-103, wherein the rAAV particle is administered in a therapeutically effective amount of about 10¹⁵, about 10¹⁴, about 10¹³, about 10¹², about 10¹¹, or less than about 10¹¹ vector genomes (vg) per kg weight of the subject.

105. The method of any one of claims 101-104, wherein the rAAV particle is administered in a therapeutically effective amount of about 8×10¹⁰, 4×10¹⁰, 1×10¹⁰, or less than about 10¹⁰ vg per kg weight of the subject.

106. The method of any one of claims 101-105, wherein the rAAV particle is administered to cardiac tissue of the subject.

107. The method of any one of claims 101-106, wherein the rAAV particle is administered to skeletal muscle tissue of the subject.

108. The method of any one of claims 101-107, wherein the rAAV particle is administered to neuronal tissue of the subject.

109. Use of the nucleic acid molecule of any one of claims 1-60, the rAAV particle of any one of claims 61-65, the composition of any one of claims 66-68, or the cell of any one of claims 69-73 in the manufacture of a medicament.

110. A method of making the rAAV particle of any one of claims 61-65.


SEQUENCE LISTING (738 sequences)

SEQ ID NO: 1-4 - skipped (4 entries)

SEQ ID NO: 5 - protein, 1082 aa, synthetic construct
MAAFKPNPINYILGLAIGIASVGWAMVEIDEEENPIRLIDLGVRVFERAEVPKTGDSLAMARRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVANNAHALQTGDFRTPAELALNKFEKESGHIRNQRGDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSSELQDEIGTAFSLFKTDEDITGRLKDRVQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLVRLNEKGYVEIDHALPFSRTWDDSFNNKVLVLGSENQNKGNQTPYEYFNGKDNSREWQEFKARVETSRFPRSKKQRILLQKFDEDGFKECNLNDTRYVNRFLCQFVADHILLTGKGKRRVFASNGQITNLLRGFWGLRKVRAENDRHHALDAVVVACSTVAMQQKITRFVRYKEMNAFDGKTIDKETGKVLHQKTHFPQPWEFFAQEVMIRVFGKPDGKPEFEEADTPEKLRTLLAEKLSSRPEAVHEYVTPLFVSRAPNRKMSGAHKDTLRSAKRFVKHNEKISVKRVWLTEIKLADLENMVNYKNGREIELYEALKARLEAYGGNAKQAFDPKDNPFYKKGGQLVKAVRVEKTQESGVLLNKKNAYTIADNGDMVRVDVFCKVDKKGKNQYFIVPIYAWQVAENILPDIDCKGYRIDDSYTFCFSLHKYDLIAFQKDEKSKVEFAYYINCDSSNGRFYLAWHDKGSKEQQFRISTQNLVLIQKYQVNELGKEIRPCRLKKRPPVR

SEQ ID NO: 6 - protein, 1082 aa, synthetic construct
MAAFKPNSINYILGLAIGIASVGWAMVEIDEEENPIRLIDLGVRVFERAEVPKTGDSLAMARRLARSVRRLTRRRAHRLLRTRRLLKREGVLQAANFDENGLIKSLPNTPWQLRAAALDRKLTPLEWSAVLLHLIKHRGYLSQRKNEGETADKELGALLKGVAGNAHALQTGDFRTPAELALNKFEKESGHIRNQRSDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSPELQDEIGTAFSLFKTDEDITGRLKDRIQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLGRLNEKGYVEIDAALPFSRTWDDSFNNKVLVLGSENQNKGNQTPYEYFNGKDNSREWQEFKARVETSRFPRSKKQRILLQKFDEDGFKERNLNDTRYVNRFLCQFVADRMRLTGKGKKRVFASNGQITNLLRGFWGLRKVRAENDRHHALDAVVVACSTVAMQQKITRFVRYKEMNAFDGKTIDKETGEVLHQKTHFPQPWEFFAQEVMIRVFGKPDGKPEFEEADTLEKLRTLLAEKLSSRPEAVHEYVTPLFVSRAPNRKMSGQGHMETVKSAKRLDEGVSVLRVPLTQLKLKDLEKMVNREREPKLYEALKARLEAHKDDPAKAFAEPFYKYDKAGNRTQQVKAVRVEQVQKTGVWVRNHNGIADNATMVRVDVFEKGDKYYLVPIYSWQVAKGILPDRAVVQGKDEEDWQLIDDSFNFKFSLHPNDLVEVITKKARMFGYFASCHRGTGNINIRIHDLDHKIGKNGILEGIGVKTALSFQKYQIDELGKEIRPCRLKKRPPVR

SEQ ID NO: 7-18 - skipped (12 entries)

SEQ ID NO: 19 - protein, 1694 aa, synthetic construct
MKRTADGSEFESPKKKRKVSFERNYDPRELRKETYLLYEIKWGKSGKLWRHWCQNNRTQHAEVYFLENIFNARRFNPSTHCSITWYLSWSPCAECSQKIVDFLKEHPNVNLEIYVARLYYHEDERNRQGLRDLVNSGVTIRIMDLPDYNYCWKTFVSDQGGDEDYWPGHFAPWIKQYSLKLSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSGGSGGSTNLSDIIEKETGKQLVIQESILMLPEEVEEVIGNKPESDILVHTAYDESTDENVMLLTSDAPEYKPWALVIQDSNGENKIKMLSGGSKRTADGSEFEPKKKRKV

SEQ ID NO: 20 - protein, 1694 aa, synthetic construct
MKRTADGSEFESPKKKRKVSFERNYDPRELRKETYLLYEIKWGKSGKLWRHWCQNNRTQHAEVYFLENIFNARRFNPSTHCSITWYLSWSPCAECSQKIVDFLKEHPNVNLEIYVARLYYPENERNRQGLRDLVNSGVTIRIMDLPDYNYCWKTFVSDQGGDEDYWPGHFAPWIKQYSLKLSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSGGSGGSTNLSDIIEKETGKQLVIQESILMLPEEVEEVIGNKPESDILVHTAYDESTDENVMLLTSDAPEYKPWALVIQDSNGENKIKMLSGGSKRTADGSEFEPKKKRKV

SEQ ID NO: 21 - protein, 1310 aa, synthetic construct
MKRTADGSEFESPKKKRKVSFERNYDPRELRKETYLLYEIKWGKSGKLWRHWCQNNRTQHAEVYFLENIFNARRFNPSTHCSITWYLSWSPCAECSQKIVDFLKEHPNVNLEIYVARLYYPENERNRQGLRDLVNSGVTIRIMDLPDYNYCWKTFVSDQGGDEDYWPGHFAPWIKQYSLKLSGGSSGGSSGSETPGTSESATPESSGGSSGGSARILAFAIGISSIGWAFSENDELKDCGVRIFTKVENPKTGESLALPRRLARSARKRLARRKARLNHLKHLIANEFKLNYEDYQSFDESLAKAYKGSLISPYELRFRALNELLSKQDFARVILHIAKRRGYDDIKNSDDKEKGAILKAIKQNEEKLANYQSVGEYLYKEYFQKFKENSKEFTNVRNKKESYERCIAQSFLKDELKLIFKKQREFGFSFSKKFEEEVLSVAFYKRALKDFSHLVGNCSFFTDEKRAPKNSPLAFMFVALTRIINLLNNLKNTEGILYTKDDLNALLNEVLKNGTLTYKQTKKLLGLSDDYEFKGEKGTYFIEFKKYKEFIKALGEHNLSQDDLNEIAKDITLIKDEIKLKKALAKYDLNQNQIDSLSKLEFKDHLNISFKALKLVTPLMLEGKKYDEACNELNLKVAINEDKKDFLPAFNETYYKDEVTNPVVLRAIKEYRKVLNALLKKYGKVHKINIELAREVGKNHSQRAKIEKEQNENYKAKKDAELECEKLGLKINSKNILKLRLFKEQKEFCAYSGEKIKISDLQDEKMLEIDHIYPYSRSFDDSYMNKVLVFTKQNQEKLNQTPFEAFGNDSAKWQKIEVLAKNLPTKKQKRILDKNYKDKEQKNFKDRNLNDTRYIARLVLNYTKDYLDFLPLSDDENTKLNDTQKGSKVHVEAKSGMLTSALRHTWGFSAKDRNNHLHHAIDAVIIAYANNSIVKAFSDFKKEQESNSAELYAKKISELDYKNKRKFFEPFSGFRQKVLDKIDEIFVSKPERKKPSGALHEETFRKEEEFYQSYGGKEGVLKALELGKIRKVNGKIVKNGDMFRVDIFKHKKTNKFYAVPIYTMDFALKVLPNKAVARSKKGEIKDWILMDENYEFCFSLYKDSLILIQTKDMQEPEFVYYNAFTSSTVSLIVSKHDNKFETLSKNQKILFKNANEKEVIAKSIGIQNLKVFEKYIVSALGEVTKAEFRQREDFKKSGGSGGSGGSTNLSDIIEKETGKQLVIQESILMLPEEVEEVIGNKPESDILVHTAYDESTDENVMLLTSDAPEYKPWALVIQDSNGENKIKMLSGGSKRTADGSEFEPKKKRKV

SEQ ID NO: 22 - protein, 1310 aa, synthetic construct
MKRTADGSEFESPKKKRKVSFERNYDPRELRKETYLLYEIKWGKSGKLWRHWCQNNRTQHAEVYFLENIFNARRFNPSTHCSITWYLSWSPCAECSQKIVDFLKEHPNVNLEIYVARLYYHEDERNRQGLRDLVNSGVTIRIMDLPDYNYCWKTFVSDQGGDEDYWPGHFAPWIKQYSLKLSGGSSGGSSGSETPGTSESATPESSGGSSGGSARILAFAIGISSIGWAFSENDELKDCGVRIFTKVENPKTGESLALPRRLARSARKRLARRKARLNHLKHLIANEFKLNYEDYQSFDESLAKAYKGSLISPYELRFRALNELLSKQDFARVILHIAKRRGYDDIKNSDDKEKGAILKAIKQNEEKLANYQSVGEYLYKEYFQKFKENSKEFTNVRNKKESYERCIAQSFLKDELKLIFKKQREFGFSFSKKFEEEVLSVAFYKRALKDFSHLVGNCSFFTDEKRAPKNSPLAFMFVALTRIINLLNNLKNTEGILYTKDDLNALLNEVLKNGTLTYKQTKKLLGLSDDYEFKGEKGTYFIEFKKYKEFIKALGEHNLSQDDLNEIAKDITLIKDEIKLKKALAKYDLNQNQIDSLSKLEFKDHLNISFKALKLVTPLMLEGKKYDEACNELNLKVAINEDKKDFLPAFNETYYKDEVTNPVVLRAIKEYRKVLNALLKKYGKVHKINIELAREVGKNHSQRAKIEKEQNENYKAKKDAELECEKLGLKINSKNILKLRLFKEQKEFCAYSGEKIKISDLQDEKMLEIDHIYPYSRSFDDSYMNKVLVFTKQNQEKLNQTPFEAFGNDSAKWQKIEVLAKNLPTKKQKRILDKNYKDKEQKNFKDRNLNDTRYIARLVLNYTKDYLDFLPLSDDENTKLNDTQKGSKVHVEAKSGMLTSALRHTWGFSAKDRNNHLHHAIDAVIIAYANNSIVKAFSDFKKEQESNSAELYAKKISELDYKNKRKFFEPFSGFRQKVLDKIDEIFVSKPERKKPSGALHEETFRKEEEFYQSYGGKEGVLKALELGKIRKVNGKIVKNGDMFRVDIFKHKKTNKFYAVPIYTMDFALKVLPNKAVARSKKGEIKDWILMDENYEFCFSLYKDSLILIQTKDMQEPEFVYYNAFTSSTVSLIVSKHDNKFETLSKNQKILFKNANEKEVIAKSIGIQNLKVFEKYIVSALGEVTKAEFRQREDFKKSGGSGGSGGSTNLSDIIEKETGKQLVIQESILMLPEEVEEVIGNKPESDILVHTAYDESTDENVMLLTSDAPEYKPWALVIQDSNGENKIKMLSGGSKRTADGSEFEPKKKRKV

SEQ ID NO: 23-32 - skipped (10 entries)

SEQ ID NO: 33 - protein, 166 aa, synthetic construct
SEVEFSHEYWMRHALTLAKRARDEGEVPVGAVLVLNNRVIGEGWNRAIGLYDPTAHAEIMALRQGGLVMQNYRLIDATLYVTFEPCVMCAGAMIHSRIGRVVFGVRNSKRGAAGSLMNVLNYPGMDHRVEITEGILANECAALLCDFYRMPRQVFNAQKKAQSSIN

SEQ ID NO: 34-73 - skipped (40 entries)

SEQ ID NO: 74 - protein, 1368 aa, Streptococcus pyogenes [sequence omitted]

SEQ ID NO: 75-77 - skipped (3 entries)

SEQ ID NO: 78 - RNA, 91 nt, Staphylococcus aureus
  source 1..91: mol_type = genomic RNA
gttttagtactctgtaatgaaaattacagaatctactaaaacaaggcaaaatgccgtgtttatctcgtcaacttgttggcgagattttttt

SEQ ID NO: 79-99 - skipped (21 entries)

SEQ ID NO: 100 - DNA, 5064 bp, synthetic construct
  source 1..5064: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcctctagaattcgctagctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggtgccaccatgaaacggacagccgacggaagcgagttcgagtcaccaaagaagaagcggaaagtctctgaggtggagttttcccacgagtactggatgagacatgccctgaccctggccaagagggcacgggatgagagggaggtgcctgtgggagccgtgctggtgctgaacaatagagtgatcggcgagggctggaacagagccatcggcctgcacgacccaacagcccatgccgaaattatggccctgagacagggcggcctggtcatgcagaactacagactgattgacgccaccctgtacgtgacattcgagccttgcgtgatgtgcgccggcgccatgatccactctaggatcggccgcgtggtgtttggcgtgaggaactcaaaaagaggcgccgcaggctccctgatgaacgtgctgaactaccccggcatgaatcaccgcgtcgaaattaccgagggaatcctggcagatgaatgtgccgccctgctgtgcgatttctatcggatgcctagacaggtgttcaatgctcagaagaaggcccagagctccatcaactccggaggatctagcggaggctcctctggctctgagacacctggcacaagcgagagcgcaacacctgaaagcagcgggggcagcagcggggggtcagggaagcgaaattacattctggggctggccattggcattacatcagtgggctatggcatcattgactacgagacaagggacgtgatcgacgccggcgtgagactgttcaaggaggccaacgtggagaacaatgagggccggagatccaagaggggagcaaggcgcctgaagcggagaaggcgccacagaatccagagagtgaagaagctgctgttcgattacaacctgctgaccgaccactccgagctgtctggcatcaatccttatgaggccagagtgaagggcctgtcccagaagctgtctgaggaggagtttagcgccgccctgctgcacctggcaaagaggagaggcgtgcacaacgtgaatgaggtggaggaggacaccggcaacgagctgtccacaaaggagcagatcagccgcaattccaaggccctggaggagaagtatgtggccgagctgcagctggagcggctgaagaaggatggcgaggtgaggggctccatcaatcgcttcaagacctctgactacgtgaaggaggccaagcagctgctgaaggtgcagaaggcctaccaccagctggatcagtcctttatcgatacatatatcgacctgctggagacaaggcgcacatactatgagggaccaggagagggctctcccttcggctggaaggacatcaaggagtggtacgagatgctgatgggccactgcacctattttccagaggagctgagaagcgtgaagtacgcctataacgccgatctgtacaacgccctgaatgacctgaacaacctggtcatcaccagggatgagaacgagaagctggagtactatgagaagttccagatcatcgagaacgtgttcaagcagaagaagaagcctacactgaagcagatcgccaaggagatcctggtgaacgaggaggacatcaagggctaccgcgtgacctccacaggcaagccagagttcaccaatctgaaggtgtatcacgatatcaaggacatcacagcccggaaggagatcatcgagaacgccgagctgctggatcagatcgccaagatcctgaccatctatcagagctccgaggacatccaggaggagctgaccaacctgaatagcgagctgacacaggaggagatcgagcagatcagcaatctgaagggctacaccggcacacacaacctgagcctgaaggccatcaatctgatcctggatgagctgtggcacacaaacgacaatcagatcgccatctttaaccggctgaagctggtgccaaagaaggtggacctgtcccagcagaaggagatcccaaccacactggtggacgatttcatcctgtctcccgtggtgaagcggagcttcatccagagcatcaaagtgatcaacgccatcatcaagaagtacggcctgcccaatgatatcatcatcgagctggccagggagaagaactccaaggacgcccagaagatgatcaatgagatgcagaagaggaaccgccagaccaatgagcggatcgaggagatcatcagaaccacaggcaaggagaacgccaagtacctgatcgagaagatcaagctgcacgatatgcaggagggcaagtgtctgtattctctggaggccatccctctggaggacctgctgaacaatccattcaactacgaggtggatcacatcatcccccggagcgtgagcttcgacaattcttttaacaataaggtgctggtgaagcaggaggagaacagcaagaagggcaataggacccctttccagtacctgtctagctccgattctaagatcagctacgagacattcaagaagcacatcctgaatctggccaagggcaagggccgcatcagcaagaccaagaaggagtacctgctggaggagcgggacatcaacagattctccgtgcagaaggacttcatcaaccggaatctggtggacaccagatacgccacacgcggcctgatgaatctgctgcggtcttatttcagagtgaacaatctggatgtgaaggtgaagagcatcaacggcggcttcacctcctttctgcggagaaagtggaagtttaagaaggagcgcaacaagggctataagcaccacgccgaggatgccctgatcatcgccaatgccgacttcatctttaaggagtggaagaagctggacaaggccaagaaagtgatggagaaccagatgttcgaggagaagcaggccgagagcatgcccgagatcgagacagagcaggagtacaaggagattttcatcacacctcaccagatcaagcacatcaaggacttcaaggactacaagtattctcacagggtggataagaagcccaaccgcgagctgatcaatgacaccctgtatagcacacggaaggacgataagggcaataccctgatcgtgaacaatctgaacggcctgtacgacaaggataatgacaagctgaagaagctgatcaacaagtctcccgagaagctgctgatgtaccaccacgatcctcagacatatcagaagctgaagctgatcatggagcagtacggcgacgagaagaacccactgtataagtactatgaggagacaggcaactacctgacaaagtatagcaagaaggataatggccccgtgatcaagaagatcaagtactatggcaacaagctgaatgcccacctggacatcaccgacgattaccctaactctcgcaataaggtggtgaagctgagcctgaagccataccggttcgacgtgtacctggacaacggcgtgtataagtttgtgacagtgaagaatctggatgtgatcaagaaggagaactactatgaggtgaacagcaagtgctacgaggaggccaagaagctgaagaagatcagcaaccaggccgagttcatcgcctctttttacaacaatgacctgatcaagatcaatggcgagctgtatagagtgatcggcgtgaacaatgatctgctgaacagaatcgaagtgaatatgatcgacatcacctacagggagtatctggagaacatgaatgataagaggccccctcgcatcatcaagaccatcgcctctaagacacagagcatcaagaagtacagcacagacatcctggggaacctgtatgaagtcaagagcaagaaacatcctcagattatcaagaaaggctctggcggctcaaaaagaaccgccgacggcagcgaattcgagcccaagaagaagaggaaagtctaatagatctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggctcgagcggcccaagcttaaaaaaatctcgccaacaagttgacgagataaacacggcattttgccttgttttagtagattctgtaattttcattacagagtactaaaaccgccgttgctccaaggtatggcggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattctaattatctctctaacagccttgtatcgtatatgcaaatatgaaggaatcatgggaaataggccctcttcctgcccgaccttgcggccgcctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 101 - DNA, 5064 bp, synthetic construct
  source 1..5064: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcctctagaattcgctagctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggtgccaccatgaaacggacagccgacggaagcgagttcgagtcaccaaagaagaagcggaaagtctctgaggtggagttttcccacgagtactggatgagacatgccctgaccctggccaagagggcacgggatgagagggaggtgcctgtgggagccgtgctggtgctgaacaatagagtgatcggcgagggctggaacagagccatcggcctgcacgacccaacagcccatgccgaaattatggccctgagacagggcggcctggtcatgcagaactacagactgattgacgccaccctgtacgtgacattcgagccttgcgtgatgtgcgccggcgccatgatccactctaggatcggccgcgtggtgtttggcgtgaggaactcaaaaagaggcgccgcaggctccctgatgaacgtgctgaactaccccggcatgaatcaccgcgtcgaaattaccgagggaatcctggcagatgaatgtgccgccctgctgtgcgatttctatcggatgcctagacaggtgttcaatgctcagaagaaggcccagagctccatcaactccggaggatctagcggaggctcctctggctctgagacacctggcacaagcgagagcgcaacacctgaaagcagcgggggcagcagcggggggtcagggaagcgaaattacattctggggctggccattggcattacatcagtgggctatggcatcattgactacgagacaagggacgtgatcgacgccggcgtgagactgttcaaggaggccaacgtggagaacaatgagggccggagatccaagaggggagcaaggcgcctgaagcggagaaggcgccacagaatccagagagtgaagaagctgctgttcgattacaacctgctgaccgaccactccgagctgtctggcatcaatccttatgaggccagagtgaagggcctgtcccagaagctgtctgaggaggagtttagcgccgccctgctgcacctggcaaagaggagaggcgtgcacaacgtgaatgaggtggaggaggacaccggcaacgagctgtccacaaaggagcagatcagccgcaattccaaggccctggaggagaagtatgtggccgagctgcagctggagcggctgaagaaggatggcgaggtgaggggctccatcaatcgcttcaagacctctgactacgtgaaggaggccaagcagctgctgaaggtgcagaaggcctaccaccagctggatcagtcctttatcgatacatatatcgacctgctggagacaaggcgcacatactatgagggaccaggagagggctctcccttcggctggaaggacatcaaggagtggtacgagatgctgatgggccactgcacctattttccagaggagctgagaagcgtgaagtacgcctataacgccgatctgtacaacgccctgaatgacctgaacaacctggtcatcaccagggatgagaacgagaagctggagtactatgagaagttccagatcatcgagaacgtgttcaagcagaagaagaagcctacactgaagcagatcgccaaggagatcctggtgaacgaggaggacatcaagggctaccgcgtgacctccacaggcaagccagagttcaccaatctgaaggtgtatcacgatatcaaggacatcacagcccggaaggagatcatcgagaacgccgagctgctggatcagatcgccaagatcctgaccatctatcagagctccgaggacatccaggaggagctgaccaacctgaatagcgagctgacacaggaggagatcgagcagatcagcaatctgaagggctacaccggcacacacaacctgagcctgaaggccatcaatctgatcctggatgagctgtggcacacaaacgacaatcagatcgccatctttaaccggctgaagctggtgccaaagaaggtggacctgtcccagcagaaggagatcccaaccacactggtggacgatttcatcctgtctcccgtggtgaagcggagcttcatccagagcatcaaagtgatcaacgccatcatcaagaagtacggcctgcccaatgatatcatcatcgagctggccagggagaagaactccaaggacgcccagaagatgatcaatgagatgcagaagaggaaccgccagaccaatgagcggatcgaggagatcatcagaaccacaggcaaggagaacgccaagtacctgatcgagaagatcaagctgcacgatatgcaggagggcaagtgtctgtattctctggaggccatccctctggaggacctgctgaacaatccattcaactacgaggtggatcacatcatcccccggagcgtgagcttcgacaattcttttaacaataaggtgctggtgaagcaggaggagaacagcaagaagggcaataggacccctttccagtacctgtctagctccgattctaagatcagctacgagacattcaagaagcacatcctgaatctggccaagggcaagggccgcatcagcaagaccaagaaggagtacctgctggaggagcgggacatcaacagattctccgtgcagaaggacttcatcaaccggaatctggtggacaccagatacgccacacgcggcctgatgaatctgctgcggtcttatttcagagtgaacaatctggatgtgaaggtgaagagcatcaacggcggcttcacctcctttctgcggagaaagtggaagtttaagaaggagcgcaacaagggctataagcaccacgccgaggatgccctgatcatcgccaatgccgacttcatctttaaggagtggaagaagctggacaaggccaagaaagtgatggagaaccagatgttcgaggagaagcaggccgagagcatgcccgagatcgagacagagcaggagtacaaggagattttcatcacacctcaccagatcaagcacatcaaggacttcaaggactacaagtattctcacagggtggataagaagcccaaccgcaagctgatcaatgacaccctgtatagcacacggaaggacgataagggcaataccctgatcgtgaacaatctgaacggcctgtacgacaaggataatgacaagctgaagaagctgatcaacaagtctcccgagaagctgctgatgtaccaccacgatcctcagacatatcagaagctgaagctgatcatggagcagtacggcgacgagaagaacccactgtataagtactatgaggagacaggcaactacctgacaaagtatagcaagaaggataatggccccgtgatcaagaagatcaagtactatggcaacaagctgaatgcccacctggacatcaccgacgattaccctaactctcgcaataaggtggtgaagctgagcctgaagccataccggttcgacgtgtacctggacaacggcgtgtataagtttgtgacagtgaagaatctggatgtgatcaagaaggagaactactatgaggtgaacagcaagtgctacgaggaggccaagaagctgaagaagatcagcaaccaggccgagttcatcgcctctttttacaagaatgacctgatcaagatcaatggcgagctgtatagagtgatcggcgtgaacaatgatctgctgaacagaatcgaagtgaatatgatcgacatcacctacagggagtatctggagaacatgaatgataagaggccccctcatatcatcaagaccatcgcctctaagacacagagcatcaagaagtacagcacagacatcctggggaacctgtatgaagtcaagagcaagaaacatcctcagattatcaagaaaggctctggcggctcaaaaagaaccgccgacggcagcgaattcgagcccaagaagaagaggaaagtctaatagatctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggctcgagcggcccaagcttaaaaaaatctcgccaacaagttgacgagataaacacggcattttgccttgttttagtagattctgtaattttcattacagagtactaaaaccgccgttgctccaaggtatggcggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattctaattatctctctaacagccttgtatcgtatatgcaaatatgaaggaatcatgggaaataggccctcttcctgcccgaccttgcggccgcctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 102 - DNA, 5088 bp, synthetic construct
  source 1..5088: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcctctagaattcgctagctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggtgccaccatgaaacggacagccgacggaagcgagttcgagtcaccaaagaagaagcggaaagtctctgaggtggagttttcccacgagtactggatgagacatgccctgaccctggccaagagggcacgggatgagagggaggtgcctgtgggagccgtgctggtgctgaacaatagagtgatcggcgagggctggaacagagccatcggcctgcacgacccaacagcccatgccgaaattatggccctgagacagggcggcctggtcatgcagaactacagactgattgacgccaccctgtacgtgacattcgagccttgcgtgatgtgcgccggcgccatgatccactctaggatcggccgcgtggtgtttggcgtgaggaactcaaaaagaggcgccgcaggctccctgatgaacgtgctgaactaccccggcatgaatcaccgcgtcgaaattaccgagggaatcctggcagatgaatgtgccgccctgctgtgcgatttctatcggatgcctagacaggtgttcaatgctcagaagaaggcccagagctccatcaactccggaggatctagcggaggctcctctggctctgagacacctggcacaagcgagagcgcaacacctgaaagcagcgggggcagcagcggggggtcaatgcaggagaaccagcagaagcagaactacatcctgggcctggccatcggaatcaccagcgtcggctacggactgatcgatagcaagacaagagaagtgatcgacgccggcgttagactctttccagaagctgatagcgagaacaactccaaccgcagaagcaagcggggcgccagacggttaaaacggagaagaatccaccggctgaaccgggtcaaagacctgctcgctgattaccagatgatcgatcttaacaatgttcctaagagcaccgacccctacaccatcagagtgaagggcctccgggagcctctgacaaaagaagaattcgccatcgccctcctgcatatcgctaagagaagaggcctgcacaacatcagtgtgtccatgggcgacgaagagcaggacaatgaactgagcaccaagcagcagctgcaaaagaatgcccagcaactgcaggacaagtatgtgtgcgaactgcagttagaacggctgaccaacatcaacaaggtcagaggcgagaagaacagatttaagacagaggactttgtgaaagaagtgaaacagctgtgcgaaacccagagacagtaccacaacatcgacgaccaattcatccagcagtacatcgacctggtgtctacaagacgggagtacttcgagggccccggcaacggctctccatacggctgggacggcgacctgctgaagtggtacgagaagctgatgggcagatgcacctatttccccgaagaactgaggtccgtgaagtacgcctacagcgccgacctcttcaacgccctgaacgacctgaacaacctcgttgtgaccagggatgacaatccaaagcttgagtactacgagaagtaccacattattgagaacgtgttcaagcaaaagaagaatcccacactcaaacaaatcgccaaagagatcggcgtgcaagattacgacatccggggctatagaatcacaaagagcggcaaacctcagttcacctcttttaagctgtatcacgacctgaagaacatcttcgagcaggccaaatacctggaagatgtggaaatgctggacgagatcgccaagatcctgaccatctaccaggatgagattagcatcaagaaagccctggaccagctgcccgaactgctgacagagagcgagaaatctcagatcgcacagctcaccggctatacaggcacccacagactgagcctgaagtgcatccacattgtgatcgacgagctgtgggagagccccgagaaccagatggaaatctttaccagactgaatctgaaacctaagaaggtggaaatgagcgagatcgacagcatacccaccaccctggtcgacgagttcatcctctcacctgtggtgaagcgggccttcatccagagcatcaaggtaatcaacgcagtgatcaatcggttcggcctgccagaggacatcatcatcgagctggccagagaaaagaatagcaaggatcggagaaagttcattaacaagctgcagaaacaaaatgaggccacaagaaagaaaatcgaacagctgctggccaagtacggcaacaccaatgccaagtacatgatcgagaagatcaagctgcacgacatgcaggagggcaagtgcctgtacagcctggaggctattcctctggaagacctgctgagcaacccgacacactacgaagttgaccacattatccccagatctgtgagctttgacaacagcctgaacaacaaagtgctggtgaaacaaagcgaaaacagcaagaagggcaatcgcaccccttaccagtacctgagcagcaacgagtctaagattagctacaaccagtttaagcagcacatcctgaacctgagcaaggccaaggacagaatcagcaagaaaaaaagagatatgctgctggaagagagagatatcaacaagttcgaagtgcagaaggaattcattaaccggaacctggtggatacacggtacgccaccagagaactgtctaacctgctgaagacctacttcagcacccatgactacgccgtgaaggtgaagaccatcaacggcggcttcactaaccacctgaggaaggtgtgggatttcaagaagcacagaaaccacggctacaagcaccacgccgaagatgccctggtgatcgccaacgccgacttcctgtttaagacacataaggccctgcggagaaccgataagatcctggaacaacctggcctggaagtgaatgatacaaccgtgaaagtggacaccgaggaaaaataccaggagctgttcgagacacctaagcaagtgaagaacatcaagcagttccgggacttcaagtacagccaccgagtggacaagaagcctaaccggcagcttatcaacgacacactgtactccaccagagagattgatggcgaaacctacgtggtgcagacccttaaggatctgtacgccaaggacaacgagaaagtgaagaagctgttcaccgaaagacctcagaagatcctgatgtaccagcacgaccctaagaccttcgagaaactgatgacaatcctgaaccagtacgctgaggccaagaaccctctggctgcttattacgaggacaaaggcgagtacgtgaccaagtacgccaagaaaggcaatggacctgccatccacaagatcaagtatatcgataagaagcttggatcttacctggatgttagcaacaagtatcctgagacacagaacaagcttgtgaagctgtccctgaagagctttagattcgacatctacaagtgtgaacagggctacaagatggtgtccatcggatacctggacgtgctgaagaaagataactactactacatccctaaggacaagtacgaggccgagaagcagaaaaagaagatcaaggaatctgatctttttgtgggcagcttctactacaacgacctcatcatgtacgaggatgaactgttcagagtgataggagtgaacagcgacatcaacaatctggttgagctaaacatggtcgacattacctacaaggacttctgcgaggtgaacaacgtgacaggcgagaaaagaatcaaaaagactatcggcaagcgcgtggtcctgatcgagaagtacaccacagatattctaggcaacctgtacaagactcccctgcctaagaagccccagcttatcttcaagcggggagaactgtctggcggctcaaaaagaaccgccgacggcagcgaattcgagcccaagaagaagaggaaagtctaatagatctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggctcgagcggcccaagcttaaaaaaatctcgccaacaagttgacgagataaacacggcattttgccttgttttagtagattctgtaattttcattacagagtactaaaaccgttgctccaaggtatgggtgcggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattctaattatctctctaacagccttgtatcgtatatgcaaatatgaaggaatcatgggaaataggccctcttcctgcccgaccttgcggccgcctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 103-106 - skipped (4 entries)

SEQ ID NO: 107 - protein, 1368 aa, Streptococcus pyogenes
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 108-132 - skipped (25 entries)

SEQ ID NO: 133 - protein, 384 aa, Homo sapiens
MKPHFRNTVERMYRDTFSYNFYNRPILSRRNTVWLCYEVKTKGPSRPPLDAKIFRGQVYSELKYHPEMRFFHWFSKWRKLHRDQEYEVTWYISWSPCTKCTRDMATFLAEDPKVTLTIFVARLYYFWDPDYQEALRSLCQKRDGPRATMKIMNYDEFQHCWSKFVYSQRELFEPWNNLPKYYILLHIMLGEILRHSMDPPTFTFNFNNEPWVRGRHETYLCYEVERMHNDTWVLLNQRRGFLCNQAPHKHGFLEGRHAELCFLDVIPFWKLDLDQDYRVTCFTSWSPCFSCAQEMAKFISKNKHVSLCIFTARIYDDQGRCQEGLRTLAEAGAKISIMTYSEFKHCWDTFVDHQGCPFQPWDGLDEHSQDLSGRLRAILQNQEN

SEQ ID NO: 134 - protein, 373 aa, Homo sapiens
MKPHFRNTVERMYRDTFSYNFYNRPILSRRNTVWLCYEVKTKGPSRPRLDAKIFRGQVYSQPEHHAEMCFLSWFCGNQLPAYKCFQITWFVSWTPCPDCVAKLAEFLAEHPNVTLTISAARLYYYWERDYRRALCRLSQAGARVKIMDDEEFAYCWENFVYSEGQPFMPWYKFDDNYAFLHRTLKEILRNPMEAMYPHIFYFHFKNLRKAYGRNESWLCFTMEVVKHHSPVSWKRGVFRNQVDPETHCHAERCFLSWFCDDILSPNTNYEVTWYTSWSPCPECAGEVAEFLARHSNVNLTIFTARLYYFWDTDYQEGLRSLSQEGASVEIMGYKDFKYCWENFVYNDDEPFKPWKGLKYNFLFLDSKLQEILE

SEQ ID NO: 135-170 - skipped (36 entries)

SEQ ID NO: 171 - protein, 1291 aa, synthetic construct
MKRTADGSEFESPKKKRKVSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSGKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKGSGGSKRTADGSEFEPKKKRKV

SEQ ID NO: 172 - protein, 1298 aa, synthetic construct
MKRTADGSEFESPKKKRKVSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSQENQQKQNYILGLAIGITSVGYGLIDSKTREVIDAGVRLFPEADSENNSNRRSKRGARRLKRRRIHRLNRVKDLLADYQMIDLNNVPKSTDPYTIRVKGLREPLTKEEFAIALLHIAKRRGLHNISVSMGDEEQDNELSTKQQLQKNAQQLQDKYVCELQLERLTNINKVRGEKNRFKTEDFVKEVKQLCETQRQYHNIDDQFIQQYIDLVSTRREYFEGPGNGSPYGWDGDLLKWYEKLMGRCTYFPEELRSVKYAYSADLFNALNDLNNLVVTRDDNPKLEYYEKYHIIENVFKQKKNPTLKQIAKEIGVQDYDIRGYRITKSGKPQFTSFKLYHDLKNIFEQAKYLEDVEMLDEIAKILTIYQDEISIKKALDQLPELLTESEKSQIAQLTGYTGTHRLSLKCIHIVIDELWESPENQMEIFTRLNLKPKKVEMSEIDSIPTTLVDEFILSPVVKRAFIQSIKVINAVINRFGLPEDIIIELAREKNSKDRRKFINKLQKQNEATRKKIEQLLAKYGNTNAKYMIEKIKLHDMQEGKCLYSLEAIPLEDLLSNPTHYEVDHIIPRSVSFDNSLNNKVLVKQSENSKKGNRTPYQYLSSNESKISYNQFKQHILNLSKAKDRISKKKRDMLLEERDINKFEVQKEFINRNLVDTRYATRELSNLLKTYFSTHDYAVKVKTINGGFTNHLRKVWDFKKHRNHGYKHHAEDALVIANADFLFKTHKALRRTDKILEQPGLEVNDTTVKVDTEEKYQELFETPKQVKNIKQFRDFKYSHRVDKKPNRQLINDTLYSTREIDGETYVVQTLKDLYAKDNEKVKKLFTERPQKILMYQHDPKTFEKLMTILNQYAEAKNPLAAYYEDKGEYVTKYAKKGNGPAIHKIKYIDKKLGSYLDVSNKYPETQNKLVKLSLKSFRFDIYKCEQGYKMVSIGYLDVLKKDNYYYIPKDKYEAEKQKKKIKESDLFVGSFYYNDLIMYEDELFRVIGVNSDINNLVELNMVDITYKDFCEVNNVTGEKRIKKTIGKRVVLIEKYTTDILGNLYKTPLPKKPQLIFKRGELSGGSKRTADGSEFEPKKKRKV

SEQ ID NO: 173-180 - skipped (8 entries)

SEQ ID NO: 181 - protein, 1291 aa, synthetic construct
MKRTADGSEFESPKKKRKVSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSGKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRKLINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYKNDLIKINGELYRVIGVNNDLLNRIEVNMIDITYREYLENMNDKRPPHIIKTIASKTQSIKKYSTDILGNLYEVKSKKHPQIIKKGSGGSKRTADGSEFEPKKKRKV

SEQ ID NO: 182 - protein, 1221 aa, synthetic construct
MKRTADGSEFESPKKKRKVSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSARILAFAIGISSIGWAFSENDELKDCGVRIFTKVENPKTGESLALPRRLARSARKRLARRKARLNHLKHLIANEFKLNYEDYQSFDESLAKAYKGSLISPYELRFRALNELLSKQDFARVILHIAKRRGYDDIKNSDDKEKGAILKAIKQNEEKLANYQSVGEYLYKEYFQKFKENSKEFTNVRNKKESYERCIAQSFLKDELKLIFKKQREFGFSFSKKFEEEVLSVAFYKRALKDFSHLVGNCSFFTDEKRAPKNSPLAFMFVALTRIINLLNNLKNTEGILYTKDDLNALLNEVLKNGTLTYKQTKKLLGLSDDYEFKGEKGTYFIEFKKYKEFIKALGEHNLSQDDLNEIAKDITLIKDEIKLKKALAKYDLNQNQIDSLSKLEFKDHLNISFKALKLVTPLMLEGKKYDEACNELNLKVAINEDKKDFLPAFNETYYKDEVTNPVVLRAIKEYRKVLNALLKKYGKVHKINIELAREVGKNHSQRAKIEKEQNENYKAKKDAELECEKLGLKINSKNILKLRLFKEQKEFCAYSGEKIKISDLQDEKMLEIDHIYPYSRSFDDSYMNKVLVFTKQNQEKLNQTPFEAFGNDSAKWQKIEVLAKNLPTKKQKRILDKNYKDKEQKNFKDRNLNDTRYIARLVLNYTKDYLDFLPLSDDENTKLNDTQKGSKVHVEAKSGMLTSALRHTWGFSAKDRNNHLHHAIDAVIIAYANNSIVKAFSDFKKEQESNSAELYAKKISELDYKNKRKFFEPFSGFRQKVLDKIDEIFVSKPERKKPSGALHEETFRKEEEFYQSYGGKEGVLKALELGKIRKVNGKIVKNGDMFRVDIFKHKKTNKFYAVPIYTMDFALKVLPNKAVARSKKGEIKDWILMDENYEFCFSLYKDSLILIQTKDMQEPEFVYYNAFTSSTVSLIVSKHDNKFETLSKNQKILFKNANEKEVIAKSIGIQNLKVFEKYIVSALGEVTKAEFRQREDFKKSGGSKRTADGSEFEPKKKRKV

SEQ ID NO: 183 - protein, 1319 aa, synthetic construct
MKRTADGSEFESPKKKRKVSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSAAFKPNPINYILGLAIGIASVGWAMVEIDEEENPIRLIDLGVRVFERAEVPKTGDSLAMARRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVANNAHALQTGDFRTPAELALNKFEKESGHIRNQRGDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSSELQDEIGTAFSLFKTDEDITGRLKDRVQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLVRLNEKGYVEIDHALPFSRTWDDSFNNKVLVLGSENQNKGNQTPYEYFNGKDNSREWQEFKARVETSRFPRSKKQRILLQKFDEDGFKECNLNDTRYVNRFLCQFVADHILLTGKGKRRVFASNGQITNLLRGFWGLRKVRAENDRHHALDAVVVACSTVAMQQKITRFVRYKEMNAFDGKTIDKETGKVLHQKTHFPQPWEFFAQEVMIRVFGKPDGKPEFEEADTPEKLRTLLAEKLSSRPEAVHEYVTPLFVSRAPNRKMSGAHKDTLRSAKRFVKHNEKISVKRVWLTEIKLADLENMVNYKNGREIELYEALKARLEAYGGNAKQAFDPKDNPFYKKGGQLVKAVRVEKTQESGVLLNKKNAYTIADNGDMVRVDVFCKVDKKGKNQYFIVPIYAWQVAENILPDIDCKGYRIDDSYTFCFSLHKYDLIAFQKDEKSKVEFAYYINCDSSNGRFYLAWHDKGSKEQQFRISTQNLVLIQKYQVNELGKEIRPCRLKKRPPVRSGGSKRTADGSEFEPKKKRKV

SEQ ID NO: 184-275 - skipped (92 entries)

SEQ ID NO: 276 - protein, 198 aa, Homo sapiens [sequence omitted]

SEQ ID NO: 277 - protein, 198 aa, Mus musculus
MDSLLMKQKKFLYHFKNVRWAKGRHETYLCYVVKRRDSATSCSLDFGHLRNKSGCHVELLFLRYISDWDLDPGRCYRVTWFTSWSPCYDCARHVAEFLRWNPNLSLRIFTARLYFCEDRKAEPEGLRRLHRAGVQIGIMTFKDYFYCWNTFVENRERTFKAWEGLHENSVRLTRQLRRILLPLYEVDDLRDAFRMLGF

SEQ ID NO: 278-280 - skipped (3 entries)

SEQ ID NO: 281 - protein, 429 aa, Rattus norvegicus
MGPFCLGCSHRKCYSPIRNLISQETFKFHFKNLRYAIDRKDTFLCYEVTRKDCDSPVSLHHGVFKNKDNIHAEICFLYWFHDKVLKVLSPREEFKITWYMSWSPCFECAEQVLRFLATHHNLSLDIFSSRLYNIRDPENQQNLCRLVQEGAQVAAMDLYEFKKCWKKFVDNGGRRFRPWKKLLTNFRYQDSKLQEILRPCYIPVPSSSSSTLSNICLTKGLPETRFCVERRRVHLLSEEEFYSQFYNQRVKHLCYYHGVKPYLCYQLEQFNGQAPLKGCLLSEKGKQHAEILFLDKIRSMELSQVIITCYLTWSPCPNCAWQLAAFKRDRPDLILHIYTSRLYFHWKRPFQKGLCSLWQSGILVDVMDLPQFTDCWTNFVNPKRPFWPWKGLEIISRRTQRRLHRIKESWGLQDLVNDFGNLQLGPPMS

SEQ ID NO: 282-291 - skipped (10 entries)

SEQ ID NO: 292 - protein, 236 aa, Homo sapiens
MTSEKGPSTGDPTLRRRIEPWEFDVFYDPRELRKEACLLYEIKWGMSRKIWRSSGKNTTNHVEVNFIKKFTSERDFHPSMSCSITWFLSWSPCWECSQAIREFLSRHPGVTLVIYVARLFWHMDQQNRQGLRDLVNSGVTIQIMRASEYYHCWRNFVNYPPGDEAHWPQYPPLWMMLYALELHCIILSLPPCLKISRRWQNHLTFFRLHLQNCHYQTIPPHILLATGLIHPSVAWR

SEQ ID NO: 293 - protein, 229 aa, Mus musculus
MSSETGPVAVDPTLRRRIEPHEFEVFFDPRELRKETCLLYEINWGGRHSVWRHTSQNTSNHVEVNFLEKFTTERYFRPNTRCSITWFLSWSPCGECSRAITEFLSRHPYVTLFIYIARLYHHTDQRNRQGLRDLISSGVTIQIMTEQEYCYCWRNFVNYPPSNEAYWPRYPHLWVKLYVLELYCIILGLPPCLKILRRKQPQLTFFTITLQTCHYQRIPPHLLWATGLK

SEQ ID NO: 294 - protein, 229 aa, Rattus norvegicus [sequence omitted]

SEQ ID NO: 295 - protein, 208 aa, Petromyzon marinus
MTDAEYVRIHEKLDIYTFKKQFFNNKKSVSHRCYVLFELKRRGERRACFWGYAVNKPQSGTERGIHAEIFSIRKVEEYLRDNPGQFTINWYSSWSPCADCAEKILEWYNQELRGNGHTLKIWACKLYYEKNARNQIGLWNLRDNGVGLNVMVSEHYQCCRKIFIQSSHNQLNENRWLEKTLKRAEKRRSELSIMIQVKILHTTKSPAV

SEQ ID NO: 296-314 - skipped (19 entries)

SEQ ID NO: 315 - protein, 166 aa, Escherichia coli
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTD

SEQ ID NO: 316 - skipped

SEQ ID NO: 317 - protein, 160 aa, Staphylococcus aureus
MGSHMTNDIYFMTLAIEEAKKAAQLGEVPIGAIITKDDEVIARAHNLRETLQQPTAHAEHIAIERAAKVLGSWRLEGCTLYVTLEPCVMCAGTIVMSRIPRVVYGADDPKGGCSGSLMNLLQQSNFNHRAIVDKGVLKEACSTLLTTFFKNLRANKKSTN

SEQ ID NO: 318 - protein, 161 aa, Bacillus subtilis
MTQDELYMKEAIKEAKKAEEKGEVPIGAVLVINGEIIARAHNLRETEQRSIAHAEMLVIDEACKALGTWRLEGATLYVTLEPCPMCAGAVVLSRVEKVVFGAFDPKGGCSGTLMNLLQEERFNHQAEVVSGVLEEECGGMLSAFFRELRKKKKAARKNLSE

SEQ ID NO: 319 - protein, 183 aa, Salmonella enterica
MPPAFITGVTSLSDVELDHEYWMRHALTLAKRAWDEREVPVGAVLVHNHRVIGEGWNRPIGRHDPTAHAEIMALRQGGLVLQNYRLLDTTLYVTLEPCVMCAGAMVHSRIGRVVFGARDAKTGAAGSLIDVLHHPGMNHRVEIIEGVLRDECATLLSDFFRMRRQEIKALKKADRAEGAGPAV

SEQ ID NO: 320 - protein, 164 aa, Shewanella putrefaciens
MDEYWMQVAMQMAEKAEAAGEVPVGAVLVKDGQQIATGYNLSISQHDPTAHAEILCLRSAGKKLENYRLLDATLYITLEPCAMCAGAMVHSRIARVVYGARDEKTGAAGTVVNLLQHPAFNHQVEVTSGVLAEACSAQLSRFFKRRRDEKKALKLAQRAQQGIE

SEQ ID NO: 321 - protein, 173 aa, Haemophilus influenzae
MDAAKVRSEFDEKMMRYALELADKAEALGEIPVGAVLVDDARNIIGEGWNLSIVQSDPTAHAEIIALRNGAKNIQNYRLLNSTLYVTLEPCTMCAGAILHSRIKRLVFGASDYKTGAIGSRFHFFDDYKMNHTLEITSGVLAEECSQKLSTFFQKRREEKKIEKALLKSLSDK

SEQ ID NO: 322 - protein, 161 aa, Caulobacter vibrioides
MRTDESEDQDHRMMRLALDAARAAAEAGETPVGAVILDPSTGEVIATAGNGPIAAHDPTAHAEIAAMRAAAAKLGNYRLTDLTLVVTLEPCAMCAGAISHARIGRVVFGADDPKGGAVVHGPKFFAQPTCHWRPEVTGGVLADESADLLRGFFRARRKAKI

SEQ ID NO: 323 - protein, 178 aa, Geobacter sulfurreducens
MSSLKKTPIRDDAYWMGKAIREAAKAAARDEVPIGAVIVRDGAVIGRGHNLREGSNDPSAHAEMIAIRQAARRSANWRLTGATLYVTLEPCLMCMGAIILARLERVVFGCYDPKGAAGSLYDLSADPRLNHQVRLSPGVCQEECGTMLSDFFRDLRRRKKAKATPALFIDERKVPPEP

SEQ ID NO: 324 - protein, 167 aa, Escherichia coli
MSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTD

SEQ ID NO: 325 - protein, 178 aa, unidentified
  source 1..178: note = wild-type ecTadA
MRRAFITGVFFLSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTD

SEQ ID NO: 326 - protein, 166 aa, synthetic construct
SEVEFSHEYWMRHALTLAKRARDEREVPVGAVLVLNNRVIGEGWNRAIGLHDPTAHAEIMALRQGGLVMQNYRLYDATLYSTFEPCVMCAGAMIHSRIGRVVFGVRNAKTGAAGSLMDVLHHPGMNHRVEITEGILADECAALLCRFFRMPRRVFNAQKKAQSSTD

SEQ ID NO: 327-338 - skipped (12 entries)

SEQ ID NO: 339 - RNA, 81 nt, Streptococcus pyogenes
  source 1..81: mol_type = genomic RNA
gttttagagctagaaatagcaagttaaaataaggctagtccgttatcaacttgaaaaagtggcaccgagtcggtgcttttt

SEQ ID NO: 340 - DNA, 80 bp, Campylobacter jejuni
  source 1..80: mol_type = genomic DNA
gttttagtccctgaaaagggactaaaataaagagtttgcgggactctgcggggttacaatcccctaaaaccgcttttttt

SEQ ID NO: 341-343 - skipped (3 entries)

SEQ ID NO: 344 - protein, 16 aa, synthetic construct
KRPAATKKAGQAKKKK

SEQ ID NO: 345 - protein, 17 aa, synthetic construct
KKTELQTTNAENKTKKL

SEQ ID NO: 346 - protein, 19 aa, synthetic construct
KRGINDRNFWRGENGRKTR

SEQ ID NO: 347 - protein, 16 aa, synthetic construct
RKSGKIAAIVVKRPRK

SEQ ID NO: 348-360 - skipped (13 entries)

SEQ ID NO: 361 - protein, 1368 aa, Streptococcus pyogenes
MDKKYSIGLXIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 362 - protein, 1368 aa, Streptococcus pyogenes
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIXMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 363 - protein, 1368 aa, Streptococcus pyogenes
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHXAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 364 - protein, 1368 aa, Streptococcus pyogenes
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHXAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 365 - protein, 1368 aa, Streptococcus pyogenes
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 366 - protein, 1368 aa, Streptococcus pyogenes
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIAMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 367 - protein, 1368 aa, Streptococcus pyogenes
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHAAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 368 - protein, 1368 aa, Streptococcus pyogenes
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHAAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 369 - skipped

SEQ ID NO: 370 - protein, 1368 aa, Streptococcus pyogenes; identical to SEQ ID NO: 107

SEQ ID NO: 371-376 - skipped (6 entries)

SEQ ID NO: 377 - protein, 1053 aa, Staphylococcus aureus
MGKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRKLINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYKNDLIKINGELYRVIGVNNDLLNRIEVNMIDITYREYLENMNDKRPPHIIKTIASKTQSIKKYSTDILGNLYEVKSKKHPQIIKK

SEQ ID NO: 378 - skipped

SEQ ID NO: 379 - protein, 984 aa, synthetic construct
MARILAFAIGISSIGWAFSENDELKDCGVRIFTKVENPKTGESLALPRRLARSARKRLARRKARLNHLKHLIANEFKLNYEDYQSFDESLAKAYKGSLISPYELRFRALNELLSKQDFARVILHIAKRRGYDDIKNSDDKEKGAILKAIKQNEEKLANYQSVGEYLYKEYFQKFKENSKEFTNVRNKKESYERCIAQSFLKDELKLIFKKQREFGFSFSKKFEEEVLSVAFYKRALKDFSHLVGNCSFFTDEKRAPKNSPLAFMFVALTRIINLLNNLKNTEGILYTKDDLNALLNEVLKNGTLTYKQTKKLLGLSDDYEFKGEKGTYFIEFKKYKEFIKALGEHNLSQDDLNEIAKDITLIKDEIKLKKALAKYDLNQNQIDSLSKLEFKDHLNISFKALKLVTPLMLEGKKYDEACNELNLKVAINEDKKDFLPAFNETYYKDEVTNPVVLRAIKEYRKVLNALLKKYGKVHKINIELAREVGKNHSQRAKIEKEQNENYKAKKDAELECEKLGLKINSKNILKLRLFKEQKEFCAYSGEKIKISDLQDEKMLEIDHIYPYSRSFDDSYMNKVLVFTKQNQEKLNQTPFEAFGNDSAKWQKIEVLAKNLPTKKQKRILDKNYKDKEQKNFKDRNLNDTRYIARLVLNYTKDYLDFLPLSDDENTKLNDTQKGSKVHVEAKSGMLTSALRHTWGFSAKDRNNHLHHAIDAVIIAYANNSIVKAFSDFKKEQESNSAELYAKKISELDYKNKRKFFEPFSGFRQKVLDKIDEIFVSKPERKKPSGALHEETFRKEEEFYQSYGGKEGVLKALELGKIRKVNGKIVKNGDMFRVDIFKHKKTNKFYAVPIYTMDFALKVLPNKAVARSKKGEIKDWILMDENYEFCFSLYKDSLILIQTKDMQEPEFVYYNAFTSSTVSLIVSKHDNKFETLSKNQKILFKNANEKEVIAKSIGIQNLKVFEKYIVSALGEVTKAEFRQREDFKK

SEQ ID NO: 380 - protein, 1087 aa, Geobacillus stearothermophilus
MRYKIGLDIGITSVGWAVMNLDIPRIEDLGVRIFDRAENPQTGESLALPRRLARSARRRLRRRKHRLERIRRLVIREGILTKEELDKLFEEKHEIDVWQLRVEALDRKLNNDELARVLLHLAKRRGFKSNRKSERSNKENSTMLKHIEENRAILSSYRTVGEMIVKDPKFALHKRNKGENYTNTIARDDLEREIRLIFSKQREFGNMSCTEEFENEYITIWASQRPVASKDDIEKKVGFCTFEPKEKRAPKATYTFQSFIAWEHINKLRLISPSGARGLTDEERRLLYEQAFQKNKITYHDIRTLLHLPDDTYFKGIVYDRGESRKQNENIRFLELDAYHQIRKAVDKVYGKGKSSSFLPIDFDTFGYALTLFKDDADIHSYLRNEYEQNGKRMPNLANKVYDNELIEELLNLSFTKFGHLSLKALRSILPYMEQGEVYSSACERAGYTFTGPKKKQKTMLLPNIPPIANPVVMRALTQARKVVNAIIKKYGSPVSIHIELARDLSQTFDERRKTKKEQDENRKKNETAIRQLMEYGLTLNPTGHDIVKFKLWSEQNGRCAYSLQPIEIERLLEPGYVEVDHVIPYSRSLDDSYTNKVLVLTRENREKGNRIPAEYLGVGTERWQQFETFVLTNKQFSKKKRDRLLRLHYDENEETEFKNRNLNDTRYISRFFANFIREHLKFAESDDKQKVYTVNGRVTAHLRSRWEFNKNREESDLHHAVDAVIVACTTPSDIAKVTAFYQRREQNKELAKKTEPHFPQPWPHFADELRARLSKHPKESIKALNLGNYDDQKLESLQPVFVSRMPKRSVTGAAHQETLRRYVGIDERSGKIQTVVKTKLSEIKLDASGHFPMYGKESDPRTYEAIRQRLLEHNNDPKKAFQEPLYKPKKNGEPGPVIRTVKIIDTKNQVIPLNDGKTVAYNSNIVRVDVFEKDGKYYCVPVYTMDIMKGILPNKAIEPNKPYSEWKEMTEDYTFRFSLYPNDLIRIELPREKTVKTAAGEEINVKDVFVYYKTIDSANGGLELISHDHRFSLRGVGSRTLKRFEKYQVDVLGNIYKVRGEKRVGLASSAHSKPGKTIRPLQSTRD

SEQ ID NO: 381 - protein, 1228 aa, unidentified
  source 1..1228: note = Lachnospiraceae bacterium
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 382 - protein, 1108 aa, unidentified
  source 1..1108: note = Bacillus hisashii
MATRSFILKIEPNEEVKKGLWKTHEVLNHGIAYYMNILKLIRQEAIYEHHEQDPKNPKKVSKAEIQAELWDFVLKMQKCNSFTHEVDKDEVFNILRELYEELVPSSVEKKGEANQLSNKFLYPLVDPNSQSGKGTASSGRKPRWYNLKIAGDPSWEEEKKKWEEDKKKDPLAKILGKLAEYGLIPLFIPYTDSNEPIVKEIKWMEKSRNQSVRRLDKDMFIQALERFLSWESWNLKVKEEYEKVEKEYKTLEERIKEDIQALKALEQYEKERQEQLLRDTLNTNEYRLSKRGLRGWREIIQKWLKMDENEPSEKYLEVFKDYQRKHPREAGDYSVYEFLSKKENHFIWRNHPEYPYLYATFCEIDKKKKDAKQQATFTLADPINHPLWVRFEERSGSNLNKYRILTEQLHTEKLKKKLTVQLDRLIYPTESGGWEEKGKVDIVLLPSRQFYNQIFLDIEEKGKHAFTYKDESIKFPLKGTLGGARVQFDRDHLRRYPHKVESGNVGRIYFNMTVNIEPTESPVSKSLKIHRDDFPKVVNFKPKELTEWIKDSKGKKLKSGIESLEIGLRVMSIDLGQRQAAAASIFEVVDQKPDIEGKLFFPIKGTELYAVHRASFNIKLPGETLVKSREVLRKAREDNLKLMNQKLNFLRNVLHFQQFEDITEREKRVTKWISRQENSDVPLVYQDELIQIRELMYKPYKDWVAFLKQLHKRLEVEIGKEVKHWRKSLSDGRKGLYGISLKNIDEIDRTRKFLLRWSLRPTEPGEVRRLEPGQRFAIDQLNHLNALKEDRLKKMANTIIMHALGYCYDVRKKKWQAKNPACQIILFEDLSNYNPYEERSRFENSKLMKWSRREIPRQVALQGEIYGLQVGEVGAQFSSRFHAKTGSPGIRCSVVTKEKLQDNRFFKNLQREGRLTLDKIAVLKEGDLYPDKGGEKFISLSKDRKCVTTHADINAAQNLQKRFWTRTHGFYKVYCKAYQVDGQTVYIPESKDQKQKIIEEFGEGYFILKDGVYEWVNAGKLKIKKGSSKQSSSELVDSDILKDSFDLASELKGEKLMLYRDPSGNVFPSDKWMAAGVFFGKLERILISKLTNQYSISTIEDDSSKQSM

SEQ ID NO: 383 - protein, 1307 aa, Acidaminococcus sp.
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 384 - protein, 1307 aa, synthetic construct
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMANSNAATGEDYINSPVRDLNGVCFDSRFQNPEWPMDADANGAYHIALKGQLLLNHLKESKDLKLQNGISNQDWLAYIQELRN

SEQ ID NO: 385 - protein, 1259 aa, unidentified
  source 1..1259: note = Lachnospiraceae bacterium
MNYKTGLEDFIGKESLSKTLRNALIPTESTKIHMEEMGVIRDDELRAEKQQELKEIMDDYYRTFIEEKLGQIQGIQWNSLFQKMEETMEDISVRKDLDKIQNEKRKEICCYFTSDKRFKDLFNAKLITDILPNFIKDNKEYTEEEKAEKEQTRVLFQRFATAFTNYFNQRRNNFSEDNISTAISFRIVNENSEIHLQNMRAFQRIEQQYPEEVCGMEEEYKDMLQEWQMKHIYSVDFYDRELTQPGIEYYNGICGKINEHMNQFCQKNRINKNDFRMKKLHKQILCKKSSYYEIPFRFESDQEVYDALNEFIKTMKKKEIIRRCVHLGQECDDYDLGKIYISSNKYEQISNALYGSWDTIRKCIKEEYMDALPGKGEKKEEKAEAAAKKEEYRSIADIDKIISLYGSEMDRTISAKKCITEICDMAGQISIDPLVCNSDIKLLQNKEKTTEIKTILDSFLHVYQWGQTFIVSDIIEKDSYFYSELEDVLEDFEGITTLYNHVRSYVTQKPYSTVKFKLHFGSPTLANGWSQSKEYDNNAILLMRDQKFYLGIFNVRNKPDKQIIKGHEKEEKGDYKKMIYNLLPGPSKMLPKVFITSRSGQETYKPSKHILDGYNEKRHIKSSPKFDLGYCWDLIDYYKECIHKHPDWKNYDFHFSDTKDYEDISGFYREVEMQGYQIKWTYISADEIQKLDEKGQIFLFQIYNKDFSVHSTGKDNLHTMYLKNLFSEENLKDIVLKLNGEAELFFRKASIKTPIVHKKGSVLVNRSYTQTVGNKEIRVSIPEEYYTEIYNYLNHIGKGKLSSEAQRYLDEGKIKSFTATKDIVKNYRYCCDHYFLHLPITINFKAKSDVAVNERTLAYIAKKEDIHIIGIDRGERNLLYISVVDVHGNIREQRSFNIVNGYDYQQKLKDREKSRDAARKNWEEIEKIKELKEGYLSMVIHYIAQLVVKYNAVVAMEDLNYGFKTGRFKVERQVYQKFETMLIEKLHYLVFKDREVCEEGGVLRGYQLTYIPESLKKVGKQCGFIFYVPAGYTSKIDPTTGFVNLFSFKNLTNRESRQDFVGKFDEIRYDRDKKMFEFSFDYNNYIKKGTILASTKWKVYTNGTRLKRIVVNGKYTSQSMEVELTDAMEKMLQRAGIEYHDGKDLKGQIVEKGIEAEIIDIFRLTVQMRNSRSESEDREYDRLISPVLNDKGEFFDTATADKTLPQDADANGAYCIALKGLYEVKQIKENWKENEQFPRNKLVQDNKTWFDFMQKKRYL

SEQ ID NO: 386 - protein, 1306 aa, Prevotella copri
MAKNFEDFKRLYSLSKTLRFEAKPIGATLDNIVKSGLLDEDEHRAASYVKVKKLIDEYHKVFIDRVLDDGCLPLENKGNNNSLAEYYESYVSRAQDEDAKKKFKEIQQNLRSVIAKKLTEDKAYANLFGNKLIESYKDKEDKKKIIDSDLIQFINTAESTQLDSMSQDEAKELVKEFWGFVTYFYGFFDNRKNMYTAEEKSTGIAYRLVNENLPKFIDNIEAFNRAITRPEIQENMGVLYSDFSEYLNVESIQEMFQLDYYNMLLTQKQIDVYNAIIGGKTDDEHDVKIKGINEYINLYNQQHKDDKLPKLKALFKQILSDRNAISWLPEEFNSDQEVLNAIKDCYERLAENVLGDKVLKSLLGSLADYSLDGIFIRNDLQLTDISQKMFGNWGVIQNAIMQNIKRVAPARKHKESEEDYEKRIAGIFKKADSFSISYINDCLNEADPNNAYFVENYFATFGAVNTPTMQRENLFALVQNAYTEVAALLHSDYPTVKHLAQDKANVSKIKALLDAIKSLQHFVKPLLGKGDESDKDERFYGELASLWAELDTVTPLYNMIRNYMTRKPYSQKKIKLNFENPQLLGGWDANKEKDYATIILRRNGLYYLAIMDKDSRKLLGKAMPSDGECYEKMVYKFFKDVTTMIPKCSTQLKDVQAYFKVNTDDYVLNSKAFNKPLTITKEVFDLNNVLYGKYKKFQKGYLTATGDNVGYTHAVNVWIKFCMDFLNSYDSTCIYDFSSLKPESYLSLDAFYQDANLLLYKLSFARASVSYINQLVEEGKMYLFQIYNKDFSEYSKGTPNMHTLYWKALFDERNLADVVYKLNGQAEMFYRKKSIENTHPTHPANHPILNKNKDNKKKESLFDYDLIKDRRYTVDKFMFHVPITMNFKSVGSENINQDVKAYLRHADDMHIIGIDRGERHLLYLVVIDLQGNIKEQYSLNEIVNEYNGNTYHTNYHDLLDVREEERLKARQSWQTIENIKELKEGYLSQVIHKITQLMVRYHAIVVLEDLSKGFMRSRQKVEKQVYQKFEKMLIDKLNYLVDKKTDVSTPGGLLNAYQLTCKSDSSQKLGKQSGFLFYIPAWNTSKIDPVTGFVNLLDTHSLNSKEKIKAFFSKFDAIRYNKDKKWFEFNLDYDKFGKKAEDTRTKWTLCTRGMRIDTFRNKEKNSQWDNQEVDLTTEMKSLLEHYYIDIHGNLKDAISAQTDKAFFTGLLHILKLTLQMRNSITGTETDYLVSPVADENGIFYDSRSCGNQLPENADANGAYNIARKGLMLIEQIKNAEDLNNVKFDISNKAWLNFAQQKPYKNG

SEQ ID NO: 387 - protein, 1142 aa, Eubacterium rectale
MFSAKLISDILPEFVIHNNNYSASEKEEKTQVIKLFSRFATSFKDYFKNRANCFSANDISSSSCHRIVNDNAEIFFSNALVYRRIVKNLSNDDINKISGDMKDSLKEMSLEEIYSYEKYGEFITQEGISFYNDICGKVNLFMNLYCQKNKENKNLYKLRKLHKQILCIADTSYEVPYKFESDEEVYQSVNGFLDNISSKHIVERLRKIGENYNGYNLDKIYIVSKFYESVSQKTYRDWETINTALEIHYNNILPGNGKSKADKVKKAVKNDLQKSITEINELVSNYKLCPDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHLKSSKDFDITFCHDLIDYFKNCIAIHPEWKNFGFDFSDTSTYEDISGFYREVELQGYKIDWTYISEKDIDLLQEKGQLYLFQIYNKDFSKKSSGNDNLHTMYLKNLFSEENLKDIVLKLNGEAEIFFRKSSIKNPIIHKKGSILVNRTYEAEEKDQFGNIQIVRKTIPENIYQELYKYFNDKSDKELSDEAAKLKNVVGHHEAATNIVKDYRYTYDKYFLHMPITINFKANKTSFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSDKNLFCFTFDYNNFITQNTVMSKSSWSVYTYGVRIKRRFVNGRFSNESDTIDITKDMEKTLEMTDINWRDGHDLRQDIIDYEIVQHIFEIFKLTVQMRNSLSELEDRDYDRLISPVLNENNIFYDSAKAGDALPKDADANGAYCIALKGLYEIKQITENWKEDGKFSRDKLKISNKDWFDFIQNKRYL

SEQ ID NO: 388 - protein, 1259 aa, Clostridium sp.
MNYKTGLEDFIGKESLSKTLRNALIPTESTKIHMEEMGVIRDDELRAEKQQELKEIMDDYYRAFIEEKLGQIQGIQWNSLFQKMEETMEDISVRKDLDKIQNEKRKEICCYFTSDKRFKDLFNAKLITDILPNFIKDNKEYTEEEKAEKEQTRVLFQRFATAFTNYFNQRRNNFSEDNISTAISFRIVNENSEIHLQNMRAFQRIEQQYPEEVCGMEEEYKDMLQEWQMKHIYLVDFYDRVLTQPGIEYYNGICGKINEHMNQFCQKNRINKNDFRMKKLHKQILCKKSSYYEIPFRFESDQEVYDALNEFIKTMKEKEIICRCVHLGQKCDDYDLGKIYISSNKYEQISNALYGSWDTIRKCIKEEYMDALPGKGEKKEEKAEAAAKKEEYRSIADIDKIISLYGSEMDRTISAKKCITEICDMAGQISTDPLVCNSDIKLLQNKEKTTEIKTILDSFLHVYQWGQTFIVSDIIEKDSYFYSELEDVLEDFEGITTLYNHVRSYVTQKPYSTVKFKLHFGSPTLANGWSQSKEYDNNAILLMRDQKFYLGIFNVRNKPDKQIIKGHEKEEKGDYKKMIYNLLPGPSKMLPKVFITSRSGQETYKPSKHILDGYNEKRHIKSSPKFDLGYCWDLIDYYKECIHKHPDWKNYDFHFSDTKDYEDISGFYREVEMQGYQIKWTYISADEIQKLDEKGQIFLFQIYNKDFSVHSTGKDNLHTMYLKNLFSEENLKDIVLKLNGEAELFFRKASIKTPVVHKKGSVLVNRSYTQTVGDKEIRVSIPEEYYTEIYNYLNHIGRGKLSTEAQRYLEERKIKSFTATKDIVKNYRYCCDHYFLHLPITINFKAKSDIAVNERTLAYIAKKEDIHIIGIDRGERNLLYISVVDVHGNIREQRSFNIVNGYDYQQKLKDREKSRDAARKNWEEIEKIKELKEGYLSMVIHYIAQLVVKYNAVVAMEDLNYGFKTGRFKVERQVYQKFETMLIEKLHYLVFKDREVCEEGGVLRGYQLTYIPESLKKVGKQCGFIFYVPAGYTSKIDPTTGFVNLFSFKNLTNRESRQDFVGKFDEIRYDRDKKMFEFSFDYNNYIKKGTMLASTKWKVYTNGTRLKRIVVNGKYTSQSMEVELTDAMEKMLQRAGIEYHDGKDLKGQIVEKGIEAEIIDIFRLTVQMRNSRSESEDREYDRLISPVLNDKGEFFDTATADKTLPQDADANGAYCIALKGLYEVKQIKENWKENEQFPRNKLVQDNKTWFDFMQKKRYL

SEQ ID NO: 389 - protein, 1108 aa, unidentified; identical to SEQ ID NO: 382
  source 1..1108: note = Bacillus hisashii

SEQ ID NO: 390 - protein, 1465 aa, Thermomonas hydrothermalis
MSEKTTQRAYTLRLNRASGECAVCQNNSCDCWHDALWATHKAVNRGAKAFGDWLLTLRGGLCHTLVEMEVPAKGNNPPQRPTDQERRDRRVLLALSWLSVEDEHGAPKEFIVATGRDSADDRAKKVEEKLREILEKRDFQEHEIDAWLQDCGPSLKAHIREDAVWVNRRALFDAAVERIKTLTWEEAWDFLEPFFGTQYFAGIGDGKDKDDAEGPARQGEKAKDLVQKAGQWLSARFGIGTGADFMSMAEAYEKIAKWASQAQNGDNGKATIEKLACALRPSEPPTLDTVLKCISGPGHKSATREYLKTLDKKSTVTQEDLNQLRKLADEDARNCRKKVGKKGKKPWADEVLKDVENSCELTYLQDNSPARHREFSVMLDHAARRVSMAHSWIKKAEQRRRQFESDAQKLKNLQERAPSAVEWLDRFCESRSMTTGANTGSGYRIRKRAIEGWSYVVQAWAEASCDTEDKRIAAARKVQADPEIEKFGDIQLFEALAADEAICVWRDQEGTQNPSILIDYVTGKTAEHNQKRFKVPAYRHPDELRHPVFCDFGNSRWSIQFAIHKEIRDRDKGAKQDTRQLQNRHGLKMRLWNGRSMTDVNLHWSSKRLTADLALDQNPNPNPTEVTRADRLGRAASSAFDHVKIKNVFNEKEWNGRLQAPRAELDRIAKLEEQGKTEQAEKLRKRLRWYVSFSPCLSPSGPFIVYAGQHNIQPKRSGQYAPHAQANKGRARLAQLILSRLPDLRILSVDLGHRFAAACAVWETLSSDAFRREIQGLNVLAGGSGEGDLFLHVEMTGDDGKRRTVVYRRIGPDQLLDNTPHPAPWARLDRQFLIKLQGEDEGVREASNEELWTVHKLEVEVGRTVPLIDRMVRSGFGKTEKQKERLKKLRELGWISAMPNEPSAETDEKEGEIRSISRSVDELMSSALGTLRLALKRHGNRARIAFAMTADYKPMPGGQKYYFHEAKEASKNDDETKRRDNQIEFLQDALSLWHDLFSSPDWEDNEAKKLWQNHIATLPNYQTPEEISAELKRVERNKKRKENRDKLRTAAKALAENDQLRQHLHDTWKERWESDDQQWKERLRSLKDWIFPRGKAEDNPSIRHVGGLSITRINTISGLYQILKAFKMRPEPDDLRKNIPQKGDDELENFNRRLLEARDRLREQRVKQLASRIIEAALGVGRIKIPKNGKLPKRPRTTVDTPCHAVVIESLKTYRPDDLRTRRENRQLMQWSSAKVRKYLKEGCELYGLHFLEVPANYTSRQCSRTGLPGIRCDDVPTGDFLKAPWWRRAINTAREKNGGDAKDRFLVDLYDHLNNLQSKGEALPATVRVPRQGGNLFIAGAQLDDTNKERRAIQADLNAAANIGLRALLDPDWRGRWWYVPCKDGTSEPALDRIEGSTAFNDVRSLPTGDNSSRRAPREIENLWRDPSGDSLESGTWSPTRAYWDTVQSRVIELLRRHAGLPTS

SEQ ID NO: 391 - protein, 1090 aa, Laceyella sacchari
MSIRSFKLKLKTKSGVNAEQLRRGLWRTHQLINDGIAYYMNWLVLLRQEDLFIRNKETNEIEKRSKEEIQAVLLERVHKQQQRNQWSGEVDEQTLLQALRQLYEEIVPSVIGKSGNASLKARFFLGPLVDPNNKTTKDVSKSGPTPKWKKMKDAGDPNWVQEYEKYMAERQTLVRLEEMGLIPLFPMYTDEVGDIHWLPQASGYTRTWDRDMFQQAIERLLSWESWNRRVRERRAQFEKKTHDFASRFSESDVQWMNKLREYEAQQEKSLEENAFAPNEPYALTKKALRGWERVYHSWMRLDSAASEEAYWQEVATCQTAMRGEFGDPAIYQFLAQKENHDIWRGYPERVIDFAELNHLQRELRRAKEDATFTLPDSVDHPLWVRYEAPGGTNIHGYDLVQDTKRNLTLILDKFILPDENGSWHEVKKVPFSLAKSKQFHRQVWLQEEQKQKKREVVFYDYSTNLPHLGTLAGAKLQWDRNFLNKRTQQQIEETGEIGKVFFNISVDVRPAVEVKNGRLQNGLGKALTVLTHPDGTKIVTGWKAEQLEKWVGESGRVSSLGLDSLSEGLRVMSIDLGQRTSATVSVFEITKEAPDNPYKFFYQLEGTEMFAVHQRSFLLALPGENPPQKIKQMREIRWKERNRIKQQVDQLSAILRLHKKVNEDERIQAIDKLLQKVASWQLNEEIATAWNQALSQLYSKAKENDLQWNQAIKNAHHQLEPVVGKQISLWRKDLSTGRQGIAGLSLWSIEELEATKKLLTRWSKRSREPGVVKRIERFETFAKQIQHHINQVKENRLKQLANLIVMTALGYKYDQEQKKWIEVYPACQVVLFENLRSYRFSFERSRRENKKLMEWSHRSIPKLVQMQGELFGLQVADVYAAYSSRYHGRTGAPGIRCHALTEADLRNETNIIHELIEAGFIKEEHRPYLQQGDLVPWSGGELFATLQKPYDNPRILTLHADINAAQNIQKRFWHPSMWFRVNCESVMEGEIVTYVPKNKTVHKKQGKTFRFVKVEGSDVYEWAKWSKNRNKNTFSSITERKPPSSMILFRDPSGTFFKEQEWVEQKTFWGKVQSMIQAYMKKTIVQRMEE

SEQ ID NO: 392 - protein, 1194 aa, unidentified
  source 1..1194: note = Dsulfonatronum thiodismutans
MVLGRKDDTAELRRALWTTHEHVNLAVAEVERVLLRCRGRSYWTLDRRGDPVHVPESQVAEDALAMAREAQRRNGWPVVGEDEEILLALRYLYEQIVPSCLLDDLGKPLKGDAQKIGTNYAGPLFDSDTCRRDEGKDVACCGPFHEVAGKYLGALPEWATPISKQEFDGKDASHLRFKATGGDDAFFRVSIEKANAWYEDPANQDALKNKAYNKDDWKKEKDKGISSWAVKYIQKQLQLGQDPRTEVRRKLWLELGLLPLFIPVFDKTMVGNLWNRLAVRLALAHLLSWESWNHRAVQDQALARAKRDELAALFLGMEDGFAGLREYELRRNESIKQHAFEPVDRPYVVSGRALRSWTRVREEWLRHGDTQESRKNICNRLQDRLRGKFGDPDVFHWLAEDGQEALWKERDCVTSFSLLNDADGLLEKRKGYALMTFADARLHPRWAMYEAPGGSNLRTYQIRKTENGLWADVVLLSPRNESAAVEEKTFNVRLAPSGQLSNVSFDQIQKGSKMVGRCRYQSANQQFEGLLGGAEILFDRKRIANEQHGATDLASKPGHVWFKLTLDVRPQAPQGWLDGKGRPALPPEAKHFKTALSNKSKFADQVRPGLRVLSVDLGVRSFAACSVFELVRGGPDQGTYFPAADGRTVDDPEKLWAKHERSFKITLPGENPSRKEEIARRAAMEELRSLNGDIRRLKAILRLSVLQEDDPRTEHLRLFMEAIVDDPAKSALNAELFKGFGDDRFRSTPDLWKQHCHFFHDKAEKVVAERFSRWRTETRPKSSSWQDWRERRGYAGGKSYWAVTYLEAVRGLILRWNMRGRTYGEVNRQDKKQFGTVASALLHHINQLKEDRIKTGADMIIQAARGFVPRKNGAGWVQVHEPCRLILFEDLARYRFRTDRSRRENSRLMRWSHREIVNEVGMQGELYGLHVDTTEAGFSSRYLASSGAPGVRCRHLVEEDFHDGLPGMHLVGELDWLLPKDKDRTANEARRLLGGMVRPGMLVPWDGGELFATLNAASQLHVIHADINAAQNLQRRFWGRCGEAIRIVCNQLSVDGSTRYEMAKAPKARLLGALQQLKNGDAPFHLTSIPNSQKPENSYVMTPTNAGKKYRAGPGEKSSGEEDELALDIVEQAEELAQGRKTFFRDPSGVFFAPDRWLPSEIYWSRIRRRIWQVTLERNSSGRQERAEMDEMPY

SEQ ID NO: 393 - protein, 162 aa, synthetic construct
MFERNYDPRELRKETYLLYEIKWGKSGKLWRHWCQNNRTQHAEVYFLENIFNARRFNPSTHCSITWYLSWSPCAECSQKIVDFLKEHPNVNLEIYVARLYYHEDERNRQGLRDLVNSGVTIRIMDLPDYNYCWKTFVSDQGGDEDYWPGHFAPWIKQYSLKL

SEQ ID NO: 394 - protein, 162 aa, synthetic construct
MFERNYDPRELRKETYLLYEIKWGKSGKLWRHWCQNNRTQHAEVYFLENIFNARRFNPSTHCSITWYLSWSPCAECSQKIVDFLKEHPNVNLEIYVARLYYPENERNRQGLRDLVNSGVTIRIMDLPDYNYCWKTFVSDQGGDEDYWPGHFAPWIKQYSLKL

SEQ ID NO: 395-397 - skipped (3 entries)

SEQ ID NO: 398 - protein, 17 aa, synthetic construct
KRTADGSEFEPKKKRKV

SEQ ID NO: 399-407 - skipped (9 entries)

SEQ ID NO: 408 - protein, 7 aa, synthetic construct
PKKKRKV

SEQ ID NO: 409 - protein, 30 aa, synthetic construct
MDSLLMNRRKFLYQFKNVRWAKGRRETYLC

SEQ ID NO: 410 - protein, 18 aa, synthetic construct
KRTADGSEFESPKKKRKV

SEQ ID NO: 411 - protein, 17 aa, synthetic construct
KRTADGSEFEPKKKRKV

SEQ ID NO: 412 - protein, 32 aa, synthetic construct
SGGSSGGSSGSETPGTSESATPESSGGSSGGS

SEQ ID NO: 413 - protein, 10 aa, synthetic construct
SGGSGGSGGS

SEQ ID NO: 414 - protein, 4 aa, synthetic construct
SGGS

SEQ ID NO: 415 - protein, 150 aa, synthetic construct
  REGION 6..150: note = amino acids 6-150 may be absent
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 416 - protein, 30 aa, synthetic construct
  REGION 2..30: note = amino acids 2-30 may be absent
GGGGGGGGGGGGGGGGGGGGGGGGGGGGGG

SEQ ID NO: 417 - protein, 150 aa, synthetic construct
  REGION 6..150: note = amino acids 6-150 may be absent
EAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAK

SEQ ID NO: 418 - protein, 90 aa, synthetic construct
  REGION 4..90: note = amino acids 4-90 may be absent
GGSGGSGGSGGSGGSGGSGGSGGSGGSGGSGGSGGSGGSGGSGGSGGSGGSGGSGGSGGSGGSGGSGGSGGSGGSGGSGGSGGSGGSGGS

SEQ ID NO: 419 - protein, 120 aa, synthetic construct
  REGION 5..120: note = amino acids 5-120 may be absent
SGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGS

SEQ ID NO: 420 - protein, 60 aa, synthetic construct
  REGION 3..40: note = amino acids 3-40 may be absent
XPXPXPXPXPXPXPXPXPXPXPXPXPXPXPXPXPXPXPXPXPXPXPXPXPXPXPXPXPXP

SEQ ID NO: 421 - protein, 21 aa, synthetic construct
  REGION 4..18: note = amino acids 4-18 mayb be absent
GGSGGSGGSGGSGGSGGSGGS

SEQ ID NO: 422 - protein, 16 aa, synthetic construct
SGSETPGTSESATPES

SEQ ID NO: 423 - protein, 24 aa, synthetic construct
SGGSSGSETPGTSESATPESSGGS

SEQ ID NO: 424 - protein, 104 aa, synthetic construct
GGSGGSPGSPAGSPTSTEEGTSESATPESGPGTSTEPSEGSAPGSPAGSPTSTEEGTSTEPSEGSAPGTSTEPSEGSAPGTSESATPESGPGSEPATSGGSGGS

SEQ ID NO: 425 - protein, 24 aa, synthetic construct
SGGSSGGSSGSETPGTSESATPES

SEQ ID NO: 426 - protein, 40 aa, synthetic construct
SGGSSGGSSGSETPGTSESATPESSGGSSGGSSGGSSGGS

SEQ ID NO: 427 - protein, 64 aa, synthetic construct
SGGSSGGSSGSETPGTSESATPESSGGSSGGSSGGSSGGSSGSETPGTSESATPESSGGSSGGS

SEQ ID NO: 428 - protein, 92 aa, synthetic construct
PGSPAGSPTSTEEGTSESATPESGPGTSTEPSEGSAPGSPAGSPTSTEEGTSTEPSEGSAPGTSTEPSEGSAPGTSESATPESGPGSEPATS

SEQ ID NO: 429 - protein, 48 aa, synthetic construct
SGGSSGGSSGGSSGGSSGSETPGTSESATPESSGGSSGGSSGGSSGGS

SEQ ID NO: 430 - DNA, 22 bp, synthetic construct
  source 1..22: mol_type = other DNA
gccaccgcagccacgcagagca

SEQ ID NO: 431 - skipped

SEQ ID NO: 432 - DNA, 23 bp, synthetic construct
  source 1..23: mol_type = other DNA
gcaagagcacaagaggaagagag

SEQ ID NO: 433 - protein, 166 aa, Escherichia coli
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSIN

SEQ ID NO: 434 - DNA, 24 bp, synthetic construct
  source 1..24: mol_type = other DNA
gccctcatctccaatatggtatgg

SEQ ID NO: 435 - skipped

SEQ ID NO: 436 - DNA, 24 bp, synthetic construct
  source 1..24: mol_type = other DNA
gaggcaagagggcggctttgggcg

SEQ ID NO: 437 - skipped

SEQ ID NO: 438 - protein, 1053 aa, Streptococcus pyogenes
MGKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRKLINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYKNDLIKINGELYRVIGVNNDLLNRIEVNMIDITYREYLENMNDKRPPHIIKTIASKTQSIKKYSTDILGNLYEVKSKKHPQIIKK

SEQ ID NO: 439 - skipped

SEQ ID NO: 440 - DNA, 24 bp, synthetic construct
  source 1..24: mol_type = other DNA
tatgttccagcttcctgggtctgc

SEQ ID NO: 441 - skipped

SEQ ID NO: 442 - DNA, 23 bp, synthetic construct
  source 1..23: mol_type = other DNA
ggaccctctccatttctacccct

SEQ ID NO: 443 - skipped

SEQ ID NO: 444 - DNA, 24 bp, synthetic construct
  source 1..24: mol_type = other DNA
gtttgtccccacagtccccaggga

SEQ ID NO: 445 - skipped

SEQ ID NO: 446 - DNA, 24 bp, synthetic construct
  source 1..24: mol_type = other DNA
gcgcaaagctgcatccaccccccg

SEQ ID NO: 447 - skipped

SEQ ID NO: 448 - protein, 171 aa, Streptococcus pyogenes
MPYSLEEQTYFMQEALKEAEKSLQKAEIPIGCVIVKDGEIIGRGHNAREESNQAIMHAEIMAINEANAHEGNWRLLDTTLFVTIEPCVMCSGAIGLARIPHVIYGASNQKFGGADSLYQILTDERLNHRVQVERGLLAADCANIMQTFFRQGRERKKIAKHLIKEQSDPFD

SEQ ID NO: 449 - protein, 151 aa, unidentified
  source 1..151: note = Aquifex aeolicus
MGKEYFLKVALREAKRAFEKGEVPVGAIIVKEGEIISKAHNSVEELKDPTAHAEMLAIKEACRRLNTKYLEGCELYVTLEPCIMCSYALVLSRIEKVIFSALDKKHGGVVSVFNILDEPTLNHRVKWEYYPLEEASELLSEFFKKLRNNII

SEQ ID NO: 450 - DNA, 24 bp, synthetic construct
  source 1..24: mol_type = other DNA
gttccccttcattgcggcgggctg

SEQ ID NO: 451 - skipped

SEQ ID NO: 452 - DNA, 22 bp, synthetic construct
  source 1..22: mol_type = other DNA
gattgtgctgtcaggagctcgg

SEQ ID NO: 453 - skipped

SEQ ID NO: 454 - DNA, 22 bp, synthetic construct
  source 1..22: mol_type = other DNA
gagtagaggcggccacgacctg

SEQ ID NO: 455 - skipped

SEQ ID NO: 456 - DNA, 22 bp, synthetic construct
  source 1..22: mol_type = other DNA
tcctaatcccttgcacagggtc

SEQ ID NO: 457 - skipped

SEQ ID NO: 458 - DNA, 22 bp, synthetic construct
  source 1..22: mol_type = other DNA
ggtccaaagcaggatgacaggc

SEQ ID NO: 459 - skipped

SEQ ID NO: 460 - DNA, 22 bp, synthetic construct
  source 1..22: mol_type = other DNA
ggagtgtgacagcctggggccc

SEQ ID NO: 461 - skipped

SEQ ID NO: 462 - DNA, 22 bp, synthetic construct
  source 1..22: mol_type = other DNA
agctgcagcaccaggatcagtg

SEQ ID NO: 463 - skipped

SEQ ID NO: 464 - DNA, 22 bp, synthetic construct
  source 1..22: mol_type = other DNA
gaggtggctaaagccagggaga

SEQ ID NO: 465 - skipped

SEQ ID NO: 466 - DNA, 22 bp, synthetic construct
  source 1..22: mol_type = other DNA
gtggggtgaggagaaggctggg

SEQ ID NO: 467 - skipped

SEQ ID NO: 468 - DNA, 22 bp, synthetic construct
  source 1..22: mol_type = other DNA
gcaggaggttttccttgtggca

SEQ ID NO: 469 - skipped

SEQ ID NO: 470 - DNA, 21 bp, synthetic construct
  source 1..21: mol_type = other DNA
ggaacacaaagcatagactgc

SEQ ID NO: 471 - skipped

SEQ ID NO: 472 - DNA, 21 bp, synthetic construct
  source 1..21: mol_type = other DNA
ggggcccagactgagcacgtg

SEQ ID NO: 473 - skipped

SEQ ID NO: 474 - DNA, 22 bp, synthetic construct
  source 1..22: mol_type = other DNA
gcagtcatcttagtcattacct

SEQ ID NO: 475 - skipped

SEQ ID NO: 476 - DNA, 22 bp, synthetic construct
  source 1..22: mol_type = other DNA
gctgagtccgagcagaagaaga

SEQ ID NO: 477 - protein, 1368 aa, synthetic construct
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESIRPKRNSDKLIARKKDWDPKKYGGFVSPTVAYSVLVVAKVEKGKSKKLKSVKELLGITIMERSSFEKNPIDFLEAKGYKEVKKDLIIKLPKYSLFELENGRKRMLASARFLQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPRAFKYFDTTIDRKVYRSTKEVLDATLIHQSITGLYETRIDLSQLGGD

SEQ ID NO: 478 - protein, 1054 aa, Staphylococcus aureus
MGKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRKLINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYKNDLIKINGELYRVIGVNNDLLNRIEVNMIDITYREYLENMNDKRPPHIIKTIASKTQSIKKYSTDILGNLYEVKSKKHPQIIKKG

SEQ ID NO: 479 - protein, 1061 aa, synthetic construct
MQENQQKQNYILGLAIGITSVGYGLIDSKTREVIDAGVRLFPEADSENNSNRRSKRGARRLKRRRIHRLNRVKDLLADYQMIDLNNVPKSTDPYTIRVKGLREPLTKEEFAIALLHIAKRRGLHNISVSMGDEEQDNELSTKQQLQKNAQQLQDKYVCELQLERLTNINKVRGEKNRFKTEDFVKEVKQLCETQRQYHNIDDQFIQQYIDLVSTRREYFEGPGNGSPYGWDGDLLKWYEKLMGRCTYFPEELRSVKYAYSADLFNALNDLNNLVVTRDDNPKLEYYEKYHIIENVFKQKKNPTLKQIAKEIGVQDYDIRGYRITKSGKPQFTSFKLYHDLKNIFEQAKYLEDVEMLDEIAKILTIYQDEISIKKALDQLPELLTESEKSQIAQLTGYTGTHRLSLKCIHIVIDELWESPENQMEIFTRLNLKPKKVEMSEIDSIPTTLVDEFILSPVVKRAFIQSIKVINAVINRFGLPEDIIIELAREKNSKDRRKFINKLQKQNEATRKKIEQLLAKYGNTNAKYMIEKIKLHDMQEGKCLYSLEAIPLEDLLSNPTHYEVDHIIPRSVSFDNSLNNKVLVKQSENSKKGNRTPYQYLSSNESKISYNQFKQHILNLSKAKDRISKKKRDMLLEERDINKFEVQKEFINRNLVDTRYATRELSNLLKTYFSTHDYAVKVKTINGGFTNHLRKVWDFKKHRNHGYKHHAEDALVIANADFLFKTHKALRRTDKILEQPGLEVNDTTVKVDTEEKYQELFETPKQVKNIKQFRDFKYSHRVDKKPNRQLINDTLYSTREIDGETYVVQTLKDLYAKDNEKVKKLFTERPQKILMYQHDPKTFEKLMTILNQYAEAKNPLAAYYEDKGEYVTKYAKKGNGPAIHKIKYIDKKLGSYLDVSNKYPETQNKLVKLSLKSFRFDIYKCEQGYKMVSIGYLDVLKKDNYYYIPKDKYEAEKQKKKIKESDLFVGSFYYNDLIMYEDELFRVIGVNSDINNLVELNMVDITYKDFCEVNNVTGEKRIKKTIGKRVVLIEKYTTDILGNLYKTPLPKKPQLIFKRGEL

SEQ ID NO: 480 - DNA, 21 bp, synthetic construct
  source 1..21: mol_type = other DNA
gtggcactgcggctggaggtg

SEQ ID NO: 481 - skipped

SEQ ID NO: 482 - protein, 19 aa, synthetic construct
NLSKRPAAIKKAGQAKKKK

SEQ ID NO: 483 - protein, 9 aa, synthetic construct
PAAKRVKLD

SEQ ID NO: 484 - protein, 11 aa, synthetic construct
RQRRNELKRSF

SEQ ID NO: 485 - protein, 38 aa, synthetic construct
NQSSNFGPMKGGNFGGRSSGPYGGGGQYFAKPRNQGGY

SEQ ID NO: 486 - protein, 16 aa, unidentified
  source 1..16: note = Xenopus nucleoplasmin
KRXXXXXXXXXXKKKL

SEQ ID NO: 487 - protein, 208 aa, synthetic construct
MTDAEYVRIHEKLDIYTFKKQFSNNKKSVSHRCYVLFELKRRGERRACFWGYAVNKPQSGTERGIHAEIFSIRKVEEYLRDNPGQFTINWYSSWSPCADCAEKILEWYNQELRGNGHTLKIWVCKLYYEKNARNQIGLWNLRDNGVGLNVMVSEHYQCCRKIFIQSSHNQLNENRWLEKTLKRAEKRRSELSIMFQVKILHTTKSPAV

SEQ ID NO: 488 - DNA, 21 bp, synthetic construct
  source 1..21: mol_type = other DNA
cagtcatcttagtcattacct

SEQ ID NO: 489 - skipped

SEQ ID NO: 490 - DNA, 22 bp, synthetic construct
  source 1..22: mol_type = other DNA
gcctacacccgcaccttggcgc

SEQ ID NO: 491 - skipped

SEQ ID NO: 492 - DNA, 22 bp, synthetic construct
  source 1..22: mol_type = other DNA
gccataccttggagcaacggcg

SEQ ID NO: 493 - skipped

SEQ ID NO: 494 - DNA, 22 bp, synthetic construct
  source 1..22: mol_type = other DNA
gcacccataccttggagcaacg

SEQ ID NO: 495 - skipped

SEQ ID NO: 496 - DNA, 22 bp, synthetic construct
  source 1..22: mol_type = other DNA
gagatacctgagtaactttctg

SEQ ID NO: 497 - skipped

SEQ ID NO: 498 - DNA, 21 bp, synthetic construct
  source 1..21: mol_type = other DNA
gcccataccttggagcaacgg

SEQ ID NO: 499 - skipped

SEQ ID NO: 500 - DNA, 22 bp, synthetic construct
  source 1..22: mol_type = other DNA
gccaccgcagccacgcagagca

SEQ ID NO: 501 - skipped

SEQ ID NO: 502 - DNA, 20 bp, synthetic construct
  source 1..20: mol_type = other DNA
atggaggagacccagaggct

SEQ ID NO: 503 - skipped

SEQ ID NO: 504 - DNA, 21 bp, synthetic construct
  source 1..21: mol_type = other DNA
gctcaactgtcaagggaaggg

SEQ ID NO: 505 - skipped

SEQ ID NO: 506 - DNA, 20 bp, synthetic construct
  source 1..20: mol_type = other DNA
gccacgaatgcccaggacca

SEQ ID NO: 507 - skipped

SEQ ID NO: 508 - DNA, 21 bp, synthetic construct
  source 1..21: mol_type = other DNA
ggtgcccatgagggccagggg

SEQ ID NO: 509 - skipped

SEQ ID NO: 510 - DNA, 22 bp, synthetic construct
  source 1..22: mol_type = other DNA
gcacccgcaccttggcgcagcg

SEQ ID NO: 511 - skipped

SEQ ID NO: 512 - DNA, 22 bp, synthetic construct
  source 1..22: mol_type = other DNA
gcctacacccgcaccttggcgc

SEQ ID NO: 513 - skipped

SEQ ID NO: 514 - DNA, 22 bp, synthetic construct
  source 1..22: mol_type = other DNA
gttgcatggggccaggatccgt

SEQ ID NO: 515 - skipped

SEQ ID NO: 516 - DNA, 22 bp, synthetic construct
  source 1..22: mol_type = other DNA
gagatgggggtcttaccggggg

SEQ ID NO: 517 - skipped

SEQ ID NO: 518 - DNA, 21 bp, synthetic construct
  source 1..21: mol_type = other DNA
gccttggaaagacggaggcag

SEQ ID NO: 519 - skipped

SEQ ID NO: 520 - DNA, 22 bp, synthetic construct
  source 1..22: mol_type = other DNA
gcgtgcttacctgtctgtggaa

SEQ ID NO: 521 - skipped

SEQ ID NO: 522 - DNA, 22 bp, synthetic construct
  source 1..22: mol_type = other DNA
gacggccgtgcttacctgtctg

SEQ ID NO: 523 - skipped

SEQ ID NO: 524 - DNA, 21 bp, synthetic construct
  source 1..21: mol_type = other DNA
gtcgagcaggccagcaagtgt

SEQ ID NO: 525 - skipped

SEQ ID NO: 526 - DNA, 22 bp, synthetic construct
  source 1..22: mol_type = other DNA
gccctgcaccaggcattgcagc

SEQ ID NO: 527 - skipped

SEQ ID NO: 528 - DNA, 21 bp, synthetic construct
  source 1..21: mol_type = other DNA
gccatcctgcttacctgcccc

SEQ ID NO: 529 - skipped

SEQ ID NO: 530 - DNA, 22 bp, synthetic construct
  source 1..22: mol_type = other DNA
gtgggtgcccatcggggcgagg

SEQ ID NO: 531 - skipped

SEQ ID NO: 532 - DNA, 22 bp, synthetic construct
  source 1..22: mol_type = other DNA
gccataccttggagcaacggcg

SEQ ID NO: 533 - skipped

SEQ ID NO: 534 - DNA, 21 bp, synthetic construct
  source 1..21: mol_type = other DNA
gaagatggctcaccaggccca

SEQ ID NO: 535 - skipped

SEQ ID NO: 536 - DNA, 22 bp, synthetic construct
  source 1..22: mol_type = other DNA
gtactggggaccttaccagggg

SEQ ID NO: 537 - skipped

SEQ ID NO: 538 - DNA, 21 bp, synthetic construct
  source 1..21: mol_type = other DNA
gccatgggaagatggaagcag

SEQ ID NO: 539 - skipped

SEQ ID NO: 540 - DNA, 22 bp, synthetic construct
  source 1..22: mol_type = other DNA
gtgtacctccaggcattgtggc

SEQ ID NO: 541 - skipped

SEQ ID NO: 542 - DNA, 21 bp, synthetic construct
  source 1..21: mol_type = other DNA
gccatcctgctcacctgtctc

SEQ ID NO: 543 - skipped

SEQ ID NO: 544 - DNA, 22 bp, synthetic construct
  source 1..22: mol_type = other DNA
gagacagggtcgggcacctcaa

SEQ ID NO: 545 - skipped

SEQ ID NO: 546 - DNA, 22 bp, synthetic construct
  source 1..22: mol_type = other DNA
gtgagacaaaaaatgcacacaa

SEQ ID NO: 547 - skipped

SEQ ID NO: 548 - DNA, 21 bp, synthetic construct
  source 1..21: mol_type = other DNA
gtgctgtgatttctacttact

SEQ ID NO: 549 - skipped

SEQ ID NO: 550 - DNA, 22 bp, synthetic construct
  source 1..22: mol_type = other DNA
gctgtgatttctacttactttg

SEQ ID NO: 551 - skipped

SEQ ID NO: 552 - DNA, 22 bp, synthetic construct
  source 1..22: mol_type = other DNA
gttcttttatcagctcagaaag

SEQ ID NO: 553 - skipped

SEQ ID NO: 554 - DNA, 22 bp, synthetic construct
  source 1..22: mol_type = other DNA
gtgtttccatgggtttacctga

SEQ ID NO: 555 - skipped

SEQ ID NO: 556 - DNA, 22 bp, synthetic construct
  source 1..22: mol_type = other DNA
gtgggtttacctgattgggtat

SEQ ID NO: 557 - skipped

SEQ ID NO: 558 - DNA, 22 bp, synthetic construct
  source 1..22: mol_type = other DNA
gctctccaggcagtccatggac

SEQ ID NO: 559 - skipped

SEQ ID NO: 560 - DNA, 22 bp, synthetic construct
  source 1..22: mol_type = other DNA
gagatacctgagtaactttctg

SEQ ID NO: 561 - skipped

SEQ ID NO: 562 - DNA, 22 bp, synthetic construct
  source 1..22: mol_type = other DNA
gcacccataccttggagcaacg

SEQ ID NO: 563 - skipped

SEQ ID NO: 564 - DNA, 22 bp, synthetic construct
  source 1..22: mol_type = other DNA
gcacccataccttggagcaacg

SEQ ID NO: 565 - skipped

SEQ ID NO: 566 - DNA, 91 bp, synthetic construct
  source 1..91: mol_type = other DNA
gttttagtactctgtaatgaaaattacagaatctactaaaacaaggcaaaatgccgtgtttatctcgtcaacttgttggcgagattttttt

SEQ ID NO: 567 - DNA, 91 bp, synthetic construct; identical to SEQ ID NO: 566
  source 1..91: mol_type = other DNA

SEQ ID NO: 568 - DNA, 128 bp, synthetic construct
  source 1..128: mol_type = other DNA
gttgtagctccctttctcatttcggaaacgaaatgagaaccgttgctacaataaggccgtctgaaaagatgtgccgcaacgctctgccccttaaagcttctgctttaaggggcatcgtttattttttt

SEQ ID NO: 569 - DNA, 80 bp, synthetic construct; identical to SEQ ID NO: 340
  source 1..80: mol_type = other DNA

SEQ ID NO: 570 - DNA, 83 bp, synthetic construct
  source 1..83: mol_type = other DNA
gttttagtactctggaaacagaatctactaaaacaaggcaaaatgccgtgtttatctcgtcaacttgttggcgagattttttt

SEQ ID NO: 571 - DNA, 91 bp, synthetic construct; identical to SEQ ID NO: 566
  source 1..91: mol_type = other DNA

SEQ ID NO: 572 - DNA, 20 bp, synthetic construct
  source 1..20: mol_type = other DNA
ggcgtccatgtccttcccga

SEQ ID NO: 573 - DNA, 22 bp, synthetic construct
  source 1..22: mol_type = other DNA
acatgtgcggcctcatcagcca

SEQ ID NO: 574 - DNA, 20 bp, synthetic construct
  source 1..20: mol_type = other DNA
gctcagaaaaagggccctga

SEQ ID NO: 575 - DNA, 20 bp, synthetic construct
  source 1..20: mol_type = other DNA
gagattcagtgtggtggggg

SEQ ID NO: 576 - DNA, 20 bp, synthetic construct
  source 1..20: mol_type = other DNA
aactttctatccgtccgcgt

SEQ ID NO: 577 - DNA, 20 bp, synthetic construct
  source 1..20: mol_type = other DNA
ggctgtagagggagacaagc

SEQ ID NO: 578 - DNA, 20 bp, synthetic construct
  source 1..20: mol_type = other DNA
gggcctggaagttcgctaat

SEQ ID NO: 579 - DNA, 20 bp, synthetic construct
  source 1..20: mol_type = other DNA
tggatcgcttttccgagctt

SEQ ID NO: 580 - DNA, 20 bp, synthetic construct
  source 1..20: mol_type = other DNA
attgctcttttctccgccca

SEQ ID NO: 581 - DNA, 20 bp, synthetic construct
  source 1..20: mol_type = other DNA
ttcacaaaacaggggtggct

SEQ ID NO: 582 - DNA, 20 bp, synthetic construct
  source 1..20: mol_type = other DNA
gacgtcttctcctgtggtgg

SEQ ID NO: 583 - DNA, 20 bp, synthetic construct
  source 1..20: mol_type = other DNA
gggtgtctggctggaatctc

SEQ ID NO: 584 - DNA, 18 bp, synthetic construct
  source 1..18: mol_type = other DNA
cccccgcactccttcttc

SEQ ID NO: 585 - DNA, 19 bp, synthetic construct
  source 1..19: mol_type = other DNA
aatctacctccgcggacct

SEQ ID NO: 586 - DNA, 21 bp, synthetic construct
  source 1..21: mol_type = other DNA
ggctccctctcccagttaccg

SEQ ID NO: 587 - DNA, 20 bp, synthetic construct
  source 1..20: mol_type = other DNA
caccaccatccgctctgccc

SEQ ID NO: 588 - DNA, 20 bp, synthetic construct
  source 1..20: mol_type = other DNA
ttcaggctgtgaaccttggt

SEQ ID NO: 589 - DNA, 21 bp, synthetic construct
  source 1..21: mol_type = other DNA
ccgctaccagccgacttttaa

SEQ ID NO: 590 - DNA, 19 bp, synthetic construct
  source 1..19: mol_type = other DNA
ggtggaaggtccctccaga

SEQ ID NO: 591 - DNA, 20 bp, synthetic construct
  source 1..20: mol_type = other DNA
ccttcaacctgacctgggac

SEQ ID NO: 592 - DNA, 20 bp, synthetic construct
  source 1..20: mol_type = other DNA
attgctcttttctccgccca

SEQ ID NO: 593 - DNA, 20 bp, synthetic construct
  source 1..20: mol_type = other DNA
ttcacaaaacaggggtggct

SEQ ID NO: 594 - DNA, 20 bp, synthetic construct
  source 1..20: mol_type = other DNA
aaagaggaagctgtctccgc

SEQ ID NO: 595 - DNA, 20 bp, synthetic construct
  source 1..20: mol_type = other DNA
agtctcagccagccactttc

SEQ ID NO: 596 - DNA, 20 bp, synthetic construct
  source 1..20: mol_type = other DNA
gctctgggcggaggaatatg

SEQ ID NO: 597 - DNA, 20 bp, synthetic construct
  source 1..20: mol_type = other DNA
tccgtgcgtcagttttacct

SEQ ID NO: 598 - DNA, 26 bp, synthetic construct
  source 1..26: mol_type = other DNA
gatctggacgaatagtgtaggttaag

SEQ ID NO: 599 - DNA, 23 bp, synthetic construct
  source 1..23: mol_type = other DNA
gcaagttggttgctgtcttttgg

SEQ ID NO: 600 - DNA, 20 bp, synthetic construct
  source 1..20: mol_type = other DNA
gaacccaggtagccagagac

SEQ ID NO: 601 - DNA, 20 bp, synthetic construct
  source 1..20: mol_type = other DNA
tcctttcaacccgaacggag

SEQ ID NO: 602 - DNA, 20 bp, synthetic construct
  source 1..20: mol_type = other DNA
gctatgcagggtggaggaag

SEQ ID NO: 603 - DNA, 22 bp, synthetic construct
  source 1..22: mol_type = other DNA
gggactagaaaggtgaagagcc

SEQ ID NO: 604 - DNA, 20 bp, synthetic construct
  source 1..20: mol_type = other DNA
gaggatcctgggagggagag

SEQ ID NO: 605 - DNA, 20 bp, synthetic construct
  source 1..20: mol_type = other DNA
gaggatcctgggagggagag

SEQ ID NO: 606 - DNA, 20 bp, synthetic construct
  source 1..20: mol_type = other DNA
ggtcttcgttcctggctgat

SEQ ID NO: 607 - DNA, 20 bp, synthetic construct
  source 1..20: mol_type = other DNA
cttttcccttggaccccagg

SEQ ID NO: 608 - DNA, 20 bp, synthetic construct
  source 1..20: mol_type = other DNA
atcaaggtcaacacaggggg

SEQ ID NO: 609 - DNA, 20 bp, synthetic construct
  source 1..20: mol_type = other DNA
tcaccatgttgaccaggctg

SEQ ID NO: 610 - DNA, 20 bp, synthetic construct
  source 1..20: mol_type = other DNA
ccagccccatctgtcaaact

SEQ ID NO: 611 - DNA, 32 bp, synthetic construct
  source 1..32: mol_type = other DNA
ccgatcttgaatggattccttggaaacaatga

SEQ ID NO: 612 - DNA, 20 bp, synthetic construct
  source 1..20: mol_type = other DNA
atgtgggctgcctagaaagg

SEQ ID NO: 613 - DNA, 20 bp, synthetic construct
  source 1..20: mol_type = other DNA
cccagccaaacttgtcaacc

SEQ ID NO: 614 - DNA, 20 bp, synthetic construct
  source 1..20: mol_type = other DNA
acgtctcatatgccccttgg

SEQ ID NO: 615 - DNA, 22 bp, synthetic construct
  source 1..22: mol_type = other DNA
acgtaggaattttggtgggaca

SEQ ID NO: 616 - DNA, 20 bp, synthetic construct
  source 1..20: mol_type = other DNA
cagctcagcctgagtgttga

SEQ ID NO: 617 - DNA, 26 bp, synthetic construct
  source 1..26: mol_type = other DNA
ccgatctctcgtgggtttgtggttgc

SEQ ID NO: 618 - DNA, 20 bp, synthetic construct
  source 1..20: mol_type = other DNA
cattgcagagaggcgtatca

SEQ ID NO: 619 - DNA, 18 bp, synthetic construct
  source 1..18: mol_type = other DNA
ggggtcccaggtgctgac

SEQ ID NO: 620 - DNA, 20 bp, synthetic construct
  source 1..20: mol_type = other DNA
gaacccaggtagccagagac

SEQ ID NO: 621 - DNA, 20 bp, synthetic construct
  source 1..20: mol_type = other DNA
tcctttcaacccgaacggag

SEQ ID NO: 622 - DNA, 20 bp, synthetic construct
  source 1..20: mol_type = other DNA
gctcagaaaaagggccctga

SEQ ID NO: 623 - DNA, 20 bp, synthetic construct
  source 1..20: mol_type = other DNA
gagattcagtgtggtggggg

SEQ ID NO: 624 - DNA, 20 bp, synthetic construct
  source 1..20: mol_type = other DNA
aactttctatccgtccgcgt

SEQ ID NO: 625 - DNA, 20 bp, synthetic construct
  source 1..20: mol_type = other DNA
ggctgtagagggagacaagc

SEQ ID NO: 626 - DNA, 24 bp, synthetic construct
  source 1..24: mol_type = other DNA
agccctctttttattggaactgtg

SEQ ID NO: 627 - DNA, 20 bp, synthetic construct
  source 1..20: mol_type = other DNA
ccgactggtccacttaccta

SEQ ID NO: 628 - DNA, 22 bp, synthetic construct
  source 1..22: mol_type = other DNA
aacggaactcaaccattaagca

SEQ ID NO: 629 - DNA, 24 bp, synthetic construct
  source 1..24: mol_type = other DNA
ccaacatacagaagtcaggaatgc

SEQ ID NO: 630 - DNA, 19 bp, synthetic construct
  source 1..19: mol_type = other DNA
gcgcacggcctctaggtct

SEQ ID NO: 631 - DNA, 20 bp, synthetic construct
  source 1..20: mol_type = other DNA
gaggaaacagcaccgcaccg

SEQ ID NO: 632 - DNA, 20 bp, synthetic construct
  source 1..20: mol_type = other DNA
gcgtccatgtccttcccgag

SEQ ID NO: 633 - DNA, 20 bp, synthetic construct
  source 1..20: mol_type = other DNA
ccaaccccaaagcaacgccg

SEQ ID NO: 634 - DNA, 20 bp, synthetic construct
  source 1..20: mol_type = other DNA
tcacctgggcagtcacgaaa

SEQ ID NO: 635 - DNA, 24 bp, synthetic construct
  source 1..24: mol_type = other DNA
acaccatcaacatagtgaggagaa

SEQ ID NO: 636 - DNA, 20 bp, synthetic construct
  source 1..20: mol_type = other DNA
ggcgtccatgtccttcccga

SEQ ID NO: 637 - DNA, 22 bp, synthetic construct
  source 1..22: mol_type = other DNA
acatgtgcggcctcatcagcca

SEQ ID NO: 638 - DNA, 22 bp, synthetic construct
  source 1..22: mol_type = other DNA
gatgaggtcatgctggggtcct

SEQ ID NO: 639 - DNA, 25 bp, synthetic construct
  source 1..25: mol_type = other DNA
gtagaaccttgatgacatagccccg

SEQ ID NO: 640 - DNA, 23 bp, synthetic construct
  source 1..23: mol_type = other DNA
aggaactgttctcctcactccca

SEQ ID NO: 641 - DNA, 23 bp, synthetic construct
  source 1..23: mol_type = other DNA
gcctgacagtgagaccttgcttc

SEQ ID NO: 642 - DNA, 23 bp, synthetic construct
  source 1..23: mol_type = other DNA
tgtgggaactggagttggctagt

SEQ ID NO: 643 - DNA, 23 bp, synthetic construct
  source 1..23: mol_type = other DNA
ctggacgctccgatgatgtcctt

SEQ ID NO: 644 - DNA, 20 bp, synthetic construct
  source 1..20: mol_type = other DNA
gaggaaacagcaccgcaccg

SEQ ID NO: 645 - DNA, 20 bp, synthetic construct
  source 1..20: mol_type = other DNA
gaggaaacagcaccgcaccg

SEQ ID NO: 646 - DNA, 25 bp, synthetic construct
  source 1..25: mol_type = other DNA
acacctagggtttgctgggtttctt

SEQ ID NO: 647 - DNA, 23 bp, synthetic construct
  source 1..23: mol_type = other DNA
tatgcagtggcccagccctatca

SEQ ID NO: 648 - DNA, 20 bp, synthetic construct
  source 1..20: mol_type = other DNA
aggctgtggctgtgtttgct

SEQ ID NO: 649 - DNA, 20 bp, synthetic construct
  source 1..20: mol_type = other DNA
ggaaaccaccagcagggagg

SEQ ID NO: 650 - DNA, 22 bp, synthetic construct
  source 1..22: mol_type = other DNA
ccagtgcctgggatgtgctctg

SEQ ID NO: 651 - DNA, 22 bp, synthetic construct
  source 1..22: mol_type = other DNA
caggagtacagctgcaacgctc

SEQ ID NO: 652 - DNA, 19 bp, synthetic construct
  source 1..19: mol_type = other DNA
cctcccaggccaggtccag

SEQ ID NO: 653 - DNA, 20 bp, synthetic construct
  source 1..20: mol_type = other DNA
gccgggccatcaccatcttt

SEQ ID NO: 654 - DNA, 20 bp, synthetic construct
  source 1..20: mol_type = other DNA
cacacagacctcccaagccc

SEQ ID NO: 655 - DNA, 20 bp, synthetic construct
  source 1..20: mol_type = other DNA
gcgtccatgtccttcccgag

SEQ ID NO: 656 - DNA, 20 bp, synthetic construct
  source 1..20: mol_type = other DNA
ccaaccccaaagcaacgccg

SEQ ID NO: 657 - DNA, 22 bp, synthetic construct
  source 1..22: mol_type = other DNA
gattgaacaaactgcccaccgc

SEQ ID NO: 658 - DNA, 22 bp, synthetic construct
  source 1..22: mol_type = other DNA
caagagccacaggcgcttactg

SEQ ID NO: 659 - DNA, 20 bp, synthetic construct
  source 1..20: mol_type = other DNA
ttgggaagaggacgggcaga

SEQ ID NO: 660 - DNA, 20 bp, synthetic construct
  source 1..20: mol_type = other DNA
tggggaggtggacagtcagg

SEQ ID NO: 661 - DNA, 20 bp, synthetic construct
  source 1..20: mol_type = other DNA
ccgtctaatgcgtggggtgg

SEQ ID NO: 662 - DNA, 20 bp, synthetic construct
  source 1..20: mol_type = other DNA
ggcacgctgttgaagtcggt

SEQ ID NO: 663 - DNA, 23 bp, synthetic construct
  source 1..23: mol_type = other DNA
ccctatattttgggaagcagggc

SEQ ID NO: 664 - DNA, 22 bp, synthetic construct
  source 1..22: mol_type = other DNA
agaaaaccttcctgtgaggcca

SEQ ID NO: 665 - DNA, 20 bp, synthetic construct
  source 1..20: mol_type = other DNA
gggtgctccctccaccctaa

SEQ ID NO: 666 - DNA, 20 bp, synthetic construct
  source 1..20: mol_type = other DNA
aggtgctgctctccagccaa

SEQ ID NO: 667 - DNA, 25 bp, synthetic construct
  source 1..25: mol_type = other DNA
ggagggagaagttccaaattgctta

SEQ ID NO: 668 - DNA, 20 bp, synthetic construct
  source 1..20: mol_type = other DNA
cagctgcaggaggccattcg

SEQ ID NO: 669 - DNA, 24 bp, synthetic construct
  source 1..24: mol_type = other DNA
tgacacacttacccagcattccct

SEQ ID NO: 670 - DNA, 25 bp, synthetic construct
  source 1..25: mol_type = other DNA
attttgtagagaaaactgcccggat

SEQ ID NO: 671 - DNA, 19 bp, synthetic construct
  source 1..19: mol_type = other DNA
gccgactgctctgccgttt

SEQ ID NO: 672 - DNA, 25 bp, synthetic construct
  source 1..25: mol_type = other DNA
cccagatttgcgtgaataaccattt

SEQ ID NO: 673 - DNA, 24 bp, synthetic construct
  source 1..24: mol_type = other DNA
gctaaaaccaaactcaaaacgggt

SEQ ID NO: 674 - DNA, 22 bp, synthetic construct
  source 1..22: mol_type = other DNA
cccatgtcagtgcaaccagcta

SEQ ID NO: 675 - DNA, 20 bp, synthetic construct
  source 1..20: mol_type = other DNA
tcacctgggcagtcacgaaa

SEQ ID NO: 676 - DNA, 24 bp, synthetic construct
  source 1..24: mol_type = other DNA
acaccatcaacatagtgaggagaa

SEQ ID NO: 677 - DNA, 20 bp, synthetic construct
  source 1..20: mol_type = other DNA
gcgtccatgtccttcccgag

SEQ ID NO: 678 - DNA, 20 bp, synthetic construct
  source 1..20: mol_type = other DNA
ccaaccccaaagcaacgccg

SEQ ID NO: 679 - DNA, 17 bp, synthetic construct
  source 1..17: mol_type = other DNA
aaccccaccaccttggc

SEQ ID NO: 680 - DNA, 23 bp, synthetic construct
  source 1..23: mol_type = other DNA
acagtccacagtctaagtgcaaa

SEQ ID NO: 681 - DNA, 24 bp, synthetic construct
  source 1..24: mol_type = other DNA
tttggagtggcatatttgacagca

SEQ ID NO: 682 - DNA, 21 bp, synthetic construct
  source 1..21: mol_type = other DNA
tgtggcacagtgggcctttac

SEQ ID NO: 683 - DNA, 21 bp, synthetic construct
  source 1..21: mol_type = other DNA
tcatgctctgccctgtaggta

SEQ ID NO: 684 - DNA, 25 bp, synthetic construct
  source 1..25: mol_type = other DNA
atggcattcatttaagcctaacttc

SEQ ID NO: 685 - DNA, 24 bp, synthetic construct
  source 1..24: mol_type = other DNA
aagtatgttgggacccttggctgg

SEQ ID NO: 686 - DNA, 24 bp, synthetic construct
  source 1..24: mol_type = other DNA
tggcctgttctactgactatgggg

SEQ ID NO: 687 - DNA, 23 bp, synthetic construct
  source 1..23: mol_type = other DNA
gacagacacagggaagccttggg

SEQ ID NO: 688 - DNA, 25 bp, synthetic construct
  source 1..25: mol_type = other DNA
aaccttccaggagagagaaacctgt

SEQ ID NO: 689 - DNA, 24 bp, synthetic construct
  source 1..24: mol_type = other DNA
ttcaagcaatcacgagacactcag

SEQ ID NO: 690 - DNA, 23 bp, synthetic construct
  source 1..23: mol_type = other DNA
cccaccacccagcagctttattg

SEQ ID NO: 691 - DNA, 22 bp, synthetic construct
  source 1..22: mol_type = other DNA
tctcaggcgacctggtttctgc

SEQ ID NO: 692 - DNA, 21 bp, synthetic construct
  source 1..21: mol_type = other DNA
tctgccagatgcgtccgatca

SEQ ID NO: 693 - DNA, 22 bp, synthetic construct
  source 1..22: mol_type = other DNA
gccagccctgcctggaagttag

SEQ ID NO: 694 - DNA, 22 bp, synthetic construct
  source 1..22: mol_type = other DNA
tgacctccgggattctcagccc

SEQ ID NO: 695 - DNA, 24 bp, synthetic construct
  source 1..24: mol_type = other DNA
gcttcctgtctgcaattggggtct

SEQ ID NO: 696 - DNA, 21 bp, synthetic construct
  source 1..21: mol_type = other DNA
agtaggttgcggggctcagga

SEQ ID NO: 697 - DNA, 23 bp, synthetic construct
  source 1..23: mol_type = other DNA
aacctccacgggggtatctgagg

SEQ ID NO: 698 - DNA, 21 bp, synthetic construct
  source 1..21: mol_type = other DNA
acctggcaagtggggtactgg

SEQ ID NO: 699 - DNA, 24 bp, synthetic construct
  source 1..24: mol_type = other DNA
gtctaaatgggcaagcaatcccct

SEQ ID NO: 700 - DNA, 23 bp, synthetic construct
  source 1..23: mol_type = other DNA
ccaggatcccacagggtccttct

SEQ ID NO: 701 - DNA, 23 bp, synthetic construct
  source 1..23: mol_type = other DNA
tccccagagcccagggaatatca

SEQ ID NO: 702 - DNA, 23 bp, synthetic construct
  source 1..23: mol_type = other DNA
tgttgctccgatggaaggatggg

SEQ ID NO: 703 - DNA, 20 bp, synthetic construct
  source 1..20: mol_type = other DNA
ccaccagaagcgccccagaa

SEQ ID NO: 704 - DNA, 20 bp, synthetic construct
  source 1..20: mol_type = other DNA
ggggaatcgcctccactgcc

SEQ ID NO: 705 - DNA, 23 bp, synthetic construct
  source 1..23: mol_type = other DNA
gttcttattggccagggagcctt

SEQ ID NO: 706 - DNA, 23 bp, synthetic construct
  source 1..23: mol_type = other DNA
ctccccaagtgacaggaaccacg

SEQ ID NO: 707 - DNA, 25 bp, synthetic construct
  source 1..25: mol_type = other DNA
gggtctggaatatactcaggttcgt

SEQ ID NO: 708 - DNA, 23 bp, synthetic construct
  source 1..23: mol_type = other DNA
tgaccagtctgttgtgaacagtg

SEQ ID NO: 709 - DNA, 23 bp, synthetic construct
  source 1..23: mol_type = other DNA
tgacctgatggagttggacatgg

SEQ ID NO: 710 - DNA, 22 bp, synthetic construct
  source 1..22: mol_type = other DNA
acttgctcttgcgtgaaggact

SEQ ID NO: 711 - DNA, 21 bp, synthetic construct
  source 1..21: mol_type = other DNA
acaggtcttccacagaagggc

SEQ ID NO: 712 - DNA, 24 bp, synthetic construct
  source 1..24: mol_type = other DNA
acttgtttacaagaggtgaccagt

SEQ ID NO: 713 - DNA, 21 bp, synthetic construct
  source 1..21: mol_type = other DNA
agccagaatcctccctggcaa

SEQ ID NO: 714 - DNA, 23 bp, synthetic construct
  source 1..23: mol_type = other DNA
cagaacaccatccgactccctgt

SEQ ID NO: 715-718 - skipped (4 entries)

SEQ ID NO: 719 - DNA, 121 bp, synthetic construct
  source 1..121: mol_type = other DNA
gttgtagctccctttctcatttcggaaacgaaatgagaaccgttgctacaataaggccgtctgaaaagatgtgccgcaacgctctgccccttaaagcttctgctttaaggggcatcgttta

SEQ ID NO: 720 - DNA, 24 bp, synthetic construct
  source 1..24: mol_type = other DNA
gaccagcccctcgaaggcaaggcc

SEQ ID NO: 721 - DNA, 24 bp, synthetic construct
  source 1..24: mol_type = other DNA
gctggccgccagcccagttgtagc

SEQ ID NO: 722 - DNA, 22 bp, synthetic construct
  source 1..22: mol_type = other DNA
gatggaatcccttctgcagcac

SEQ ID NO: 723-732 - skipped (10 entries)

SEQ ID NO: 733 - DNA, 21 bp, synthetic construct
  source 1..21: mol_type = other DNA
ccagccccagcaagagcacaa

SEQ ID NO: 734 - DNA, 21 bp, synthetic construct
  source 1..21: mol_type = other DNA
cctcatctccaatatggtatg

SEQ ID NO: 735 - DNA, 21 bp, synthetic construct
  source 1..21: mol_type = other DNA
ggacaaaccagaagccgctcc

SEQ ID NO: 736 - DNA, 20 bp, synthetic construct
  source 1..20: mol_type = other DNA
tggacttcggcttttgtccc

SEQ ID NO: 737 - DNA, 22 bp, synthetic construct
  source 1..22: mol_type = other DNA
gggactagaaaggtgaagagcc

SEQ ID NO: 738 - DNA, 21 bp, synthetic construct
  source 1..21: mol_type = other DNA
tccagccacctgctgatttgt